(19) United States
(12) Patent Application Publication
(10) Pub. No.: US 2024/0216515 A1
(43) Pub. Date: Jul. 4, 2024

(54) COMPOSITIONS AND METHODS FOR TARGETED DELIVERY TO CELLS

(71) Applicants: ReCode Therapeutics, Inc., Menlo Park, CA (US); The Board of Regents of the University of Texas System, Austin, TX (US)

(72) Inventors: Michael Torres, Menlo Park, CA (US); Daniel J. Siegwart, Dallas, TX (US); Jackson Eby, Menlo Park, CA (US); Rumpa Bhattacherjee, Menlo Park, CA (US); Brandon Wustman, Menlo Park, CA (US); Vladimir Kharitonov, Menlo Park, CA (US); Qiang Cheng, Dallas, TX (US); Tuo Wei, Dallas, TX (US)

(21) Appl. No.: 18/283,406

(22) PCT Filed: Mar. 21, 2022
(86) PCT No.: PCT/US2022/021176

§ 371 Date: Sep. 21, 2023

Related U.S. Application Data
(60) Provisional application No. 63/164,526, filed on Mar. 22, 2021.

(51) Int. Cl. A61K 47/54; A61K 47/14; A61K 47/24; A61K 47/28; C12N 15/113

(52) U.S. Cl. CPC A61K 47/543; A61K 47/14; A61K 47/24; A61K 47/28; A61K 47/545; C12N 15/113; C12N 2310/11; C12N 2310/14; C12N 2310/141; C12N 2310/531

(57) ABSTRACT

Described herein are compositions, kits, and methods for potent delivery to a ceil of a subject. The cell can be of a particular cell type, such as a basal cell. In some cases, the cell can be a lung cell of a particular cell type. Also described herein are pharmaceutical compositions comprising a therapeutic or prophylactic agent assembled with a lipid composition. Tire lipid composition can comprise an ionizable cationic lipid, and a selective organ targeting lipid. The lipid composition can further comprise a phospholipid. Further described herein are high-potency intravenous dosage forms of a therapeutic or prophylactic agent formulated with a lipid composition.

### CROSS-REFERENCE

This application claims the benefit of U.S. Provisional Application No. 63/164,526, filed on Mar. 22, 2021, which is entirely incorporated herein by reference for all purposes.

### BACKGROUND

The CRISPR/Cas (clustered regularly interspaced short palindromic repeat/CRISPR-associated protein (Cas)) technology can edit the genome in a precise, sequence dependent manner, resulting in a permanent change. Because of the ability to target disease causing mutations, it holds incredible promise for one-time cures of genetic diseases and many other applications in diverse fields. To date, successful editing has been mediated mainly by viral vectors, which require laborious customization for every target and present challenges for clinical translation due to immunogenicity, generation of antibodies that prevent repeat administration, and concerns about rare but dangerous integration events. There remains a clear need to accomplish CRISPR/Cas editing via synthetic nanoparticles (NPs) to expand the safe and effective applications of gene editing.

CRISPR/Cas editing using viruses, membrane deformation, and hydrodynamic injection are functional, but have limitations that could hinder in vivo therapeutic use in the clinic, including persistent expression of Cas9 and off target editing. Furthermore, these delivery systems generally are not selective for the specific cells or organs in which editing is needed. For example, most lipid nanoparticles accumulate through the biological processes in the liver thus reducing the efficacy of the composition on delivery into the target cell(s) or organ.

Similarly, other therapeutic agents such as proteins and small molecules could benefit from targeted delivery. There remains a need for improving safety, potency, and targeting efficacy of lipid delivery systems.

### SUMMARY

The present application generally concerns safe, efficacious, and potent delivery of a therapeutic or prophylactic agent such as a polynucleotide, a polypeptide, or a small molecule compound in lipid nanoparticles to target cell(s).

In certain aspects, the present application provides a method for potent delivery to a (e.g., non-liver, such as lung) cell of a subject, comprising: intravenously administering to the subject a composition comprising a therapeutic agent assembled with a lipid composition which comprises: (i) an ionizable cationic lipid; and (ii) a selective organ targeting (SORT) lipid separate from the ionizable cationic lipid wherein (e.g., an amount of) the SORT lipid effects delivery of the therapeutic agent to the cell ofthe subject characterized by a (e.g., about 1.1- or 10-fold) greater therapeutic effect (e.g., a greater amount or activity of said therapeutic agent) in said cell compared to that achieved with a reference lipid composition (e.g., without the amount of the SORT lipid). In some embodiments, the lipid composition further comprises a phospholipid. In some embodiments, the cell is a lung basal cell. In some embodiments, the reference lipid composition comprises 13,16,20-tris(2-hydroxydodecyl)-13,16,20,23-tetraazapentatricontane-11,25-diol (“LF92”). In some embodiments, the reference lipid composition comprises or essentially consists of 13,16,20-tris(2-hydroxydodecyl)-13,16,20,23-tetraazapentatricontane-11,25-diol (“LF92”), a phospholipid, cholesterol, and a PEG-lipid.

In certain aspect, the present application provides a method for potent delivery to (e.g., non-liver, such as lung) cells of a subject, comprising: intravenously administering to the subject a composition comprising a therapeutic agent assembled with a lipid composition which comprises: (i) an ionizable cationic lipid; and (ii) a selective organ targeting (SORT) lipid separate from the ionizable cationic lipid, wherein (e.g., an amount of) the SORT lipid effects delivery of the therapeutic agent to cells of the subject characterized by a therapeutic effect (e.g., an effective amount or activity of said therapeutic agent) in a (e.g., about 1.1- or 10-fold) greater plurality or proportion of said (e.g., non-liver, such as lung) cells compared to that achieved with a reference lipid composition (e.g., without the amount of the SORT lipid). In some embodiments, the lipid composition further comprises a phospholipid. In some embodiments, the cells are non-liver basal cells. In some embodiments, the cells are lung basal cells. In some embodiments, the cell is a lung basal cell. In some embodiments, the reference lipid composition comprises 13,16,20-tris(2-hydroxydodecyl)-13,16,20,23-tetraazapentatricontane-11,25-diol (“LF92”). In some embodiments, the reference lipid composition comprises or essentially consists of 13,16,20-tris(2-hydroxydodecyl)-13,16,20,23-tetraazapentatricontane-1,25-diol (“LF92”), a phospholipid, cholesterol, and a PEG-lipid.

In certain aspect, the present application provides a method for targeted delivery to (e.g., lung) cells of a subject, comprising: intravenously administering to the subject a composition comprising a therapeutic agent assembled with a lipid composition which comprises: (i) an ionizable cationic lipid; and (ii) a selective organ targeting (SORT) lipid separate from the ionizable cationic lipid, wherein (e.g., an amount of) the SORT lipid effects delivery of the therapeutic agent to a greater proportion of cell types as compared to that achieved with a reference lipid composition. In some embodiments, the lipid composition further comprises a phospholipid. In some embodiments, the greater proportion of cell types comprises (e.g., lung) basal cells. In some embodiments, the greater proportion of cell types comprises (e.g., lung) basal cells, (e.g., lung) epithelial cells, (e.g., lung) ciliated cells, (e.g., lung) club cells, or (e.g., lung) goblet cells, or a combination thereof. In some embodiments, the reference lipid composition comprises 13,16,20-tris(2-hydroxydodecyl)-13,16,20,23-tetraazapentatricontane-11,25-diol (“LF92”). In some embodiments, the reference lipid composition comprises or essentially consists of 13,16,20-tris(2-hydroxydodecyl)-13,16,20,23-tetraazapentatricontane-11,25-diol (“LF92”), a phospholipid, cholesterol, and a PEG-lipid.

In certain aspect, the present application provides a method for targeted delivery to (e.g., lung) cells of a subject, comprising: intravenously administering to the subject a composition comprising a therapeutic agent assembled with a lipid composition which comprises: (i) an ionizable cationic lipid; and (ii) a selective organ targeting (SORT) lipid separate from the ionizable cationic lipid, wherein (e.g., an amount of) the SORT lipid effects delivery of the therapeutic agent to cells of the subject characterized by a therapeutic effect (e.g., an effective amount or activity of said therapeutic agent) in a first plurality or proportion of (e.g., lung) cells of a first cell type and in a (e.g., about 1.1- or 10-fold) greater second plurality or proportion of (e.g., lung) cells of a second cell type. In some embodiments, the lipid composition further comprises a phospholipid. In some embodiments, the first cell type is different from the second cell type. In some embodiments, the second cell type is a (e.g., lung) basal cell. In some embodiments, the first cell type is a non-basal cell. In some embodiments, the first cell type is a (e.g., lung) epithelial cell, a (e.g., lung) ciliated cell, a (e.g., lung) club cell, or a (e.g., lung) goblet cell.

In certain aspect, the present application provides a method for targeted delivery to (e.g., lung) cells of a subject, comprising: intravenously administering to the subject a composition comprising a therapeutic agent assembled with a lipid composition which comprises: (i) an ionizable cationic lipid; (; and (ii) a selective organ targeting (SORT) lipid separate from the ionizable cationic lipid, wherein (e.g., an amount of) the SORT lipid effects a delivery of the therapeutic agent to cells of the subject characterized by a (e.g., about 1.1- or 10-fold) greater therapeutic effect (e.g., a (e.g., about 1.1- or 10-fold) greater amount or activity of said therapeutic agent) in a first (e.g., lung) cell of a first cell type of the subject compared to that in a second (e.g., lung) cell of a second cell type of the subject, wherein the first cell type is different from the second cell type. In some embodiments, the second cell type is a (e.g., lung) basal cell. In some embodiments, the first cell type is a non-basal cell. In some embodiments, the first cell type is a (e.g., lung) epithelial cell, a (e.g., lung) ciliated cell, a (e.g., lung) club cell, or a (e.g., lung) goblet cell. In some embodiments, the lipid composition further comprises a phospholipid.

In certain aspect, the present application provides a method for delivery to (e.g., lung) basal cells of a subject, comprising: intravenously administering to the subject a therapeutic agent assembled with a lipid composition that comprises: (i) an ionizable cationic lipid; and (ii) a selective organ targeting (SORT) lipid separate from the ionizable cationic lipid, thereby delivering the therapeutic agent to an organ or tissue (e.g., lung) of the subject to result in a therapeutic effect (e.g., an effective amount or activity of said therapeutic agent) detectable in at least about 5%. 10%, or 15% basal cells in the organ or tissue of the subject. In some embodiments, the lipid composition further comprises a phospholipid.

In certain aspect, the present application provides a high-potency intravenous dosage form of a therapeutic agent formulated with a selective organ targeting (SORT) lipid, the dosage form comprising: the therapeutic agent assembled with a lipid composition that comprises: (i) an ionizable cationic lipid and (ii) the SORT lipid separate from the ionizable cationic lipid, wherein the SORT lipid is present in the dosage form in an amount sufficient to achieve a therapeutic effect at a dose of the therapeutic agent (e.g., at least about 1.1- or 10-fold) lower than that required with a reference lipid composition. In some embodiments, the lipid composition further comprises a phospholipid. In some embodiments, the cell is a lung basal cell. In some embodiments, the reference lipid composition comprises 13,16,20-tris(2-hydroxydodecyl)-13,16,20,23-tetraazapentatricontane-11,25-diol (“LF92”). In some embodiments, the reference lipid composition comprises or essentially consists of 13,16,20-tris(2-hydroxydodecyl)-13,16,20,23-tetraazapentatricontane-11,25-diol (“LF92”), a phospholipid, cholesterol, and a PEG-lipid.

In certain aspect, the present application provides a high-potency intravenous dosage form of a therapeutic agent formulated with a selective organ targeting (SORT) lipid, the dosage form comprising: the therapeutic agent assembled with a lipid composition that comprises: (i) an ionizable cationic lipid; and (ii) the SORT lipid separate from the ionizable cationic lipid, wherein the therapeutic agent (e.g., heterologous polynucleotide) is present in the dosage form at a dose of no more than about 2 milligram per kilogram (mg/kg, or mpk) body weight. In some embodiments, the lipid composition further comprises a phospholipid.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows example structures of SORT lipids.

FIG. 2 shows example structures of dendrimers or dendrons of the ionizable cationic lipids.

FIGS. 3A-3C. FIG. 3A shows a schematic of gene editing through the delivery of SORT LNPs.

FIG. 3B shows a schematic of the timeline for a mouse study. FIG. 3C shows relative expression of TdTom in basal cells after delivery of mRNA by SORT LNPs.

FIG. 4 shows a chart of tissue specific radiance overtime in a mouse of an LNP composition (“lung-SORT”; 5A-SC8 DOTAP).

FIG. 5 shows images of tissue specific radiance over time in a mouse of an LNP composition (“lung-SORT”; 5A-SC8 DOTAP).

FIG. 6 shows images of tissue specific radiance of a dog and a Cynomolgus macaque of an LNP composition

### DETAILED DESCRIPTION

Before the embodiments of the disclosure are described, it is to be understood that such embodiments are provided by way of example only, and that various alternatives to the embodiments of the disclosure described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

In the context of the present application, the following terms have the meanings ascribed to them unless specified otherwise:

As used throughout the specification and claims, the terms “a”, “an” and “the” are generally used in the sense that they mean “at least one”, “at least a first”, “one or more” or “a plurality” of the referenced components or steps, except in instances wherein an upper limit is thereafter specifically stated. For example, a “cleavage sequence”, as used herein, means “at least a first cleavage sequence” but includes a plurality of cleavage sequences. The operable limits and parameters of combinations, as with the amounts of any single agent, will be known to those of ordinary skill in the art in light of the present application.

The terms “polypeptide”, “peptide”, and “protein” are used interchangeably herein to generally refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein in the context of the structure of a polypeptide, “N-terminus” (or “amino terminus”) and “C-terminus” (or “carboxyl terminus”) generally refer to the extreme amino and carboxyl ends of the polypeptide, respectively.

The term “N′-terminal end sequence,” as used herein with respect to a polypeptide or polynucleotide sequence of interest, generally means that no other amino acid or nucleotide residues precede the N-terminal end sequence in the polypeptide or polynucleotide sequence of interest at the N-terminal end. The term “C-terminal end sequence,” as used herein with respect to a polypeptide or polynucleotide sequence of interest, generally means that no other amino acid or nucleotide residues follows the C-terminal end sequence in the polypeptide or polynucleotide sequence of interest at the C-terminal end.

The terms “non-naturally occurring” and “non-natural” are used interchangeably herein. The term “non-naturally occurring” or “non-natural,” as used herein with respect to a therapeutic agent or prophylactic agent, generally means that the agent is not biologically derived in mammals (including but not limited to human). The term “non-naturally occurring” or “non-natural,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

“Physiological conditions” refers to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers are listed in Sambrook et al. (2001). Physiologically relevant temperature ranges from about 25° C. to about 38° C., and preferably from about 35° C. to about 37° C.

As used herein, the terms “treatment” or “treating,” or “palliating” or “ameliorating” are used interchangeably herein. These terms generally refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms or improvement in one or more clinical parameters associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect” or “therapeutic benefit,” as used herein, generally refers to a physiologic effect, including but not limited to the mitigation, amelioration, or prevention of disease or an improvement in one or more clinical parameters associated with the underlying disorder in humans or other animals, or to otherwise enhance physical or mental wellbeing of humans or animals, resulting from administration of a polypeptide of the disclosure other than the ability to induce the production of an antibody against an antigenic epitope possessed by the biologically active protein. For prophylactic benefit, the compositions may be administered to a subject at risk of developing a particular disease, a recurrence of a former disease, condition or symptom of the disease, or to a subject reporting one or more of the physiological symptoms of a disease, even though a diagnosis of this disease may not have been made.

The terms “therapeutically effective amount” and “therapeutically effective dose”, as used herein, generally refer to an amount of a drug or a biologically active protein, either alone or as a part of a polypeptide composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

The term “equivalent molar dose” generally means that the amounts of materials administered to a subject have an equivalent amount of moles, based on the molecular weight of the material used in the dose.

The term “therapeutically effective and non-toxic dose,” as used herein, generally refers to a tolerable dose of the compositions as defined herein that is high enough to cause depletion of tumor or cancer cells, tumor elimination, tumor shrinkage or stabilization of disease without or essentially without major toxic effects in the subject. Such therapeutically effective and non-toxic doses may be determined by dose escalation studies described in the art and should be below the dose inducing severe adverse side effects.

The terms “cancer” and “cancerous” refer to or describe the physiological condition in mammals that is typically characterized by unregulated cell growth/proliferation.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —Ni; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

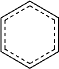

includes

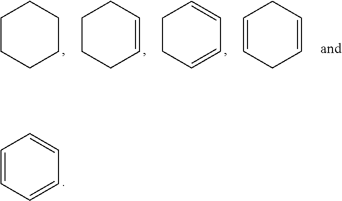

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

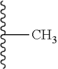

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

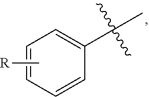

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

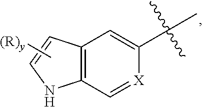

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃(Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN. —SH, —OCH₃, —OCH₂CH₃, —C(O)CH₃, —NHCH₃, —NHCH₂CH₃, —N(CH₃), —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, the carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

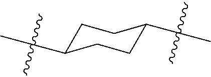 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃. —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂—C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, the carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, the carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

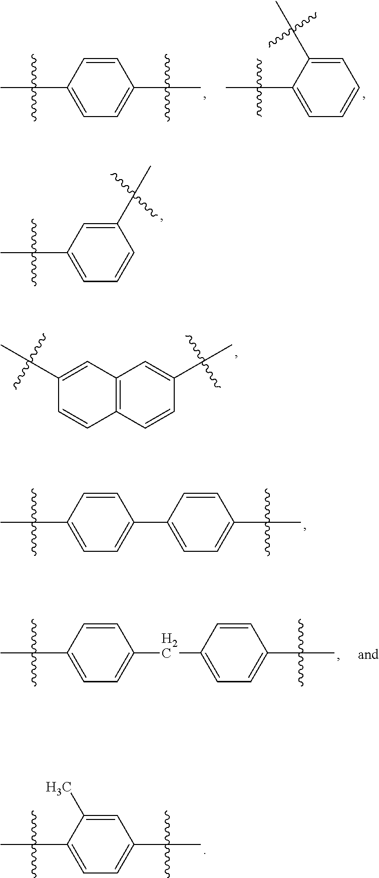

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂. —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

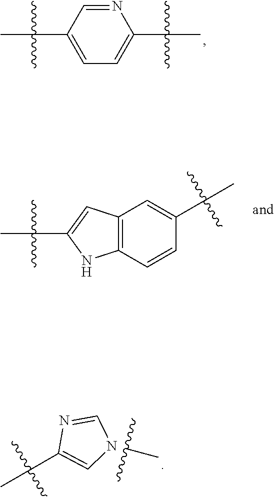

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH—, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, the carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, the atoms forming part of one or more ring structure(s) wherein at least one of the ring atoms is nitrogen, oxygen or sulfur and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

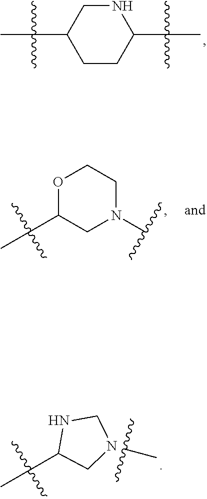

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy” “aralkoxy”, “heteroaryloxy”, “heterocycloalkoxy”, and “acyloxy”, when used without the “substituted” modifier, refers to groups, defined as —OR, in which R is cycloalkyl, alkenyl, alkynl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —COCH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃.

The term “dialkylamino” when used without the “substituted” modifier refers to the group —NRR′ in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NIH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

Throughout this application, the term “about” is used to indicate that a value includes the inherent variation of error for the device, the method being employed to determine the value, or the variation that exists among the study subjects. Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about,” as used herein when referring to a measurable value such as an amount of weight, time, dose, etc. is meant to encompass in one example variations of ±20% or ±10%, in another example ±5%, in another example ±3%, in another example ±1%, and in yet another example ±0.1% from the specified amount, as such variations are appropriate to perform the disclosed method.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate (e.g., non-human primate). In certain embodiments, the patient or subject is a human. Non-limiting examples of human subjects are adults, juveniles, infants and fetuses.

The term “assemble” or “assembled,” as used herein, in context of delivery of a payload to target cell(s) generally refers to covalent or non-covalent interaction(s) or association(s), for example, such that a therapeutic or prophylactic agent be complexed with or encapsulated in a lipid composition.

As used herein, the term “lipid composition” generally refers to a composition comprising lipid compound(s), including but not limited to, a lipoplex, a liposome, a lipid particle. Example of lipid compositions include suspensions, emulsions, and vesicular compositions.

As used herein, the term “detectable” refers to an occurrence of, or a change in, a signal that is directly or indirectly detectable either by observation or by instrumentation. Typically, a detectable response is an occurrence of a signal wherein the fluorophore is inherently fluorescent and does not produce a change in signal upon binding to a metal ion or biological compound. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density

The term “potent” or “potency,” as used herein in connection with delivery of therapeutic agent(s), generally refers to a greater ability of a delivery system (e.g., a lipid composition) to achieve or bring about a desired amount, activity, or effect of a therapeutic agent or prophylactic agent (such as a desired level of translation, transcription, production, expression, or activity of a protein or gene) in cells (e.g., targeted cells) to any measurable extent, e.g., relative to a reference delivery system. For example, a lipid composition with a higher potency may achieve a desired therapeutic effect in a greater population of relevant cells, within a shorter response time, or that last a longer period of time.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present application which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta. 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not vet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer or dendron, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present application.

### Compositions

### Lipid Compositions

In one aspect, provided herein is a lipid composition comprising: (i) an ionizable cationic lipid; and (iii) a selective organ targeting (SORT) lipid separate from the ionizable cationic lipid. The lipid composition may further comprise a phospholipid.

### Ionizable Cationic Lipids

In some embodiments of the lipid composition of the present application, the lipid composition comprises an ionizable cationic lipid. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some embodiments of the lipid composition of the present application, the ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C₆-C₂₄ alkyl or alkenyl groups, but may have at least 1 or more that 6 tails. In some embodiments, these cationic ionizable lipids are dendrimers, which are a polymer exhibiting regular dendritic branching, formed by the sequential or generational addition of branched layers to or from a core and are characterized by a core, at least one interior branched layer, and a surface branched layer. (See Petar R. Dvornic and Donald A. Tomalia in Chem. in Britain. 641-645. August 1994.) In other embodiments, the term “dendrimer” as used herein is intended to include, but is not limited to, a molecular architecture with an interior core, interior layers (or “generations”) of repeating units regularly attached to this initiator core, and an exterior surface of terminal groups attached to the outermost generation. A “dendron” is a species of dendrimer having branches emanating from a focal point which is or can be joined to a core, either directly or through a linking moiety to form a larger dendrimer. In some embodiments, the dendrimer structures have radiating repeating groups from a central core which doubles with each repeating unit for each branch. In some embodiments, the dendrimers described herein may be described as a small molecule, medium-sized molecules, lipids, or lipid-like material. These terms may be used to described compounds described herein which have a dendron like appearance (e.g. molecules which radiate from a single focal point).

While dendrimers are polymers, dendrimers may be preferable to traditional polymers because they have a controllable structure, a single molecular weight, numerous and controllable surface functionalities, and traditionally adopt a globular conformation after reaching a specific generation. Dendrimers can be prepared by sequentially reactions of each repeating unit to produce monodisperse, tree-like and/or generational structure polymeric structures. Individual dendrimers consist of a central core molecule, with a dendritic wedge attached to one or more functional sites on that central core. The dendrimeric surface layer can have a variety of functional groups disposed thereon including anionic, cationic, hydrophilic, or lipophilic groups, according to the assembly monomers used during the preparation.

Modifying the functional groups and/or the chemical properties of the core, repeating units, and the surface or terminating groups, their physical properties can be modulated. Some properties which can be varied include, but are not limited to, solubility, toxicity, immunogenicity and bioattachment capability. Dendrimers are often described by their generation or number of repeating units in the branches. A dendrimer consisting of only the core molecule is referred to as Generation 0, while each consecutive repeating unit along all branches is Generation 1, Generation 2, and so on until the terminating or surface group. In some embodiments, half generations are possible resulting from only the first condensation reaction with the amine and not the second condensation reaction with the thiol.

Preparation of dendrimers requires a level of synthetic control achieved through series of stepwise reactions comprising building the dendrimer by each consecutive group. Dendrimer synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. Thus, in some embodiments, the dendrimers of G1-G10 generation are specifically contemplated. In some embodiments, the dendrimers comprise 1, 2, 3, 4, 5, 6, 7, 8, 9, or 10 repeating units, or any range derivable therein. In some embodiments, the dendrimers used herein are G0, G1, G2, or G3. However, the number of possible generations (such as 11, 12, 13, 14, 15, 20, or 25) may be increased by reducing the spacing units in the branching polymer.

Additionally, dendrimers have two major chemical environments: the environment created by the specific surface groups on the termination generation and the interior of the dendritic structure which due to the higher order structure can be shielded from the bulk media and the surface groups. Because of these different chemical environments, dendrimers have found numerous different potential uses including in therapeutic applications.

In some embodiments of the lipid composition of the present application, the dendrimers or dendrons are assembled using the differential reactivity of the acrylate and methacrylate groups with amines and thiols. The dendrimers or dendrons may include secondary or tertiary amines and thioethers formed by the reaction of an acrylate group with a primary or secondary amine and a methacrylate with a mercapto group. Additionally, the repeating units of the dendrimers or dendrons may contain groups which are degradable under physiological conditions. In some embodiments, these repeating units may contain one or more germinal diethers, esters, amides, or disulfides groups. In some embodiments, the core molecule is a monoamine which allows dendritic polymerization in only one direction. In other embodiments, the core molecule is a polyamine with multiple different dendritic branches which each may comprise one or more repeating units. The dendrimer or dendron may be formed by removing one or more hydrogen atoms from this core. In some embodiments, these hydrogen atoms are on a heteroatom such as a nitrogen atom. In some embodiments, the terminating group is a lipophilic groups such as a long chain alkyl or alkenyl group. In other embodiments, the terminating group is a long chain haloalkyl or haloalkenyl group. In other embodiments, the terminating group is an aliphatic or aromatic group containing an ionizable group such as an amine (—NH₂) or a carboxylic acid (—CO₂H). In still other embodiments, the terminating group is an aliphatic or aromatic group containing one or more hydrogen bond donors such as a hydroxide group, an amide group, or an ester.

The cationic ionizable lipids of the present application may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Cationic ionizable lipids may occur as racemates and racemic mixtures, single enantiomers, diastereomeric mixtures and individual diastereomers. In some embodiments, a single diastereomer is obtained. The chiral centers of the cationic ionizable lipids of the present application can have the S or the R configuration. Furthermore, it is contemplated that one or more of the cationic ionizable lipids may be present as constitutional isomers. In some embodiments, the compounds have the same formula but different connectivity to the nitrogen atoms of the core. Without wishing to be bound by any theory, it is believed that such cationic ionizable lipids exist because the starting monomers react first with the primary amines and then statistically with any secondary amines present. Thus, the constitutional isomers may present the fully reacted primary amines and then a mixture of reacted secondary amines.

Chemical formulas used to represent cationic ionizable lipids of the present application will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given formula, and regardless of which one is most prevalent, all tautomers of a given chemical formula are intended.

The cationic ionizable lipids of the present application may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the cationic ionizable lipids of the present application are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a cationic ionizable lipids provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

In some embodiments of the lipid composition of the present application, the ionizable cationic lipid is a dendrimer or dendron. In some embodiments, the ionizable cationic lipid comprises an ammonium group which is positively charged at physiological pH and contains at least two hydrophobic groups. In some embodiments, the ammonium group is positively charged at a pH from about 6 to about 8. In some embodiments, the ionizable cationic lipid is a dendrimer or dendron. In some embodiments, the ionizable cationic lipid comprises at least two C₆-C₂₄ alkyl or alkenyl groups.

### Dendrimers of Formula (I)

In some embodiments of the lipid composition, the ionizable cationic lipid comprises at least two C₈-C₂₄ alkyl groups. In some embodiments, the ionizable cationic lipid is a dendrimer or dendron further defined by the formula:

### Core-Repeating Unit-Terminating Group (D-I)

wherein the core is linked to the repeating unit by removing one or more hydrogen atoms from the core and replacing the atom with the repeating unit and wherein:
   the core has the formula:

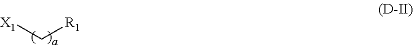

wherein:
   X₁ is amino or alkylamino_{(C≤12)}, dialkylamino_{(C≤12)}, heterocycloalkyl_{(C≤12)}, heteroaryl_{(C≤12)}, or a substituted version thereof:
   R₁ is amino, hydroxy, or mercapto, or alkylamino_{(C≤12)}, dialkylamino_{(C≤12)}, or a substituted version of either of these groups; and
   a is 1, 2, 3, 4, 5, or 6; or
the core has the formula:

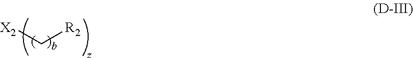

wherein:
   X₂ is N(R₅)_{y};
   R₅ is hydrogen, alkyl_{(C≤18)}, or substituted alkyl_{(C≤18)}; and
   y is 0, 1, or 2, provided that the sum of y and z is 3;
   R₂ is amino, hydroxy, or mercapto, or alkylamino_{(C≤12)}, dialkylamino_{(C≤12)}, or a substituted version of either of these groups;
   b is 1, 2, 3, 4, 5, or 6; and
   z is 1, 2, 3; provided that the sum of z and y is 3; or
the core has the formula:

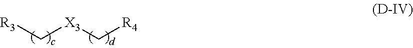

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

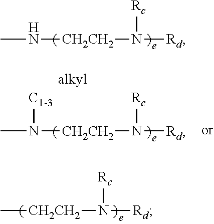

wherein:
e and f are each independently 1, 2, or 3; provided that the sum of e and f is 3;
R_{c}, R_{d}, and R_{f} are each independently hydrogen, alkyl_{(C≤6)}, or substituted alkyl_{(C≤6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker;
   the degradable diacyl group has the formula:

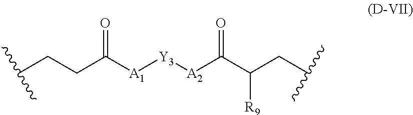

wherein:
   A₁ and A₂ are each independently —O—, —S—, or —NRₐ—, wherein:
   Rₐ is hydrogen, alkyl_{(C≤6)}, or substituted alkyl_{(C≤6)};
   Y₃ is alkanediyl_{(C≤12)}, alkenediyl_{(C≤12)}, arenediyl_{(C≤12)}, or a substituted version of any of these groups; or a group of the formula:

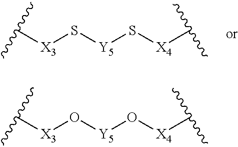

wherein:
X₃ and X₄ are alkanediyl_{(C≤12)}, alkenediyl_{(C≤12)}, arenediyl_{(C≤12)}, or a substituted version of any of these groups;
Y₅ is a covalent bond, alkanediyl_{(C≤12)}, alkenediyl_{(C≤12)}, arenediyl_{(C≤12)}, or a substituted version of any of these groups; and
R₉ is alkyl_{(C≤8)} or substituted alkyl_{(C≤8)};
the linker group has the formula:

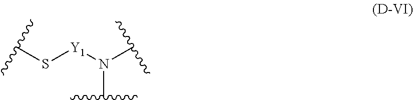

wherein:
   Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula:

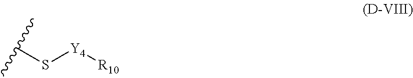

wherein:
   Y₄ is alkanediyl_{(C≤18)} or an alkanediyl_{(C≤18)} wherein one or more of the hydrogen atoms on the alkanediyl_{(C≤18)} has been replaced with —OH, —F, —Cl, —Br, —I, —SH, —OCH₃, —OCH₂CH₃, —SCH₃, or —OC(O)CH₃;
   R₁₀ is hydrogen, carboxy, hydroxy, or aryl_{(C≤12)}, alkylamino_{(C≤12)}, dialkylamino_{(C≤12)}, N-heterocycloalkyl_{(C≤12)}, —C(O)N(R₁₁)-alkanediyl_{(C≤6)}-heterocycloalkyl_{(C≤12)}, —C(O)-alkylamino_{(C≤12)}, —C(O)-dialkylamino_{(C≤12)}, —C(O)—N-heterocycloalkyl_{(C≤12)}, wherein:
   R₁₁ is hydrogen, alkyl_{(C≤6)}, or substituted alkyl_{(C≤6)}:
   wherein the final degradable diacyl in the chain is attached to a terminating group;
   n is 0, 1, 2, 3, 4, 5, or 6;
      or a pharmaceutically acceptable salt thereof. In some embodiments, the terminating group is further defined by the formula:

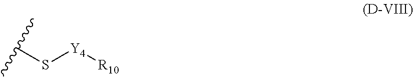

wherein:
Y₄ is alkanediyl_{(C≤18)}; and
R₁₀ is hydrogen. In some embodiments, A₁ and A₂ are each independently —O— or —NRₐ—.

In some embodiments of the dendrimer or dendron of formula (D-I), the core is further defined by the formula:

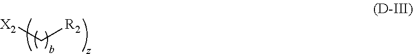

wherein:
X₂ is N(R₅)_{y};
   R₅ is hydrogen or alkyl_{(C≤8)}, or substituted alkyl_{(C≤18)}; and
   y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3.

In some embodiments of the dendrimer or dendron of formula (D-1), the core is further defined by the formula:

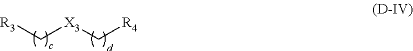

wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))_{c}R_{d},

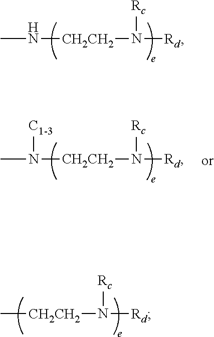

wherein:
   e and f are each independently 1, 2, or 3; provided that the sum of e and f is 3;
   R_{c}, R_{d}, and R_{f} are each independently hydrogen, alkyl_{(C≤8)}, or substituted alkyl_{(C≤8)};
c and d are each independently 1, 2, 3, 4, 5, or 6.

In some embodiments of the dendrimer or dendron of formula (I), the terminating group is represented by the formula:

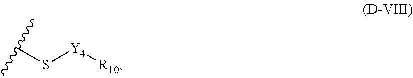

wherein:
Y₄ is alkanediyl_{(C≤8)}; and
R₁₀ is hydrogen.

In some embodiments of the dendrimer or dendron of formula (D-I), the core is further defined as:

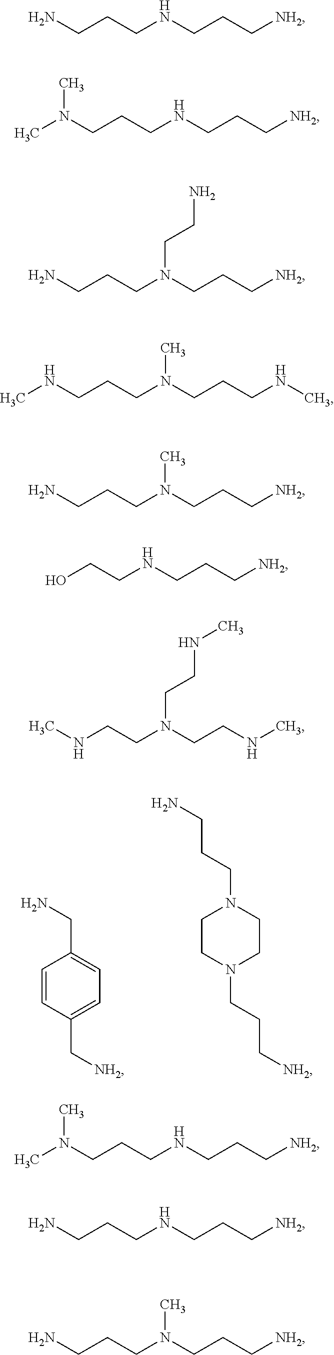

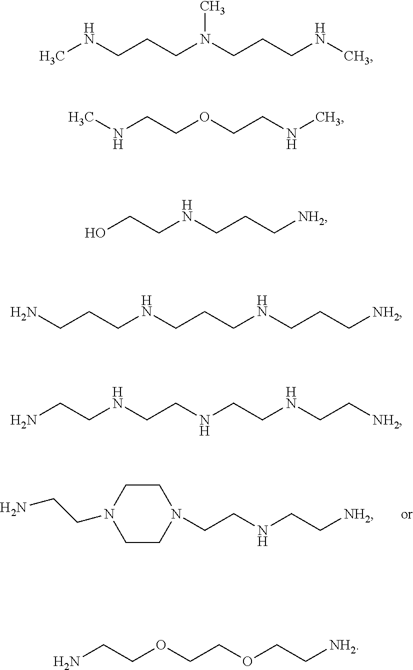

In some embodiments of the dendrimer or dendron of formula (D-I), the degradable diacyl is further defined as:

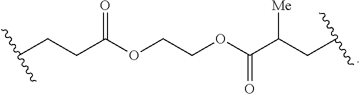 [SMILES: *CCC(=O)OCCOC(=O)C(C)C*]

In some embodiments of the dendrimer or dendron of formula (D-I), the linker is further defined as

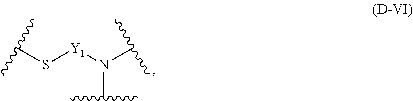

wherein Y₁ is alkanediyl_{(C≤8)} or substituted alkanediyl_{(C≤8)}.

In some embodiments of the dendrimer or dendron of formula (D-I), the dendrimer or dendron is selected from the group consisting of:

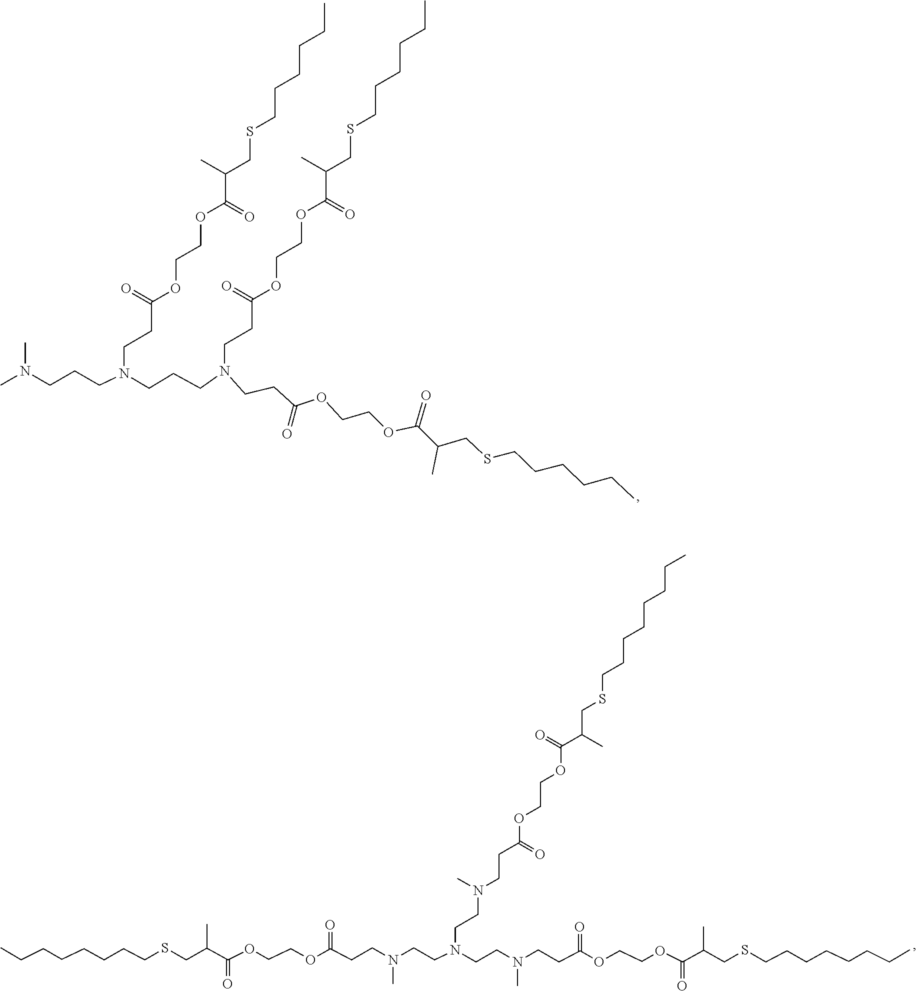

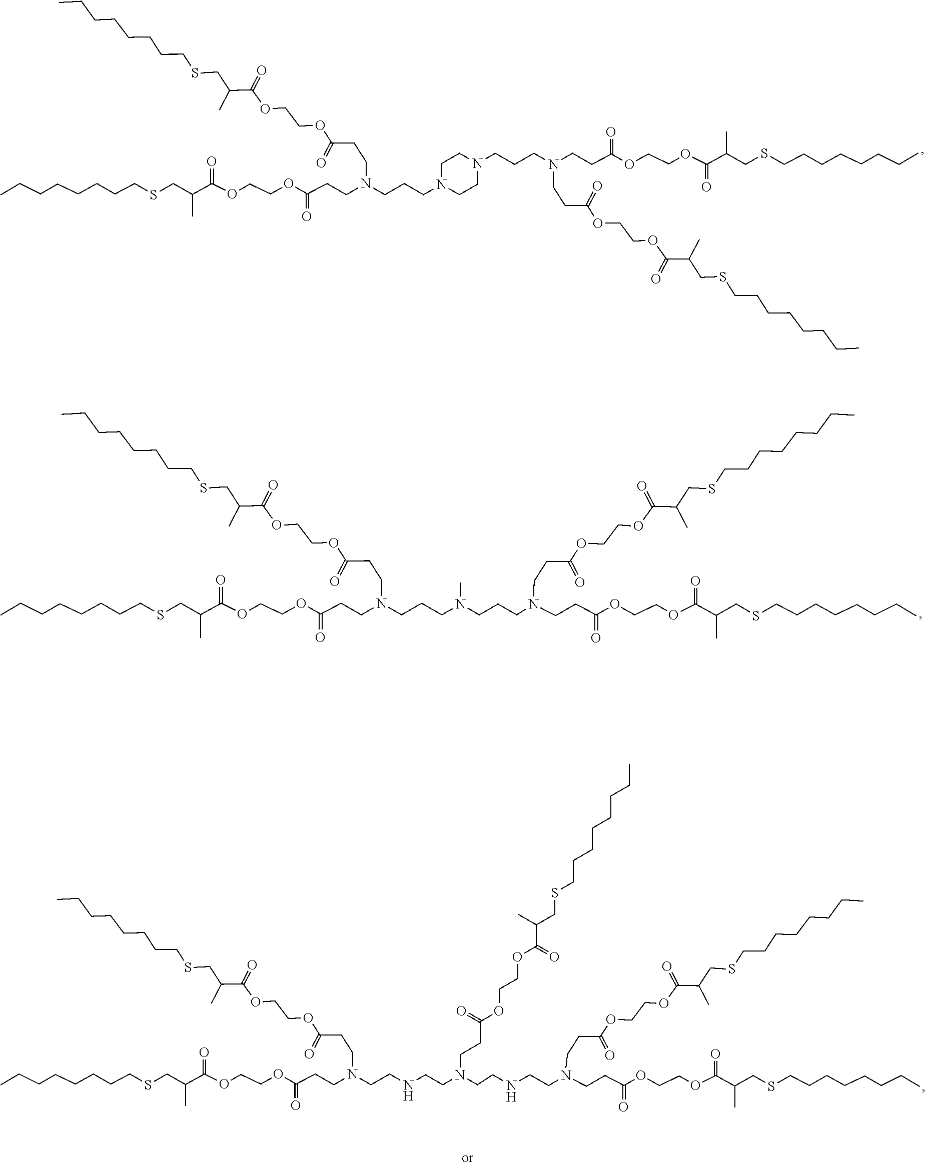

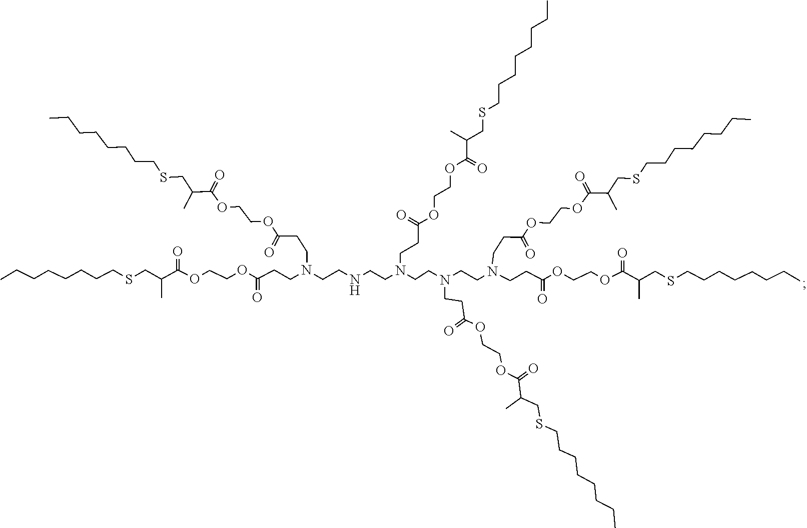 [SMILES: CCCCCCCCSCC(C)C(=O)OCCOC(=O)CCN(CCNCCN(CCC(=O)OCCOC(=O)C(C)CSCCCCCCCC)CCN(CCC(=O)OCCOC(=O)C(C)CSCCCCCCCC)CCN(CCC(=O)OCCOC(=O)C(C)CSCCCCCCCC)CCC(=O)OCCOC(=O)C(C)CSCCCCCCCC)CCC(=O)OCCOC(=O)C(C)CSCCCCCCCC]

and pharmaceutically acceptable salts thereof.

### Dendrimers or Dendrons of Formula (X)

In some embodiments of the lipid composition, the ionizable cationic lipid is a dendrimer or dendron of the formula Core-(Branch)_{N}. In some embodiments, the ionizable cationic lipid is a dendrimer or dendron of the formula

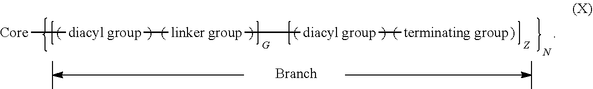

In some embodiments of the lipid composition, the ionizable cationic lipid is a dendrimer or dendron of a generation (g) having a structural formula:

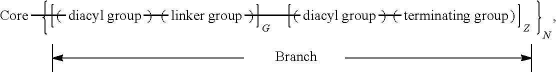

or a pharmaceutically acceptable salt thereof, wherein:
(a) the core comprises a structural formula (X_{Core}):

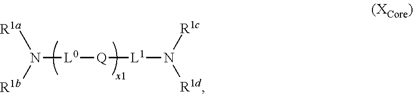

wherein:
Q is independently at each occurrence a covalent bond, —O—, —S—, —NR²—, or —CR^{3a}R^{3b}—;
R² is independently at each occurrence R^{1g} or -L²-NR^{1e}R^{1f};
R^{3a} and R^{3b} are each independently at each occurrence hydrogen or an optionally substituted (e.g., C₁-C₆, such as C₁-C₃) alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or.
alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
x¹ is 0, 1, 2, 3, 4, 5, or 6; and
(b) each branch of the plurality (N) of branches independently comprises a structural formula (X_{Branch}):

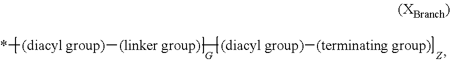

wherein:
* indicates a point of attachment of the branch to the core;
g is 1, 2, 3, or 4;
Z=2^{(g-1)};
G=0, when g=1; or G=Σᵢ₌₀^{i=g-2}2ⁱ, when g≠1;
(c) each diacyl group independently comprises a structural formula

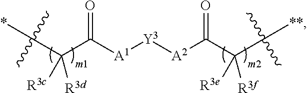

wherein:
* indicates a point of attachment of the diacyl group at the proximal end thereof;
** indicates a point of attachment of the diacyl group at the distal end thereof;
Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR₄—, wherein: R₄ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula

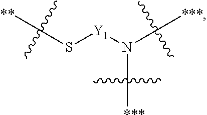

wherein:
   ** indicates a point of attachment of the linker to a proximal diacyl group:
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(c) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

In some embodiments of X_{Core}, Q is independently at each occurrence a covalent bond, —O—, —S—, —NR²— or —CR^{3a}R^{3b}. In some embodiments of X_{Core} Q is independently at each occurrence a covalent bond. In some embodiments of X_{Core} Q is independently at each occurrence an —O—. In some embodiments of X_{Core} Q is independently at each occurrence a —S—. In some embodiments of X_{Core} Q is independently at each occurrence a —NR² and R₂ is independently at each occurrence R^{1g} or -L²-NR^{1e}R^{1f}. In some embodiments of X_{Core} Q is independently at each occurrence a —CR^{3a}R^{3b}R^{3a}, and R^{3a} and R^{3b} are each independently at each occurrence hydrogen or an optionally substituted alkyl (e.g., C₁-C₆, such as C₁-C₃).

In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl. In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen. In some embodiments of X_{Core}, R^{1a}, R^{1b}, R^{1c}, R^{1d}, R^{1e}, R^{1f}, and R^{1g} (if present) are each independently at each occurrence a point of connection to a branch an optionally substituted alkyl (e.g., C₁-C₁₂).

In some embodiments of X_{Core}, L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or, alternatively, part of L¹ form a heterocycloalkyl (e.g., C₄-C₆ and containing one or two nitrogen atoms and, optionally, an additional heteroatom selected from oxygen and sulfur) with one of R^{1c} and R^{1d}. In some embodiments of X_{core}, L⁰, L¹, and L² are each independently at each occurrence can be a covalent bond. In some embodiments of X_{Core}, L⁰, L¹, and L² are each independently at each occurrence can be a hydrogen. In some embodiments of X_{Core}, L⁰, L¹, and L² are each independently at each occurrence can be an alkylene (e.g., C₁-C₁₂, such as C₁-C₆ or C₁-C₃). In some embodiments of X_{Core}, L⁰, L¹, and L² are each independently at each occurrence can be a heteroalkylene (e.g. C₁-C₁₂, such as C₁-C₈ or C₁-C₆). In some embodiments of X_{Core}, L₀, L¹, and L² are each independently at each occurrence can be a heteroalkylene (e.g., C₂-C₈ alkyleneoxide, such as oligo(ethyleneoxide)). In some embodiments of X_{Core}, L⁰, L¹, and L² are each independently at each occurrence can be a [alkylene]-[heterocycloalkyl]-[alkylene] [(e.g., C₁-C₆) alkylene]-[(e.g., C₄-C₆) heterocycloalkyl]-[(e.g., C₁-C₆) alkylene]. In some embodiments of X_{Core}, L⁰, L¹, and L² are each independently at each occurrence can be a [alkylene]-(arylene)-[alkylene] [(e.g., C₁-C₆) alkylene]-(arylene)-[(e.g., C₁-C₆) alkylene]. In some embodiments of X_{Core}, L⁰, L¹, and L² are each independently at each occurrence can be a [alkylene]-(arylene)-[alkylene] (e.g., [(e.g., C₁-C₆) alkylene]-phenylene-[(e.g., C₁-C₆) alkylene]). In some embodiments of X_{Core}, L⁰, L¹, and L² are each independently at each occurrence can be a heterocycloalkyl (e.g., C₄-C₆ heterocycloalkyl). In some embodiments of X_{Core}, L⁰, L¹, and L² are each independently at each occurrence can be an arylene (e.g., phenylene). In some embodiments of X_{Core}, part of L¹ form a heterocycloalkyl with one of R^{1c} and R^{1d}. In some embodiments of X_{Core}, part of L¹ form a heterocycloalkyl (e.g., C₄-C₆ heterocycloalkyl) with one of R^{1c} and R^{1d} and the heterocycloalkyl can contain one or two nitrogen atoms and, optionally, an additional heteroatom selected from oxygen and sulfur.

In some embodiments of X_{Core}, L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene] (e.g.,

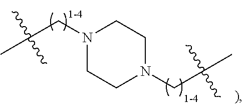

and [(C₁-C₄) alkylene]-phenylene-[(C₁-C₄) alkylene] (e.g.,

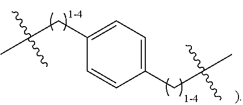

In some embodiments of X_{Core}, L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments of X_{Core}, L⁰, L¹, and L² are each independently at each occurrence C₁-C₄ alkylene (e.g., C₁-C₃ alkylene). In some embodiments, L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)). In some embodiments of X_{Core}, L⁰, L¹, and L² are each independently at each occurrence selected from [(C₁-C₄) alkylene]-[(C₄-C₆) heterocycloalkyl]-[(C₁-C₆) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

In some embodiments of X_{Core}, x¹ is 0, 1, 2, 3, 4, 5, or 6. In some embodiments of X_{Core}, x¹ is 0. In some embodiments of X_{Core}, x¹ is 1. In some embodiments of X_{Core}, x¹ is 2. In some embodiments of X_{Core}, x¹ is 0, 3. In some embodiments of X_{Core} x¹ is 4. In some embodiments of X_{Core} x¹ is 5. In some embodiments of X_{Core}, x¹ is 6.

In some embodiments of X_{Core}, the core comprises a structural formula:

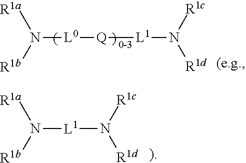

In some embodiments of X_{Core}, the core comprises a structural formula:

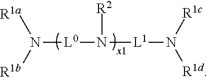

In some embodiments of X_{Core}, the core comprises a structural formula:

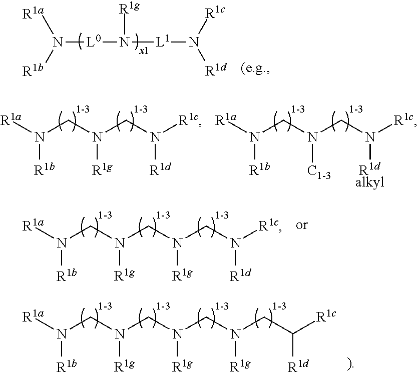

In some embodiments of X_{Core}, the core comprises a structural formula:

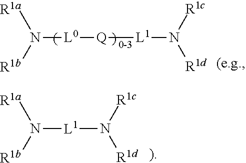

In some embodiments of X_{Core}, the core comprises a structural formula:

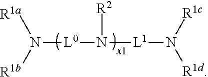

In some embodiments of X_{Core}, the core comprises a structural formula:

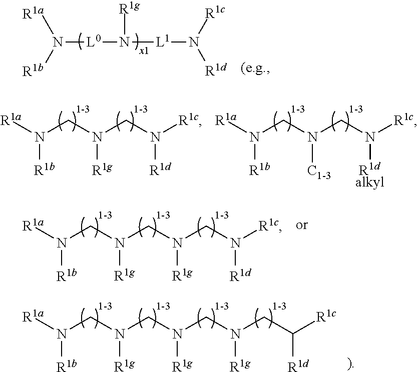

In some embodiments of X_{Core}, the core comprises a structural formula:

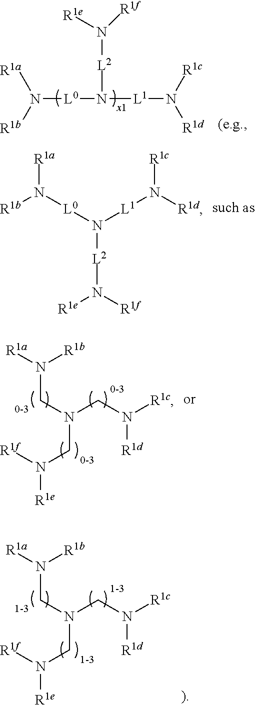

In some embodiments of X_{Core}, the core comprises a structural formula:

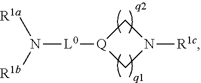

wherein Q′ is —NR²— or —CR^{3a}R^{3b}—; q¹ and q² are each independently 1 or 2. In some embodiments of X_{Core}, the core comprises a structural formula:

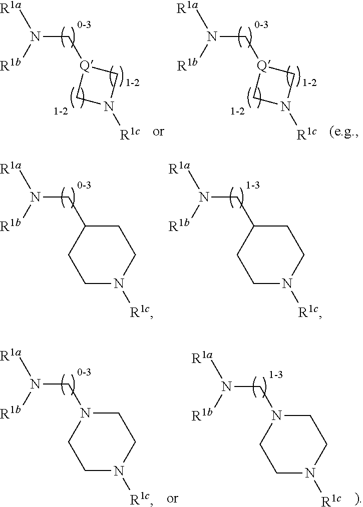

In some embodiments of X_{Core}, the core comprises a structural formula

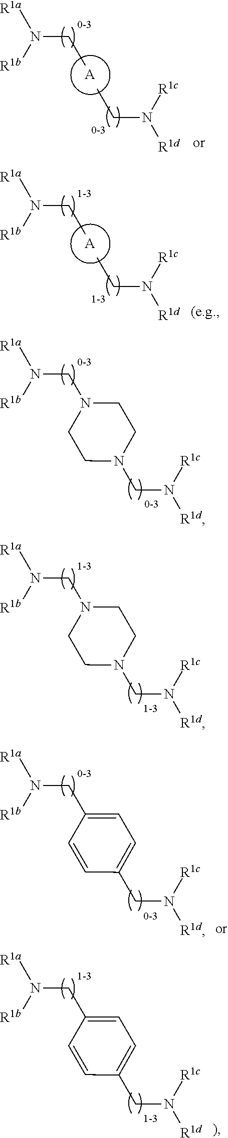

wherein ring A is an optionally substituted aryl or an optionally substituted (e.g., C₃-C₁₂, such as C₃-C₅) heteroaryl. In some embodiments of X_{Core}, the core comprises has a structural formula

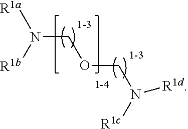

In some embodiments of X_{Core}, the core comprises a structural formula set forth in Table. 1 and pharmaceutically acceptable salts thereof, wherein * indicates a point of attachment of the core to a branch of the plurality of branches. In some embodiments, the example cores of Table. 1 are not limited to the stereoisomers (i.e. enantiomers, diastereomers) listed.

TABLE 1
Example core structures

| ID # | Structure |
|---|---|
| 1A1 | 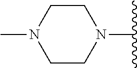 [SMILES: *N1CCN(C)CC1] |
| 1A2-1 | 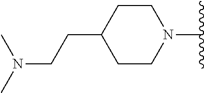 [SMILES: *N1CCC(CCN(C)C)CC1] |
| 1A2-2 | 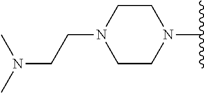 [SMILES: *N1CCN(CCN(C)C)CC1] |
| 1A3-1 | 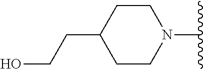 [SMILES: *N1CCC(CCO)CC1] |
| 1A3-2 | 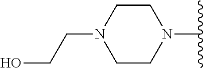 [SMILES: *N1CCN(CCO)CC1] |
| 1A4 | 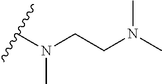 [SMILES: *N(C)CCN(C)C] |
| 1A5-1 | 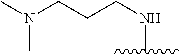 [SMILES: *NCCCN(C)C] |
| 1A5-2 | 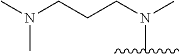 |
| 2A1-1 | 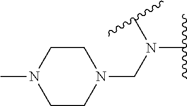 [SMILES: *N(*)CN1CCN(C)CC1] |
| 2A1-2 | 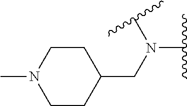 [SMILES: *N(*)CC1CCN(C)CC1] |
| 2A2-1 | 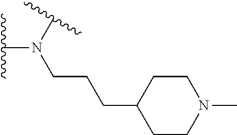 [SMILES: *N(*)CCCC1CCN(C)CC1] |
| 2A2-2 | 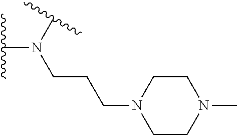 [SMILES: *N(*)CCCN1CCN(C)CC1] |
| 2A3 | 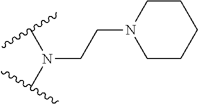 [SMILES: *N(*)CCN1CCCCC1] |
| 2A4 | 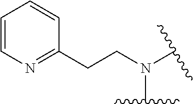 [SMILES: *N(*)CCc1ccccn1] |
| 2A5 | 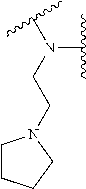 [SMILES: *N(*)CCN1CCCC1] |
| 2A6 | 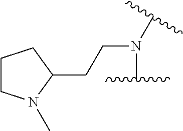 [SMILES: *N(*)CCC1CCCN1C] |
| 2A7-1 | 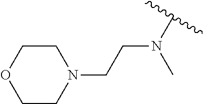 [SMILES: *N(C)CCN1CCOCC1] |
| 2A7-2 | 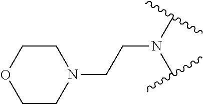 [SMILES: *N(*)CCN1CCOCC1] |
| 2A8 | 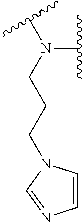 [SMILES: *N(*)CCCn1ccnc1] |
| 2A9 | 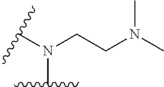 [SMILES: *N(*)CCN(C)C] |
| 2A9V | 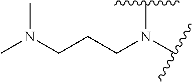 [SMILES: *N(*)CCCN(C)C] |
| 2A10 | 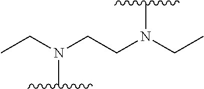 [SMILES: *N(CC)CCN(*)CC] |
| 2A11 | 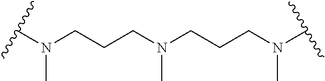 [SMILES: *N(C)CCCN(C)CCCN(*)C] |
| 2A12 | 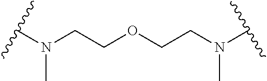 [SMILES: *N(C)CCOCCN(*)C] |
| 3A1 | 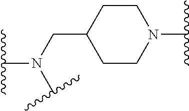 [SMILES: *N(*)CC1CCN(*)CC1] |
| 3A2 | 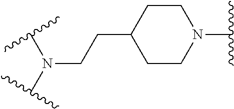 [SMILES: *N(*)CCC1CCN(*)CC1] |
| 3A3 | 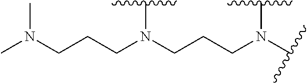 [SMILES: *N(*)CCCN(*)CCCN(C)C] |
| 3A4 | 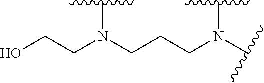 [SMILES: *N(*)CCCN(*)CCO] |
| 3A5 | 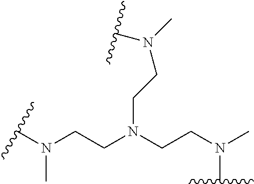 [SMILES: *N(C)CCN(CCN(*)C)CCN(*)C] |
| 3A6 | 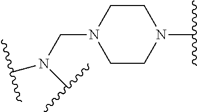 [SMILES: *N(*)CN1CCN(*)CC1] |
| 3A7 | 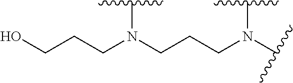 [SMILES: *N(*)CCCN(*)CCCO] |
| 4A1 | 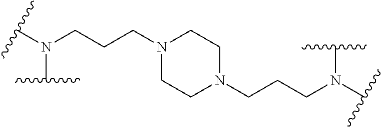 [SMILES: *N(*)CCCN1CCN(CCCN(*)*)CC1] |
| 4A2 | 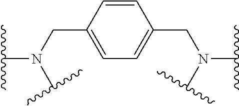 [SMILES: *N(*)Cc1ccc(CN(*)*)cc1] |
| 4A3 | 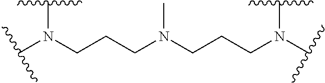 [SMILES: *N(*)CCCN(C)CCCN(*)*] |
| 4A4 | 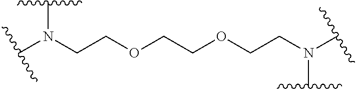 [SMILES: *N(*)CCOCCOCCN(*)*] |
| 5A1 | 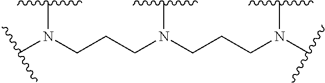 [SMILES: *N(*)CCCN(*)CCCN(*)*] |
| 5A2-1 (5-arm) | 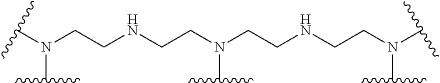 [SMILES: *N(*)CCNCCN(*)CCNCCN(*)*] |
| 5A2-2 (5-arm) | 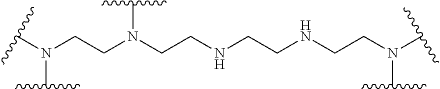 [SMILES: *N(*)CCNCCNCCN(*)CCN(*)*] |
| 5A2-3 (5-arm) | 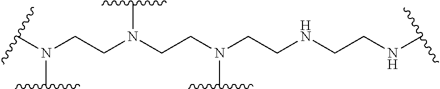 [SMILES: *NCCNCCN(*)CCN(*)CCN(*)*] |
| 5A2-4 (5-arm) | 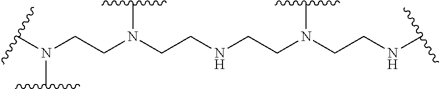 [SMILES: *NCCN(*)CCNCCN(*)CCN(*)*] |
| 5A3-1 (5-arm) | 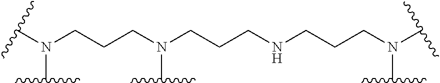 [SMILES: *N(*)CCCNCCCN(*)CCCN(*)*] |
| 5A4-1 (5-arm) | 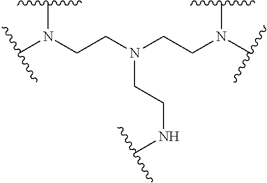 [SMILES: *NCCN(CCN(*)*)CCN(*)*] |
| 5A5 | 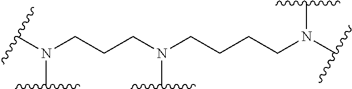 [SMILES: *N(*)CCCCN(*)CCCN(*)*] |
| 5A6 | 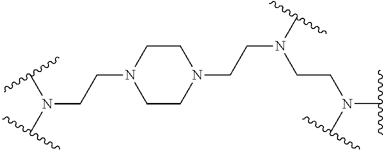 [SMILES: *N(*)CCN(*)CCN1CCN(CCN(*)*)CC1] |
| 5A2-4 (6 arm) | 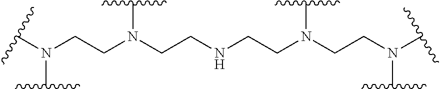 [SMILES: *N(*)CCN(*)CCNCCN(*)CCN(*)*] |
| 5A2-5 (6 arm) | 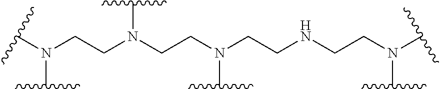 [SMILES: *N(*)CCNCCN(*)CCN(*)CCN(*)*] |
| 5A2-6 (6 arm) | 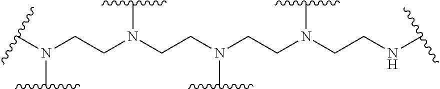 [SMILES: *NCCN(*)CCN(*)CCN(*)CCN(*)*] |
| 5A3-2 (6 arm) | 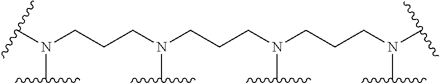 [SMILES: *N(*)CCCN(*)CCCN(*)CCCN(*)*] |
| 5A4-2 (6 arm) | 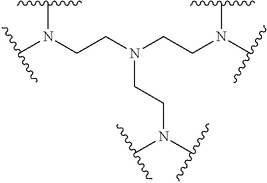 [SMILES: *N(*)CCN(CCN(*)*)CCN(*)*] |
| 6A4 | 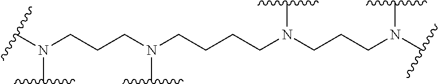 [SMILES: *N(*)CCCN(*)CCCCN(*)CCCN(*)*] |
| 1H1 | 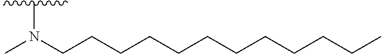 [SMILES: *N(C)CCCCCCCCCCCC] |
| 1H2 | 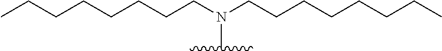 [SMILES: *N(CCCCCCCC)CCCCCCCC] |
| 1H3 | 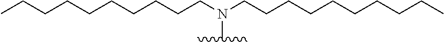 [SMILES: *N(CCCCCCCCCC)CCCCCCCCCC] |
| 2H1 | 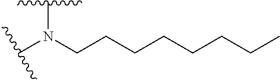 [SMILES: *N(*)CCCCCCCC] |
| 2H2 | 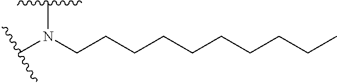 [SMILES: *N(*)CCCCCCCCCC] |
| 2H3 | 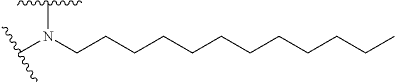 [SMILES: *N(*)CCCCCCCCCCCC] |
| 2H4 | 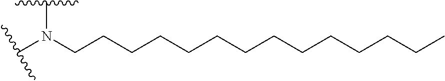 [SMILES: *N(*)CCCCCCCCCCCCCC] |
| 2H5 | 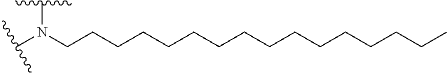 [SMILES: *N(*)CCCCCCCCCCCCCCCC] |
| 2H6 | 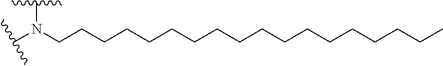 [SMILES: *N(*)CCCCCCCCCCCCCCCCCC] |

In some embodiments of X_{core}, the core comprises a structural formula selected from the group consisting of:

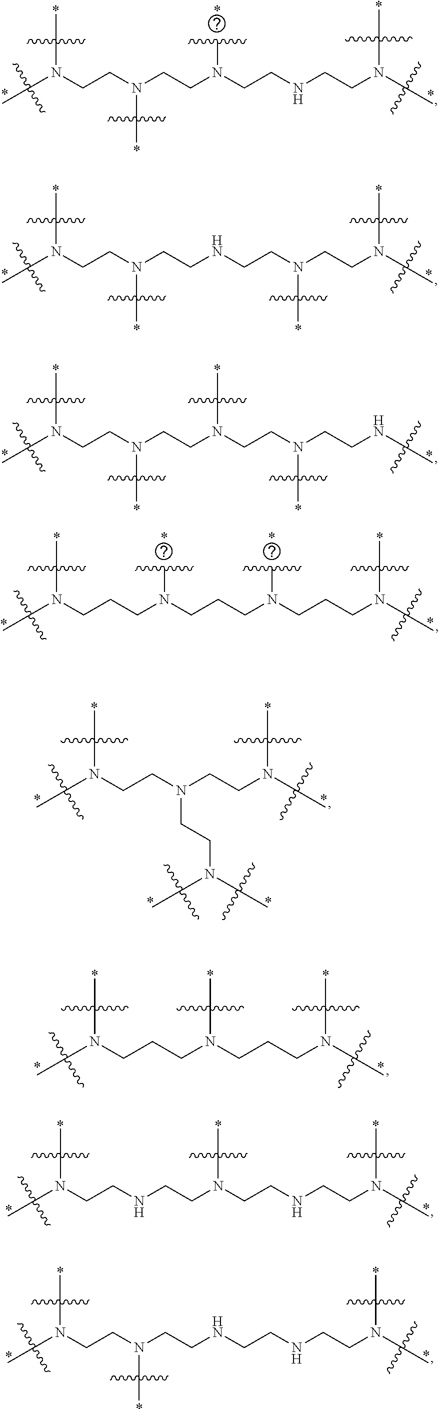

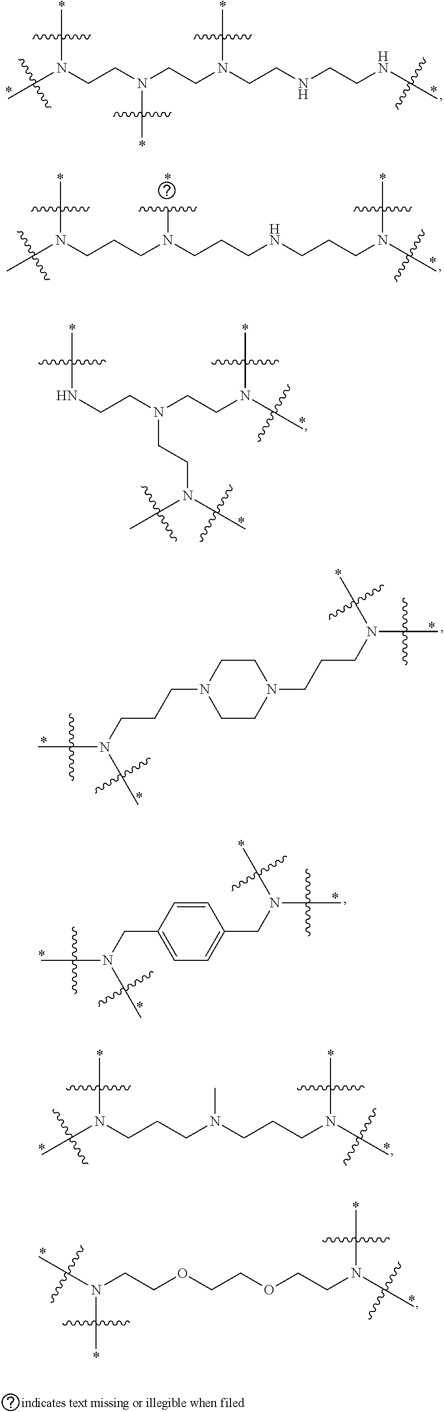

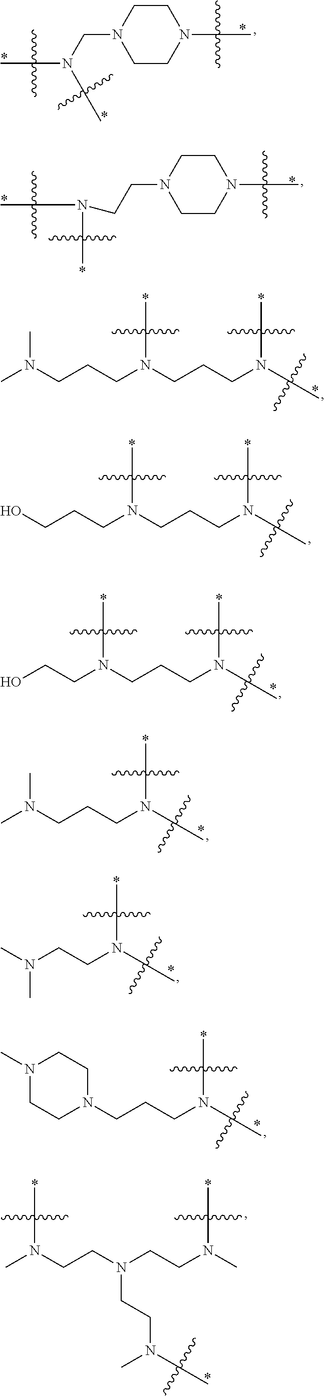

and pharmaceutically acceptable salts thereof, wherein * indicates a point of attachment of the core to a branch of the plurality of branches or H. In some embodiments, wherein * indicates a point of attachment of the core to a branch of the plurality of branches.

In some embodiments of X_{Core}, the core has the structure

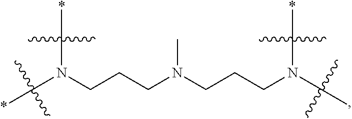

wherein * indicates a point of attachment of the core to a branch of the plurality of branches or H. In some embodiments, at least 2 branches are attached to the core. In some embodiments, at least 3 branches are attached to the core. In some embodiments, at least 4 branches are attached to the core.

In some embodiments of X_{Core}, the core has the structure

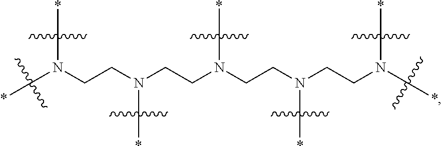

wherein * indicates a point of attachment of the core to a branch of the plurality of branches or H. In some embodiments, at least 4 branches are attached to the core. In some embodiments, at least 5 branches are attached to the core. In some embodiments, at least 6 branches are attached to the core.

In some embodiments, the plurality (N) of branches comprises at least 3 branches, at least 4 branches, at least 5 branches. In some embodiments, the plurality (N) of branches comprises at least 3 branches. In some embodiments, the plurality (N) of branches comprises at least 4 branches. In some embodiments, the plurality (N) of branches comprises at least 5 branches.

In some embodiments of X_{Branch}, g is 1, 2, 3, or 4. In some embodiments of X_{Branch}, g is 1. In some embodiments of X_{Branch}, g is 2. In some embodiments of X_{Branch}, g is 3. In some embodiments of X_{Branch}, g is 4.

In some embodiments of X_{Branch}, Z=2^{(g-1)} and when g=1, G=0. In some embodiments of X_{Branch}, Z=2^{(g-1)} and G=Σᵢ₌₀^{g-2}2ⁱ, when g≠1.

In some embodiments of X_{Branch}, g=1, G=0, Z=1, and each branch of the plurality of branches comprises a structural formula each branch of the plurality of branches comprises a structural formula *-(diacyl group)-(terminating group).

In some embodiments of X_{Branch}, g=2, G=1, Z=2, and each branch of the plurality of branches comprises a structural formula

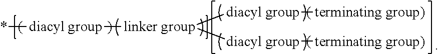

In some embodiments of X_{Branch}, g=3, G=3, Z=4, and each branch of the plurality of branches comprises a structural formula

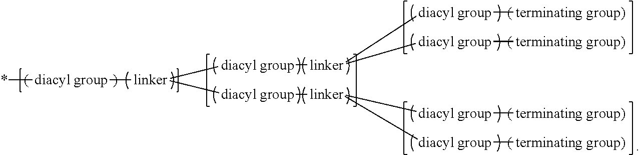

In some embodiments of X_{Branch}, g=4, G=7, Z=8, and each branch of the plurality of branches comprises a structural formula

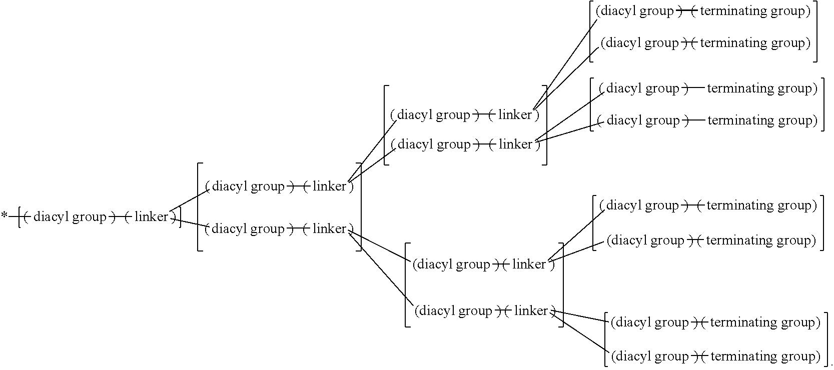

In some embodiments, the dendrimers or dendrons described herein with a generation (g)=1 has the structure:

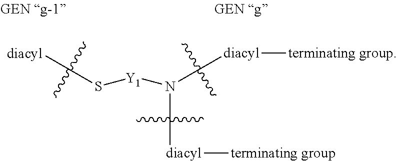

In some embodiments, the dendrimers or dendrons described herein with a generation (g)=1 has the structure:

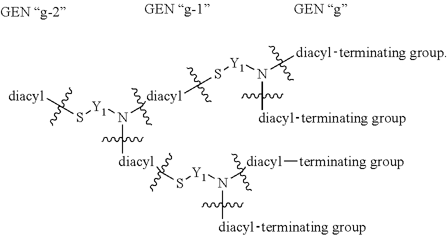

Example formulation of the dendrimers or dendrons described herein for generations 1-4 is shown in Table 2. The number of diacyl groups, linker groups, and terminating groups can be calculated based on g.

TABLE 2
Formulation of Dendrimer or Dendron Groups Based on Generation (g)

| | g = 1 | g = 2 | g = 3 | g = 4 | |
|---|---|---|---|---|---|
| # of diacyl grp | 1 | 1 + 2 = 3 | 1 + 2 + 2² = 7 | 1 + 2 + 2² + 2³ = 15 | 1 + 2 + . . . + 2^{g-1} |
| # of linker grp | 0 | 1 | 1 + 2 | 1 + 2 + 2² | 1 + 2 + . . . + 2^{g-2} |
| # of terminating | 1 | 2 | 2² | 2³ | 2^{(g-1)} |
| grp | | | | | |

In some embodiments, the diacyl group independently comprises a structural formula

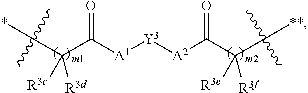

* indicates a point of attachment of the diacyl group at the proximal end thereof, and ** indicates a point of attachment of the diacyl group at the distal end thereof.

In some embodiments of the diacyl group of X_{Branch}, Y³ is independently at each occurrence an optionally substituted; alkylene, an optionally substituted alkenylene, or an optionally substituted arenylene. In some embodiments of the diacyl group of X_{Branch}, Y³ is independently at each occurrence an optionally substituted alkylene (e.g., C₁-C₁₂). In some embodiments of the diacyl group of X_{Branch}, Y³ is independently at each occurrence an optionally substituted alkenylene (e.g. C₁-C₁₂). In some embodiments of the diacyl group of X_{Branch}, Y³ is independently at each occurrence an optionally substituted arenylene (e.g., C₁-C₁₂).

In some embodiments of the diacyl group of X_{Branch}, A¹ and A² are each independently at each occurrence —O—, —S—, or —NR⁴—. In some embodiments of the diacyl group of X_{Branch}. A¹ and A² are each independently at each occurrence —O—. In some embodiments of the diacyl group of X_{Branch}, A¹ and A² are each independently at each occurrence —S—. In some embodiments of the diacyl group of X_{Branch}, A¹ and A² are each independently at each occurrence —NR₄— and R⁴ is hydrogen or optionally substituted alkyl (e.g., C₁-C₆). In some embodiments of the diacyl group of X_{Branch}, m¹ and m² are each independently at each occurrence 1, 2, or 3. In some embodiments of the diacyl group of X Branch, m¹ and m² are each independently at each occurrence 1. In some embodiments of the diacyl group of X_{Branch}, m¹ and m² are each independently at each occurrence 2. In some embodiments of the diacyl group of X_{Branch}, m¹ and m² are each independently at each occurrence 3. In some embodiments of the diacyl group of X_{Branch}, R^{3c}, R^{3d}, R^{3e}, and R^{3f} are each independently at each occurrence hydrogen or an optionally substituted alkyl. In some embodiments of the diacyl group of X_{Branch}, R^{3c}, R^{3d}, R^{3e}, and R^{3f} are each independently at each occurrence hydrogen. In some embodiments of the diacyl group of X_{Branch}, R^{3c}, R^{3d}, R^{3e}, and R^{3f} are each independently at each occurrence an optionally substituted (e.g., C₁-C₈) alkyl.

In some embodiments of the diacyl group, A¹ is —O— or —NH—. In some embodiments of the diacyl group, A¹ is —O—. In some embodiments of the diacyl group, A² is —O— or —NH—. In some embodiments of the diacyl group, A² is —O—. In some embodiments of the diacyl group, Y³ is C₁-C₁₂ (e.g., C₁-C₆, such as C₁-C₃) alkylene.

In some embodiments of the diacyl group, the diacyl group independently at each occurrence comprises a structural formula

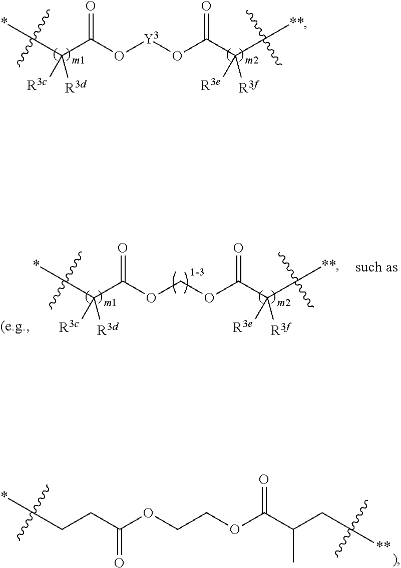

and optionally R^{3c}, R^{3d}, R^{3e}, and R^{3f} are each independently at each occurrence hydrogen or C₁-C₃ alkyl.

In some embodiments, linker group independently comprises a structural formula

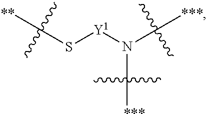

** indicates a point of attachment of the linker to a proximal diacyl group, and *** indicates a point of attachment of the linker to a distal diacyl group.

In some embodiments of the linker group of X_{Branch} if present, Y₁ is independently at each occurrence an optionally substituted alkylene, an optionally substituted alkenylene, or an optionally substituted arenylene. In some embodiments of the linker group of X_{Branch} if present. Y₁ is independently at each occurrence an optionally substituted alkylene (e.g., C₁-C₁₂). In some embodiments of the linker group of X_{Branch} if present, Y₁ is independently at each occurrence an optionally substituted alkenylene (e.g., C₁-C₁₂). In some embodiments of the linker group of X_{Branch} if present, Y₁ is independently at each occurrence an optionally substituted arenylene (e.g., C₁-C₁₂).

In some embodiments of the terminating group of X_{Branch}, each terminating group is independently selected from optionally substituted alkylthiol and optionally substituted alkenylthiol. In some embodiments of the terminating group of X_{Branch}, each terminating group is an optionally substituted alkylthiol (e.g., C₁-C₁₈, such as C₄-C₁₈). In some embodiments of the terminating group of X_{Branch}, each terminating group is optionally substituted alkenylthiol (e.g., C₁-C₁₈, such as C₄-C₁₈).

In some embodiments of the terminating group of X_{Branch}, each terminating group is independently C₁-C₁₈ alkenylthiol or C₁-C₁₈ alkylthiol, and the alkyl or alkenyl moiety is optionally substituted with one or more substituents each independently selected from halogen, C₆-C₁₂ aryl, C₁-C₁₂ alkylamino. C₄-C₆ N-heterocycloalkyl, —OH, —C(O)OH, —C(O)N(C₁-C₃ alkyl)-(C₁-C₆ alkylene)-(C₁-C₁₂ alkylamino), —C(O)N(C₁-C₃ alkyl)-(C₁-C₆ alkylene)-(C₄-C₆ N-heterocycloalkyl), —C(O)—(C₁-C₁₂ alkylamino), and —C(O)—(C₄-C₆ N-heterocycloalkyl), and the C₄-C₆ N-heterocycloalkyl moiety of any of the preceding substituents is optionally substituted with C₁-C₃ alkyl or C₁-C₃ hydroxyalkyl.

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino

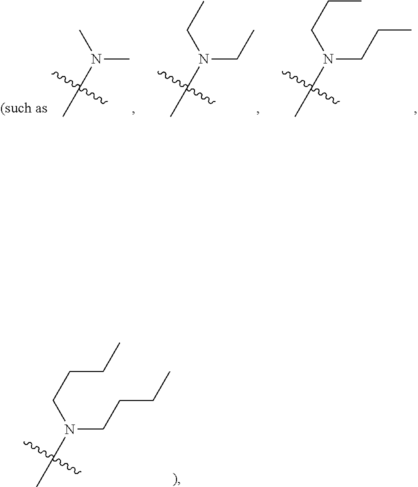

C₄-C₆ N-heterocycloalkyl (e.g., N-pyrrolidinyl

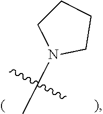 [SMILES: *N1CCCC1]

### N-piperidinyl

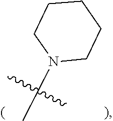 [SMILES: *N1CCCCC1]

### N-azepanyl

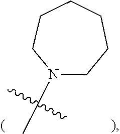 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

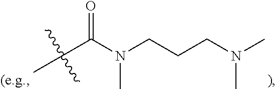 [SMILES: *C(=O)N(C)CCCN(C)C]

—C(O)N(C₁-C₃ alkyl)-(C₁-C₆ alkylene)-(C₄-C₆ N-heterocycloalkyl)

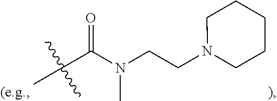 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

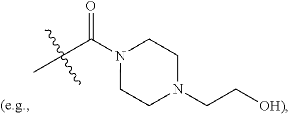

wherein the C₄-C₆ N-heterocycloalkyl moiety of any of the preceding substituents is optionally substituted with C₁-C₃ alkyl or C₁-C₃ hydroxyalkyl. In some embodiments of the terminating group of X_{Branch}, each terminating group is independently C₁-C₁₈ (e.g., C₄-C₁₈) alkylthiol, wherein the alkyl moiety is optionally substituted with one substituent —OH. In some embodiments of the terminating group of X_{Branch}, each terminating group is independently C₁-C₁₈ (e.g., C₄-Cis) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino

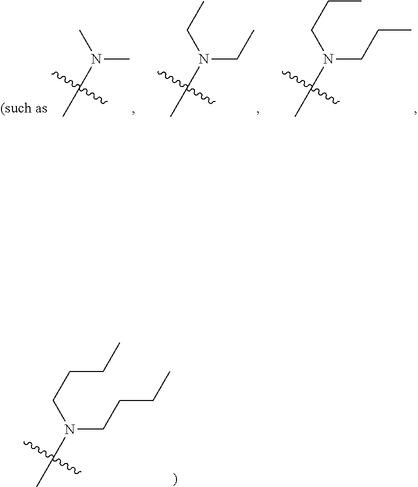

and C₄-C₆ N-heterocycloalkyl (e.g., N-pyrrolidinyl

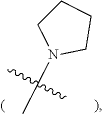 [SMILES: *N1CCCC1]

### N-piperidinyl

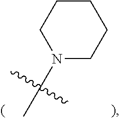 [SMILES: *N1CCCCC1]

### N-azepanyl

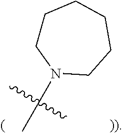 [SMILES: *N1CCCCCC1]

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol. In some embodiments of the terminating group of X_{Branch}, each terminating group is independently C₁-C₁₈ (e.g., C₄-C₁₈) alkylthiol.

In some embodiments of the terminating group of X_{Branch}, each terminating group is independently a structural set forth in Table 3. In some embodiments, the dendrimers or dendrons described herein can comprise a terminating group or pharmaceutically acceptable salt, or thereof selected in Table 3. In some embodiments, the example terminating group of Table 3 are not limited to the stereoisomers (i.e. enantiomers, diastereomers) listed.

TABLE 3
Example terminating group/peripheries structures

| ID # | Structure |
|---|---|
| SC1 | 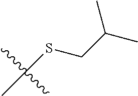 [SMILES: *SCC(C)C] |
| SC2 | 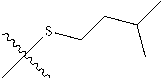 [SMILES: *SCCC(C)C] |
| SC3 | 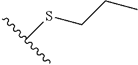 [SMILES: *SCCC] |
| SC4 | 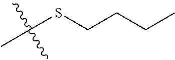 [SMILES: *SCCCC] |
| SC5 | 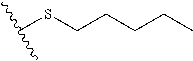 [SMILES: *SCCCCC] |
| SC6 | 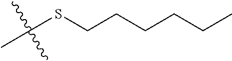 [SMILES: *SCCCCCC] |
| SC7 | 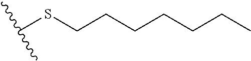 [SMILES: *SCCCCCCC] |
| SC8 | 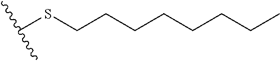 [SMILES: *SCCCCCCCC] |
| SC9 | 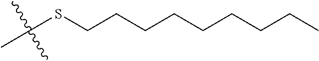 [SMILES: *SCCCCCCCCC] |
| SC10 | 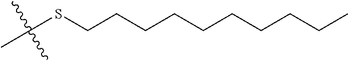 [SMILES: *SCCCCCCCCCC] |
| SC11 | 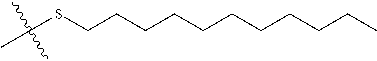 [SMILES: *SCCCCCCCCCCC] |
| SC12 | 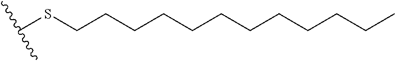 [SMILES: *SCCCCCCCCCCCC] |
| SC14 | 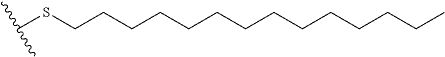 [SMILES: *SCCCCCCCCCCCCCC] |
| SC16 | 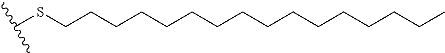 [SMILES: *SCCCCCCCCCCCCCCCC] |
| SC18 | 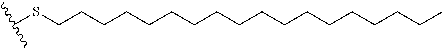 [SMILES: *SCCCCCCCCCCCCCCCCCC] |
| SC19 | 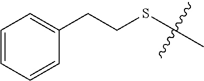 [SMILES: *SCCc1ccccc1] |
| SO1 | 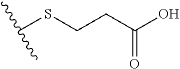 [SMILES: *SCCC(=O)O] |
| SO2 | 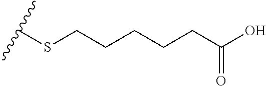 [SMILES: *SCCCCCC(=O)O] |
| SO3 | 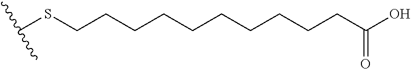 [SMILES: *SCCCCCCCCCCC(=O)O] |
| SO4 | 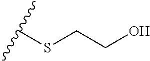 [SMILES: *SCCO] |
| SO5 | 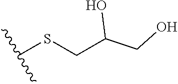 [SMILES: *SCC(O)CO] |
| SO6 | 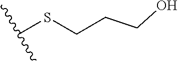 [SMILES: *SCCCO] |
| SO7 | 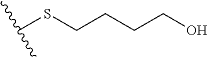 [SMILES: *SCCCCO] |
| SO8 | 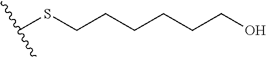 [SMILES: *SCCCCCCO] |
| SO9 | 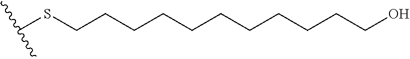 [SMILES: *SCCCCCCCCCCCO] |
| SN1 | 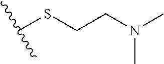 [SMILES: *SCCN(C)C] |
| SN2 | 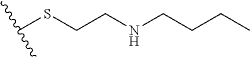 [SMILES: *SCCNCCCC] |
| SN3 | 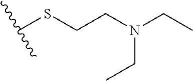 [SMILES: *SCCN(CC)CC] |
| SN4 | 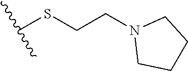 [SMILES: *SCCN1CCCC1] |
| SN5 | 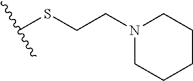 [SMILES: *SCCN1CCCCC1] |
| SN6 | 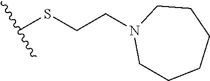 [SMILES: *SCCN1CCCCCC1] |
| SN7 | 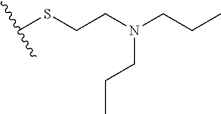 [SMILES: *SCCN(CCC)CCC] |
| SN8 | 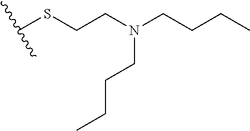 [SMILES: *SCCN(CCCC)CCCC] |
| SN9 | 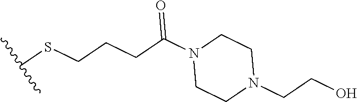 [SMILES: *SCCCC(=O)N1CCN(CCO)CC1] |
| SN10 | 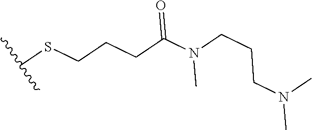 [SMILES: *SCCCC(=O)N(C)CCCN(C)C] |
| SN11 | 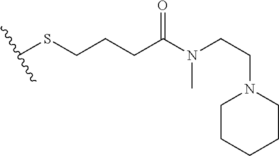 [SMILES: *SCCCC(=O)N(C)CCN1CCCCC1] |

In some embodiments, the dendrimer or dendrons of Formula (X) is selected from those set forth in Table 4 and pharmaceutically acceptable salts thereof.

TABLE 4
Example ionizable cationic lipo-dendrimers or lipo-dendrons

| ID # | Structure |
|---|---|
| 2A2- SC14 | 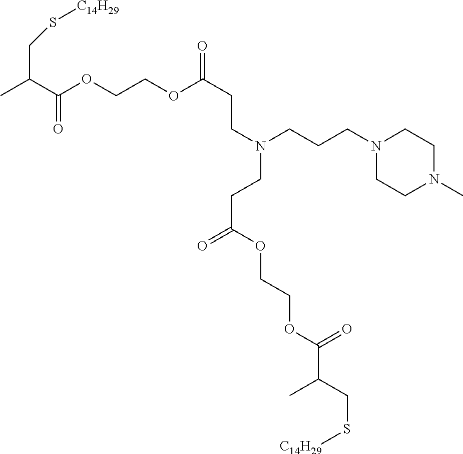 |
| 2A6- SC14 | 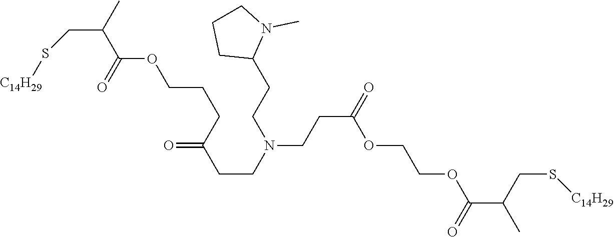 |
| 2A9- SC14 | 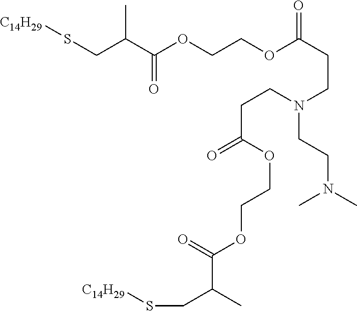 |
| 3A3- SC10 | 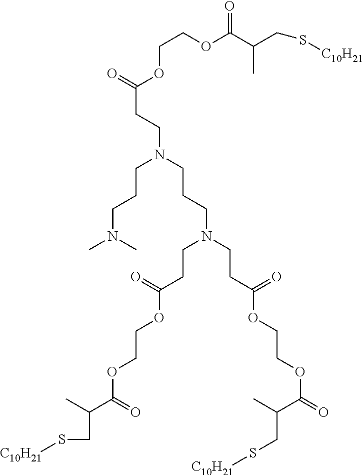 |
| 3A3- SC14 | 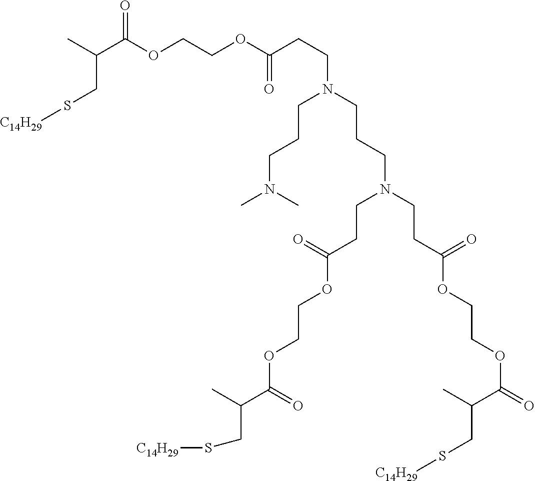 |
| 3A5- SC10 | 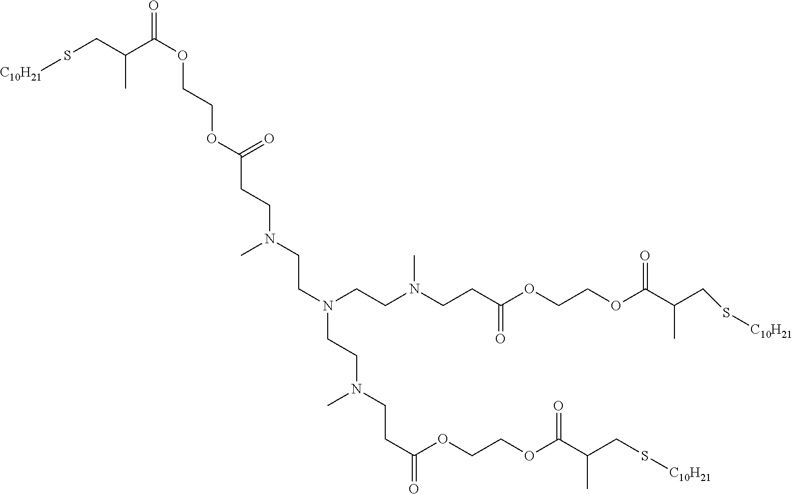 |
| 3A5- SC14 | 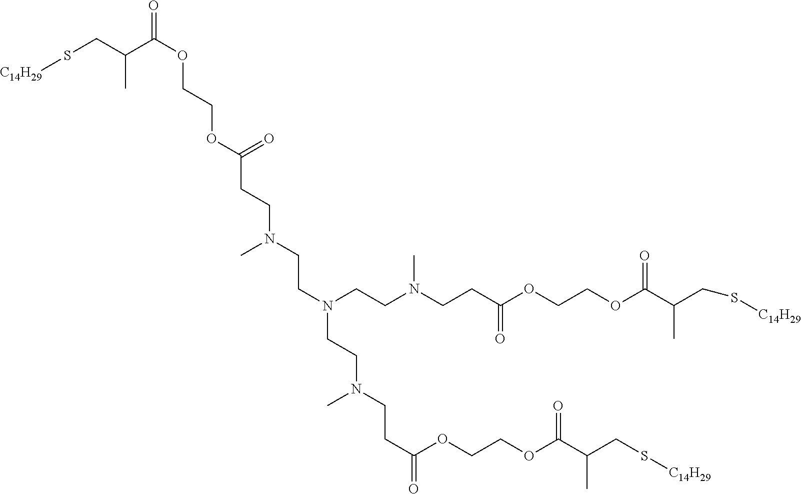 |
| 4A1- SC12 | 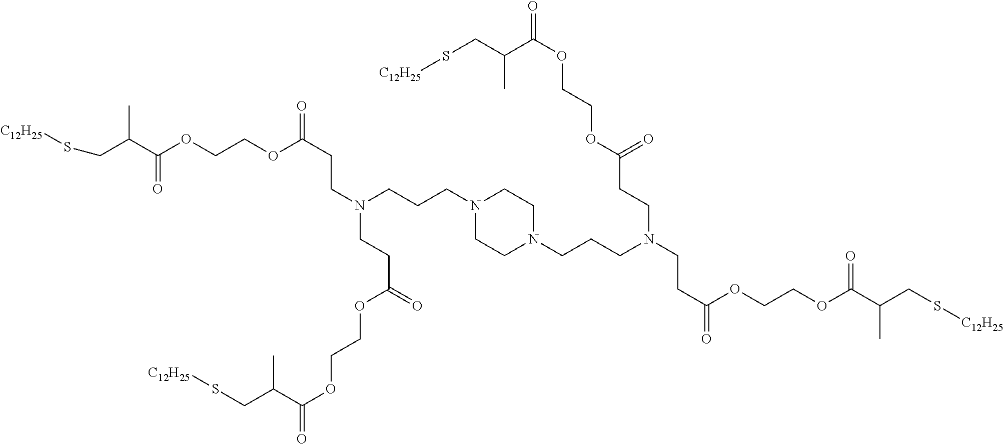 |
| 4A3- SC12 | 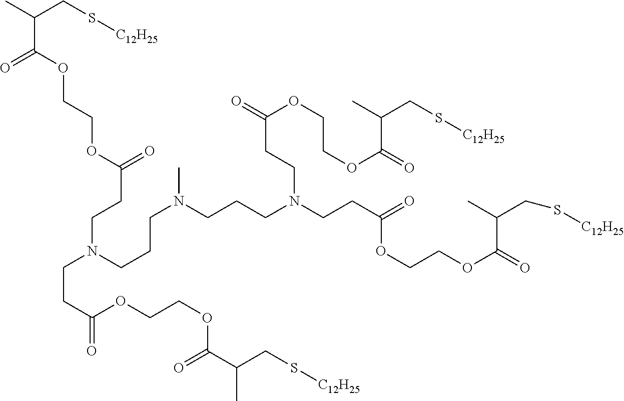 |
| 5A1- SC12 | 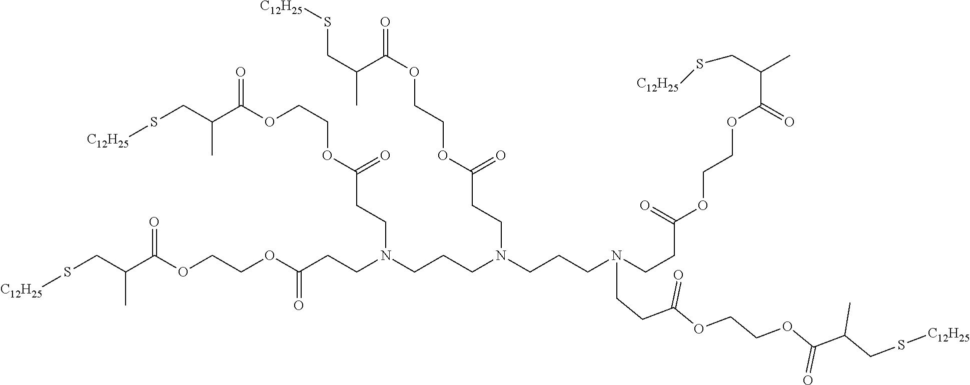 |
| 5A1- SC8 | 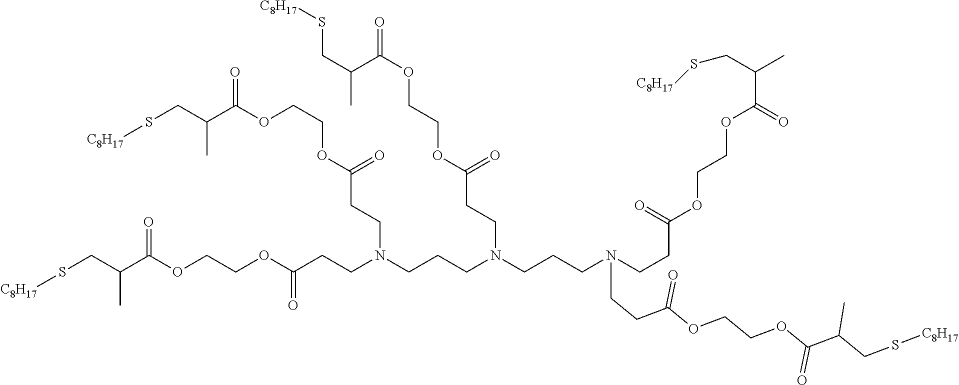 |
| 5A2-2- SC12 (5-arm) | 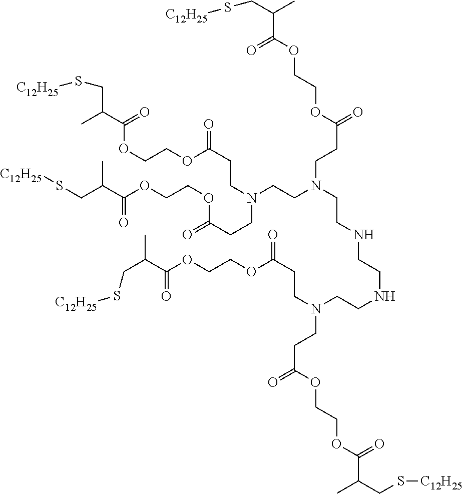 |
| 5A3-1- SC12 (5 arm) | 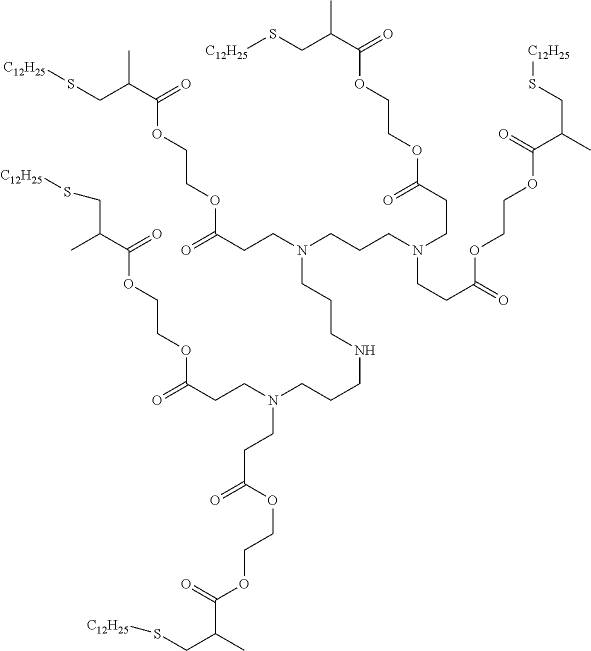 |
| 5A3-1- SC8 (5-arm) | 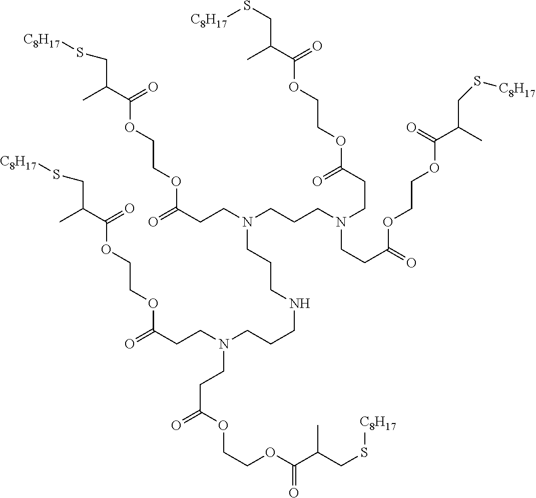 |
| 5A4-1- SC12 (5-arm) | 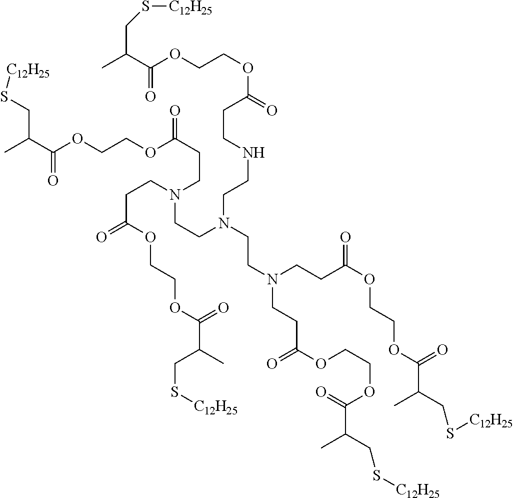 |
| 5A4-1- SC8 (5-arm) | 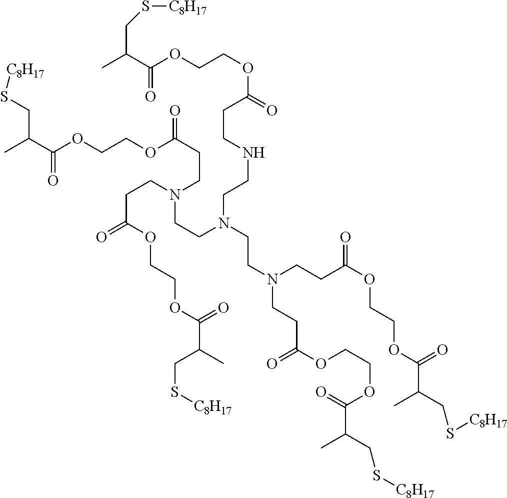 |
| 5A5- SC8 | 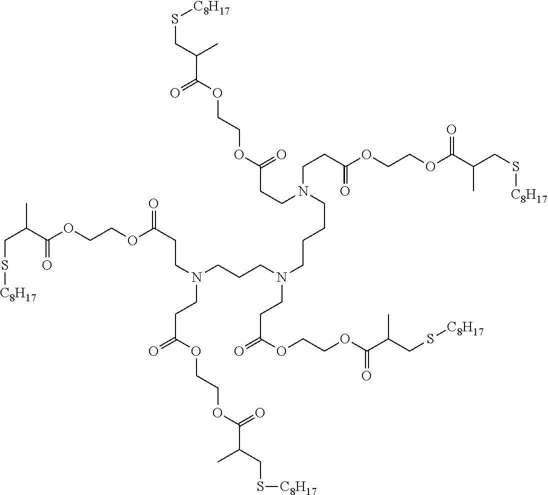 |
| 5A5- SC12 | 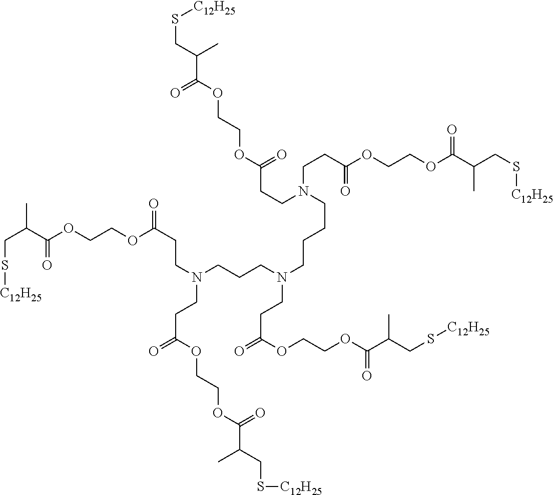 |
| 5A2-4- SC12 (6-arm) | 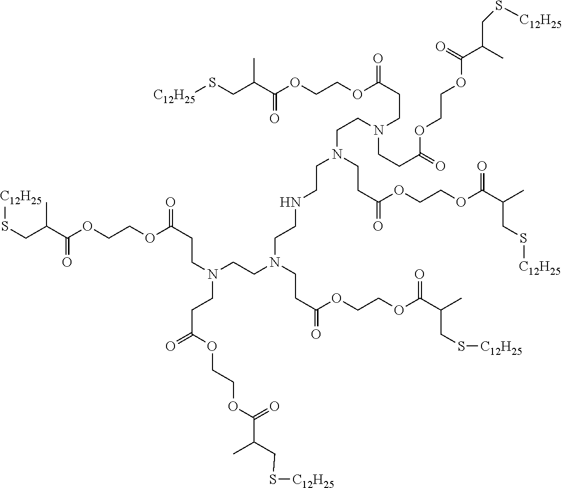 |
| 5A2-4- SC10 (6-arm) | 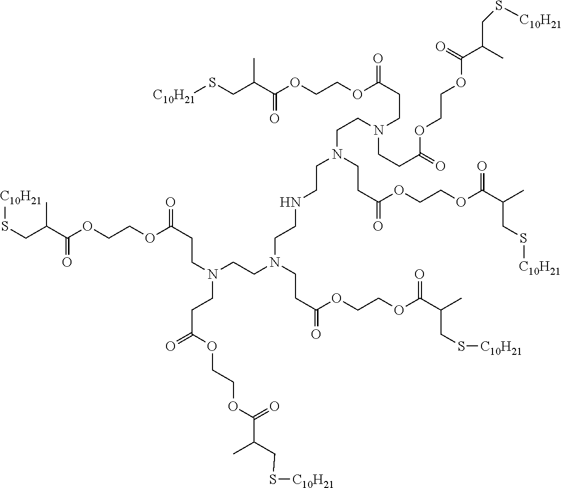 |
| 5A3-2- SC8 (6-arm) | 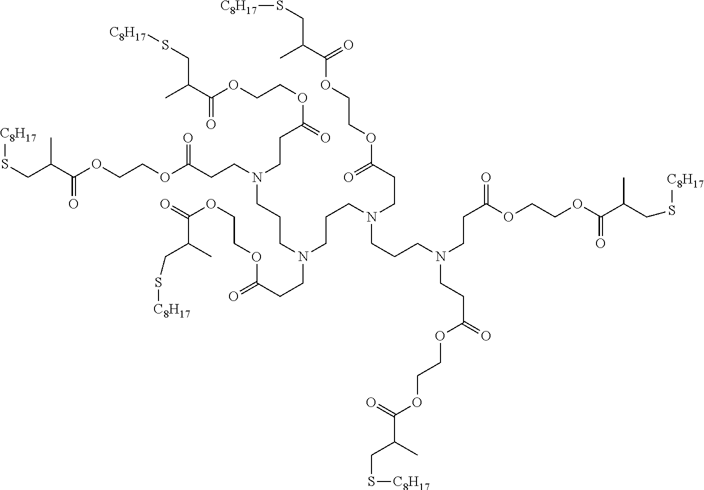 |
| 5A3-2- SC12 (6-arm) | 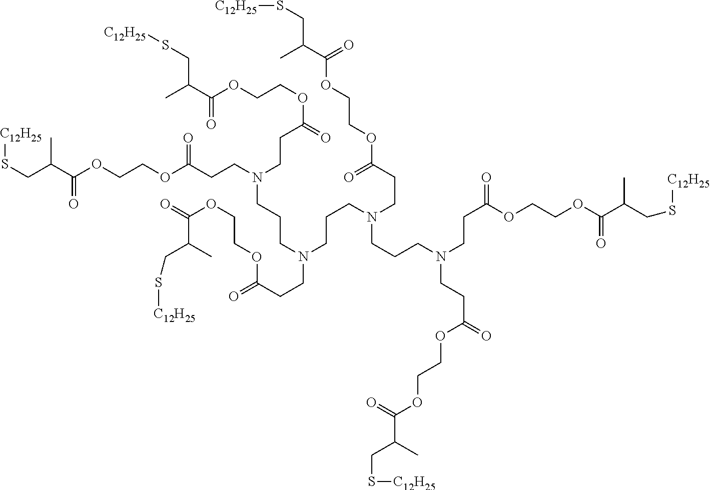 |
| 5A4-2- SC8 (6-arm) | 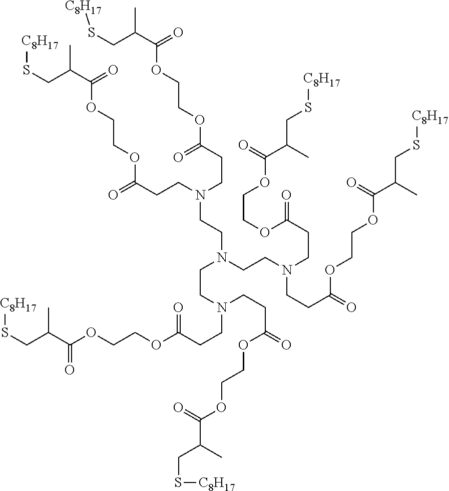 |
| 5A4-2- SC12 (6-arm) | 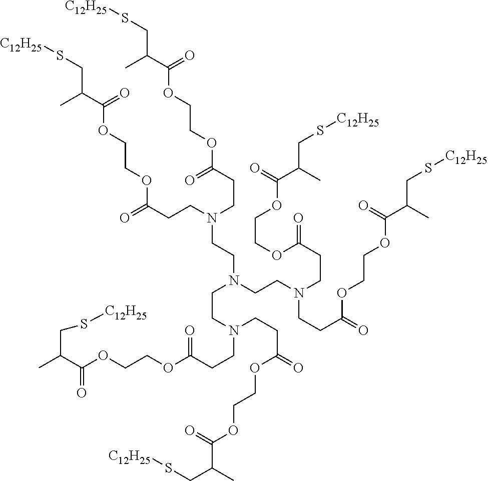 |
| 6A4- SC8 | 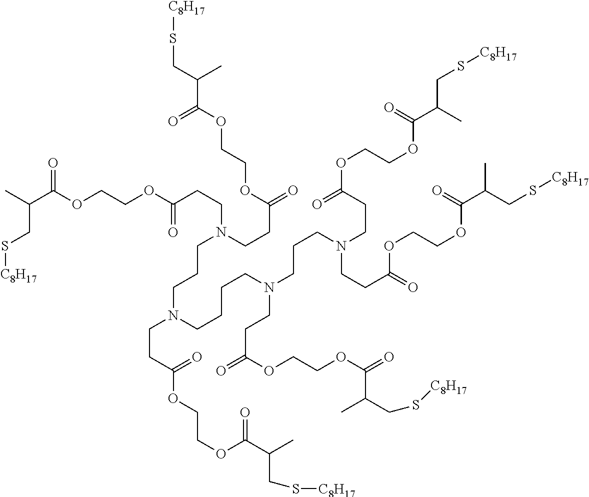 |
| 6A4- SC12 | 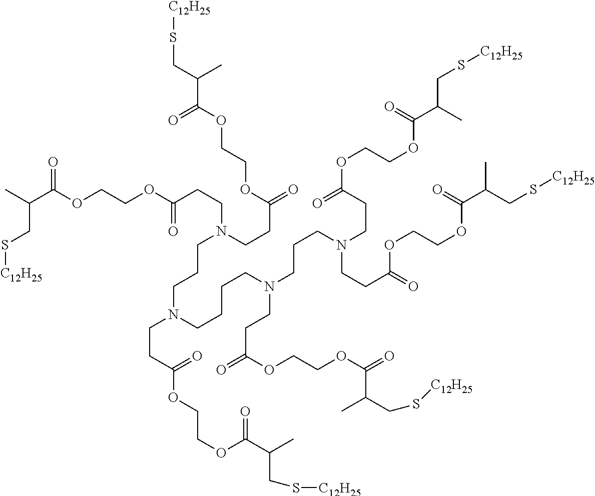 |
| 2A2- g2- SC12 | 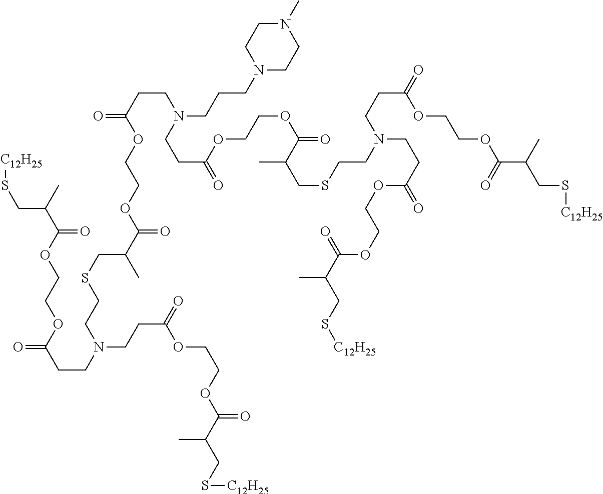 |
| 2A2- g2-SC8 | 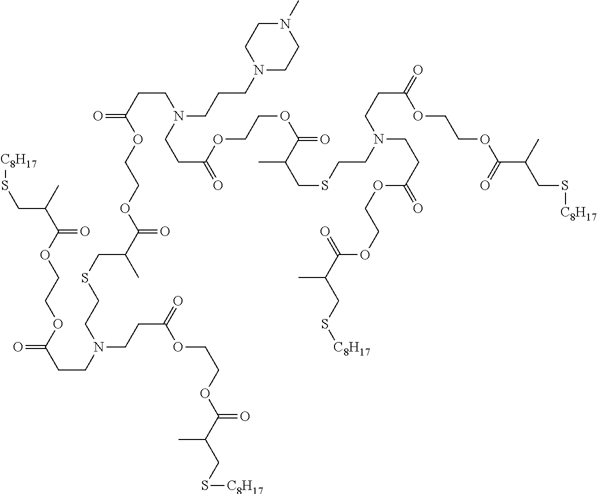 |
| 2A11- g2- SC12 | 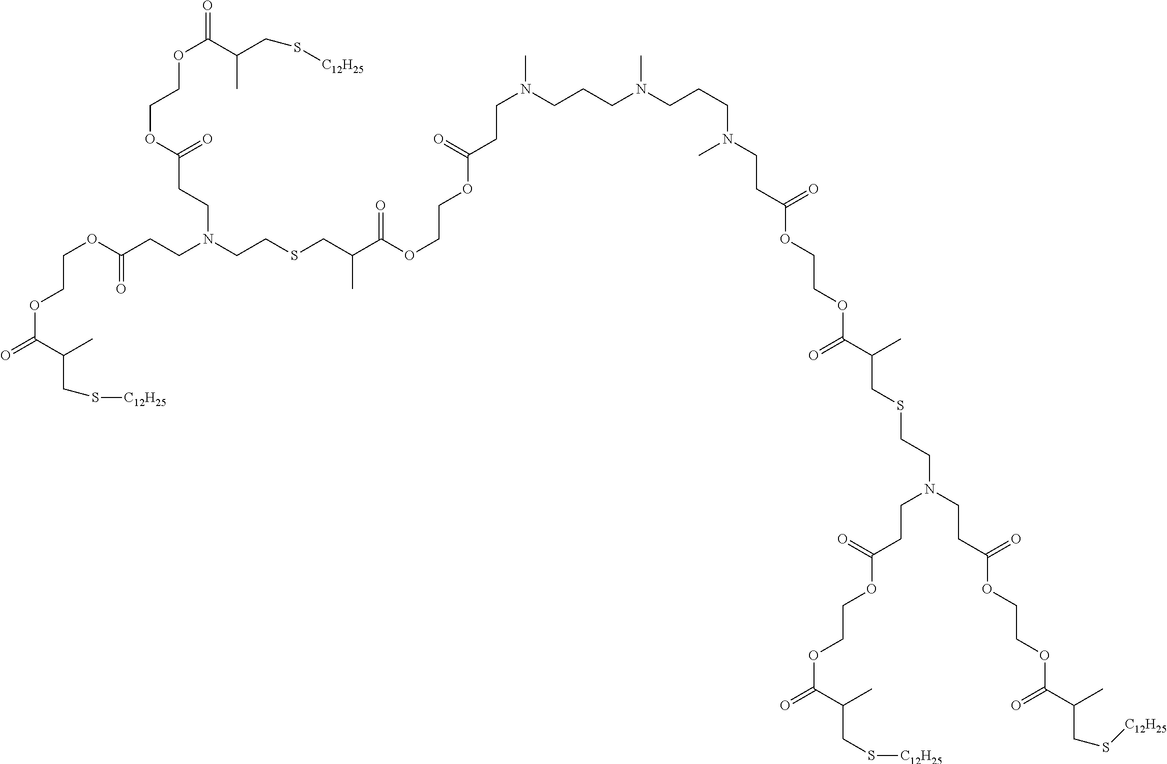 |
| 2A11- g2-SC8 | 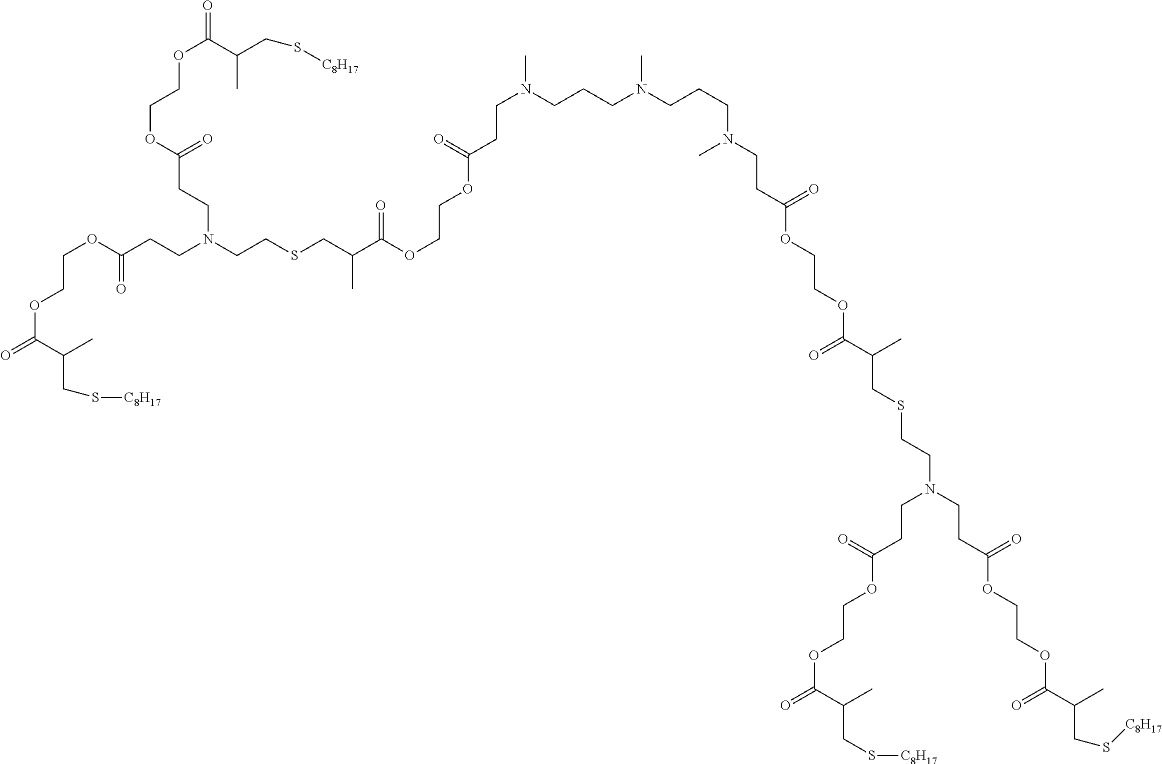 |
| 3A3- g2- SC12 | 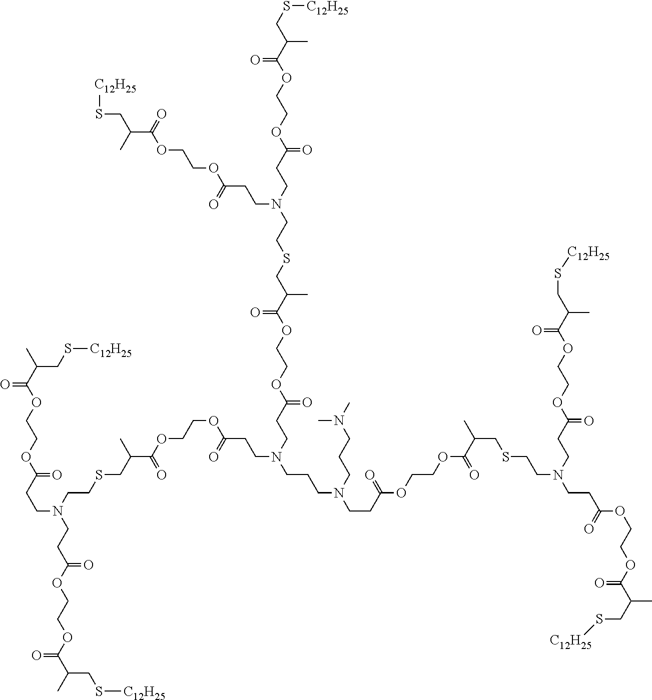 |
| 3A3- g2-SC8 | 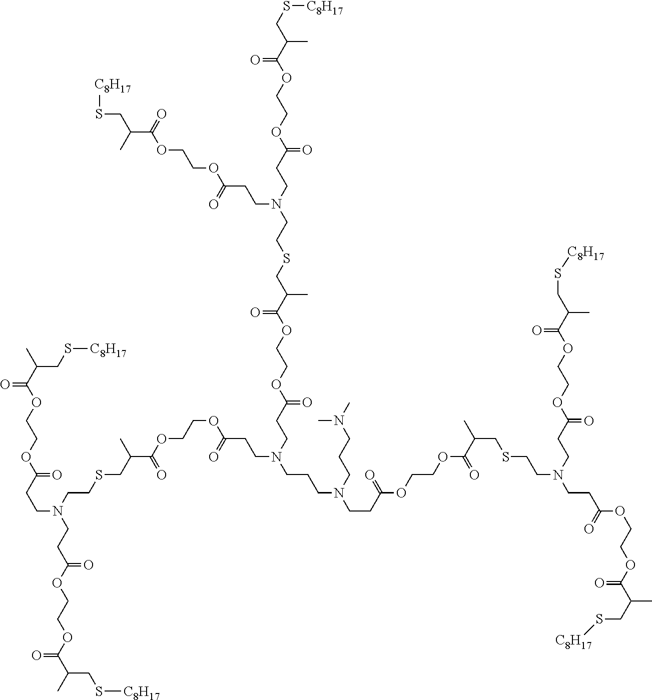 |
| 3A5- g2- SC12 | 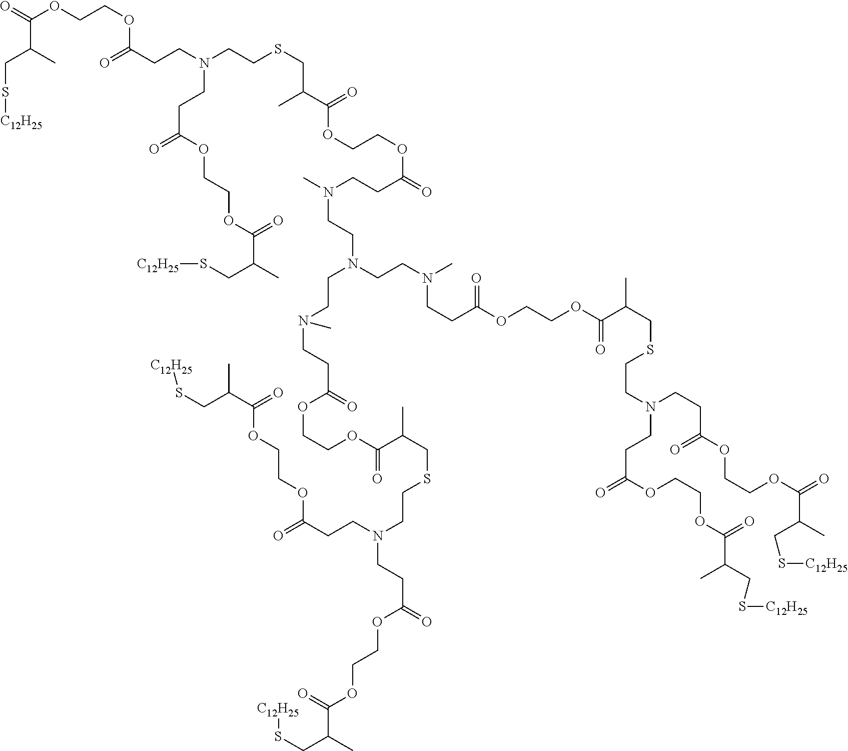 |
| 2A11- g3- SC12 | 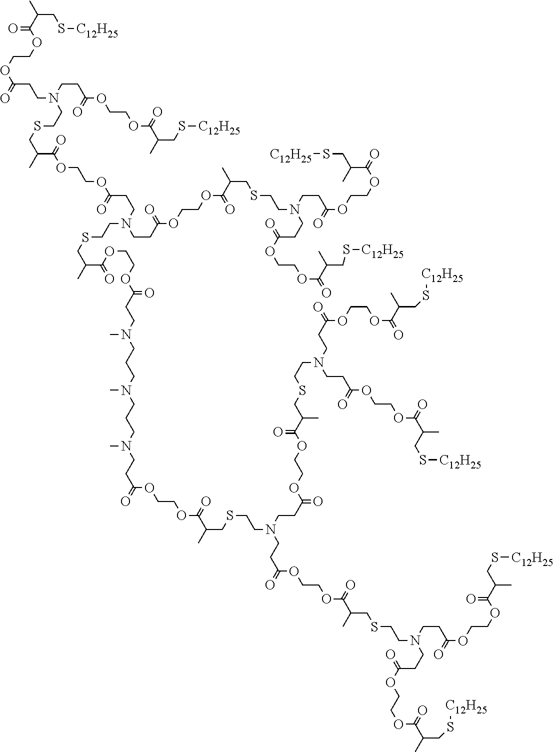 |
| 2A11- g3-SC8 | 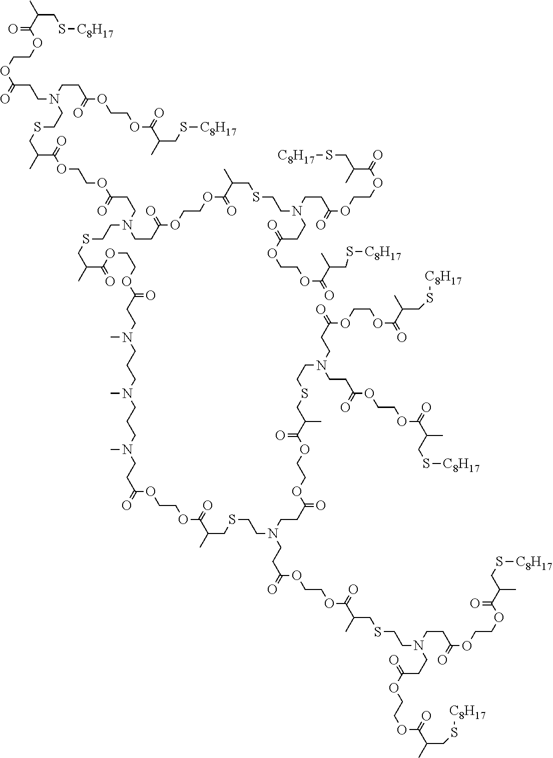 |
| 1A2- g4- SC12 | 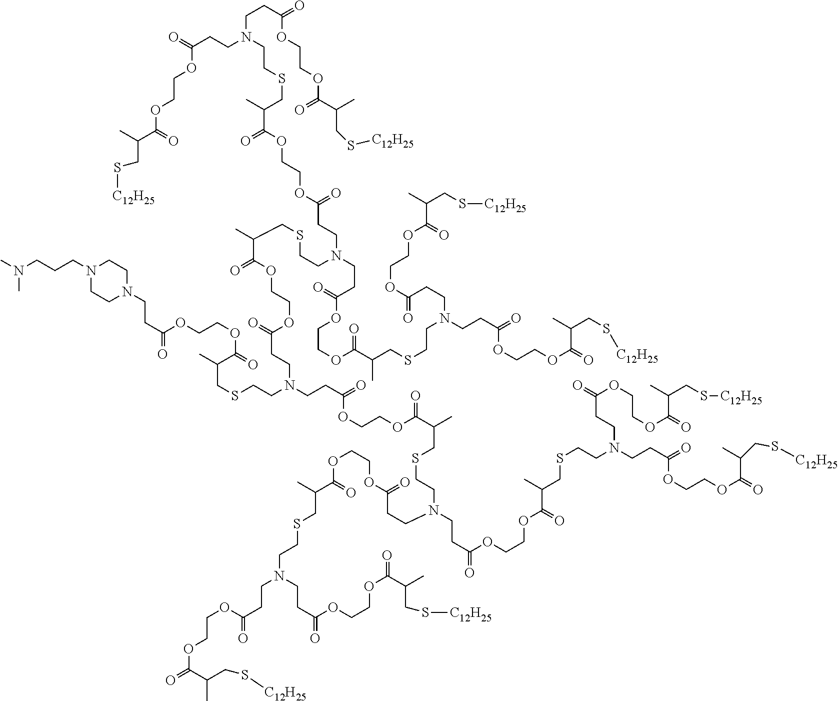 |
| 4A1- g2- SC12 | 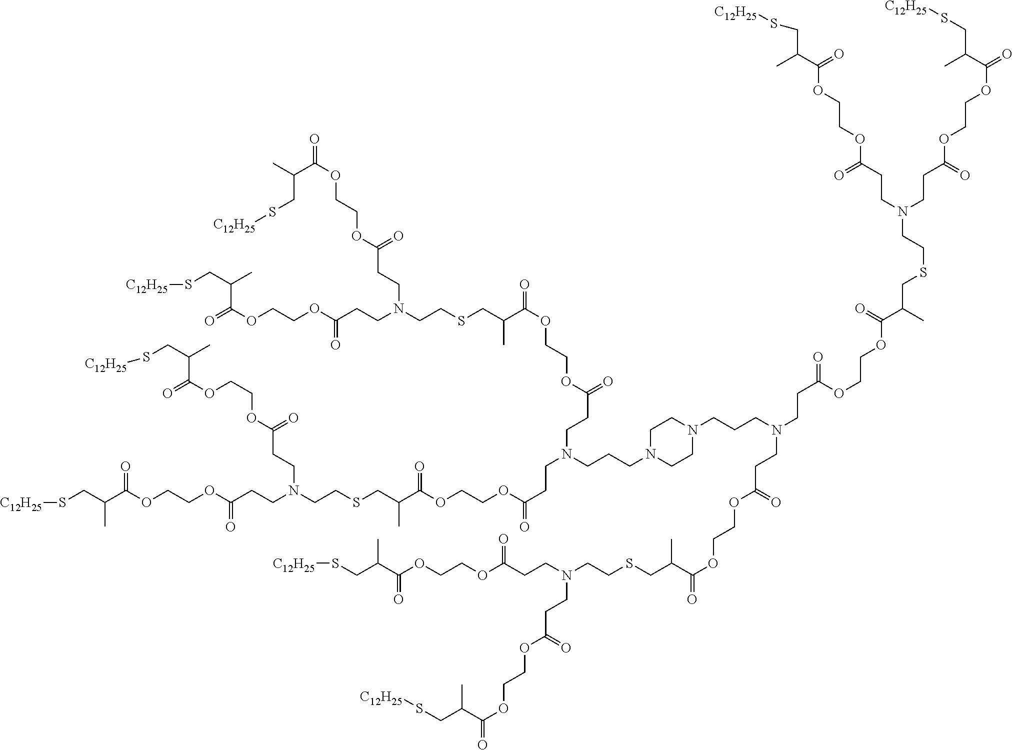 |
| 1A2- g4-SC8 | 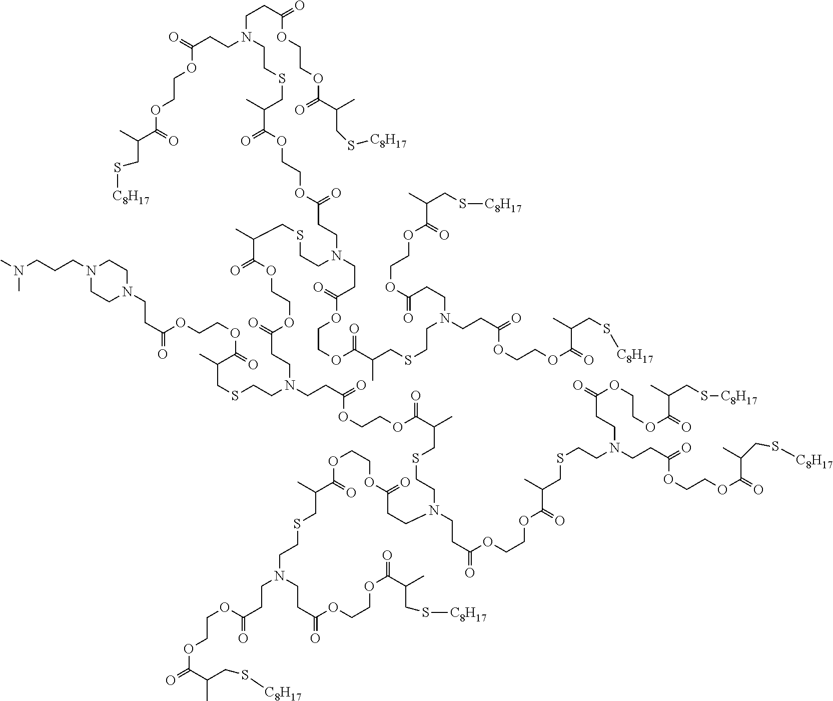 |
| 4A1- g2-SC8 | 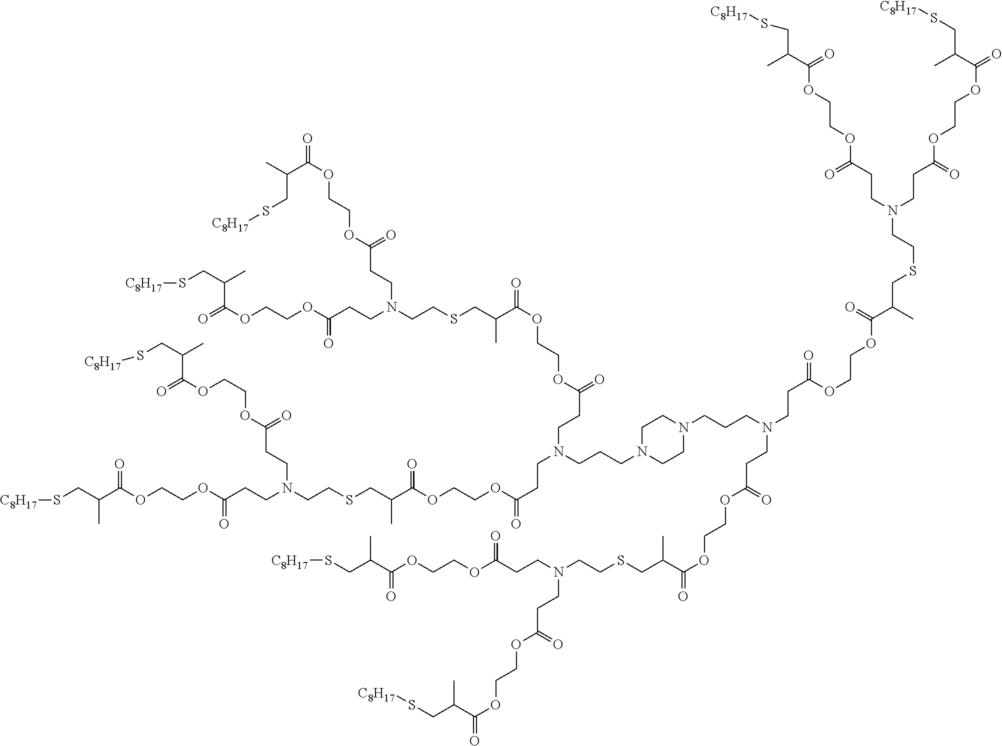 |
| 4A3- g2- SC12 | 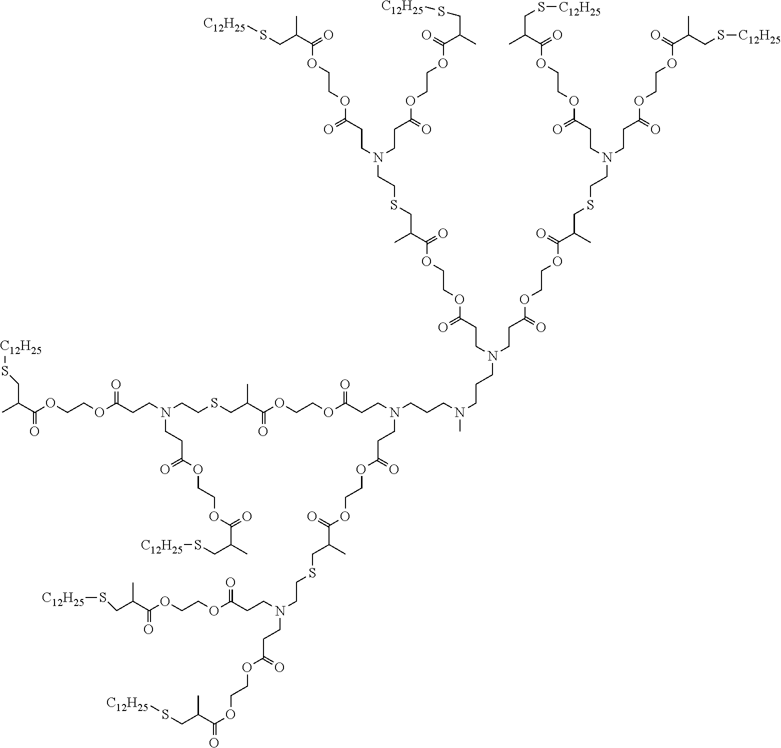 |
| 4A3- g2-SC8 | 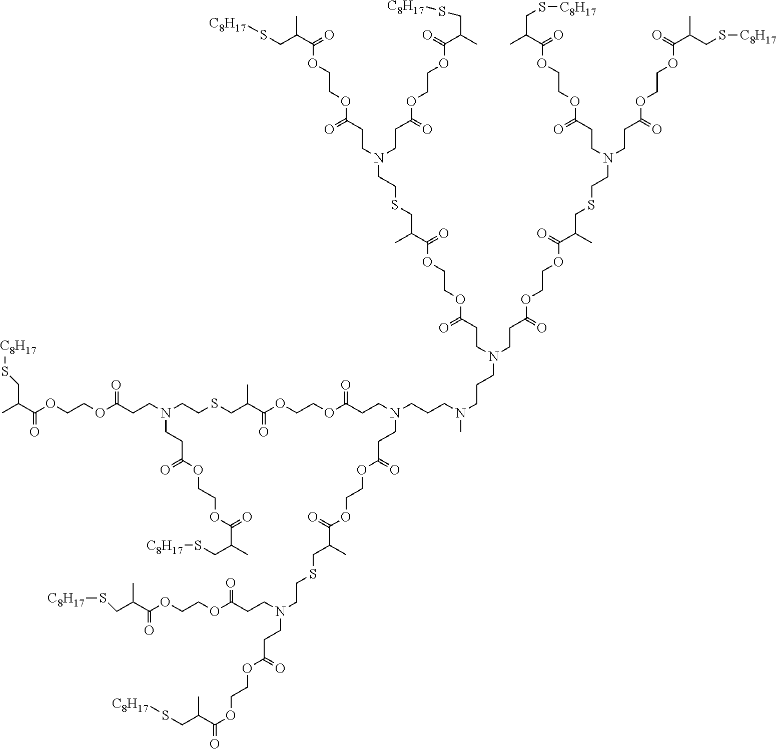 |
| 1A2- g3- SC12 | 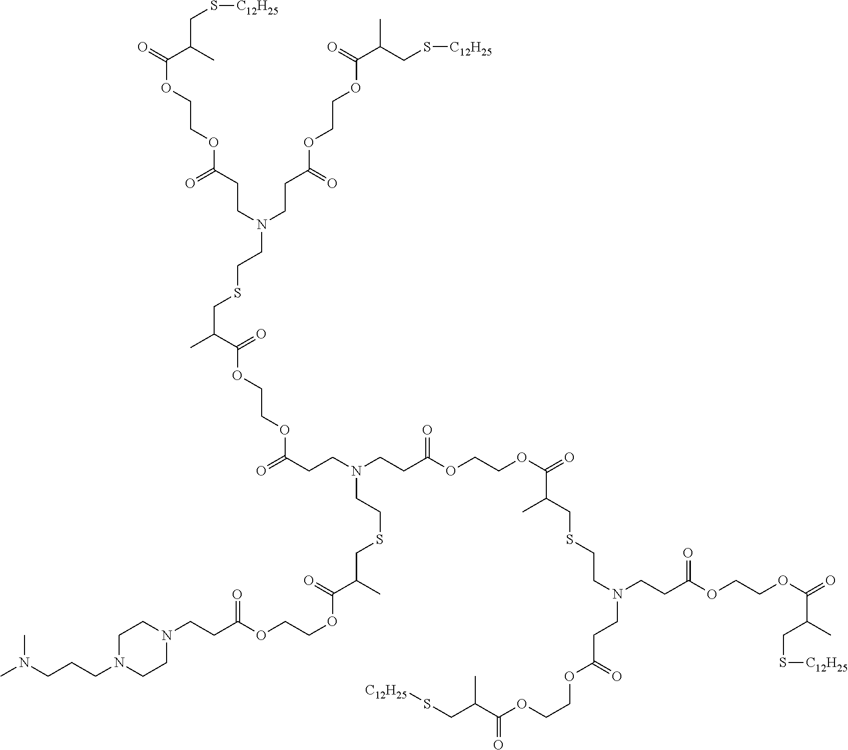 |
| 1A2- g3-SC8 | 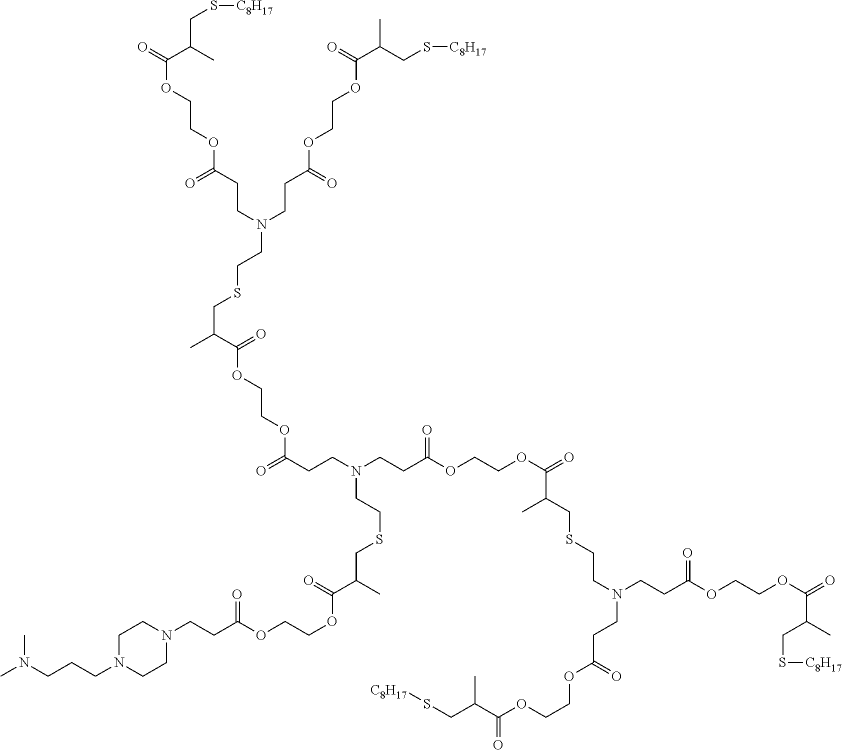 |
| 2A2- g3- SC12 | 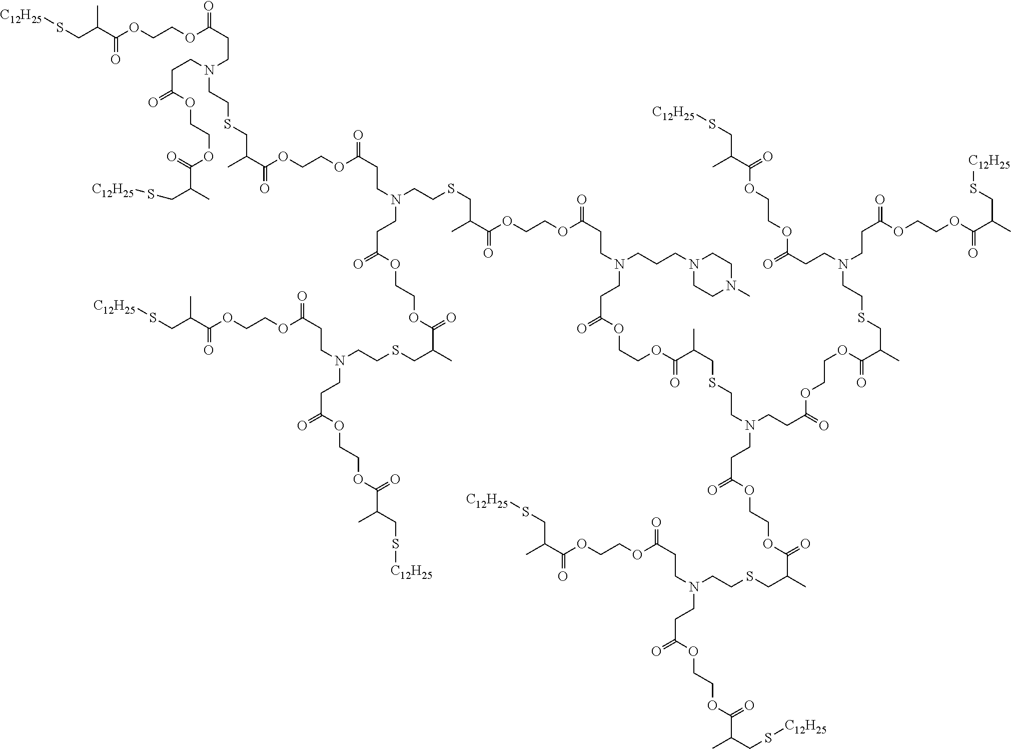 |
| 2A2- g3-SC8 | 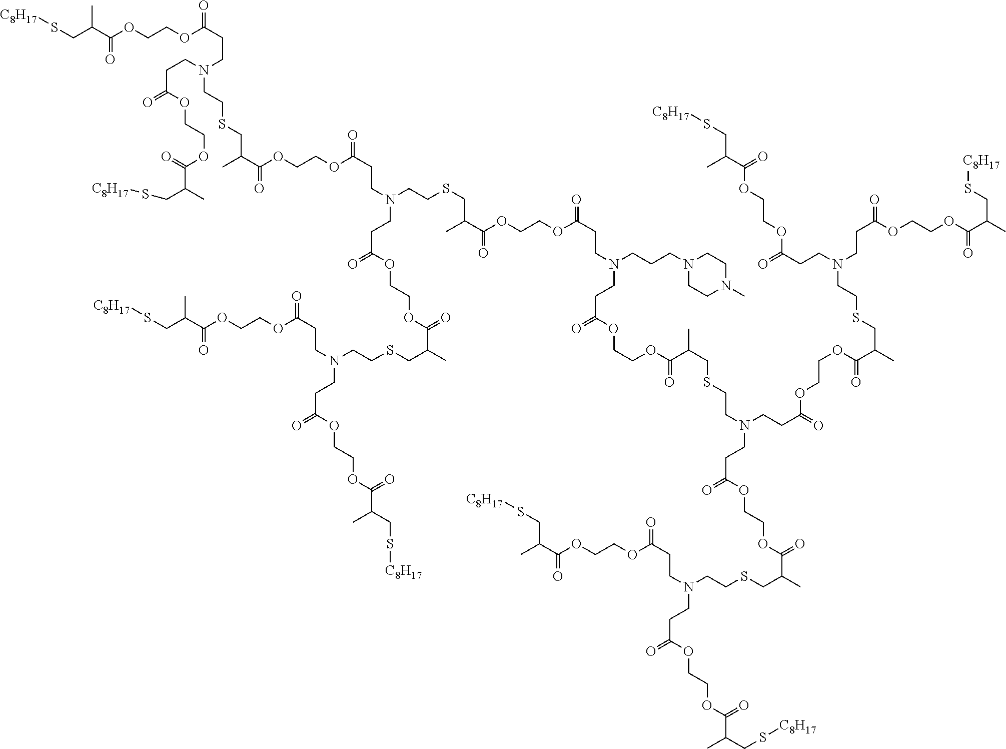 |
| 5A2-4- SC8 (6-arm) | 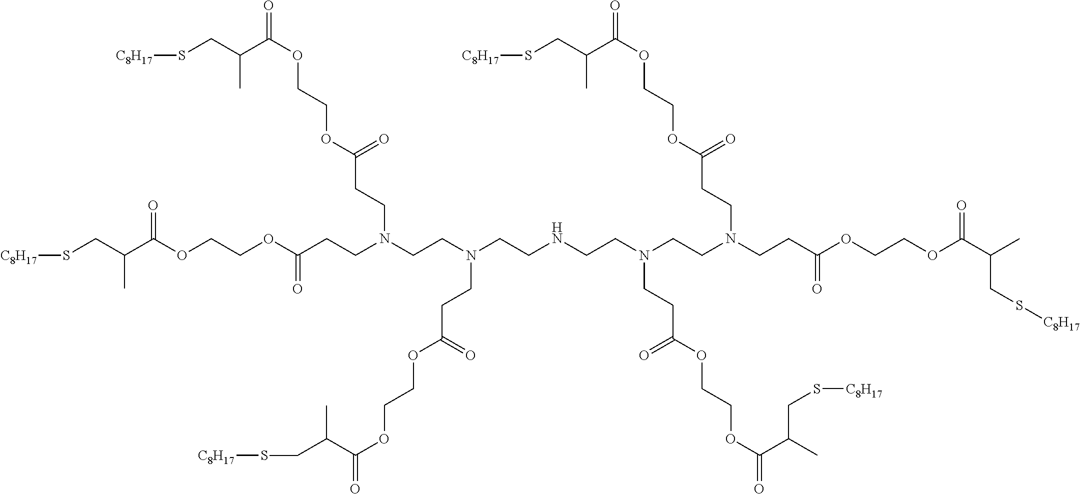 |
| 5A-5- SC8 (6 arm) | 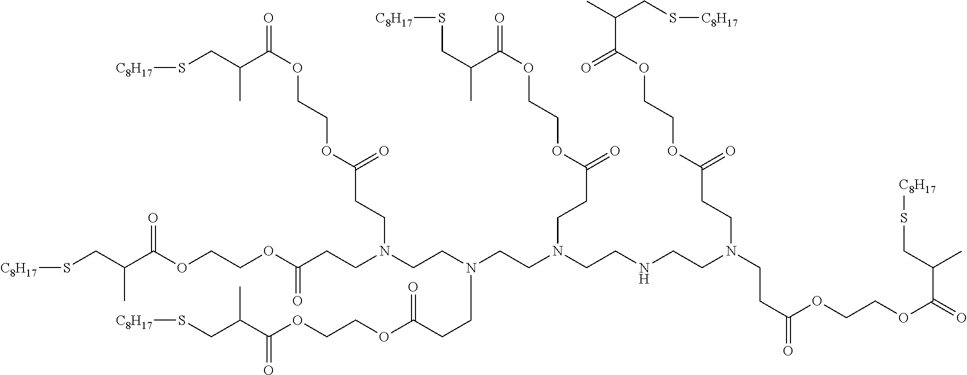 |
| 5A2-6- SC8 (6 arm) | 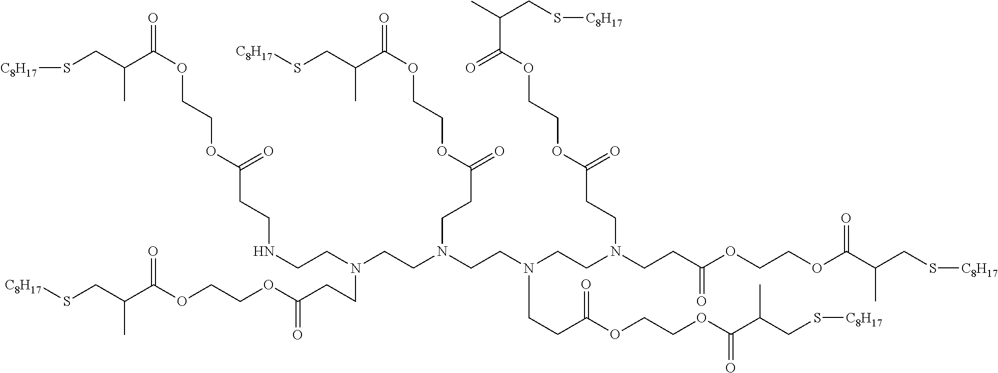 |
| 5A2-1- SC8 (5-arm) | 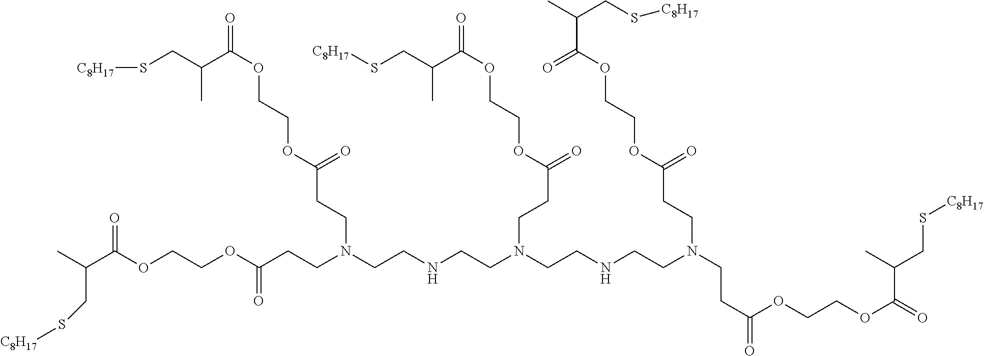 |
| 5A2-2- SC8 | 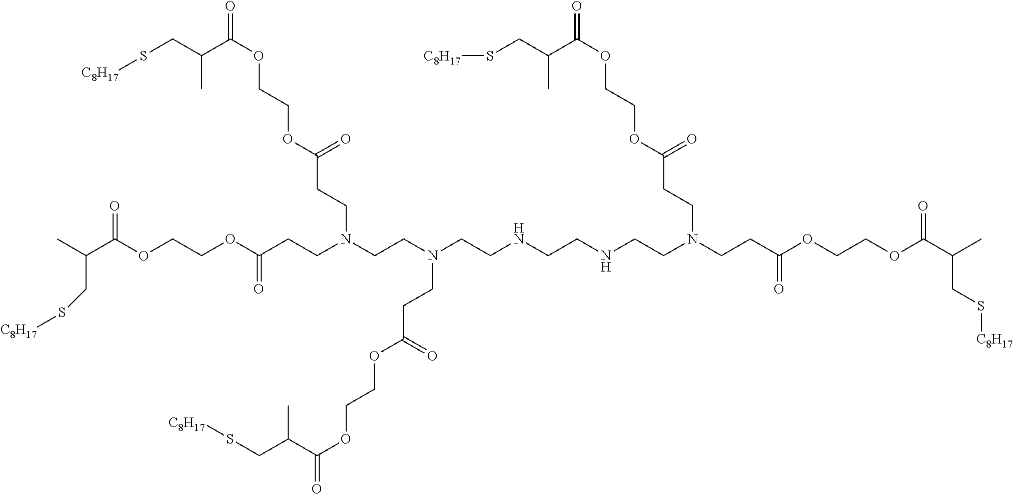 |
| 4A1- SC5 | 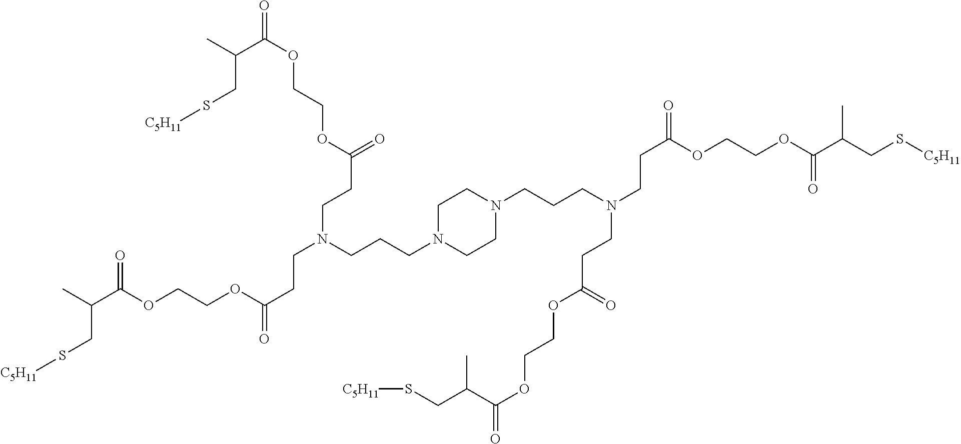 |
| 4A1- SC8 | 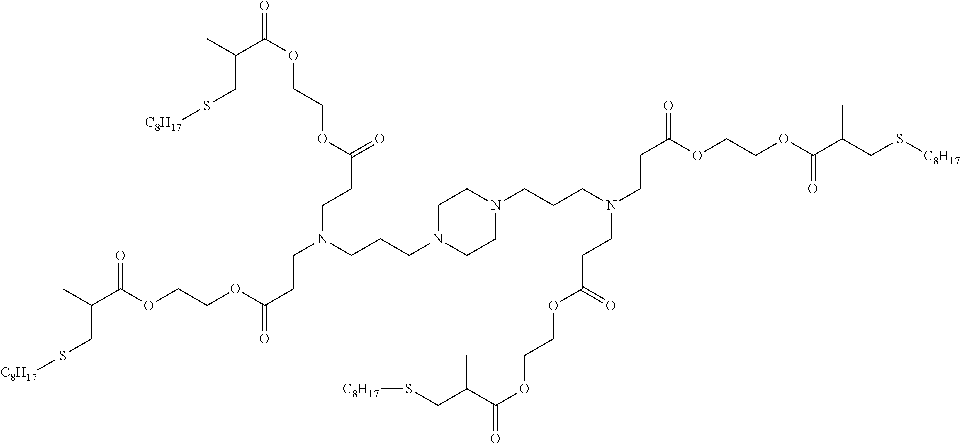 |
| 4A3- SC6 | 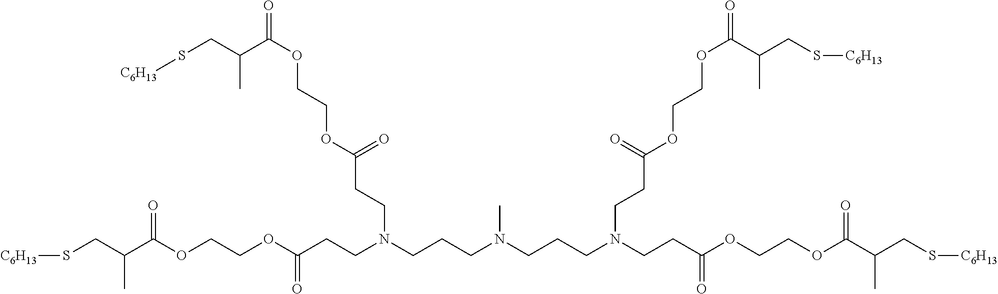 |
| 4A3- SC7 | 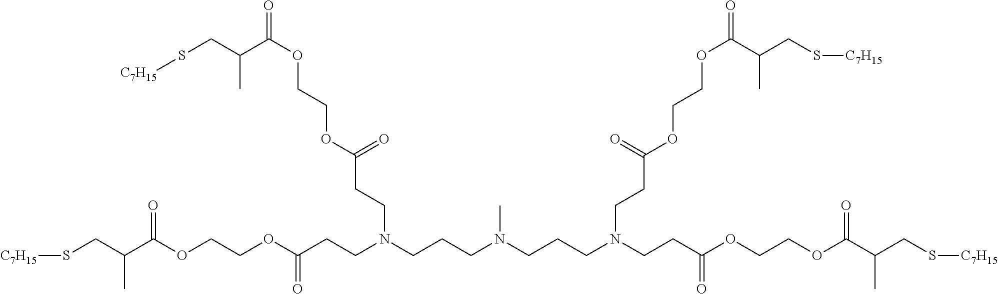 |
| 4A3- SC8 | 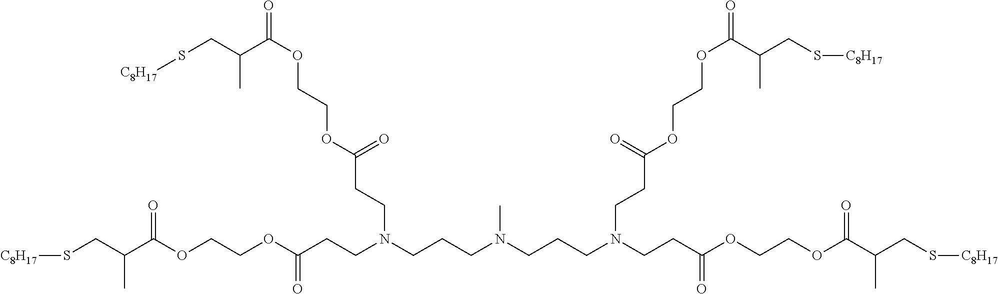 |
| 5A4-2- SC5 (6 arm) | 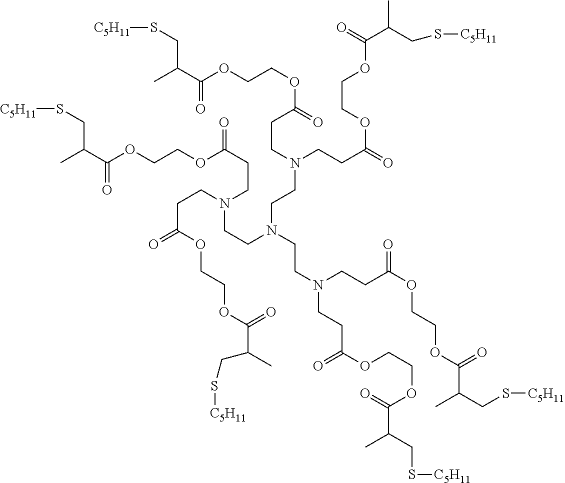 |
| 5A4-2- SC6 (6 arm) | 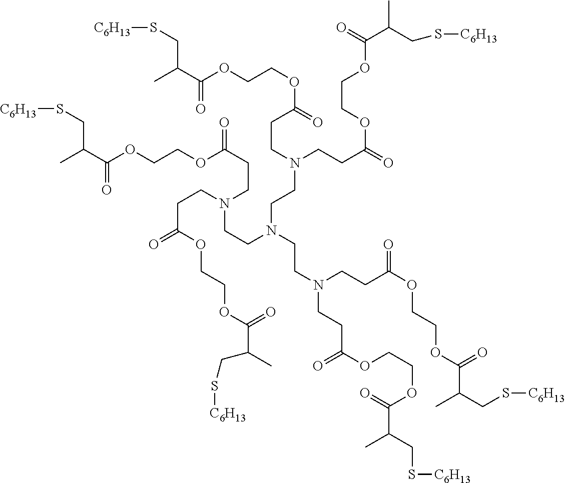 |
| 5A2-4- SC8 (5-arm) | 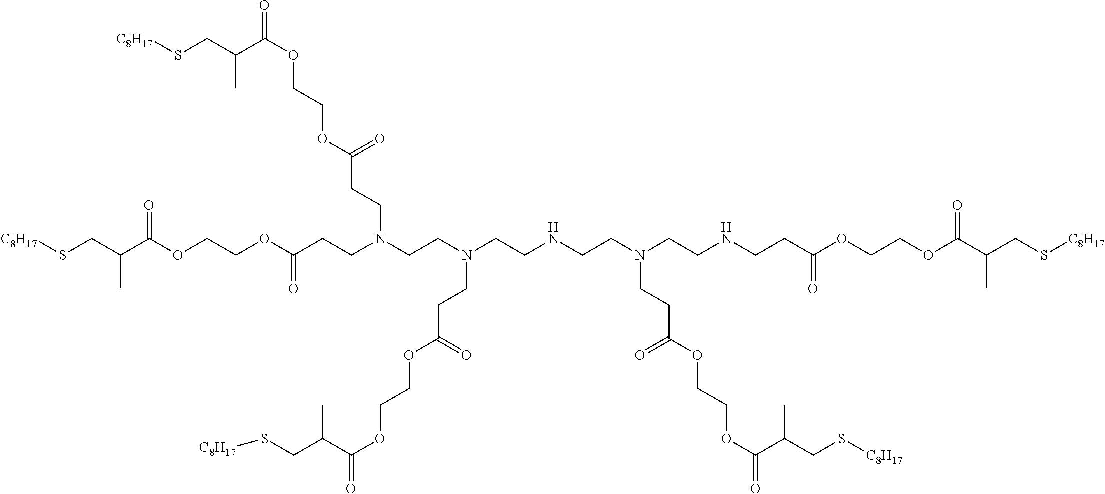 |
| 3A5- g2-SC8 | 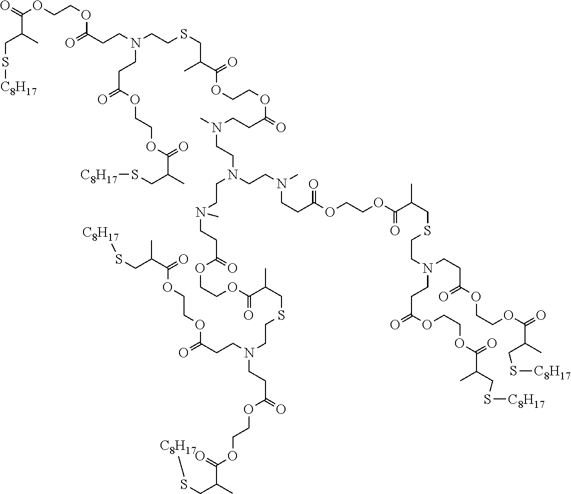 |

### Other Ionizable Cationic Lipids

In some embodiments of the lipid composition, the cationic lipid comprises a structural formula (D-I′):

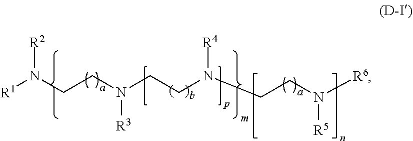

wherein:
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
wherein one or more of the nitrogen atoms indicated in formula (D-I′) may be protonated to provide a cationic lipid.

In some embodiments of the cationic lipid of formula (D-I′), a is 1. In some embodiments of the cationic lipid of formula (D-I′), b is 2. In some embodiments of the cationic lipid of formula (D-I′), m is 1. In some embodiments of the cationic lipid of formula (D-I′), n is 1. In some embodiments of the cationic lipid of formula (D-I′), R¹, R², R³, R⁴, R⁵, and R⁶ are each independently H or —CH₂CH(OH)R⁷. In some embodiments of the cationic lipid of formula (D-I′), R¹, R², R³, R⁴, R⁵, and R⁶ are each independently H or

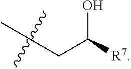

In some embodiments of the cationic lipid of formula (D-I′), R¹, R², R³, R⁴, R⁵, and R⁶ are each independently H or

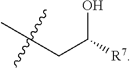

In some embodiments of the cationic lipid of formula (D-I′), R⁷ is C₃-C₁₈ alkyl (e.g., C₆-C₁₂ alkyl).

In some embodiments, the cationic lipid of formula (D-I′) is 13,16,20-tris(2-hydroxydodecyl)-13,16,20,23-tetraazapentatricontane-11,25-diol:

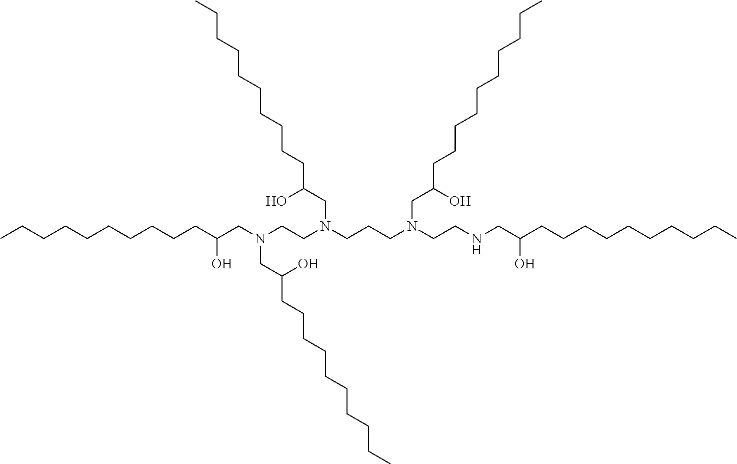 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

In some embodiments, the cationic lipid of formula (D-I′) is (11R,25R)-13,16,20-tris((R)-2-hydroxydodecyl)-13,16,20,23-tetraazapentatricontane-11,25-diol:

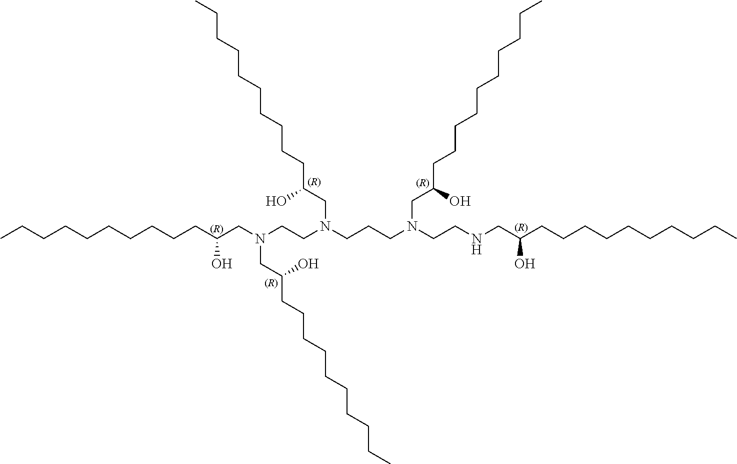

Additional cationic lipids that can be used in the compositions and methods of the present application include those cationic lipids as described in J. Mcclellan, M. C. King, Cell 2010, 141, 210-217, and International Patent Publication WO 2010/144740, WO 2013/149140, WO 2016/118725, WO 2016/118724, WO 2013/063468, WO 2016/205691, WO 2015/184256, WO 2016/004202, WO 2015/199952, WO 2017/004143, WO 2017/075531, WO 2017/117528, WO 2017/049245, WO 2017/173054 and WO 2015/095340, which are incorporated herein by reference for all purposes. Examples of those ionizable cationic lipids include but are not limited to those as shown in Table 5.

TABLE 5
Example ionizable cationic lipids

| # | Structure of example ionizable cationic lipid |
|---|---|
| 1 | 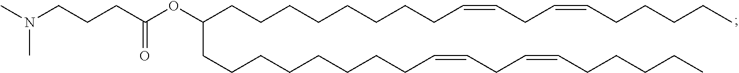 [SMILES: CCCCC/C=C\C/C=C\CCCCCCCCC(CCCCCCCC/C=C\C/C=C\CCCCC)OC(=O)CCCN(C)C] |
| 2 | 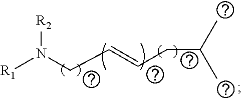 |
| 3 | 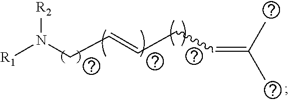 |
| 4 | 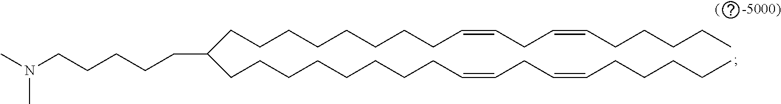 [SMILES: CCCCC/C=C\C/C=C\CCCCCCCCC(CCCCCCCC/C=C\C/C=C\CCCCC)CCCCCN(C)C] |
| 5 | 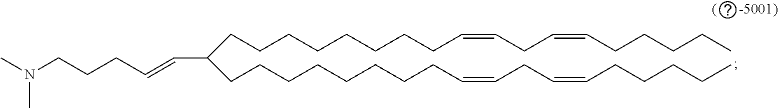 [SMILES: CCCCC/C=C\C/C=C\CCCCCCCCC(/C=C/CCCN(C)C)CCCCCCCC/C=C\C/C=C\CCCCC] |
| 6 | 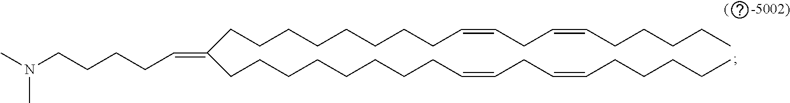 [SMILES: CCCCC/C=C\C/C=C\CCCCCCCCC(=CCCCCN(C)C)CCCCCCCC/C=C\C/C=C\CCCCC] |
| 7 | 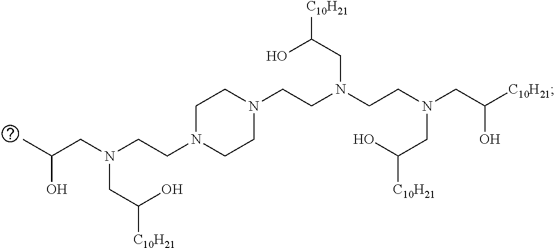 |
| 9 | 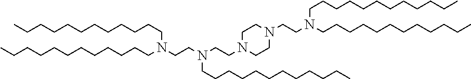 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1] |
| 10 | 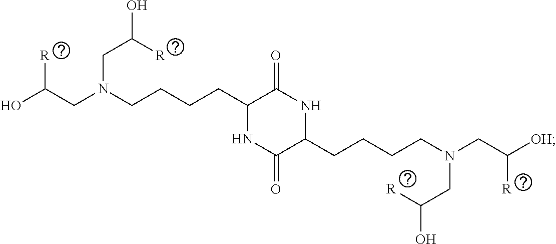 |
| 11 | 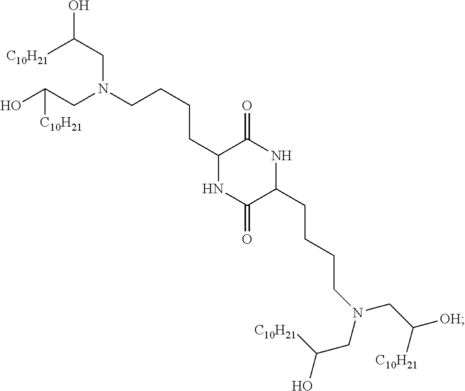 |
| 12 | 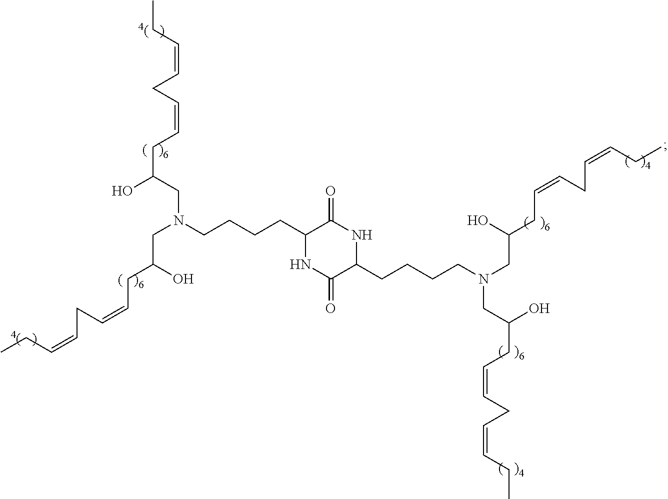 |
| 13 | 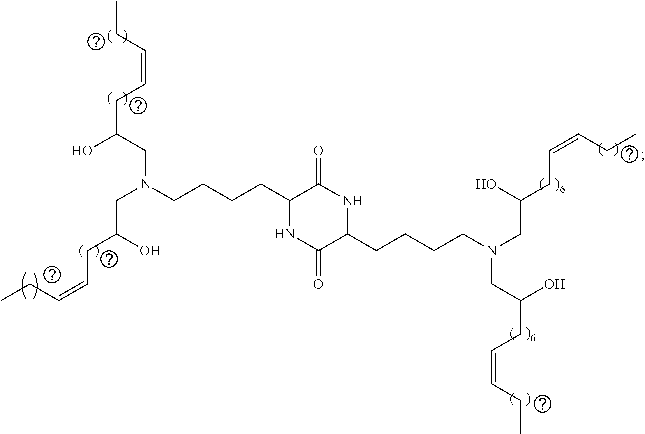 |
| 14 | 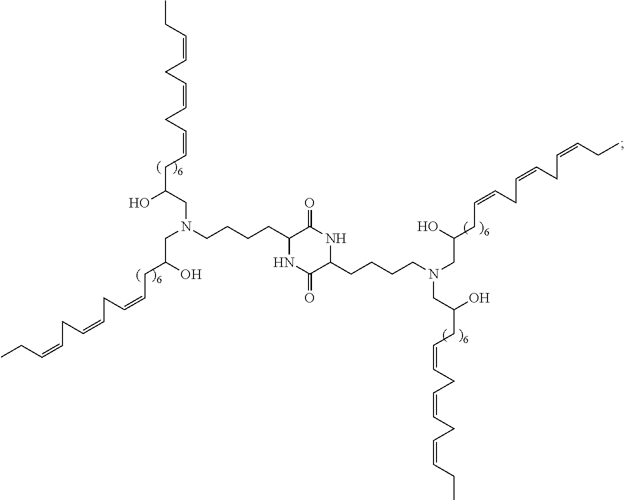 |
| 15 | 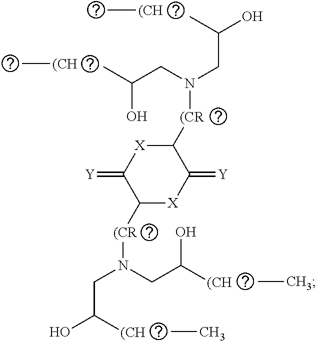 |
| 16 | 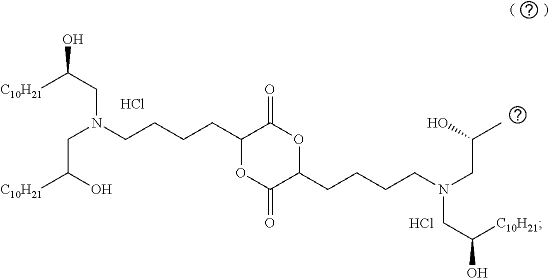 |
| 17 | 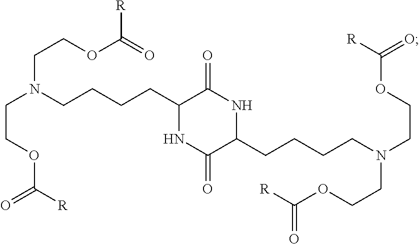 |
| 18 | 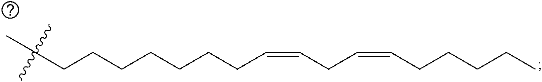 [SMILES: *CCCCCCC/C=C\C/C=C\CCCCC] |
| 19 | 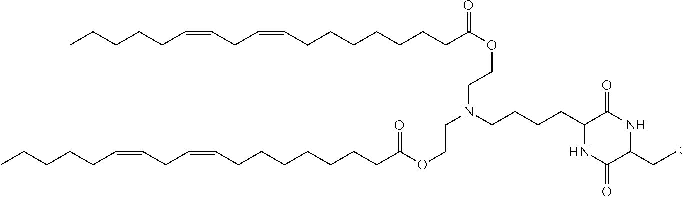 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC] |
| 20 | 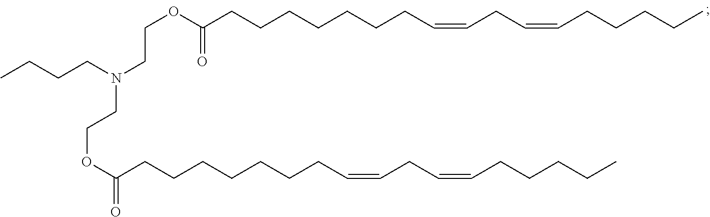 [SMILES: CCCCC/C=C\C/C=C\CCCCCCCC(=O)OCCN(CCCC)CCOC(=O)CCCCCCC/C=C\C/C=C\CCCCC] |
| 21 | 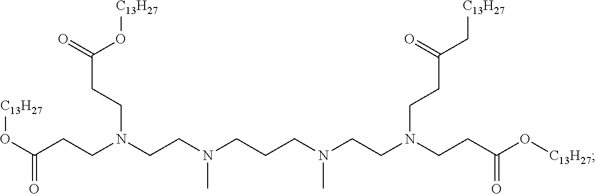 |
| 22 | 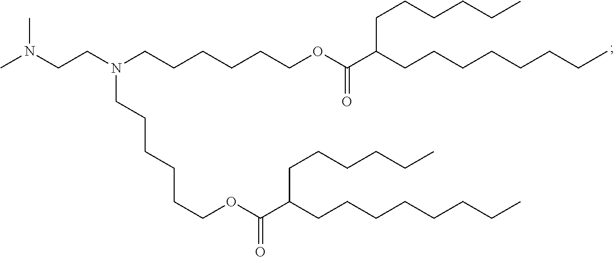 [SMILES: CCCCCCCCC(CCCCCC)C(=O)OCCCCCCN(CCCCCCOC(=O)C(CCCCCC)CCCCCCCC)CCN(C)C] |
| 23 | 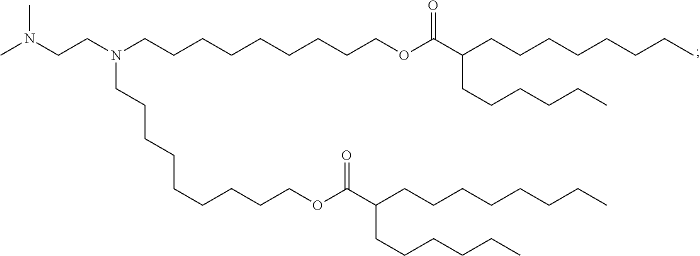 [SMILES: CCCCCCCCC(CCCCCC)C(=O)OCCCCCCCCCN(CCCCCCCCCOC(=O)C(CCCCCC)CCCCCCCC)CCN(C)C] |
| 24 | 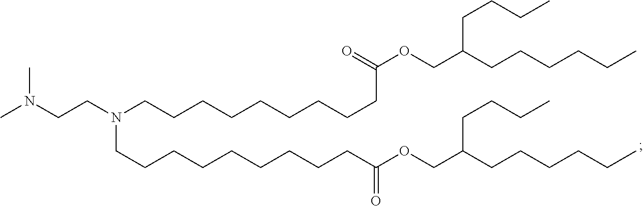 [SMILES: CCCCCCC(CCCC)COC(=O)CCCCCCCCCN(CCCCCCCCCC(=O)OCC(CCCC)CCCCCC)CCN(C)C] |
| 25 | 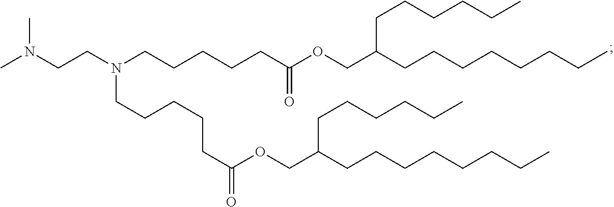 [SMILES: CCCCCCCCC(CCCCCC)COC(=O)CCCCCN(CCCCCC(=O)OCC(CCCCCC)CCCCCCCC)CCN(C)C] |
| 26 | 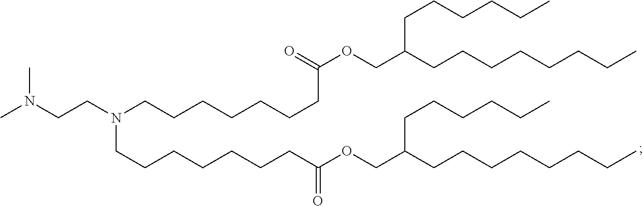 [SMILES: CCCCCCCCC(CCCCCC)COC(=O)CCCCCCCN(CCCCCCCC(=O)OCC(CCCCCC)CCCCCCCC)CCN(C)C] |
| 27 | 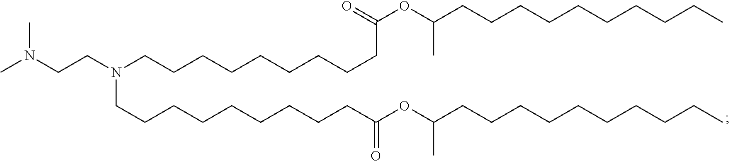 [SMILES: CCCCCCCCCCC(C)OC(=O)CCCCCCCCCN(CCCCCCCCCC(=O)OC(C)CCCCCCCCCC)CCN(C)C] |
| 28 | 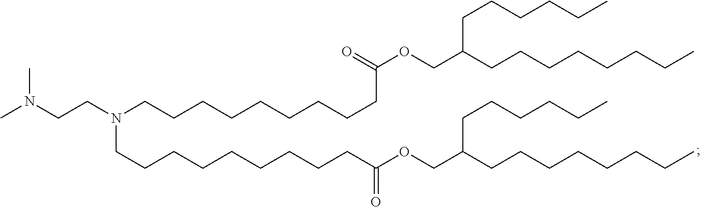 [SMILES: CCCCCCCCC(CCCCCC)COC(=O)CCCCCCCCCN(CCCCCCCCCC(=O)OCC(CCCCCC)CCCCCCCC)CCN(C)C] |
| 29 | 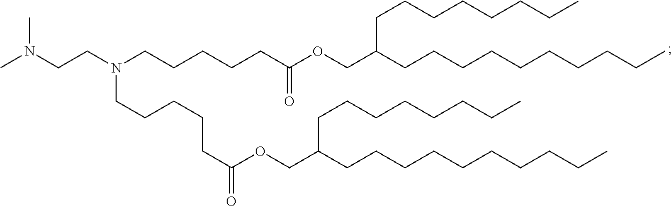 [SMILES: CCCCCCCCCCC(CCCCCCCC)COC(=O)CCCCCN(CCCCCC(=O)OCC(CCCCCCCC)CCCCCCCCCC)CCN(C)C] |
| 30 | 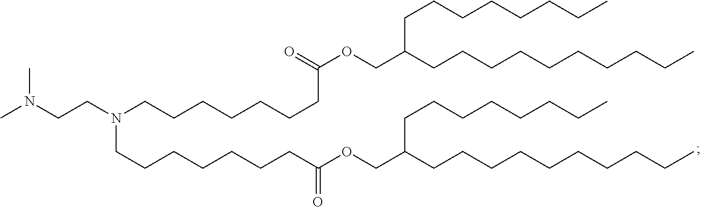 [SMILES: CCCCCCCCCCC(CCCCCCCC)COC(=O)CCCCCCCN(CCCCCCCC(=O)OCC(CCCCCCCC)CCCCCCCCCC)CCN(C)C] |
| 31 | 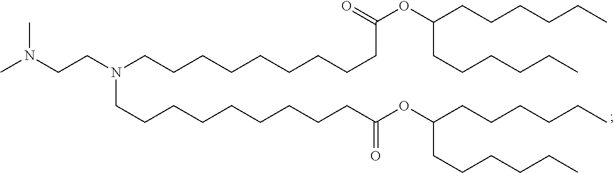 [SMILES: CCCCCCC(CCCCCC)OC(=O)CCCCCCCCCN(CCCCCCCCCC(=O)OC(CCCCCC)CCCCCC)CCN(C)C] |
| 32 | 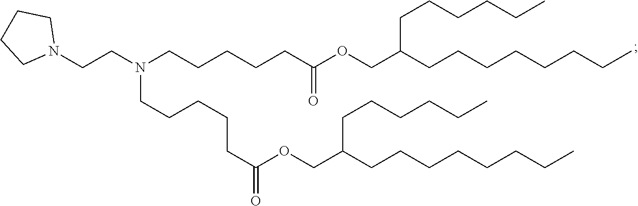 [SMILES: CCCCCCCCC(CCCCCC)COC(=O)CCCCCN(CCCCCC(=O)OCC(CCCCCC)CCCCCCCC)CCN1CCCC1] |
| 33 | 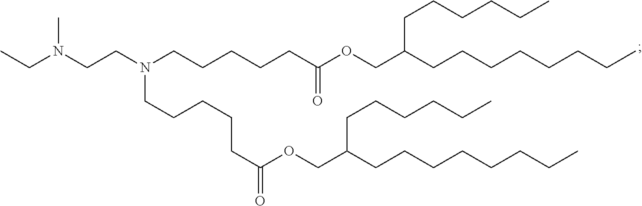 [SMILES: CCCCCCCCC(CCCCCC)COC(=O)CCCCCN(CCCCCC(=O)OCC(CCCCCC)CCCCCCCC)CCN(C)CC] |
| 34 | 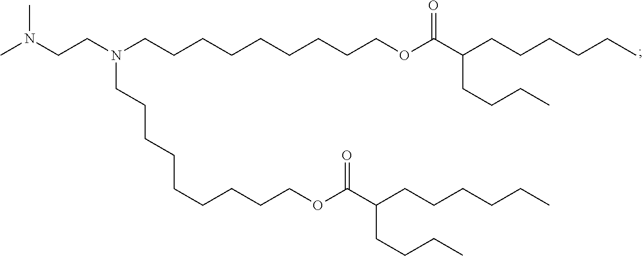 [SMILES: CCCCCCC(CCCC)C(=O)OCCCCCCCCCN(CCCCCCCCCOC(=O)C(CCCC)CCCCCC)CCN(C)C] |
| 35 | 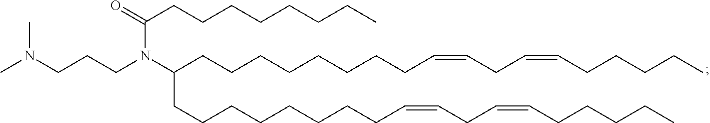 [SMILES: CCCCC/C=C\C/C=C\CCCCCCCCC(CCCCCCCC/C=C\C/C=C\CCCCC)N(CCCN(C)C)C(=O)CCCCCCCC] |
| 36 | 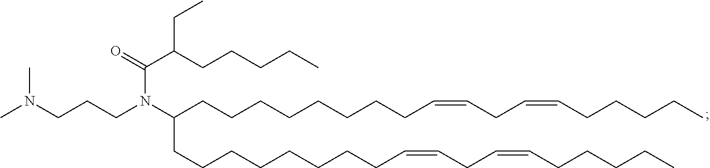 [SMILES: CCCCC/C=C\C/C=C\CCCCCCCCC(CCCCCCCC/C=C\C/C=C\CCCCC)N(CCCN(C)C)C(=O)C(CC)CCCCC] |
| 37 | 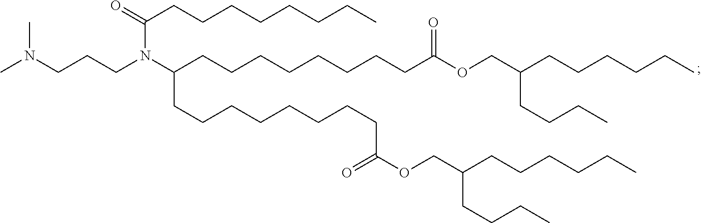 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] |
| 38 | 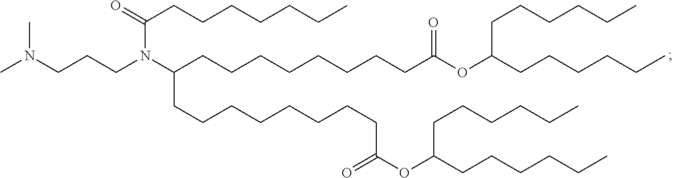 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |
| 39 | 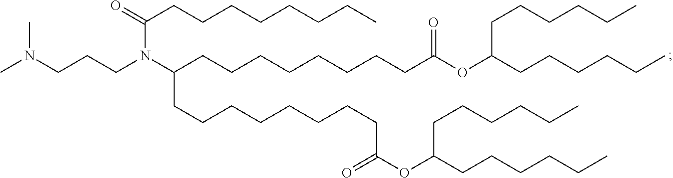 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] |
| 40 | 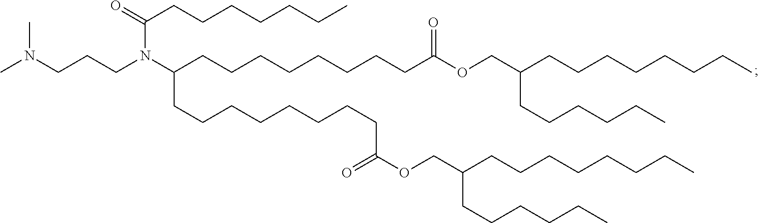 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] |
| 41 | 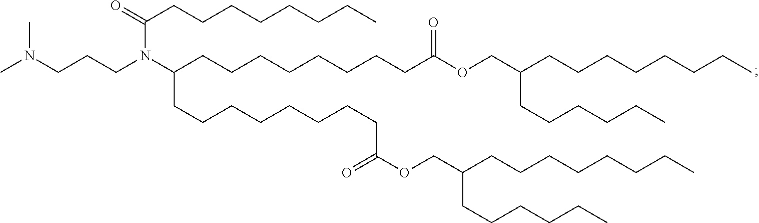 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] |
| 42 | 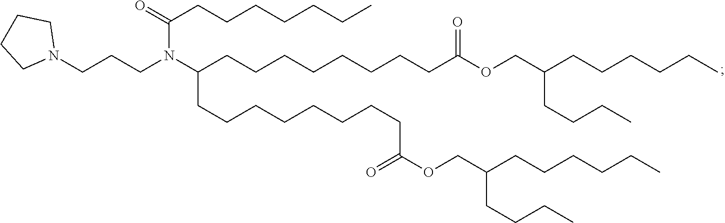 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] |
| 43 | 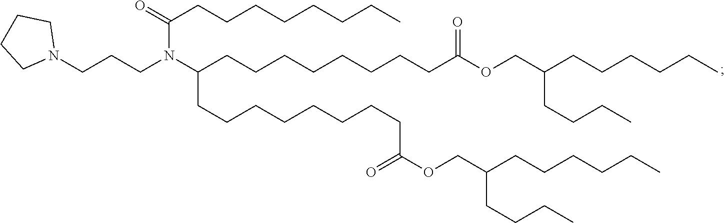 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] |
| 44 | 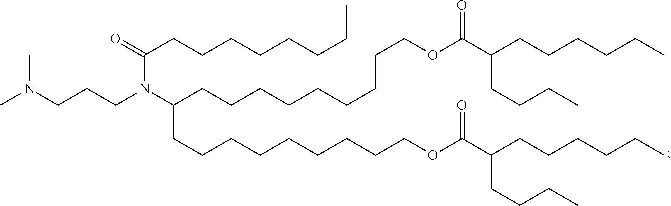 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] |
| 45 | 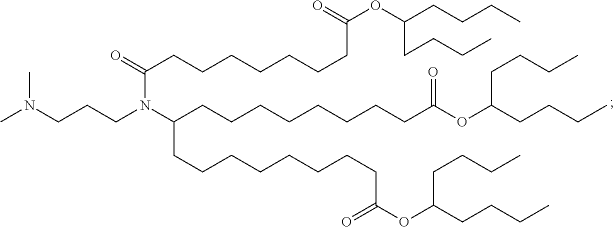 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] |
| 46 | 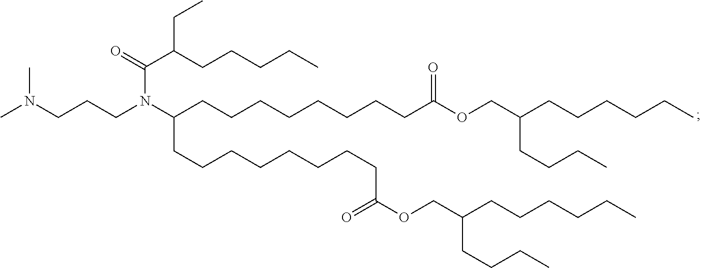 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC] |
| 47 | 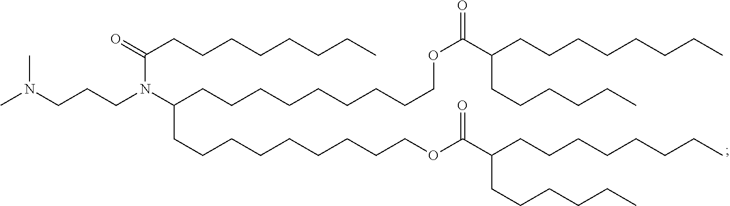 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC] |
| 48 | 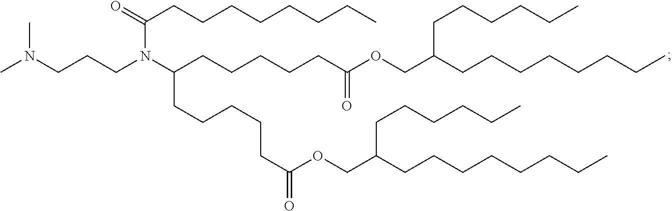 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC] |
| 49 | 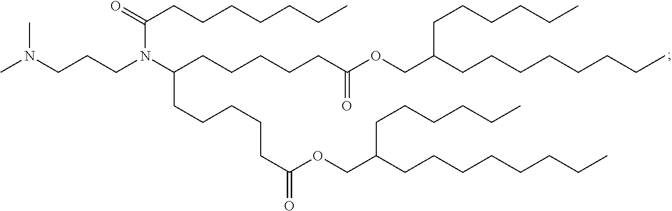 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC] |
| 50 | 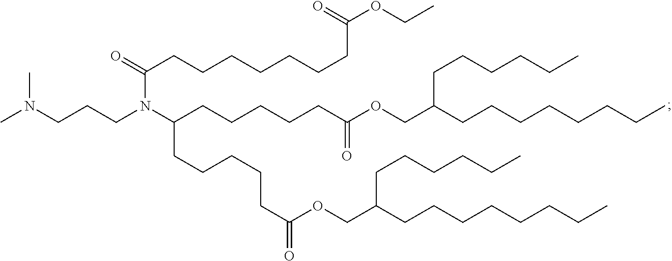 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC] |
| 51 | 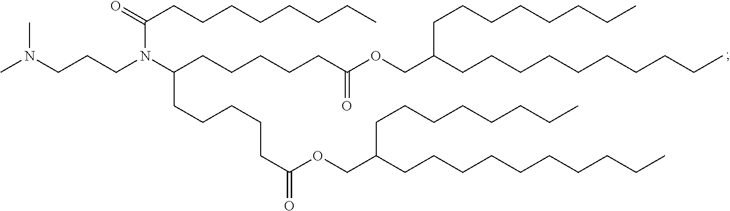 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC] |
| 52 | 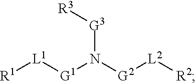 |
| 53 | 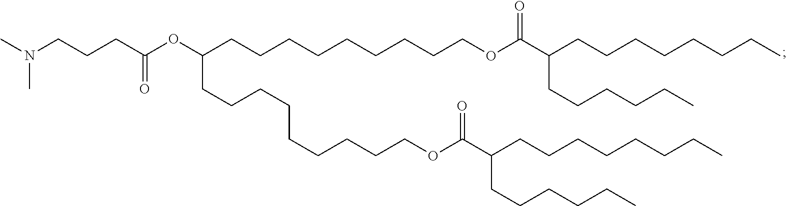 [SMILES: CCCCCCCCC(CCCCCC)C(=O)OCCCCCCCCCC(CCCCCCCCCOC(=O)C(CCCCCC)CCCCCCCC)OC(=O)CCCN(C)C] |
| 54 | 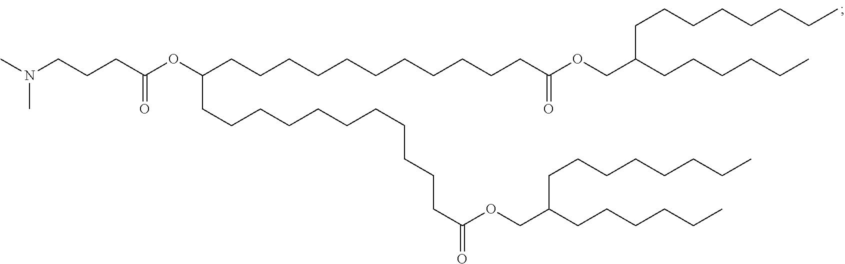 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C] |
| 55 | 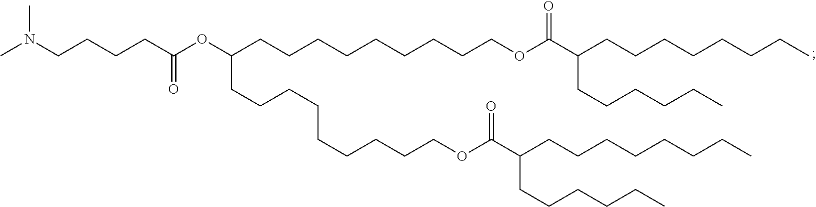 [SMILES: CCCCCCCCC(CCCCCC)C(=O)OCCCCCCCCCC(CCCCCCCCCOC(=O)C(CCCCCC)CCCCCCCC)OC(=O)CCCCN(C)C] |
| 56 | 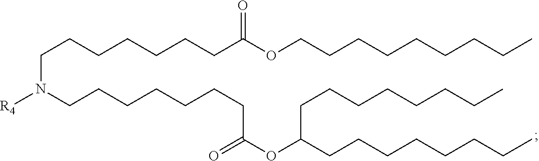 |
| 57 | 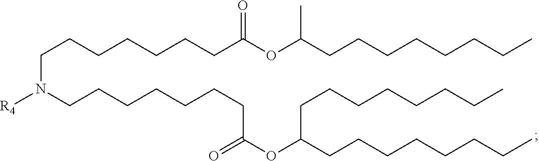 |
| 58 | 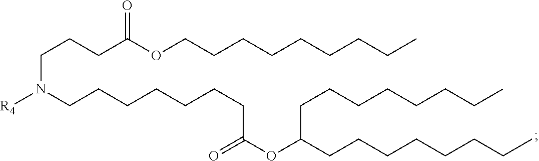 |
| 59 | 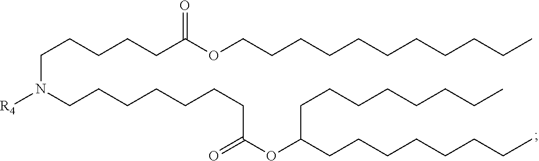 |
| 60 | 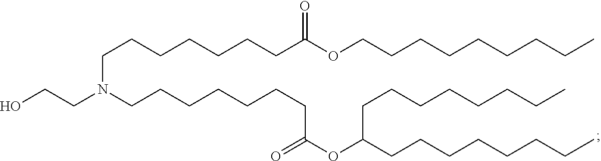 [SMILES: CCCCCCCCCOC(=O)CCCCCCCN(CCO)CCCCCCCC(=O)OC(CCCCCCCC)CCCCCCCC] |
| 61 | 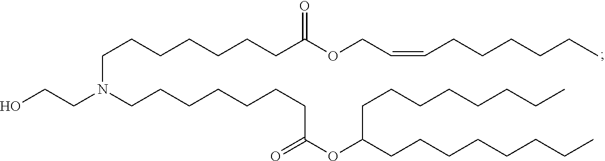 [SMILES: CCCCCC/C=C\COC(=O)CCCCCCCN(CCO)CCCCCCCC(=O)OC(CCCCCCCC)CCCCCCCC] |
| 62 | 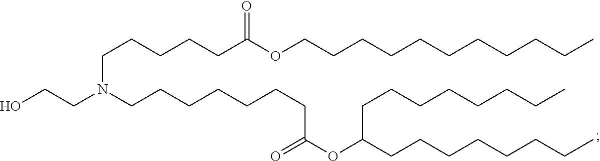 [SMILES: CCCCCCCCCCCOC(=O)CCCCCN(CCO)CCCCCCCC(=O)OC(CCCCCCCC)CCCCCCCC] |
| 63 | 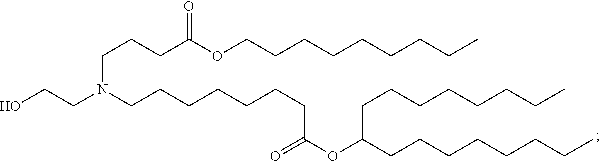 [SMILES: CCCCCCCCCOC(=O)CCCN(CCO)CCCCCCCC(=O)OC(CCCCCCCC)CCCCCCCC] |
| 64 | 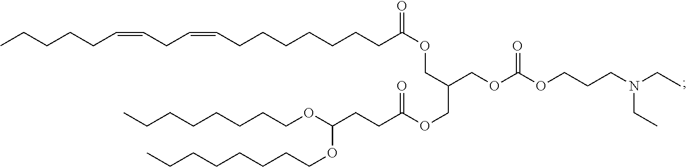 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 65 | 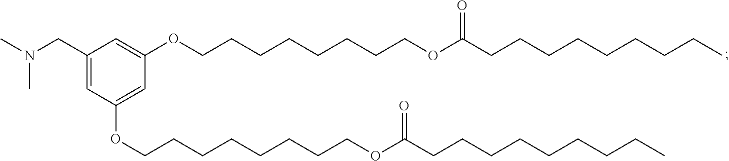 [SMILES: CCCCCCCCCC(=O)OCCCCCCCCOc1cc(CN(C)C)cc(OCCCCCCCCOC(=O)CCCCCCCCC)c1] |
| 66 | 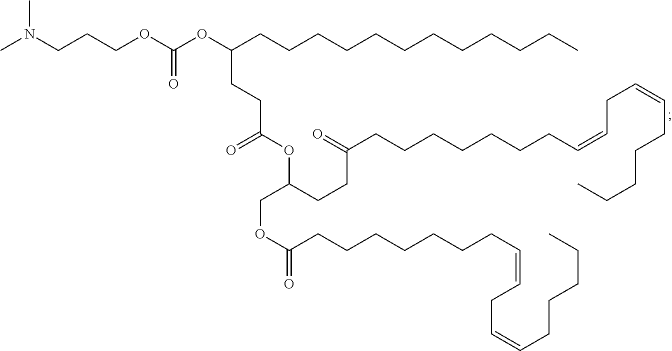 [SMILES: CCCCC/C=C\C/C=C\CCCCCCCC(=O)CCC(COC(=O)CCCCCCC/C=C\C/C=C\CCCCC)OC(=O)CCC(CCCCCCCCCCCC)OC(=O)OCCCN(C)C] |
| 67 | 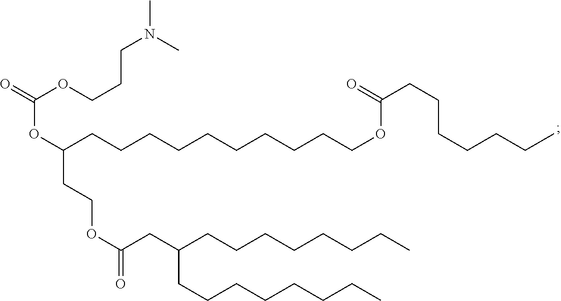 [SMILES: CCCCCCCCC(CCCCCCCC)CC(=O)OCCC(CCCCCCCCCCOC(=O)CCCCCCC)OC(=O)OCCCN(C)C] |
| 68 | 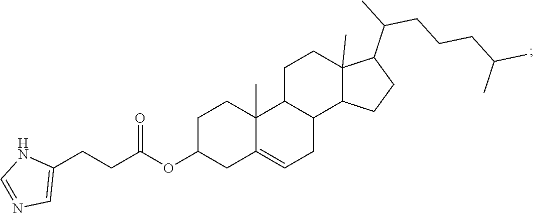 [SMILES: CC(C)CCCC(C)C1CCC2C3CC=C4CC(OC(=O)CCc5cnc[nH]5)CCC4(C)C3CCC12C] |
| 69 | 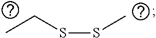 |
| 70 | 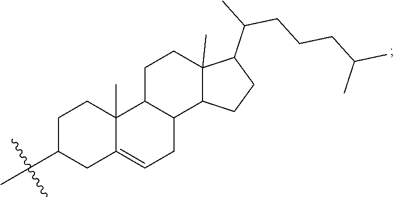 [SMILES: *C1CCC2(C)C(=CCC3C2CCC2(C)C(C(C)CCCC(C)C)CCC32)C1] |
| 71 | 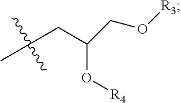 |
| 72 | 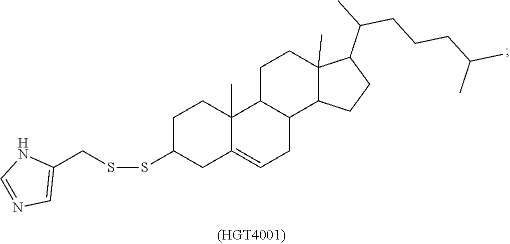 [SMILES: CC(C)CCCC(C)C1CCC2C3CC=C4CC(SSCc5cnc[nH]5)CCC4(C)C3CCC12C] |
| 73 | 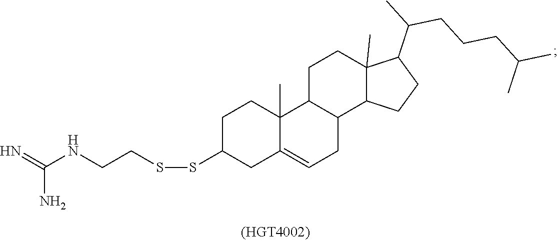 [SMILES: CC(C)CCCC(C)C1CCC2C3CC=C4CC(SSCCNC(=N)N)CCC4(C)C3CCC12C] |
| 74 | 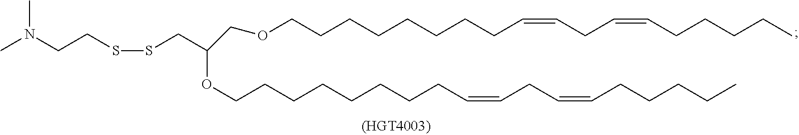 [SMILES: CCCCC/C=C\C/C=C\CCCCCCCCOCC(CSSCCN(C)C)OCCCCCCCC/C=C\C/C=C\CCCCC] |
| 75 | 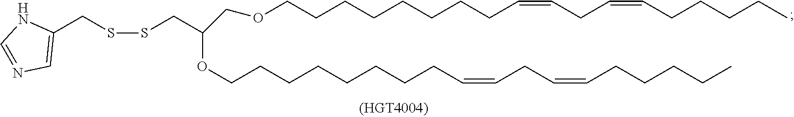 [SMILES: CCCCC/C=C\C/C=C\CCCCCCCCOCC(CSSCc1cnc[nH]1)OCCCCCCCC/C=C\C/C=C\CCCCC] |
| 76 | 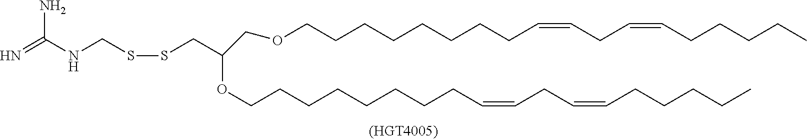 [SMILES: CCCCC/C=C\C/C=C\CCCCCCCCOCC(CSSCNC(=N)N)OCCCCCCCC/C=C\C/C=C\CCCCC] |

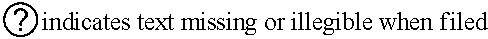

In some embodiments of the lipid composition of the present application, the ionizable cationic lipid is present in an amount from about from about 20 to about 23. In some embodiments, the molar percentage is from about 20, 20.5, 21, 21.5, 22, 22.5, to about 23 or any range derivable therein. In other embodiments, the molar percentage is from about 7.5 to about 20. In some embodiments, the molar percentage is from about 7.5, 8, 9, 10, 11, 12, 13, 14, 15, 16, 17, 18, 19, to about 20 or any range derivable therein.

In some embodiments of the lipid composition of the present application, the lipid composition comprises the ionizable cationic lipid at a molar percentage from about 5% to about 30%. In some embodiments of the lipid composition of the present application, the lipid composition comprises the ionizable cationic lipid at a molar percentage from about 10% to about 25%. In some embodiments of the lipid composition of the present application, the lipid composition comprises the ionizable cationic lipid at a molar percentage from about 15% to about 20%. In some embodiments of the lipid composition of the present application, the lipid composition comprises the ionizable cationic lipid at a molar percentage from about 10% to about 20%. In some embodiments of the lipid composition of the present application, the lipid composition comprises the ionizable cationic lipid at a molar percentage from about 20% to about 30%. In some embodiments of the lipid composition of the present application, the lipid composition comprises the ionizable cationic lipid at a molar percentage of at least (about) 5%, at least (about) 10%, at least (about) 15%, at least (about) 20%, at least (about) 25%, or at least (about) 30%. In some embodiments of the lipid composition of the present application, the lipid composition comprises the ionizable cationic lipid at a molar percentage of at most (about) 5%, at most (about) 10%, at most (about) 15%, at most (about) 20%, at most (about) 25%, or at most (about) 30%.

### Selective Organ Targeting (SORT) Lipids

In some embodiments of the lipid composition of the present application, the lipid (e.g., nanoparticle) composition is preferentially delivered to a target organ. In some embodiments, the target organ is a lung, a lung tissue or a lung cell. As used herein, the term “preferentially delivered” is used to refer to a composition, upon being delivered, which is delivered to the target organ (e.g., lung), tissue, or cell in at least 25% (e.g., at least 30%, 35%, 40%, 45%, 50%, 55%, 60%, 65%, 70%, or 75%) of the amount administered.

In some embodiments of the lipid composition, the lipid composition comprises one or more selective organ targeting (SORT) lipid which leads to the selective delivery of the composition to a particular organ. In some embodiments, the SORT lipid may have two or more alkyl or alkenyl chains of C₆-C₂₄.

In some embodiments of the lipid compositions, the SORT lipid comprises permanently positively charged moiety. The permanently positively charged moiety may be positively charged at a physiological pH such that the SORT lipid comprises a positive charge upon delivery of a polynucleotide to a cell. In some embodiments the positively charged moiety is quaternary amine or quaternary ammonium ion. In some embodiments, the SORT lipid comprises, or is otherwise complexed to or interacting with, a counterion.

In some embodiments of the lipid compositions, the SORT lipid is a permanently cationic lipid (i.e., comprising one or more hydrophobic components and a permanently cationic group). The permanently cationic lipid may contain a group which has a positive charge regardless of the pH. One permanently cationic group that may be used in the permanently cationic lipid is a quaternary ammonium group. The permanently cationic lipid may comprise a structural formula:

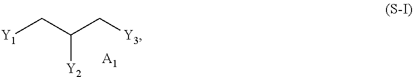

wherein:
Y₁, Y₂, or Y₃ are each independently X₁C(O)R₁ or X₂N⁺R₃R₄R₅;
provided at least one of Y₁, Y₂, and Y₃ is X₂N⁺R₃R₄R₅;
R₁ is C₁-C₂₄ alkyl, C₁-C₂₄ substituted alkyl, C₁-C₂₄ alkenyl, C₁-C₂₄ substituted alkenyl;
X₁ is O or NRₐ, wherein Rₐ is hydrogen, C₁-C₄ alkyl, or C₁-C₄ substituted alkyl;
X₂ is C₁-C₆ alkanediyl or C₁-C₆ substituted alkanediyl;
R₃, R₄, and R₅ are each independently C₁-C₂₄ alkyl, C₁-C₂₄ substituted alkyl, C₁-C₂₄ alkenyl, C₁-C₂₄ substituted alkenyl; and
A¹ is an anion with a charge equal to the number of X₂N⁺R₃R₄R₅ groups in the compound.

In some embodiments of the SORT lipids, the permanently cationic SORT lipid has a structural formula:

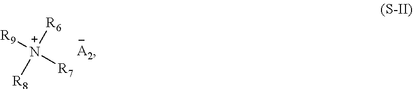

wherein:
R₆-R₉ are each independently C₁-C₂₄ alkyl, C₁-C₂₄ substituted alkyl, C₁-C₂₄ alkenyl, C₁-C₂₄ substituted alkenyl; provided at least one of R₆-R₉ is a group of C₈-C₂₄; and
A² is a monovalent anion.

In some embodiments of the lipid compositions, the SORT lipid is ionizable cationic lipid (i.e., comprising one or more hydrophobic components and an ionizable cationic group). The ionizable positively charged moiety may be positively charged at a physiological pH. One ionizable cationic group that may be used in the ionizable cationic lipid is a tertiary ammine group. In some embodiments of the lipid compositions, the SORT lipid has a structural formula:

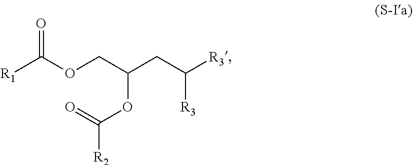

wherein:
R₁ and R₂ are each independently alkyl_{(C8-C24)}, alkenyl_{(C8-C24)}, or a substituted version of either group; and
R₃ and R_{3′} are each independently alkyl(co) or substituted alkyl_{(C≤6)}.

In some embodiments of the lipid compositions, the SORT lipid comprises a head group of a particular structure. In some embodiments, the SORT lipid comprises a headgroup having a structural formula:

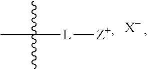

wherein L is a linker; Z⁺ is positively charged moiety and X⁻ is a counterion. In some embodiment, the linker is a biodegradable linker. The biodegradable linker may be degradable under physiological pH and temperature. The biodegradable linker may be degraded by proteins or enzymes from a subject. In some embodiments, the positively charged moiety is a quaternary ammonium ion or quaternary amine.

In some embodiments of the lipid compositions, the SORT lipid has a structural formula:

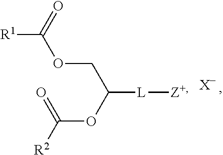

wherein R¹ and R² are each independently an optionally substituted C₆-C₂₄ alkyl, or an optionally substituted C₆-C₂₄ alkenyl.

In some embodiments of the lipid compositions, the SORT lipid has a structural formula:

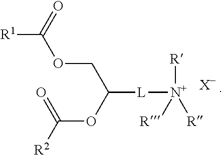

In some embodiments of the lipid compositions, the SORT lipid comprises a Linker (L). In some embodiments, L is

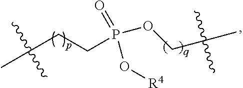

wherein:
p and q are each independently 1, 2, or 3; and
R₄ is an optionally substituted C₁-C₆ alkyl

In some embodiments of the lipid compositions, the SORT lipid has a structural formula:

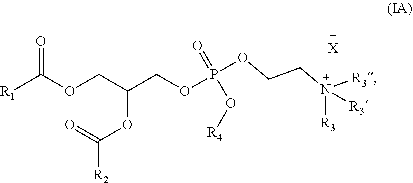

wherein:

R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
R₄ is alkyl_{(C≤6)} or substituted alkyl_{(C≤6)}; and
X⁻ is a monovalent anion.

In some embodiments of the lipid compositions, the SORT lipid is a phosphotidylcholine (e.g., 14:0 EPC). In some embodiments, the phosphatidylcholine compound is further defined

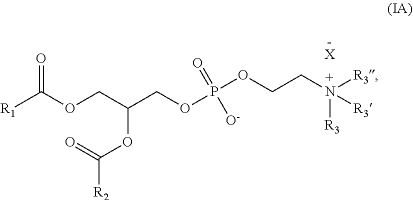

wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)}; and
X⁻ is a monovalent anion.

In some embodiments of the lipid compositions, the SORT lipid is a phosphocholine lipid. In some embodiments, the SORT lipid is an ethylphosphocholine. The ethylphosphocholine may be, by way of example, without being limited to, 1,2-dimyristoleoyl-sn-glycero-3-ethylphosphocholine, 1,2-diolcoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, 1-palmitoyl-2-oleoyl-sn-glycero-3-ethylphosphocholine.

In some embodiments of the lipid compositions, the SORT lipid has a structural formula:

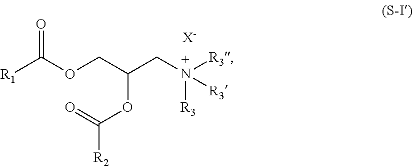

wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)}; and
X⁻ is a monovalent anion.

By way of example, and without being limited thereto, a SORT lipid of the structural formula of the immediately preceding paragraph is 1,2-dioleoyl-3-trimethylammonium-propane (18:1 DOTAP) (e.g., chloride salt).

In some embodiments of the lipid compositions, the SORT lipid has a structural formula:

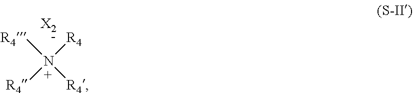

wherein:
R₄ and R_{4′} are each independently alkyl_{(C6-C24)}, alkenyl_{(C6-C24)}, or a substituted version of either group;
R_{4″} is alkyl_{(C≤24)}, alkenyl_{(C≤24)}, or a substituted version of either group;
R_{4″} is alkyl_{(C2-C8)}, alkenyl_{(C2-C8)}, or a substituted version of either group; and
X₂ is a monovalent anion.

By way of example, and without being limited thereto, a SORT lipid of the structural formula of the immediately preceding paragraph is dimethyldioctadecylammonium (DDAB) (e.g., bromide salt).

In some embodiments of the lipid compositions, the SORT lipid has a structural formula:

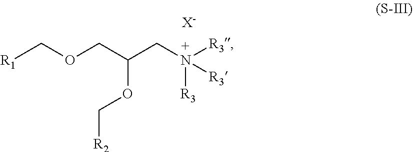

wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)}; and
X⁻ is a monovalent anion.

By way of example, and without being limited thereto, a SORT lipid of the structural formula of the immediately preceding paragraph is N-[1-(2,3-dioleyloxy)propyl]-N,N,N-trimethylammonium chloride (DOTMA).

In some embodiments of the lipid compositions, the SORT lipid is an anionic lipid. In some embodiments of the lipid compositions, the SORT lipid has a structural formula:

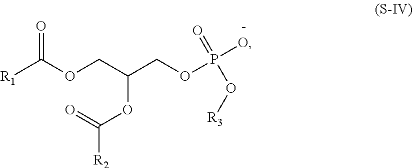

wherein:
R₁ and R₂ are each independently alkyl_{(C8-24)}, alkenyl_{(C8-C24)}, or a substituted version of either group;
R₃ is hydrogen, alkyl_{(C≤6)}, or substituted alkyl_{(C≤6)}, or —Y₁—R₄, wherein:
   Y₁ is alkanediyl_{(C≤6)} or substituted alkanediyl_{(C≤6)}; and
   R₄ is acyloxy_{(C≤8-24)} or substituted acyloxy_{(C≤8-24)}.

In some embodiments of the lipid compositions, the SORT lipid comprises one or more selected from the lipids set forth in Table 6.

TABLE 6
Example SORT lipids

| Lipid Name | Structure |
|---|---|
| 1,2-Dioleoyl-3- dimethylammonium-propane (18:1 DODAP) | 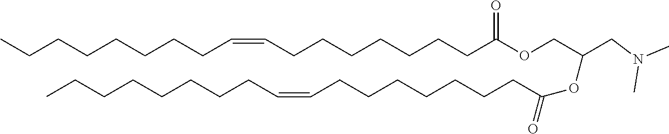 [SMILES: CCCCCCCC/C=C\CCCCCCCC(=O)OCC(CN(C)C)OC(=O)CCCCCCC/C=C\CCCCCCCC] |
| 1,2-dimyristoyl-3- trimethylammonium-propane (14:0 TAP) (e.g., chloride salt) | 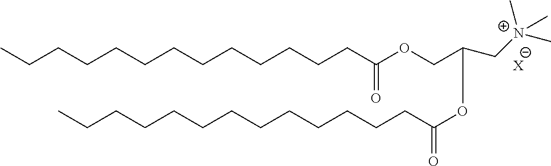 |
| 1,2-dipalmitoyl-3- trimethylammonium-propane (16:0 TAP) (e.g., chloride salt) | 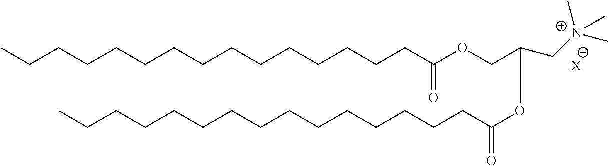 |
| 1,2-stearoyl-3- trimethylammonium-propane (18:0 TAP) (e.g., chloride salt) | 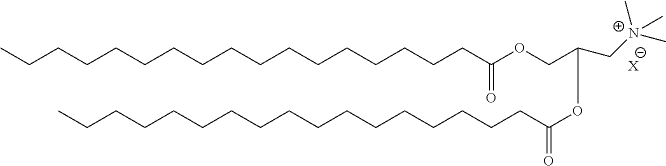 |
| 1,2-Dioleoy1-3- trimethylammonium-propane (18:1 DOTAP) (e.g., chloride salt) | 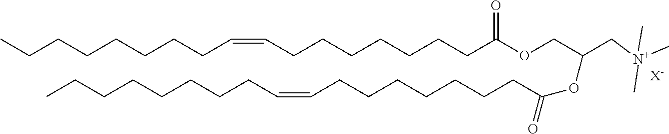 |
| 1,2-Di-O-octadeceny1-3- trimethylammonium propane (DOTMA) (e.g., chloride salt) | 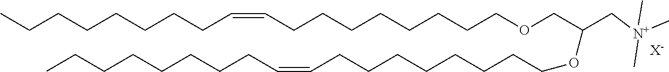 |
| Dimethyldioctadecylammonium (DDAB) (e.g., bromide salt) | 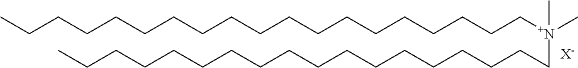 |
| 1,2-dilauroyl-sn-glycero-3- ethylphosphocholine (12:0 EPC) (e.g., chloride salt) | 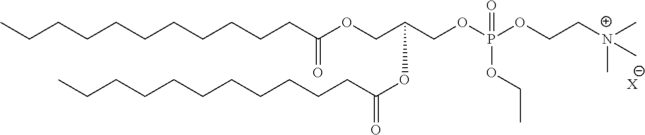 |
| 1,2-Dioleoyl-sn-glycero-3- ethylphosphocholine (14:0 EPC) (c.g., chloride salt) | 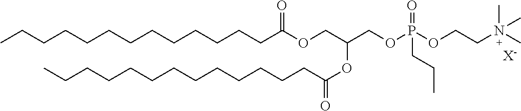 |
| 1,2-dimyristoleoyl-sn- glycero-3- ethylphosphocholine (14:1 EPC) (e.g., triflate salt) | 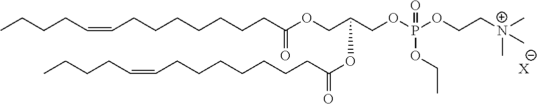 |
| 1,2-dipalmitoyl-sn-glycero-3- ethylphosphocholine (16:0 EPC) (e.g., chloride salt) | 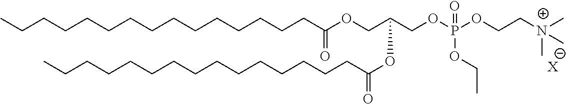 |
| 1,2-distearoyl-sn-glycero-3- ethylphosphocholine (18:0 EPC) (e.g., chloride salt) | 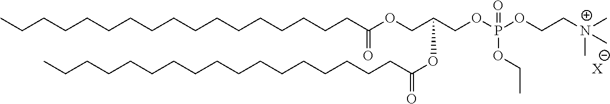 |
| 1,2-dioleoyl-sn-glycero-3- ethylphosphocholine (18:1 EPC) (e.g., chloride salt) | 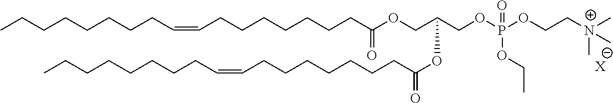 |
| 1-palmitoyl-2-oleoyl-sn- glycero-3- ethylphosphocholine (16:0-18:1 EPC) (e.g., chloride salt) | 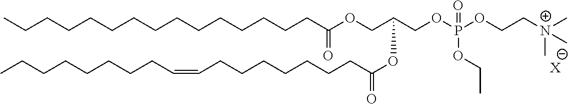 |
| 1,2-di-O-octadecenyl-3- trimethylammonium propane (18:1 DOTMA) (e.g., chloride salt) | 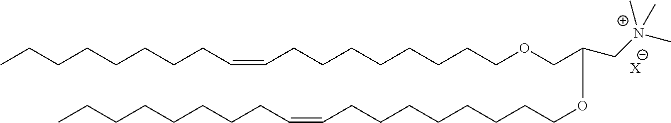 |
| 1,2-dioleoyl-sn-glycero-3- phosphate (18.1 PA) | 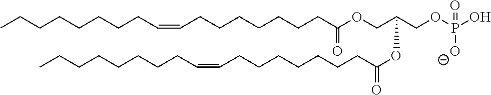 [SMILES: CCCCCCCC/C=C\CCCCCCCC(=O)OC[C@@H](COP(=O)([O-])O)OC(=O)CCCCCCC/C=C\CCCCCCCC] |

X⁻ is a counterion (e.g., Cl⁻, Br⁻, etc.)

In some embodiments of the lipid composition of the present application, the lipid composition comprises the SORT lipid at a molar percentage from about 20% to about 65%. In some embodiments of the lipid composition of the present application, the lipid composition comprises the SORT lipid at a molar percentage from about 25% to about 60%. In some embodiments of the lipid composition of the present application, the lipid composition comprises the SORT lipid at a molar percentage from about 30% to about 55%. In some embodiments of the lipid composition of the present application, the lipid composition comprises the SORT lipid at a molar percentage from about 20% to about 50%. In some embodiments of the lipid composition of the present application, the lipid composition comprises the SORT lipid at a molar percentage from about 30% to about 60%. In some embodiments of the lipid composition of the present application, the lipid composition comprises the SORT lipid at a molar percentage from about 25% to about 60%. In some embodiments of the lipid composition of the present application, the lipid composition comprises the SORT lipid at a molar percentage of at least (about) 25%, at least (about) 30%, at least (about) 35%, at least (about) 40%, at least (about) 45%, at least (about) 50%, at least (about) 55%, at least (about) 60%, or at least (about) 65%. In some embodiments of the lipid composition of the present application, the lipid composition comprises the SORT lipid at a molar percentage of at most (about) 25%, at most (about) 30%, at most (about) 35%, at most (about) 40%, at least (about) 45%, at most (about) 50%, at most (about) 55%, at most (about) 60%, or at most (about) 65%. In some embodiments of the lipid composition of the present application, the lipid composition comprises the SORT lipid at a molar percentage of about 20%, 25%, 30%, 35%, 40%, 45%, 50%, 55%, 60%, or 65%, or of a range between (inclusive) any two of the foregoing values.

### Additional Lipids

In some embodiments of the lipid composition of the present application, the lipid composition further comprises an additional lipid including but not limited to a steroid or a steroid derivative, a PEG lipid, and a phospholipid.

### Phospholipids or Other Zwitterionic Lipids

In some embodiments of the lipid composition of the present application, the lipid composition further comprises a phospholipid. In some embodiments, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some embodiments of the lipid compositions, the phospholipid is not an ethylphosphocholine.

In some embodiments of the lipid composition of the present application, the compositions may further comprise a molar percentage of the phospholipid to the total lipid composition from about 20 to about 23. In some embodiments, the molar percentage is from about 20, 20.5, 21, 21.5, 22, 22.5, to about 23 or any range derivable therein. In other embodiments, the molar percentage is from about 7.5 to about 60. In some embodiments, the molar percentage is from about 7.5, 8, 9, 10, 11, 12, 13, 14, 15, 16, 17, 18, 19, to about 20 or any range derivable therein.

In some embodiments of the lipid composition of the present application, the lipid composition comprises the phospholipid at a molar percentage from about 8% to about 23%. In some embodiments of the lipid composition of the present application, the lipid composition comprises the phospholipid at a molar percentage from about 10% to about 20%. In some embodiments of the lipid composition of the present application, the lipid composition comprises the phospholipid at a molar percentage from about 15% to about 20%. In some embodiments of the lipid composition of the present application, the lipid composition comprises the phospholipid at a molar percentage from about 8% to about 15%. In some embodiments of the lipid composition of the present application, the lipid composition comprises the phospholipid at a molar percentage from about 10% to about 15%. In some embodiments of the lipid composition of the present application, the lipid composition comprises the phospholipid at a molar percentage from about 12% to about 18%. In some embodiments of the lipid composition of the present application, the lipid composition comprises the phospholipid at a molar percentage of at least (about) 8%, at least (about) 10%, at least (about) 12%, at least (about) 15%, at least (about) 18%, at least (about) 20%, or at least (about) 23%. In some embodiments of the lipid composition of the present application, the lipid composition comprises the phospholipid at a molar percentage of at most (about) 8%, at most (about) 10%, at most (about) 12%, at most (about) 15%, at most (about) 18%, at most (about) 20%, or at most (about) 23%.

### Steroids or Steroid Derivatives

In some embodiments of the lipid composition of the present application, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

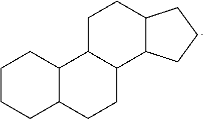 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

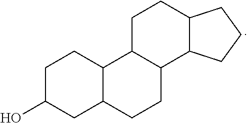 [SMILES: OC1CCC2C(CCC3C4CCCC4CCC23)C1]

In some embodiments of the present application, the steroid or steroid derivative is a cholestane or cholestane derivative. In a cholestane, the ring structure is further defined by the formula:

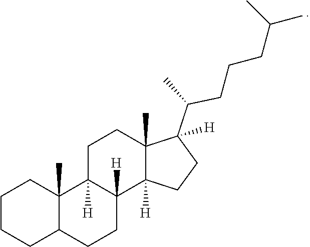

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestene and a sterol or a derivative thereof.

In some embodiments of the lipid composition, the compositions may further comprise a molar percentage of the steroid to the total lipid composition from about 40 to about 46. In some embodiments, the molar percentage is from about 40, 41, 42, 43, 44, 45, to about 46 or any range derivable therein. In other embodiments, the molar percentage of the steroid relative to the total lipid composition is from about 15 to about 40. In some embodiments, the molar percentage is 15, 16, 18, 20, 22, 24, 26, 28, 30, 32, 34, 36, 38, or 40, or any range derivable therein.

In some embodiments of the lipid composition of the present application, the lipid composition comprises the steroid or steroid derivative at a molar percentage from about 15% to about 46%. In some embodiments of the lipid composition of the present application, the lipid composition comprises the steroid or steroid derivative at a molar percentage from about 20% to about 40%. In some embodiments of the lipid composition of the present application, the lipid composition comprises the steroid or steroid derivative at a molar percentage from about 25% to about 35%. In some embodiments of the lipid composition of the present application, the lipid composition comprises the steroid or steroid derivative at a molar percentage from about 30% to about 40%. In some embodiments of the lipid composition of the present application, the lipid composition comprises the steroid or steroid derivative at a molar percentage from about 20% to about 30%. In some embodiments of the lipid composition of the present application, the lipid composition comprises the steroid or steroid derivative at a molar percentage of at least (about) 15%, of at least (about) 20%, of at least (about) 25%, of at least (about) 30%, of at least (about) 35%, of at least (about) 40%, of at least (about) 45%, or of at least (about) 46%. In some embodiments of the lipid composition of the present application, the lipid composition comprises the steroid or steroid derivative at a molar percentage of at most (about) 15%, of at most (about) 20%, of at most (about) 25%, of at most (about) 30%, of at most (about) 35%, of at most (about) 40%, of at most (about) 45%, or of at most (about) 46%.

### Polymer-Conjugated Lipids

In some embodiments of the lipid composition of the present application, the lipid composition further comprises a polymer conjugated lipid. In some embodiments, the polymer conjugated lipid is a PEG lipid. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1.200 to about 3.000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present application are taught by U.S. Pat. No. 5,820,873, WO 2010/141069, or U.S. Pat. No. 8,450,298, which is incorporated herein by reference.

In some embodiments of the lipid composition of the present application, the PEG lipid has a structural formula:

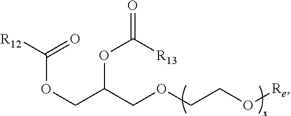

wherein: R₁₂ and R₁₃ are each independently alkyl_{(C≤4)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)}, such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments of the lipid composition of the present application, the PEG lipid has a structural formula:

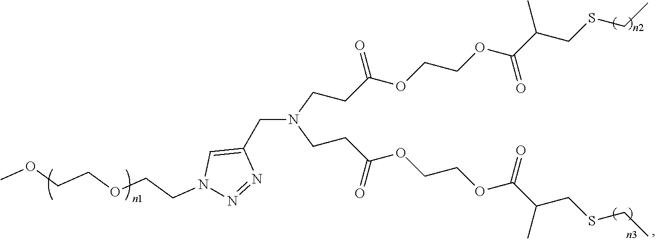

wherein: n₁ is an integer between 1 and 100 and n₂ and n₃ are each independently selected from an integer between 1 and 29. In some embodiments, n) is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments of the lipid composition of the present application, the compositions may further comprise a molar percentage of the PEG lipid to the total lipid composition from about 4.0 to about 4.6. In some embodiments, the molar percentage is from about 4.0, 4.1, 4.2, 4.3, 4.4, 4.5, to about 4.6 or any range derivable therein. In other embodiments, the molar percentage is from about 1.5 to about 4.0. In some embodiments, the molar percentage is from about 1.5, 1.75, 2, 2.25, 2.5, 2.75, 3, 3.25, 3.5, 3.75, to about 4.0 or any range derivable therein.

In some embodiments of the lipid composition of the present application, the lipid composition comprises the polymer-conjugated lipid at a molar percentage from about 0.5% to about 10%. In some embodiments of the lipid composition of the present application, the lipid composition comprises the polymer-conjugated lipid at a molar percentage from about 1% to about 10%. In some embodiments of the lipid composition of the present application, the lipid composition comprises the polymer-conjugated lipid at a molar percentage from about 2% to about 10%. In some embodiments of the lipid composition of the present application, the lipid composition comprises the polymer-conjugated lipid at a molar percentage from about 1% to about 8%. In some embodiments of the lipid composition of the present application, the lipid composition comprises the polymer-conjugated lipid at a molar percentage from about 2% to about 7%. In some embodiments of the lipid composition of the present application, the lipid composition comprises the polymer-conjugated lipid at a molar percentage from about 3% to about 5%. In some embodiments of the lipid composition of the present application, the lipid composition comprises the polymer-conjugated lipid at a molar percentage from about 5% to about 10%. In some embodiments of the lipid composition of the present application, the lipid composition comprises the polymer-conjugated lipid at a molar percentage of at least (about) 0.5%, at least (about) 1%, at least (about) 1.5%, at least (about) 2%, at least (about) 2.5%, at least (about) 3%, at least (about) 3.5%, at least (about) 4%, at least (about) 4.5%, at least (about) 5%, at least (about) 5.5%, at least (about) 6%, at least (about) 6.5%, at least (about) 7%, at least (about) 7.5%, at least (about) 8%, at least (about) 8.5%, at least (about) 9%, at least (about) 9.5%, or at least (about) 10%. In some embodiments of the lipid composition of the present application, the lipid composition comprises the polymer-conjugated lipid at a molar percentage of at most (about) 0.5%, at most (about) 1%, at most (about) 1.5%, at most (about) 2%, at most (about) 2.5%, at most (about) 3%, at most (about) 3.5%, at most (about) 4%, at most (about) 4.5%, at most (about) 5%, at most (about) 5.5%, at most (about) 6%, at most (about) 6.5%, at most (about) 7%, at most (about) 7.5%, at most (about) 8%, at most (about) 8.5%, at most (about) 9%, at most (about) 9.5%, or at most (about) 10%.

### Pharmaceutical Compositions

### Therapeutic or Prophylactic Agents

In another aspect, provided herein is a pharmaceutical composition comprising a therapeutic agent (or prophylactic agent) assembled with a lipid composition as described herein.

In some embodiments of the pharmaceutical composition, the therapeutic agent (or prophylactic agent) comprises a compound, a polynucleotide, a polypeptide, or a combination thereof. In some embodiments, the compound, the polynucleotide, the polypeptide, or a combination thereof is exogenous or heterologous to the cell or the subject being treated by the pharmaceutical compositions described herein. In some embodiments, the therapeutic agent (or prophylactic agent) comprises a compound described herein. In some embodiments, the therapeutic agent (or prophylactic agent) comprises a polynucleotide described herein. In some embodiments, the therapeutic agent (or prophylactic agent) comprises a polypeptide described herein. In some embodiments, the therapeutic agent (or prophylactic agent) comprises a compound, a polynucleotide, a polypeptide, or a combination thereof.

In some embodiments, the pharmaceutical composition comprises a therapeutic agent (or prophylactic agent) for treating a lung disease such as asthma, COPD, or lung cancer. In some embodiments, the therapeutic agent (or prophylactic agent) comprises a steroid such as prednisone, hydrocortisone, prednisolone, methylprednisolone, or dexamethasone. In some embodiments, the therapeutic agent (or prophylactic agent) comprises Abraxane, Afatinib Dimaleate, Afinitor, Afinitor Disperz. Alccensa. Alectinib, Alimta, Alunbrig, Atezolizumab, Avastin, Bevacizumab, Brigatinib, Capmatinib Hydrochloride, Carboplatin, Ceritinib, Crizotinib, Cyramza, Dabrafenib Mesylate, Dacomitinib, Docetaxel, Doxorubicin Hydrochloride, Durvalumab, Entrectinib, Erlotinib Hydrochloride, Everolimus, Gavreto, Gefitinib, Gilotrif, Gemcitabine, Ipilimumab, Iressa, Keytruda, Lorbrena, Mekinist, Methotrexate Sodium. Necitumumab, Nivolumab, Osimertinib Mesylate, Paclitaxel. Pembrolizumab, Pemetrexed Disodium, Pralsctinib, Ramucirumab, Retevmo, Selpercatinib, Tabrecta, Tafinlar, Tagrisso, Trametinib Dimethyl Sulfoxide, Vizimpro, Vinorelbine Tartrate, Xalkori, Yervoy, Zirabev, Zykadia, Carboplatin, Gemcitabine-cisplatin, Afinitor, Atezolizumab, Durvalumab, Etopophos, Etoposide, Hycamtin, Imfinzi, Keytruda, Lurbinectedin, Methotrexate Sodium, Nivolumab, Opdivo, Pembrolizumab, Tecentriq, Topotecan Hydrochloride, Trexall. or Zepzelca. Other non-limiting examples of the therapeutic agents (or prophylactic agents) comprising compounds include small molecule selected from 7-Methoxypteridine, 7 Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albendazole, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinonc, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin, doxycycline, dromostanolone propionate, droperidol, dyphylline, cchinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabinc, fluconazole, fluconazole, flucytosine, fludiovonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidonc, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexcthal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, mebendazole, Meclizine HCl, meclofenamic acid, medazepam, medigoxin, medroxyprogesterone acetate, mefenamic acid. Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin. pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, or zopiclone.

### Polynucleotides

In some embodiments of the pharmaceutical compositions of the present application, the therapeutic agent (or prophylactic agent) assembled with the lipid composition comprises one or more polynucleotides. The present application is not limited in scope to any particular source, sequence, or type of polynucleotide; however, as one of ordinary skill in the art could readily identify related homologs in various other sources of the polynucleotide including nucleic acids from non-human species (e.g., mouse, rat, rabbit, dog, monkey, gibbon, chimp, ape, baboon, cow, pig, horse, sheep, cat and other species). It is contemplated that the polynucleotide used in the present application can comprises a sequence based upon a naturally-occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally-occurring sequence. In another embodiment, the polynucleotide comprises nucleic acid sequence that is a complementary sequence to a naturally occurring sequence, or complementary to 75%, 80%, 85%, 90%, 95% and 100%. Longer polynucleotides encoding 250, 500, 1000, 1212, 1500, 2000, 2500, 3000 or longer are contemplated herein.

In some embodiments, the polynucleotide used herein may be derived from genomic DNA, i.e., cloned directly from the genome of a particular organism. In preferred embodiments, however, the polynucleotide would comprise complementary DNA (cDNA). Also contemplated is a cDNA plus a natural intron or an intron derived from another gene: such engineered molecules are sometime referred to as “mini-genes.” At a minimum, these and other nucleic acids of the present application may be used as molecular weight standards in, for example, gel electrophoresis. The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

In some embodiments, the polynucleotide comprises one or more segments comprising a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a or double stranded ribonucleic acid (dsRNA). In some embodiments, the polynucleotide encodes at least one of the therapeutic agents (or prophylactic agent) described herein. In some embodiments, the polynucleotide encodes at least one guide polynucleotide, such as guide RNA (gRNA) or guide DNA (gDNA), for complexing with a guide RNA guided nuclease described herein. In some embodiments, the polynucleotide encodes at least one guide polynucleotide guided heterologous nuclease. The nuclease may be an endonuclease. Non-limiting example of the guide polynucleotide guided heterologous endonuclease may be selected from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides: zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archacal Argonaute (aAgo), eukaryotic Argonaute (cAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof.

In some embodiments, the therapeutic (or prophylactic) agent is a transfer ribonucleic acid (tRNA) that introduces an amino acid into a growing peptide chain of a protein of a target gene. The target gene can be one set forth in Table A.

TABLE A
Example target genes for transfer RNA therapy

| Target | Gene Name | Corresponding Protein | Disease |
|---|---|---|---|
| CFTR | CFTR | Cystic fibrosis transmembrane conductance | Cystic fibrosis |
| | | regulator | |
| DNAH5 | DNAH5 | Dynein axonemal heavy chain 5 | Primary ciliary dyskinesia |
| DNAH11 | DNAH11 | Dynein axonemal heavy chain 11 | Primary ciliary dyskinesia |
| BMPR2 | BMPR2 | Bone morphogenetic protein receptor type 2 | Pulmonary arterial hypertension |
| FAH | FAH | Fumarylacetoacetate hydrolase | Tyrosinemia |
| PAH | PAH | Phenylalanine hydroxylase | Phenylketonuria |
| IDUA | IDUA | Alpha-L-iduronidase | Mucopolysaccharidosis i |
| COL4A3 | COL4A3 | Collagen type IV alpha 3 chain | Alport syndrome |
| COL4A4 | COL4A4 | Collagen type IV alpha 4 chain | Alport syndrome |
| COL4A5 | COL4A5 | Collagen type IV alpha 5 chain | Alport syndrome |
| PKD1 | PKD1 | Polycystin 1 | Polycystic kidney disease |
| PKD2 | PKD2 | Polycystin 2 | Polycystic kidney disease |
| PKHD1 | PKHD1 | Fibrocystin (or polyductin) | Polycystic kidney disease |
| SLC3A1 | SLC3A1 | Solute carrier family 3 member 1 | Cystinuria |
| SLC7A9 | SLC7A9 | Solute carrier family 7 member 9 | Cystinuria |
| PAX9 | PAX9 | Paired box gene 9 | Aniridia |
| MYO7A | MYO7A | Myosin VIIA | Usher syndrome |
| CDH23 | CDH23 | Cadherin related 23 | Usher syndrome |
| USH2A | USH2A | Usherin | Usher syndrome |
| CLRN1 | CLRN1 | Clarin 1 | Usher syndrome |
| GJB2 | GJB2 | Gap junction beta-2 protein | Non-syndromic hearing loss |
| GJB6 | GJB6 | Gap junction beta-6 protein | Non-syndromic hearing loss |
| RHO | RHO | Rhodopsin | Retinitis pigmentosa |
| DMPK | DMPK | dystrophia myotonica protein kinase | Myotonic dystrophy type 1 |
| DMD | DMD | Dystrophin | Muscular dystrophy |
| SCN1A | SCN1A | Sodium voltage-gated channel alpha subunit 1 | Dravet syndrome |
| SCN1B | SCN1B | Sodium voltage-gated channel beta subunit 1 | Dravet syndrome |
| F8 | F8 | Coagulation factor VIII | Hemophilia a |
| F9 | F9 | Coagulation factor IX | Hemophilia b |
| NGLY1 | NGLY1 | N-glycanase 1 | N-glycanase 1 deficiency |
| p53 | p53 | Tumor protein p53 | Cancer |
| CLN1 | PPT1 | Palmitoyl-protein thioesterase 1 | Batten disease |
| CLN2 | TPP1 | Tripeptidyl peptidase 1 | Late infantile ceroid lipofuscinoses |
| HERG | hERG | Kv11.1 (alpha subunit of potassium ion | Long qt syndrome |
| | | channel) | |
| PPT1 | PPT1 | Palmitoyl-protein thioesterase 1 | Infantile ceriod lipofuscinoses |
| ATM | ATM | ATM serine/threonine kinase | Ataxia telangiectasia |
| FBN1 | FBN1 | Fibrillin 1 | Usher syndrome type 1 |

Some embodiments of the therapeutic agent (or prophylactic agent) provided herein comprise a heterologous polypeptide comprising an actuator moiety. The actuator moiety can be configured to complex with a target polynucleotide corresponding to a target gene. In some embodiments, administration of the therapeutic agent (or prophylactic agent) results in a modified expression or activity of the target gene. The modified expression or activity of the target gene can be detectable, for example, in at least about 1% (e.g., at least about 2%, 5%, 10%. 15%, or 20%) cells (e.g., lung cells, such as lung basal cells) of the subject. The therapeutic agent (or prophylactic agent) may comprise a heterologous polynucleotide encoding an actuator moiety. The actuator moiety may be configured to complex with a target polynucleotide corresponding to a target gene. The heterologous polynucleotide may encode a guide polynucleotide configured to direct the actuator moiety to the target polynucleotide. The actuator moiety may comprise a heterologous endonuclease or a fragment thereof (e.g., directed by a guide polynucleotide to specifically bind the target polynucleotide). The heterologous endonuclease may be (1) part of a ribonucleoprotein (RNP) and (2) complexed with the guide polynucleotide. The heterologous endonuclease may be part of a clustered regularly interspaced short palindromic repeats (CRISPR)/CRISPR-associated (Cas) protein complex. The heterologous endonuclease may be a clustered regularly interspaced short palindromic repeats (CRISPR)-associated (Cas) endonuclease. The heterologous endonuclease may comprise a deactivated endonuclease. The deactivated endonuclease may be fused to a regulatory moiety. The regulatory moiety may comprise a transcription activator, a transcription repressor, an epigenetic modifier, or a fragment thereof.

In some embodiments, the polynucleotide encodes at least one guide polynucleotide (such as guide RNA (gRNA) or guide DNA (gDNA)) guided heterologous endonuclease. In some embodiments, the polynucleotide encodes at least one guide polynucleotide and at least one heterologous endonuclease, where the guide polynucleotide can be complexed with and guides the at least one heterologous endonuclease to cleave a genetic locus of any one of the genes described herein. In some embodiments, the polynucleotide encodes at least one guide polynucleotide guided heterologous endonuclease such as Cas9, Cas12, Cas13, Cpf1 (or Cas12a), C2C1, C2C2 (or Cas13a), Cas13b, Cas13c, Cas13d, Cas14, C2C3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Csn1, Csx12, Cas10, Cas10d, Cas1O, Cas1Od, CasF, CasG, CasH, Csy1, Csy2, Csy3, Cse1 (CasA), Cse2 (CasB). Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, or Cul966; any derivative thereof: any variant thereof, or any fragment thereof. In some embodiments. Cas13 can include, but are not limited to, Cas13a, Cas13b, Cas13c, and Cas 13d (e.g., CasRx).

In some embodiments, the heterologous endonuclease comprises a deactivated endonuclease, optionally fused to a regulatory moiety, such as an epigenetic modifier to remodel the epigenome that mediates the expression of the selected genes of interest. In some cases, the epigenetic modifier can include methyltransferase, demethylase, dismutase, an alkylating enzyme, depurinase, oxidase, photolyase, integrase, transposase, recombinase, polymerase, ligase, helicase, glycosylase, acetyltransferase, deacetylase, kinase, phosphatase, ubiquitin-activating enzymes, ubiquitin-conjugating enzymes, ubiquitin ligase, deubiquitinating enzyme, adenylate-forming enzyme, AMPylator, de-AMPylator, SUMOylating enzyme, deSUMOylating enzyme, ribosylase, deribosylase, N-myristoyltransferase, chromotine remodeling enzyme, protease, oxidoreductase, transferase, hydrolase, lyase, isomerase, synthase, synthetase, or demyristoylation enzyme. In some instances, the epigenetic modifier can comprise one or more selected from the group consisting of p300, TET1, LSD1, HDAC1, HDAC8, HDAC4, HDACH1, HDT1, SIRT3, HST2, CobB, SIRT5, SIR2A, SIRT6, NUE, vSET, SUV39H1, DIMS, KYP, SUVR4, Set4, Set1, SETD8, and TgSET8.

In some embodiments, the polynucleotide encodes a guide polynucleotide (such as guide RNA (gRNA) or guide DNA (gDNA)) that is at least partially complementary to the genomic region of a gene, where upon binding of the guide polynucleotide to the gene the guide polynucleotide recruits the guide polynucleotide guided nuclease to cleave and genetically modified the region. Examples of the genes that may be modified by the guide polynucleotide guided nuclease include CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCNIA, SCNIB, F8, F9. NGLY1, p53, PPT1, TPP1, hERG, PPT1, ATM, or FBN1.

In some embodiments, the polynucleotide comprises or encodes at least one mRNA that, upon expression of the mRNA, restores the function of a defective gene in a subject being treated by the pharmaceutical composition described herein. For example, the polynucleotide comprises or encodes an mRNA that expresses a wild type CFTR protein, which may be used to rescue a subject who is afflicted with inborn mutation in CFTR protein. Other examples of mRNA that can be expressed from the polynucleotide includes mRNA that encodes DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCNIA, SCNIB, F8, F9, NGLY1, p53, PPT1, TPP1, hERG, PPT1, ATM, or FBN1.

In some embodiments, the polynucleotides of the present application comprise at least one chemical modifications of the one or more nucleotides. In some embodiments, the chemical modification increases specificity of the guide polynucleotide (such as guide RNA (gRNA) or guide DNA (gDNA)) binding to a complementary genomic locus (e.g., the genomic locus of any one of the genes described herein). In some embodiments, the at least one chemical modification increases resistance to nuclease digestion, when then polynucleotide is administered to a subject in need thereof. In some embodiments, the at least one chemical modification decreases immunogenicity, when then polynucleotide is administered to a subject in need thereof. In some embodiments, the at least one chemical modification stabilizes scaffold such as a tRNA scaffold. Such chemical modification may have desirable properties, such as enhanced resistance to nuclease digestion or increased binding affinity with a target genomic locus relative to a polynucleotide without the at least one chemical modification.

In some embodiments, the at least one chemical modification comprises modification to sugar moiety. In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties.

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)—O—N(Rₘ)(Rₙ), O(CH₂)₂O(CH₂)₂N(CH₃)₂, and N-substituted acetamide (O)—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂-2′, 4′-(CH₂)₂-2′, 4′-(CH₂)—O-2′ (LNA): 4′-(CH₂)—S-2′; 4′-(CH₂)₂—O-2′ (ENA); 4′- CH(CH₃)—O-2′ (cEt) and 4′-CH(CH₂OCH₃)—O-2′, and analogs thereof: 4′-C(CH₃)(CH₃)—O-2′ and analogs thereof; 4′-CH₂—N(OCH₃)-2′ and analogs thereof; 4′-CH₂—O—N(CH₃)-2′; 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group: 4′-CH₂—C(H)(CH₃)-2′; and 4′-CH₂—C(═CH₂)-2′ and analogs thereof.

In some embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl. C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJI, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) Methoxy(ethyleneoxy) (4′-CH(CH₂OMe)-O-2′) BNA (also referred to as constrained MOE or cMOE).

In some embodiments, bicyclic sugar moieties and nucleosides incorporating such bicyclic sugar moieties are further defined by isomeric configuration. For example, a nucleoside comprising a 4′-2′ methylene-oxy bridge, may be in the .alpha.-L configuration or in the .beta.-D configuration. Previously, α-L-methyleneoxy (4′-CH, —O-2′) bicyclic nucleosides have been incorporated into antisense polynucleotides that showed antisense activity.

In some embodiments, substituted sugar moieties comprise one or more non-bridging sugar substituent and one or more bridging sugar substituent (e.g., 5′-substituted and 4′-2′ bridged sugars, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfur, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position and/or the 5′ position. By way of additional example, carbocyclic bicyclic nucleosides having a 4′-2′ bridge have been described.

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA), and fluoro HNA (F-HNA).

In some embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In some embodiments, at least one of q₁, q₂, q₃, q₄, q₅, q₆ and q₇ is methyl. In some embodiments, THP nucleosides of Formula VII are provided wherein one of R₁ and R₂ is F. In certain embodiments. R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds.

Combinations of modifications are also provided without limitation, such as 2′-F-5′-methyl substituted nucleosides and replacement of the ribosyl ring oxygen atom with S and further substitution at the 2′-position or alternatively 5′-substitution of a bicyclic nucleic acid. In some embodiments, a 4′-CH₂—O-2′ bicyclic nucleoside is further substituted at the S′ position with a 5′-methyl or a 5′-vinyl group). The synthesis and preparation of carbocyclic bicyclic nucleosides along with their oligomerization and biochemical studies have also been described.

In some embodiments, the present application provides polynucleotide comprising modified nucleosides. Those modified nucleotides may include modified sugars, modified nucleobases, and/or modified linkages. The specific modifications are selected such that the resulting polynucleotide possesses desirable characteristics. In some embodiments, polynucleotide comprises one or more RNA-like nucleosides. In some embodiments, polynucleotide comprises one or more DNA-like nucleotides.

In some embodiments, nucleosides of the present application comprise one or more unmodified nucleobases. In certain embodiments, nucleosides of the present application comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil: 5-propynylcytosine: 5-hydroxymethyl cytosine, xanthine, hypoxanthine, 2-aminoadenine, 6-methyl and other alkyl derivatives of adenine and guanine, 2-propyl and other alkyl derivatives of adenine and guanine. 2-thiouracil, 2-thiothymine and 2-thiocytosine. 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo. 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (2H-pyrimido[4,5-b]indol-2-one), pyridoindole cytidine (H-pyrido[3′,2′; 4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone.

In some embodiments, the present application provides polynucleotide comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The polynucleotides described herein contain one or more asymmetric centers and thus give rise to enantiomers, diastereomers, and other stereoisomeric configurations that may be defined, in terms of absolute stereochemistry, as (R) or (S), α or β such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates. MMI (3′-CH, —N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook. Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated polynucleotides of the present application involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid. e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

In some embodiments, the polynucleotides described herein comprise or encode at least one tRNA described herein. In some embodiments, the tRNA expressed from the polynucleotide restores the function of at least one defective tRNA in a subject who is being treated by the pharmaceutical composition described herein. In some embodiments, the at least one tRNA expressed by the polynucleotide described herein may include tRNA that encodes alanine, arginine, asparagine, aspartic acid, cysteine, glutamic acid, glutamine, glycine, histidine, hydroxyproline, isoleucine, leucin, lysine, methionine, phenylaniline, proline, pyroglutamic acid, serine, threonine, tryptophan, tyrosine, or valine. In some embodiments, the at least one tRNA expressed by the polynucleotide described herein may include tRNA that encodes arginine, tryptophan, glutamic acid, glutamine, serine, tyrosine, lysine, leucine, glycine, or cysteine. In some embodiments, the tRNA encoded by the polynucleotide described herein may restore the expression of any one of the genes described herein. In some embodiments, the tRNA encoded by the polynucleotide described herein may restore the expression of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCNIA, SCNIB, F8, F9, NGLY1, p53, PPT1, TPP1, hERG, PPT1, ATM, or FBN1.

### Polypeptides

In some embodiments of the pharmaceutical compositions of the present application, the therapeutic agent (or prophylactic agent) assembled with the lipid composition comprises one or more one or more polypeptides. Some polypeptide may include enzymes such as any one of the nuclease enzymes described herein. For example, the nuclease enzyme may include from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (cAgo), and Natronobacterium gregoryi Argonaute (NgAgo)); Adenosine deaminases acting on RNA (ADAR): CIRT, PUF, homing endonuclease, or any functional fragment thereof, any derivative thereof; any variant thereof; and any fragment thereof. In some embodiments, the nuclease enzyme may include Cas proteins such as Cas1, Cas1B, Cas2. Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4. Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein may be complexed with a guide polynucleotide described herein to be form a CRISPR ribonucleoprotein (RNP).

The nuclease in the compositions described herein may be Cas9 (e.g., from S. pyogenes or S. pneumonia). The CRISPR enzyme can direct cleavage of one or both strands at the location of a target sequence, such as within the target sequence and/or within the complement of the target sequence of any one of the genes described herein. For example, the CRISPR enzyme may be directed and cleaved a genomic locus of CFTR, DNAH5, DNAH11, BMPR2, FAH, PAH, IDUA, COL4A3, COL4A4, COL4A5, PKD1, PKD2, PKHD1·SLC3A1, SLC7A9, PAX9, MYO7A, CDH23, USH2A, CLRN1, GJB2, GJB6, RHO, DMPK, DMD, SCNIA, SCNIB, F8, F9, NGLY1, p53, PPT1, TPP1, hERG, PPT1, ATM, or FBN1.

The CRISPR enzyme may be mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g. two guide sequences, which target respectively sense and antisense strands of the DNA target. This combination allows both strands to be nicked and used to induce NHEJ or HDR.

In some embodiments, the present application provides polypeptide containing one or more therapeutic proteins. The therapeutic proteins that may be included in the composition include a wide range of molecules such as cytokines, chemokines, interleukins, interferons, growth factors, coagulation factors, anti-coagulants, blood factors, bone morphogenic proteins, immunoglobulins, and enzymes. Some non-limiting examples of particular therapeutic proteins include Erythropoietin (EPO), Granulocyte colony-stimulating factor (G-CSF), Alpha-galactosidase A, Alpha-L-iduronidase, Thyrotropin α, N-acetylgalactosamine-4-sulfatase (rhASB), Dornase alfσ. Tissue plasminogen activator (TPA) Activase, Glucocerebrosidase, Interferon (IF) β-1a, Interferon β-1b, Interferon γ, Interferon α, TNF-α, IL-1 through IL-36, Human growth hormone (rHGH), Human insulin (BHI), Human chorionic gonadotropin α, Darbepoetin α, Follicle-stimulating hormone (FSH), and Factor VIII.

In some embodiments, the polypeptide comprises a peptide sequence that is at least partially identical to any of the therapeutic agent (or prophylactic agent) comprising a peptide sequence. For example, the polypeptide may comprise a peptide sequence that is at least partially identical to an antibody (e.g., a monoclonal antibody) for treating a lung disease such as lung cancer.

In some embodiments, the polypeptide comprises a peptide or protein that restores the function of a defective protein in a subject being treated by the pharmaceutical composition described herein. For example, the polynucleotide comprises a peptide or protein that restores function of cystic fibrosis transmembrane conductance regulator (CFTR) protein, which may be used to rescue a subject who is afflicted with inborn error leading to the expression of the mutated CFTR protein. Other examples of the rescue may include administering to a subject in need thereof a polypeptide comprising a peptide or protein of wild type Dynein axonemal heavy chain 5, Dynein axonemal heavy chain 11, Bone morphogenetic protein receptor type 2, Fumarylacetoacetate hydrolase, Phenylalanine hydroxylase, Alpha-L-iduronidase, Collagen type IV alpha 3 chain, Collagen type IV alpha 4 chain, Collagen type IV alpha 5 chain, Polycystin 1, Polycystin 2, Fibrocystin (or polyductin), Solute carrier family 3 member 1, Solute carrier family 7 member 9, Paired box gene 9, Myosin VIIA, Cadherin related 23, Usherin, Clarin 1. Gap junction beta-2 protein. Gap junction beta-6 protein, Rhodopsin, dystrophia myotonica protein kinase, Dystrophin, Sodium voltage-gated channel alpha subunit 1. Sodium voltage-gated channel beta subunit 1, Coagulation factor VIII, Coagulation factor IX, N-glycanase 1, Tumor protein p53, Palmitoyl-protein thioesterase 1, Tripeptidyl peptidase 1, Kv 11.1 (alpha subunit of potassium ion channel), Palmitoyl-protein thioesterase 1, ATM serine/threonine kinase, or Fibrillin 1.

In some embodiments, the pharmaceutical composition of the present application comprises a plurality of payloads assembled with (e.g., encapsulated within) a lipid composition. The plurality of payloads assembled with the lipid composition may be configured for gene-editing or gene-expression modification. The plurality of payloads assembled with the lipid composition may comprise a polynucleotide encoding an actuator moiety (e.g., comprising a heterologous endonuclease such as Cas) or a polynucleotide encoding the actuator moiety. The plurality of payloads assembled with the lipid composition may further comprise one or more (e.g., one or two) guide polynucleotides. The plurality of payloads assembled with the lipid composition may further comprise one or more donor or template polynucleotides. The plurality of payloads assembled with the lipid composition may comprise a ribonucleoprotein (RNP).

In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no more than (about) 20:1, no more than (about) 15:1, no more than (about) 10:1, or no more than (about) 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no less than (about) 20:1, no less than (about) 15:1, no less than (about) 10:1, or no less than (about) 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 10:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 10:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 15:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1.

In some embodiments of the pharmaceutical composition of the present application, a molar ratio of the therapeutic agent to total lipids of the lipid composition is from about 1:1 to about 1:100. In some embodiments of the pharmaceutical composition of the present application, a molar ratio of the therapeutic agent to total lipids of the lipid composition is from about 1:1 to about 1:50. In some embodiments of the pharmaceutical composition of the present application, a molar ratio of the therapeutic agent to total lipids of the lipid composition is from about 50:1 to about 1:100. In some embodiments of the pharmaceutical composition of the present application, a molar ratio of the therapeutic agent to total lipids of the lipid composition is from about 1:1 to about 1:20. In some embodiments of the pharmaceutical composition of the present application, a molar ratio of the therapeutic agent to total lipids of the lipid composition is from about 20:1 to about 1:50. In some embodiments of the pharmaceutical composition of the present application, a molar ratio of the therapeutic agent to total lipids of the lipid composition is from about 50:1 to about 1:70. In some embodiments of the pharmaceutical composition of the present application, a molar ratio of the therapeutic agent to total lipids of the lipid composition is from about 70:1 to about 1:100. In some embodiments of the pharmaceutical composition of the present application, a molar ratio of the therapeutic agent to total lipids of the lipid composition is no more than (about) 1:1, no more than (about) 1:5, no more than (about) 1:10, no more than (about) 1:15, no more than (about) 1:20, no more than (about) 1:25, no more than (about) 1:30, no more than (about) 1:35, no more than (about) 1:40, no more than (about) 1:45, no more than (about) 1:50, no more than (about) 1:60, no more than (about) 1:70, no more than (about) 1:80, no more than (about) 1:90, or more than (about) 1:100. In some embodiments of the pharmaceutical composition of the present application, a molar ratio of the therapeutic agent to total lipids of the lipid composition is no less than (about) 1:1, no less than (about) 1:5, no less than (about) 1:10, no less than (about) 1:15, no less than (about) 1:20, no less than (about) 1:25, no less than (about) 1:30, no less than (about) 1:35, no less than (about) 1:40, no less than (about) 1:45, no less than (about) 1:50, no less than (about) 1:60, no less than (about) 1:70, no less than (about) 1:80, no less than (about) 1:90, or less than (about) 1:100.

In some embodiments of the pharmaceutical composition of the present application, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or (about) 100% of the therapeutic agent is encapsulated in particles of the lipid compositions.

In some embodiments of the pharmaceutical composition of the present application, the lipid composition comprises a plurality of particles characterized by one or more characteristics of the following: (1) a (e.g., average) size of 100 nanometers (nm) or less: (2) a polydispersity index (PDI) of no more than about 0.2; and (3) a zeta potential of −10 millivolts (mV) to 10 mV.

In some embodiments of the pharmaceutical composition of the present application, the lipid composition comprises a plurality of particles with a (e.g., average) size from about 50 nanometers (nm) to about 100 nanometers (nm). In some embodiments of the pharmaceutical composition of the present application, the lipid composition comprises a plurality of particles with a (e.g., average) size from about 70 nanometers (nm) to about 100 nanometers (nm). In some embodiments of the pharmaceutical composition of the present application, the lipid composition comprises a plurality of particles with a (e.g., average) size from about 50 nanometers (nm) to about 80 nanometers (nm). In some embodiments of the pharmaceutical composition of the present application, the lipid composition comprises a plurality of particles with a (e.g., average) size from about 60 nanometers (nm) to about 80 nanometers (nm). In some embodiments of the pharmaceutical composition of the present application, the lipid composition comprises a plurality of particles with a (e.g., average) size of at most about 100 nanometers (nm), at most about 90 nanometers (nm), at most about 85 nanometers (nm), at most about 80 nanometers (nm), at most about 75 nanometers (nm), at most about 70 nanometers (nm), at most about 65 nanometers (nm), at most about 60 nanometers (nm), at most about 55 nanometers (nm), or at most about 50 nanometers (nm). In some embodiments of the pharmaceutical composition of the present application, the lipid composition comprises a plurality of particles with a (e.g., average) size of at least about 100 nanometers (nm), at least about 90 nanometers (nm), at least about 85 nanometers (nm), at least about 80 nanometers (nm), at least about 75 nanometers (nm), at least about 70 nanometers (nm), at least about 65 nanometers (nm), at least about 60 nanometers (nm), at least about 55 nanometers (nm), or at least about 50 nanometers (nm). The (e.g., average) size may be determined by spectroscopic method(s) or image-based method(s), for example, dynamic light scattering, static light scattering, multi-angle light scattering, laser light scattering, or dynamic image analysis, or a combination thereof.

In some embodiments of the pharmaceutical composition of the present application, the lipid composition comprises a plurality of particles with a polydispersity index (PDI) from about 0.05 to about 0.5. In some embodiments of the pharmaceutical composition of the present application, the lipid composition comprises a plurality of particles with a polydispersity index (PDI) from about 0.1 to about 0.5. In some embodiments of the pharmaceutical composition of the present application, the lipid composition comprises a plurality of particles with a polydispersity index (PDI) from about 0.1 to about 0.3. In some embodiments of the pharmaceutical composition of the present application, the lipid composition comprises a plurality of particles with a polydispersity index (PDI) from about 0.2 to about 0.5. In some embodiments of the pharmaceutical composition of the present application, the lipid composition comprises a plurality of particles with a polydispersity index (PDI) of no more than about 0.5, no more than about 0.4, no more than about 0.3, no more than about 0.2, no more than about 0.1, or no more than about 0.05.

In some embodiments of the pharmaceutical composition of the present application, the lipid composition comprises a plurality of particles with a negative zeta potential of −5 millivolts (mV) or less. In some embodiments of the pharmaceutical composition of the present application, the lipid composition comprises a plurality of particles with a negative zeta potential of −10 millivolts (mV) or less. In some embodiments of the pharmaceutical composition of the present application, the lipid composition comprises a plurality of particles with a negative zeta potential of −15 millivolts (mV) or less. In some embodiments of the pharmaceutical composition of the present application, the lipid composition comprises a plurality of particles with a negative zeta potential of −20 millivolts (mV) or less. In some embodiments of the pharmaceutical composition of the present application, the lipid composition comprises a plurality of particles with a negative zeta potential of −30 millivolts (mV) or less. In some embodiments, the lipid composition comprises a plurality of particles with a zeta potential of 0 millivolts (mV) or less. In some embodiments, the lipid composition comprises a plurality of particles with a zeta potential of 5 millivolts (mV) or less. In some embodiments, the lipid composition comprises a plurality of particles with a zeta potential of 10 millivolts (mV) or less. In some embodiments of the pharmaceutical composition of the present application, the lipid composition comprises a plurality of particles with a negative zeta potential of 15 millivolts (mV) or less. In some embodiments of the pharmaceutical composition of the present application, the lipid composition comprises a plurality of particles with a negative zeta potential of 20 millivolts (mV) or less.

In some embodiments of the pharmaceutical composition of the present application, the lipid composition comprises a plurality of particles with a negative zeta potential of −5 millivolts (m V) or more. In some embodiments of the pharmaceutical composition of the present application, the lipid composition comprises a plurality of particles with a negative zeta potential of −10 millivolts (mV) or more In some embodiments of the pharmaceutical composition of the present application, the lipid composition comprises a plurality of particles with a negative zeta potential of −15 millivolts (mV) or more. In some embodiments of the pharmaceutical composition of the present application, the lipid composition comprises a plurality of particles with a negative zeta potential of −20 millivolts (mV) or more. In some embodiments of the pharmaceutical composition of the present application, the lipid composition comprises a plurality of particles with a negative zeta potential of −30 millivolts (mV) or more. In some embodiments, the lipid composition comprises a plurality of particles with a zeta potential of 0 millivolts (mV) or more. In some embodiments, the lipid composition comprises a plurality of particles with a zeta potential of 5 millivolts (mV) or more. In some embodiments, the lipid composition comprises a plurality of particles with a zeta potential of 10 millivolts (mV) or more. In some embodiments of the pharmaceutical composition of the present application, the lipid composition comprises a plurality of particles with a negative zeta potential of 15 millivolts (mV) or more. In some embodiments of the pharmaceutical composition of the present application, the lipid composition comprises a plurality of particles with a negative zeta potential of 20 millivolts (mV) or more.

In some embodiments of the pharmaceutical composition of the present application, the lipid composition has an apparent ionization constant (pKa) outside a range of 6 to 7. In some embodiments of the pharmaceutical composition of the present application, the lipid composition has an apparent pKa of about 8 or higher, about 9 or higher, about 10 or higher, about 11 or higher, about 12 or higher, or about 13 or higher. In some embodiments of the pharmaceutical composition of the present application, the lipid composition has an apparent pKa of about 8 to about 13. In some embodiments of the pharmaceutical composition of the present application, the lipid composition has an apparent pKa of about 8 to about 10. In some embodiments of the pharmaceutical composition of the present application, the lipid composition has an apparent pKa of about 9 to about 11. In some embodiments of the pharmaceutical composition of the present application, the lipid composition has an apparent pKa of about 10 to about 13. In some embodiments of the pharmaceutical composition of the present application, the lipid composition has an apparent pKa of about 8 to about 12. In some embodiments of the pharmaceutical composition of the present application, the lipid composition has an apparent pKa of about 10 to about 12.

In some embodiments of the pharmaceutical composition of the present application, the SORT lipid in the pharmaceutical composition effects a delivery of the therapeutic agent characterized by one or more of the following: (a) a greater therapeutic effect in a cell of the subject compared to that achieved with a reference lipid composition: (b) a therapeutic effect in a greater plurality of cells of the subject compared to that achieved with a reference lipid composition: (c) a therapeutic effect in a first plurality of cells of a first cell type and in a greater second plurality of cells of a second cell type; and (d) a greater therapeutic effect in a first cell of a first cell type of the subject compared to that in a second cell of a second cell type of the subject. In some embodiments, the first cell type is different from the second cell type.

In some embodiments of the pharmaceutical composition of the present application, the cell is a lung cell. In some embodiments, the lung cell is a lung airway cell. Example lung airway cells that can be targeted by the delivery of the present application includes but is not limited to basal cell.

In some embodiments of the pharmaceutical composition of the present application, the therapeutic effect is characterized by a therapeutically effective amount of the therapeutic agent, for example, in a lung, a lung cell, a plurality of lung cells, or a lung cell type of the subject. In some embodiments, the therapeutic effect is characterized by an activity of the therapeutic agent, for example, in a lung, a lung cell, a plurality of lung cells, or a lung cell type of the subject. In some embodiments, the therapeutic effect is characterized by an effect of the therapeutic agent, for example, in a lung, a lung cell, a plurality of lung cells, or a lung cell type of the subject. In some embodiments, the greater therapeutic effect is characterized by a greater therapeutic amount of the therapeutic agent. In some embodiments, the greater therapeutic effect is characterized by a greater activity of the therapeutic agent. In some embodiments, the greater therapeutic effect is characterized by a greater effect of the therapeutic agent.

In some embodiments of the pharmaceutical composition of the present application, the SORT lipid in the pharmaceutical composition effects delivery of the therapeutic agent to the cell of the subject characterized by a greater therapeutic effect compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises or essentially consists of 13,16,20-tris(2-hydroxydodecyl)-13,16,20,23-tetraazapentatricontane-11,25-diol (“LF92”), a phospholipid, cholesterol, and a PEG-lipid.

In some embodiments of the pharmaceutical composition of the present application, the SORT lipid in the pharmaceutical composition achieves about 1.1-fold to about 20-fold therapeutic effect compared to that achieved with a reference lipid composition. In some embodiments, the SORT lipid achieves about 1.1-fold to about 10-fold therapeutic effect compared to that achieved with a reference lipid composition. In some embodiments, the SORT lipid achieves about 5-fold to about 10-fold therapeutic effect compared to that achieved with a reference lipid composition. In some embodiments, the SORT lipid achieves about 10-fold to about 20-fold therapeutic effect compared to that achieved with a reference lipid composition. In some embodiments, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect compared to that achieved with a reference lipid composition.

In some embodiments of the pharmaceutical composition of the present application, the SORT lipid in the pharmaceutical composition achieves about 1.1-fold to about 20-fold therapeutic effect compared to that achieved with a reference lipid composition in basal cell. In some embodiments, the SORT lipid achieves about 1.1-fold to about 10-fold therapeutic effect compared to that achieved with a reference lipid composition in basal cell. In some embodiments, the SORT lipid achieves about 5-fold to about 10-fold therapeutic effect compared to that achieved with a reference lipid composition in basal cell. In some embodiments, the SORT lipid achieves about 10-fold to about 20-fold therapeutic effect compared to that achieved with a reference lipid composition in basal cell. In some embodiments, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect compared to that achieved with a reference lipid composition in basal cell.

In some embodiments of the pharmaceutical composition of the present application, the SORT lipid in the pharmaceutical composition effects delivery of the therapeutic agent to cells of the subject characterized by a therapeutic effect in a greater plurality of cells compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the pharmaceutical composition of the present application, the SORT lipid in the pharmaceutical composition achieves therapeutic effect in about 1.1-fold to about 20-fold cells compared to that achieved with a reference lipid composition. In some embodiments, the SORT lipid achieves therapeutic effect in about 1.1-fold to about 10-fold cells compared to that achieved with a reference lipid composition. In some embodiments, the SORT lipid achieves therapeutic effect in about 5-fold to about 10-fold cells compared to that achieved with a reference lipid composition. In some embodiments, the SORT lipid achieves therapeutic effect in about 10-fold to about 20-fold cells compared to that achieved with a reference lipid composition. In some embodiments, the SORT lipid achieves therapeutic effect in at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold cells compared to that achieved with a reference lipid composition.

In some embodiments of the pharmaceutical composition of the present application, the SORT lipid in the pharmaceutical composition achieves therapeutic effect in about 1.1-fold to about 20-fold cells compared to that achieved with a reference lipid composition in basal cell. In some embodiments, the SORT lipid achieves therapeutic effect in about 1.1-fold to about 10-fold more cells compared to that achieved with a reference lipid composition in basal cell. In some embodiments, the SORT lipid achieves therapeutic effect in about 5-fold to about 10-fold more cells compared to that achieved with a reference lipid composition in basal cell. In some embodiments, the SORT lipid achieves therapeutic effect in about 10-fold to about 20-fold more cells compared to that achieved with a reference lipid composition in basal cell. In some embodiments, the SORT lipid achieves therapeutic effect in about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold more cells compared to that achieved with a reference lipid composition in basal cell.

In some embodiments of the pharmaceutical composition of the present application, the SORT lipid in the pharmaceutical composition effects delivery of the therapeutic agent to cells of the subject characterized by a therapeutic effect in a first plurality of cells of a first cell type and in a greater therapeutic effect in a second plurality of cells of a second cell type. In some embodiments, the first cell type is different from the second cell type.

In some embodiments of the pharmaceutical composition of the present application, the first cell type is a lung cell. In some embodiments, the first cell type is a lung airway cell. Example lung airway cells that can be targeted by the delivery of the present application includes but is not limited to basal cell.

In some embodiments of the pharmaceutical composition of the present application, the second cell type is a lung cell. In some embodiments, the second cell type is a lung airway cell.

In some embodiments of the pharmaceutical composition of the present application, the SORT lipid in the pharmaceutical composition achieves therapeutic effect in about 1.1-fold to about 20-fold greater second plurality of cells of the second cell type compared to the first plurality of cells of the first cell type. In some embodiments, the SORT lipid achieves therapeutic effect in about 1.1-fold to about 10-fold greater second plurality of cells of the second cell type compared to the first plurality of cells of the first cell type. In some embodiments, the SORT lipid achieves therapeutic effect in about 5-fold to about 10-fold greater second plurality of cells of the second cell type compared to the first plurality of cells of the first cell type. In some embodiments, the SORT lipid achieves therapeutic effect in about 10-fold to about 20-fold greater second plurality of cells of the second cell type compared to the first plurality of cells of the first cell type. In some embodiments, the SORT lipid achieves therapeutic effect in at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold greater second plurality of cells of the second cell type compared to the first plurality of cells of the first cell type.

In some embodiments of the pharmaceutical composition of the present application, the SORT lipid in the pharmaceutical composition effects delivery of the therapeutic agent to cells of the subject characterized by a greater therapeutic effect in a first cell of a first cell type compared to that in a second cell of a second cell type. In some embodiments, the first cell type is different from the second cell type.

In some embodiments of the pharmaceutical composition of the present application, the first cell type is a lung cell. In some embodiments, the first cell type is a lung airway cell. Examples of lung airway cells that can be targeted by the delivery of the present application include but is not limited to basal cells.

In some embodiments of the pharmaceutical composition of the present application, the second cell type is a lung cell. In some embodiments, the second cell type is a lung airway cell.

In some embodiments of the pharmaceutical composition of the present application, the SORT lipid in the pharmaceutical composition achieves about 1.1-fold to about 20-fold therapeutic effect in first cell of the first cell type compared to that achieved in the second cell of the second cell type. In some embodiments, the SORT lipid achieves about 1.1-fold to about 10-fold therapeutic effect in first cell of the first cell type compared to that achieved in the second cell of the second cell type. In some embodiments, the SORT lipid achieves about 5-fold to about 10-fold therapeutic effect in first cell of the first cell type compared to that achieved in the second cell of the second cell type. In some embodiments, the SORT lipid achieves about 10-fold to about 20-fold therapeutic effect in first cell of the first cell type compared to that achieved in the second cell of the second cell type. In some embodiments of the method, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect in first cell of the first cell type compared to that achieved in the second cell of the second cell type.

In some embodiments, provided herein are (e.g., pharmaceutical) compositions that comprise components that allow for an improved efficacy or outcome based on the delivery of the polynucleotide. The compositions described elsewhere herein may be more effective at delivery to a particular cell, cell type, organ, or bodily region as compared to a reference composition or compound. The compositions described elsewhere herein may be more effective at generating increase expression of a corresponding polypeptide of a delivered polynucleotide. The compositions described elsewhere herein may be more effective at generating a larger number of cells that express a corresponding polypeptide of a delivered polynucleotide. The compositions described elsewhere herein may result in an increase uptake of the polynucleotide as compared to a reference polynucleotide. The increased uptake may be result of improved stability of polynucleotide or an improved targeting of the composition to a particular cell type or organ. In some embodiments, the SORT lipid is present in an amount in the lipid composition to effect a greater expression or activity of the polynucleotide (or corresponding polypeptide of the polynucleotide) in a cell compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid. In some embodiments, the SORT lipid is present in an amount in the lipid composition to effect at least a 1.1 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 2-fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 5 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 10-fold greater expression or activity of the polynucleotide (or corresponding polypeptide of the polynucleotide) in a cell compared to that achieved with a reference lipid composition comprising LF92, a phospholipid, cholesterol, and a PEG-lipid.

In some embodiments, the SORT lipid is present in an amount in the lipid composition to effect an expression or activity of the polynucleotide (or corresponding polypeptide of the polynucleotide) in a greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 1.1-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 2-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 5-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 10-fold greater plurality of cells compared to that achieved with a reference lipid composition comprising LF92, a phospholipid, cholesterol, and a PEG-lipid.

In some embodiments, the SORT lipid is present in an amount in the lipid composition to effect an uptake of the polynucleotide in a greater plurality of cells compared to that achieved with a reference lipid composition comprising LF92, a phospholipid, cholesterol, and a PEG-lipid. In some embodiments, the SORT lipid is present in an amount in the lipid composition to effect an uptake of the polynucleotide in a greater amount to a cell compared to that achieved with a reference lipid composition comprising LF92, a phospholipid, cholesterol, and a PEG-lipid.

### Methods

In another aspect, provided herein is a method for potent delivery to a cell of a subject comprising intravenously administrating to the subject the pharmaceutical composition as described in the present application. In some embodiments of the method, the pharmaceutical composition comprises a therapeutic agent (or prophylactic agent) assembled with a lipid composition as described in the present application, wherein the lipid composition comprises (i) an ionizable cationic lipid; and (ii) a selective organ targeting (SORT) lipid separate from the ionizable cationic lipid. The lipid composition may further comprise a phospholipid.

In some embodiments of the method, the cell is a lung cell. In some embodiments, the lung cell is a lung airway cell. Examples of lung airway cells that can be targeted by the delivery of the present application includes but is not limited to basal cells.

In some embodiments of the method, the SORT lipid effects delivery of the therapeutic agent to the cell of the subject characterized by a greater therapeutic effect compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the method, the SORT lipid achieves about 1.1-fold to about 20-fold therapeutic effect compared to that achieved with a reference lipid composition. In some embodiments of the method, the SORT lipid achieves about 1.1-fold to about 10-fold therapeutic effect compared to that achieved with a reference lipid composition. In some embodiments of the method, the SORT lipid achieves about 1.1-fold to about 5-fold therapeutic effect compared to that achieved with a reference lipid composition. In some embodiments of the method, the SORT lipid achieves about 5-fold to about 10-fold therapeutic effect compared to that achieved with a reference lipid composition. In some embodiments of the method, the SORT lipid achieves about 10-fold to about 20-fold therapeutic effect compared to that achieved with a reference lipid composition. In some embodiments of the method, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect compared to that achieved with a reference lipid composition.

In some embodiments of the method, the SORT lipid achieves about 1.1-fold to about 20-fold therapeutic effect compared to that achieved with a reference lipid composition in basal cell. In some embodiments of the method, the SORT lipid achieves about 1.1-fold to about 10-fold therapeutic effect compared to that achieved with a reference lipid composition in basal cell. In some embodiments of the method, the SORT lipid achieves about 1.1-fold to about 5-fold therapeutic effect compared to that achieved with a reference lipid composition in basal cell. In some embodiments of the method, the SORT lipid achieves about 10-fold to about 20-fold therapeutic effect compared to that achieved with a reference lipid composition in basal cell. In some embodiments of the method, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect compared to that achieved with a reference lipid composition in basal cell.

In some embodiments of the method, the SORT lipid effects delivery of the therapeutic agent to cells of the subject characterized by a therapeutic effect in a greater plurality of cells compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the method, the SORT lipid achieves therapeutic effect in about 1.1-fold to about 20-fold cells compared to that achieved with a reference lipid composition. In some embodiments of the method, the SORT lipid achieves therapeutic effect in about 1.1-fold to about 10-fold cells compared to that achieved with a reference lipid composition. In some embodiments of the method, the SORT lipid achieves therapeutic effect in about 1.1-fold to about 5-fold cells compared to that achieved with a reference lipid composition. In some embodiments of the method, the SORT lipid achieves therapeutic effect in about 10-fold to about 20-fold cells compared to that achieved with a reference lipid composition. In some embodiments of the method, the SORT lipid achieves therapeutic effect in at least about 1.1-fold, at least about 5-fold, at least about 10-fold, at least about 20-fold, at least about 50-fold, or at least about 100-fold cells compared to that achieved with a reference lipid composition.

In some embodiments of the method, the SORT lipid achieves therapeutic effect in about 1.1-fold to about 20-fold more cells compared to that achieved with a reference lipid composition, wherein the cells are basal cells. In some embodiments of the method, the SORT lipid achieves therapeutic effect in about 1.1-fold to about 10-fold more cells compared to that achieved with a reference lipid composition, wherein the cells are basal cells. In some embodiments of the method, the SORT lipid achieves therapeutic effect in about 5-fold to about 10-fold more cells compared to that achieved with a reference lipid composition, wherein the cells are basal cells. In some embodiments of the method, the SORT lipid achieves therapeutic effect in about 10-fold to about 20-fold more cells compared to that achieved with a reference lipid composition, wherein the cells are basal cells. In some embodiments of the method, the SORT lipid achieves therapeutic effect in at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold more cells compared to that achieved with a reference lipid composition, wherein the cells is basal cell.

In another aspect, provided herein is a method for targeted delivery to cells of a subject, comprising intravenously administrating to the subject the pharmaceutical composition as described in the present application. In some embodiments of the method, the pharmaceutical composition comprises a therapeutic agent (or prophylactic agent) assembled with a lipid composition as described in the present application, wherein the lipid composition comprises (i) an ionizable cationic lipid; and (iii) a selective organ targeting (SORT) lipid separate from the ionizable cationic lipid. The lipid composition may further comprise a phospholipid.

In some embodiments of the method, the SORT lipid effects delivery of the therapeutic agent to a greater proportion of cell types as compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the method, the cell is a lung cell. In some embodiments, the lung cell is a lung airway cell. Examples of lung airway cells that can be targeted by the delivery of the present application includes but is not limited to basal cell.

In some embodiments of the method, the SORT lipid effects delivery of the therapeutic agent to cells of the subject characterized by a therapeutic effect in a first plurality of cells of a first cell type and in a greater therapeutic effect in a second plurality of cells of a second cell type. In some embodiments, the first cell type is different from the second cell type.

In some embodiments of the method, the first cell type is a lung cell. In some embodiments, the first cell type is a lung airway cell. Examples of lung airway cells that can be targeted by the delivery of the present application includes but is not limited to basal cell.

In some embodiments of the method, the second cell type is a lung cell. In some embodiments, the second cell type is a lung airway cell.

In some embodiments of the method, the SORT lipid achieves therapeutic effect in about 1.1-fold to about 20-fold greater second plurality of cells of the second cell type compared to the first plurality of cells of the first cell type. In some embodiments of the method, the SORT lipid achieves therapeutic effect in about 1.1-fold to about 10-fold greater second plurality of cells of the second cell type compared to the first plurality of cells of the first cell type. In some embodiments of the method, the SORT lipid achieves therapeutic effect in about 1.1-fold to about 5-fold greater second plurality of cells of the second cell type compared to the first plurality of cells of the first cell type. In some embodiments of the method, the SORT lipid achieves therapeutic effect in about 10-fold to about 20-fold greater second plurality of cells of the second cell type compared to the first plurality of cells of the first cell type. In some embodiments of the method, the SORT lipid achieves therapeutic effect in at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold greater second plurality of cells of the second cell type compared to the first plurality of cells of the first cell type.

In some embodiments of the method, the SORT lipid effects delivery of the therapeutic agent to cells of the subject characterized by a greater therapeutic effect in a first cell of a first cell type compared to that in a second cell of a second cell type. In some embodiments, the first cell type is different from the second cell type.

In some embodiments of the method, the first cell type is a lung cell. In some embodiments, the first cell type is a lung airway cell. Examples of lung airway cells that can be targeted by the delivery of the present application includes but is not limited to basal cell.

In some embodiments of the method, the second cell type is a lung cell. In some embodiments, the second cell type is a lung airway cell.

In some embodiments of the method, the SORT lipid achieves about 1.1-fold to about 20-fold therapeutic effect in first cell of the first cell type compared to that achieved in the second cell of the second cell type. In some embodiments of the method, the SORT lipid achieves about 1.1-fold to about 10-fold therapeutic effect in first cell of the first cell type compared to that achieved in the second cell of the second cell type. In some embodiments of the method, the SORT lipid achieves about 5-fold to about 10-fold therapeutic effect in first cell of the first cell type compared to that achieved in the second cell of the second cell type. In some embodiments of the method, the SORT lipid achieves about 10-fold to about 20-fold therapeutic effect in first cell of the first cell type compared to that achieved in the second cell of the second cell type. In some embodiments of the method, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect in first cell of the first cell type compared to that achieved in the second cell of the second cell type.

In some embodiments, the delivery of the therapeutic to a cell may alter the genome, transcriptome, or expression levels. The cell may be allowed to, or able to, propagate and the alteration may be passed on to the cells derived (e.g., differentiated) from the cell that the therapeutic was delivered to. In this manner, the therapeutic effect may be propagated to a larger number of cells. The alteration to the genome, transcriptome or expression level may also persist in a given cell.

### Basal Cells

Basal cells are derived from undifferentiated columnar epithelium in the developing airway. They are characterized by position basal in the columnar epithelium, the presence of hemidesmosomes (characterized by alpha 6 beta 4 integrins), cytokeratins 5 and 14, and the nuclear protein p63. The distribution of basal cells varies by airway level and animal species. Airways that are larger in diameter have more basal cells than airways with smaller diameters. As the airway decreases in diameter, the number of basal cells also decreases, and none are present in the terminal bronchioles.

In another aspect, provided herein is a method for delivery to basal cells of a subject, comprising intravenously administrating to the subject the pharmaceutical composition as described in the present application. In some embodiments of the method, the pharmaceutical composition comprises a therapeutic agent (or prophylactic agent) assembled with a lipid composition as described in the present application, wherein the lipid composition comprises (i) an ionizable cationic lipid; and (ii) a selective organ targeting (SORT) lipid separate from the ionizable cationic lipid. In some embodiments, the basal cell is a lung basal cell. The lipid composition may further comprise a phospholipid.

In some embodiments of the method, the method delivers the therapeutic agent to an organ or tissue of the subject to result in a therapeutic effect detectable in basal cells in the organ or tissue of the subject. In some embodiments, the method delivers the therapeutic agent to an organ or tissue of the subject to result in a therapeutic effect detectable in at least about 5%, at least about 10%, at least about 15%, at least about 20%, at least about 30%, at least about 40%, at least about 50% basal cells in the organ or tissue of the subject.

In some embodiments of the method, the organ is lung. In some embodiments, the tissue is lung tissue. In some embodiment, the tissue is lung airway tissue. In some embodiments, the method delivers the therapeutic agent to the subject's lung to result in a therapeutic effect detectable in at least about 5%, at least about 10%, at least about 15%, at least about 20%, at least about 30%, at least about 40%, at least about 50% basal cells in the subject's lung.

### Dosing Level

In another aspect, provided is high-potency intravenous dosage form of a therapeutic agent (or prophylactic agent) formulated with a selective organ targeting (SORT) lipid, the dosage form comprising a therapeutic agent (or prophylactic agent) assembled with a lipid composition as described herein. In some embodiments, the lipid composition comprises: (i) an ionizable cationic lipid; and (iii) a selective organ targeting (SORT) lipid separate from the ionizable cationic lipid. The lipid composition may further comprise a phospholipid.

In some embodiments of the high-potency intravenous dosage form of the present application, the SORT lipid is present in the dosage form in an amount sufficient to achieve a therapeutic effect at a dose of the therapeutic agent lower than that required with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the high-potency intravenous dosage form of the present application, the SORT lipid is present in the dosage form in an amount sufficient to achieve a therapeutic effect at a dose of the therapeutic agent about 1.1-fold to about 20-fold lower than that required with a reference lipid composition. In some embodiments of the high-potency intravenous dosage form of the present application, the SORT lipid is present in the dosage form in an amount sufficient to achieve a therapeutic effect at a dose of the therapeutic agent about 1.1-fold to about 10-fold lower than that required with a reference lipid composition. In some embodiments of the high-potency intravenous dosage form of the present application, the SORT lipid is present in the dosage form in an amount sufficient to achieve a therapeutic effect at a dose of the therapeutic agent about 1.1-fold to about 5-fold lower than that required with a reference lipid composition. In some embodiments of the high-potency intravenous dosage form of the present application, the SORT lipid is present in the dosage form in an amount sufficient to achieve a therapeutic effect at a dose of the therapeutic agent about 10-fold to about 20-fold lower than that required with a reference lipid composition. In some embodiments of the high-potency intravenous dosage form of the present application, the SORT lipid is present in the dosage form in an amount sufficient to achieve a therapeutic effect at a dose of the therapeutic agent at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold lower than that required with a reference lipid composition.

In some embodiments, the therapeutic agent is present in the dosage form at a dose of about 2.0, 1.5, 1.0, 0.5, 0.2, or 0.1 milligram per kilogram (mg/kg, or mpk) body weight, or of a range between (inclusive) any two of the foregoing values.

In some embodiments, the therapeutic agent is present in the intravenous dosage form at a dose of no more than about 2 milligram per kilogram (mg/kg, or mpk) body weight. In some embodiments, the therapeutic agent is present in the intravenous dosage form at a dose of no more than about 1 milligram per kilogram (mg/kg, or mpk) body weight. In some embodiments, the therapeutic agent is present in the intravenous dosage form at a dose of no more than about 0.5 milligram per kilogram (mg/kg, or mpk) body weight. In some embodiments, the therapeutic agent is present in the intravenous dosage form at a dose of no more than about 0.2 milligram per kilogram (mg/kg, or mpk) body weight. In some embodiments, the therapeutic agent is present in the intravenous dosage form at a dose of no more than about 0.1 milligram per kilogram (mg/kg, or mpk) body weight.

In some embodiments, the therapeutic agent is present in the dosage form at a concentration of about 5, 4, 3, 2, 1, 0.5, 0.2, or 0.1 milligram per milliliter (mg/mL), or of a range between (inclusive) any two of the foregoing values.

In some embodiments, the therapeutic agent is present in the intravenous dosage form at a concentration of no more than about 5 milligram per milliliter (mg/mL). In some embodiments, the therapeutic agent is present in the intravenous dosage form at a concentration of no more than about 2 milligram per milliliter (mg/mL). In some embodiments, the therapeutic agent is present in the intravenous dosage form at a concentration of no more than about 1 milligram per milliliter (mg/mL). In some embodiments, the therapeutic agent is present in the intravenous dosage form at a concentration of no more than about 0.5 milligram per milliliter (mg/mL). In some embodiments, the therapeutic agent is present in the intravenous dosage form at a concentration of no more than about 0.1 milligram per milliliter (mg/mL).

In some embodiments, the dosage may be administered over a particular amount of time. The intravenous administration may be performed as an IV bolus. A dosage may be administered over a duration, e.g., a short period of time. The duration may be of no more than about 10 min. The duration may be of no more than about 5 min.

In some embodiments, the administration is performed via infusion with a premedication. In some embodiments, the administration is performed via infusion without a premedication.

In some embodiments, the administration of a dose of the therapeutic agent may be repeated.

### Subject

Any subject in need thereof can be treated with the method of the present application. In some embodiments, the subject has been determined to likely respond to the therapeutic agent. For example, the subject may have, is suffering from, or suspected of having a disease or condition. For the example, the disease or disorder may be selected from the group consisting of genetic respiratory disease, chronic inflammatory lung disease, pulmonary fibrosis, central nervous system (CNS) disorder, immuno-deficiency, autoimmune disease, cancer, infectious disease, liver fibrosis, cirrhosis, metabolic disorder, muscular dystrophy, and viral infection. The therapeutic or prophylactic agent(s) as described elsewhere herein may be effective for providing a therapeutic effect for the subject by a variety of mechanisms, for example, via gene therapy (e.g., requiring repeated administration), altered (e.g., increased) protein production, (e.g., in vivo) chimeric antigen receptor (CAR) T-cell generation, immuno-oncology, vaccine-based approach, reactivation of tumor suppressors, or other mechanisms.

In some embodiments, the subject has been determined to have a (e.g., missense or nonsense) mutation in a target gene. In some embodiments, the mutation in the target gene is associated with a genetic disease or disorder. In some embodiments, the target gene encodes a protein selected from the group consisting of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, and FBN1.

In some embodiments, the subject has been determined to exhibit an aberrant expression or activity of a protein or polynucleotide that corresponds to a target gene. In some embodiments, the aberrant expression or activity of the protein or polynucleotide is associated with a genetic disease or disorder. In some embodiments, the protein is selected from the group consisting of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, and FBN1. In some embodiments, the polynucleotide encodes a protein selected from the group consisting of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, and FBN1.

In some embodiments, the subject is selected from the group consisting of mouse, rat, monkey, and human. In some embodiments, the subject is a human.

In another aspect, provided herein is a method for potent delivery of a therapeutic agent (or prophylactic agent) to a cell comprising contacting the cell with the pharmaceutical composition of the present application. In some embodiments of the method, the pharmaceutical composition comprises a therapeutic agent (or prophylactic agent) assembled with a lipid composition as described in the present application, wherein the lipid composition comprises (i) an ionizable cationic lipid; and (iii) a selective organ targeting (SORT) lipid separate from the ionizable cationic lipid. The lipid composition may further comprise a phospholipid.

In some embodiments of the method, the cell is isolated from the subject. In some embodiments of the method, the cell is a cell line. In some embodiments of the method, the cell is a lung cell. In some embodiments, the lung cell is a lung airway cell. Examples of lung airway cells that can be targeted by the delivery of the present application includes but is not limited to basal cell.

In some embodiments of the method, the SORT lipid effects a delivery of the therapeutic agent to the cell characterized by a greater therapeutic effect compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the method, the SORT lipid achieves about 1.1-fold to about 20-fold therapeutic effect compared to that achieved with a reference lipid composition. In some embodiments of the method, the SORT lipid achieves about 1.1-fold to about 10-fold therapeutic effect compared to that achieved with a reference lipid composition. In some embodiments of the method, the SORT lipid achieves about 1.1-fold to about 5-fold therapeutic effect compared to that achieved with a reference lipid composition. In some embodiments of the method, the SORT lipid achieves about 10-fold to about 20-fold therapeutic effect compared to that achieved with a reference lipid composition. In some embodiments of the method, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect compared to that achieved with a reference lipid composition.

In some embodiments of the method, the SORT lipid effects delivery of the therapeutic agent to cells of the subject characterized by a therapeutic effect in a greater plurality of cells compared to that achieved with a reference lipid composition. In some embodiments of the method, the SORT lipid achieves therapeutic effect in about 1.1-fold to about 20-fold cells compared to that achieved with a reference lipid composition. In some embodiments of the method, the SORT lipid achieves therapeutic effect in about 1.1-fold to about 10-fold cells compared to that achieved with a reference lipid composition. In some embodiments of the method, the SORT lipid achieves therapeutic effect in about 5-fold to about 10-fold cells compared to that achieved with a reference lipid composition. In some embodiments of the method, the SORT lipid achieves therapeutic effect in about 10-fold to about 20-fold cells compared to that achieved with a reference lipid composition. In some embodiments of the method, the SORT lipid achieves therapeutic effect in at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold cells compared to that achieved with a reference lipid composition.

In another aspect, provided herein is a method for targeted delivery of a therapeutic agent (or prophylactic agent) to a cell type comprising contacting the cell with the pharmaceutical composition of the present application. In some embodiments of the method, the pharmaceutical composition comprises a therapeutic agent (or prophylactic agent) assembled with a lipid composition as described in the present application, wherein the lipid composition comprises (i) an ionizable cationic lipid; and (iii) a selective organ targeting (SORT) lipid separate from the ionizable cationic lipid. The lipid composition may further comprise a phospholipid.

In some embodiments of the method, the cell is isolated from the subject. In some embodiments of the method, the cell is a cell line. In some embodiments of the method, the cell is a lung cell. In some embodiments, the lung cell is a lung airway cell. Examples of lung airway cells that can be targeted by the delivery of the present application includes but is not limited to basal cell.

In some embodiments of the method, the SORT lipid effects delivery of the therapeutic agent to cells of the subject characterized by a therapeutic effect in a first plurality of cells of a first cell type and in a greater therapeutic effect in a second plurality of cells of a second cell type. In some embodiments, the first cell type is different from the second cell type. In some embodiments of the method, the SORT lipid achieves therapeutic effect in about 1.1-fold to about 20-fold greater second plurality of cells of the second cell type compared to the first plurality of cells of the first cell type. In some embodiments of the method, the SORT lipid achieves therapeutic effect in about 1.1-fold to about 10-fold greater second plurality of cells of the second cell type compared to the first plurality of cells of the first cell type. In some embodiments of the method, the SORT lipid achieves therapeutic effect in about 1.1-fold to about 5-fold greater second plurality of cells of the second cell type compared to the first plurality of cells of the first cell type. In some embodiments of the method, the SORT lipid achieves therapeutic effect in about 10-fold to about 20-fold greater second plurality of cells of the second cell type compared to the first plurality of cells of the first cell type. In some embodiments of the method, the SORT lipid achieves therapeutic effect in at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold greater second plurality of cells of the second cell type compared to the first plurality of cells of the first cell type.

In some embodiments of the method, the SORT lipid effects delivery of the therapeutic agent to cells of the subject characterized by a greater therapeutic effect in a first cell of a first cell type compared to that in a second cell of a second cell type. In some embodiments, the second cell type is different from the first cell type. In some embodiments of the method, the SORT lipid achieves about 1.1-fold to about 20-fold therapeutic effect in first cell of the first cell type compared to that achieved in the second cell of the second cell type. In some embodiments of the method, the SORT lipid achieves about 1.1-fold to about 10-fold therapeutic effect in first cell of the first cell type compared to that achieved in the second cell of the second cell type. In some embodiments of the method, the SORT lipid achieves about 5-fold to about 10-fold therapeutic effect in first cell of the first cell type compared to that achieved in the second cell of the second cell type. In some embodiments of the method, the SORT lipid achieves about 10-fold to about 20-fold therapeutic effect in first cell of the first cell type compared to that achieved in the second cell of the second cell type. In some embodiments of the method, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect in first cell of the first cell type compared to that achieved in the second cell of the second cell type.

In some embodiments of the method, the first cell type is a lung cell. In some embodiments, the first cell type is a lung airway cell. Examples of lung airway cells that can be targeted by the delivery of the present application includes but is not limited to basal cell.

In some embodiments of the method, the second cell type is a lung cell. In some embodiments, the second cell type is a lung airway cell.

In some embodiments, the contacting is ex vivo. In some embodiments, the contacting is in vitro. In some embodiments, the contacting is in vivo. In some embodiments, the contacting comprises intravenously administering to a subject the composition comprising the therapeutic agent assembled with the lipid composition.

The following are examples of compositions and evaluations of compositions of the disclosure. It is understood that various other embodiments may be practiced, given the general description provided above.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for potent delivery to a (e.g., non-liver, such as lung) cell (e.g., a lung basal cell) of a subject, comprising:
intravenously administering to said subject a composition comprising a therapeutic agent assembled with a lipid composition which comprises:
   (i) an ionizable cationic lipid; and
   (ii) a selective organ targeting (SORT) lipid separate from said ionizable cationic lipid, wherein (e.g., an amount of) said SORT lipid effects delivery of said therapeutic agent to said cell of said subject characterized by a (e.g., about 1.1- or 10-fold) greater therapeutic effect compared to that achieved with a reference lipid composition (e.g., without said amount of said SORT lipid).

Embodiment 2. A method for potent delivery to (e.g., non-liver, such as lung) cells (e.g., lung basal cells) of a subject, comprising:
intravenously administering to said subject a composition comprising a therapeutic agent assembled with a lipid composition which comprises:
   (i) an ionizable cationic lipid; and
   (ii) a selective organ targeting (SORT) lipid separate from said ionizable cationic lipid, wherein (e.g., an amount of) said SORT lipid effects delivery of said therapeutic agent to cells of said subject characterized by a therapeutic effect in a (e.g., about 1.1- or 10-fold) greater plurality or proportion of (e.g., lung) cells compared to that achieved with a reference lipid composition (e.g., without said amount of said SORT lipid).

Embodiment 3. A method for targeted delivery to (e.g., lung) cells of a subject, comprising:
intravenously administering to said subject a composition comprising a therapeutic agent assembled with a lipid composition which comprises:
   (i) an ionizable cationic lipid; and
   (ii) a selective organ targeting (SORT) lipid separate from said ionizable cationic lipid, wherein (e.g., an amount of) said SORT lipid effects delivery of said therapeutic agent to a greater proportion of cell types (e.g., comprising (e.g., lung) basal cells) as compared to that achieved with a reference lipid composition.

Embodiment 4. A method for targeted delivery to (e.g., lung) cells of a subject, comprising:
intravenously administering to said subject a composition comprising a therapeutic agent assembled with a lipid composition which comprises:
   (i) an ionizable cationic lipid; and
   (ii) a selective organ targeting (SORT) lipid separate from said ionizable cationic lipid, wherein (e.g., an amount of) said SORT lipid effects delivery of said therapeutic agent to cells of said subject characterized by a therapeutic effect in a first plurality or proportion of (e.g., lung) cells of a first cell type (e.g., (e.g., lung) non-basal cell type, such as (e.g., lung) epithelial cell. (e.g., lung) ciliated cell, (e.g., lung) club cell, or (e.g., lung) goblet cell) and in a (e.g., about 1.1- or 10-fold) greater second plurality or proportion of (e.g., lung) cells of a second cell type (e.g., (e.g., lung) basal cell).

Embodiment 5. A method for targeted delivery to (e.g., lung) cells of a subject, comprising:
intravenously administering to said subject a composition comprising a therapeutic agent assembled with a lipid composition which comprises:
   (i) an ionizable cationic lipid; and
   (ii) a selective organ targeting (SORT) lipid separate from said ionizable cationic lipid, wherein (e.g., an amount of) said SORT lipid effects a delivery of said therapeutic agent to cells of said subject characterized by a (e.g., about 1.1- or 10-fold) greater therapeutic effect in a first (e.g., lung) cell of a first cell type (e.g., non-basal cell type, such as (e.g., lung) epithelial cell, (e.g., lung) ciliated cell, (e.g., lung) club cell, or (e.g., lung) goblet cell) of said subject compared to that in a second (e.g., lung) cell of a second cell type of said subject, wherein said first cell type is different from said second cell type (e.g., (e.g., lung) basal cell).

Embodiment 6. A method for delivery to (e.g., lung) basal cells of a subject, comprising:
intravenously administering to said subject a therapeutic agent assembled with a lipid composition that comprises:
   (i) an ionizable cationic lipid; and
   (ii) a selective organ targeting (SORT) lipid separate from said ionizable cationic lipid, thereby delivering said therapeutic agent to an organ or tissue (e.g., lung) of said subject to result in a therapeutic effect detectable in at least about 1%. 2%, 3%, 4%, 5%, 6%, 7%, 8%, 9%, 10%, 11%, 12%, 13%, 14%, or 15% basal cells in said organ or tissue of said subject.

Embodiment 7. The method of Embodiment 6, wherein said therapeutic effect is characterized by an amount or activity of said agent detectable in said at least about 10% (e.g., at least about 15%) basal cells in said organ or tissue of said subject.

Embodiment 8. The method of any one of Embodiments 1-7, wherein said lipid composition further comprises (iii) a phospholipid.

Embodiment 9. The method of any one of Embodiments 1-8, wherein said lipid composition comprises said SORT lipid at a molar percentage from about 20% to about 65%.

Embodiment 10. The method of any one of Embodiments 1-9, wherein said lipid composition comprises said ionizable cationic lipid at a molar percentage from about 5% to about 30%.

Embodiment 11. The method of any one of Embodiments 1-10, wherein said lipid composition comprises said phospholipid at a molar percentage from about 8% to about 23%.

Embodiment 12. The method of any one of Embodiments 1-11, wherein said phospholipid is not an ethylphosphocholine.

Embodiment 13. The method of any one of Embodiments 1-12, wherein said lipid composition further comprises a steroid or steroid derivative.

Embodiment 14. The method of Embodiment 13, wherein said lipid composition comprises said steroid or steroid derivative at a molar percentage from about 15% to about 46%.

Embodiment 15. The method of any one of Embodiments 1-14, wherein said lipid composition further comprises a polymer-conjugated lipid (e.g., poly(ethylene glycol) (PEG)-conjugated lipid).

Embodiment 16. The method of Embodiment 15, wherein said lipid composition comprises said polymer-conjugated lipid at a molar percentage from about 0.5% to about 10%.

Embodiment 17. The method of Embodiment 15, wherein said lipid composition comprises said polymer-conjugated lipid at a molar percentage from about 1% to about 10%.

Embodiment 18. The method of Embodiment 15, wherein said lipid composition comprises said polymer-conjugated lipid at a molar percentage from about 2% to about 10%.

Embodiment 19. The method of any one of Embodiments 1-18, wherein said therapeutic agent is a polynucleotide; and wherein a molar ratio of nitrogen in said lipid composition to phosphate in said polynucleotide (N/P ratio) is no more than about 20:1.

Embodiment 20. The method of Embodiment 19, wherein said N/P ratio is from about 5:1 to about 20:1.

Embodiment 21. The method of any one of Embodiments 1-20, wherein a molar ratio of said therapeutic agent to total lipids of said lipid composition is no more than about 1:1, 1:10, 1:50, or 1:100.

Embodiment 22. The method of any one of Embodiments 1-21, wherein at least about 85% of said therapeutic agent is encapsulated in particles of said lipid compositions.

Embodiment 23. The method of any one of Embodiments 1-22, wherein said lipid composition comprises a plurality of particles characterized by one or more characteristics of the following:
(1) a (e.g., average) size of 100 nanometers (nm) or less;
(2) a polydispersity index (PDI) of no more than about 0.2; and
(3) a negative zeta potential of −10 millivolts (mV) to 10 mV.

Embodiment 24. The method of any one of Embodiments 1-23, wherein said lipid composition has an apparent ionization constant (pKa) outside a range of 6 to 7.

Embodiment 25. The method of Embodiment 24, wherein said apparent pKa of said lipid composition is of about 7 or higher.

Embodiment 26. The method of Embodiment 24, wherein said apparent pKa of said lipid composition is of about 8 or higher.

Embodiment 27. The method of Embodiment 26, wherein said apparent pKa of said lipid composition is from about 8 to about 13.

Embodiment 28. The method of any one of Embodiments 1-27, wherein said SORT lipid comprises a permanently positively charged moiety (e.g., a quaternary ammonium ion).

Embodiment 29. The method of Embodiment 28, wherein said SORT lipid comprises a counterion.

Embodiment 30. The method of any one of Embodiments 1-29, wherein said SORT lipid is a phosphocholine lipid (e.g., saturated or unsaturated).

Embodiment 31. The method of any one of Embodiments 30, wherein said SORT lipid is an ethylphosphocholine.

Embodiment 32. The method of any one of Embodiments 1-31, wherein said SORT lipid comprises a headgroup having a structural formula:

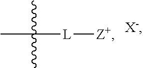

wherein L is a (e.g., biodegradable) linker; Z⁺ is positively charged moiety (e.g., a quaternary ammonium ion); and X⁻ is a counterion.

Embodiment 33. The method of Embodiment 32, wherein said SORT lipid has a structural formula:

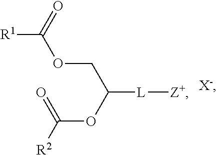

wherein R¹ and R² are each independently an optionally substituted C₆-C₂₄ alkyl, or an optionally substituted C₆-C₂₄ alkenyl.

Embodiment 34. The method of Embodiment 32, wherein said SORT lipid has a structural formula:

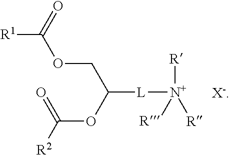

Embodiment 35. The method of any one of Embodiments 32-34, wherein L is

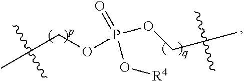

wherein:
p and q are each independently 1, 2, or 3; and
R₄ is an optionally substituted C₁-C₆ alkyl.

Embodiment 36. The method of Embodiment 32, wherein said SORT lipid has a structural formula:

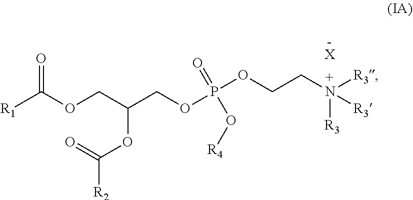

wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
R₄ is alkyl_{(C≤6)} or substituted alkyl_{(C≤6)}; and
X⁻ is a monovalent anion.

Embodiment 37. The method of any one of Embodiments 1-29, wherein said SORT lipid has a structural formula:

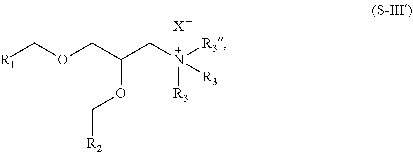

wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
X⁻ is a monovalent anion.

Embodiment 38. The method of any one of Embodiments 1-29, wherein said SORT lipid has a structural formula:

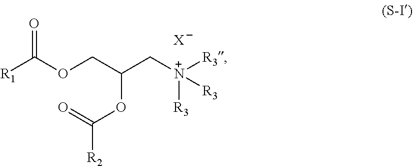

wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
X⁻ is a monovalent anion.

Embodiment 39. The method of any one of Embodiments 1-29, wherein said SORT lipid has a structural formula:

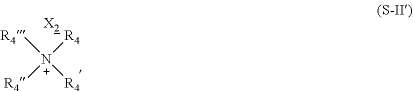

wherein:
R₄ and R_{4′} are each independently alkyl_{(C6-C24)}, alkenyl_{(C6-C24)}, or a substituted version of either group;
R_{4″} is alkyl_{(C≤24)}, alkenyl_{(C≤24)}, or a substituted version of either group;
R_{4″} is alkyl_{(C2-C8)}, alkenyl_{(C2-C8)}, or a substituted version of either group; and
X₂ is a monovalent anion.

Embodiment 40. The method of any one of Embodiments 1-27, wherein said SORT lipid is selected from those set forth in Table 6, or pharmaceutically acceptable salts thereof, or a subset of the lipids and the pharmaceutically acceptable salts thereof.

Embodiment 41. The method of any one of Embodiments 1-40, wherein the ionizable cationic lipid is a dendrimer or dendron of a generation (g) having a structural formula:

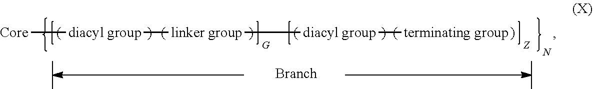

or a pharmaceutically acceptable salt thereof, wherein:
(a) the core comprises a structural formula (X_{Core}):

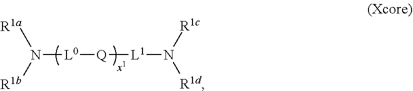

wherein:
Q is independently at each occurrence a covalent bond, —O—, —S—, —NR²—, or —CR^{3a}R^{3b}—;
R² is independently at each occurrence Rig or -L²-NR^{1e}R^{1f}.
R^{3a} and R^{3b} are each independently at each occurrence hydrogen or an optionally substituted (e.g., C₁-C₆, such as C₁-C₃) alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
x¹ is 0, 1, 2, 3, 4, 5, or 6; and
(b) each branch of the plurality (N) of branches independently comprises a structural formula (X_{Branch}):

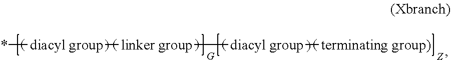

wherein:
* indicates a point of attachment of the branch to the core;
g is 1, 2, 3, or 4;
Z=2^{(g-1)};
G=0, when g=1; or G=Σᵢ₌₀^{i=g-2}2ⁱ, when g≠1;
(c) each diacyl group independently comprises a structural formula

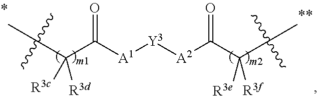

wherein:
* indicates a point of attachment of the diacyl group at the proximal end thereof;
** indicates a point of attachment of the diacyl group at the distal end thereof;
Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR⁴—, wherein:
   R⁴ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula

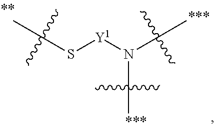

wherein:
** indicates a point of attachment of the linker to a proximal diacyl group;
*** indicates a point of attachment of the linker to a distal diacyl group; and
Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

Embodiment 42. The method of Embodiment 41, wherein x¹ is 0, 1, 2, or 3.

Embodiment 43. The method of Embodiment 41 or 42, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl

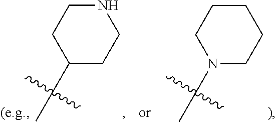 [SMILES: *C1CCNCC1 ; *N1CCCCC1]

N—(C₁-C₃ alkyl)-piperidinyl

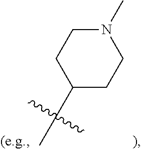 [SMILES: *C1CCN(C)CC1]

piperazinyl

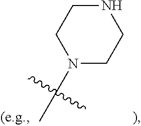 [SMILES: *N1CCNCC1]

N—(C₁-C₃ alkyl)-piperadizinyl

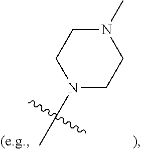 [SMILES: *N1CCN(C)CC1]

morpholinyl

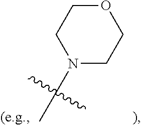 [SMILES: *N1CCOCC1]

### N-pyrrolidinyl

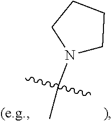 [SMILES: *N1CCCC1]

pyrrolidinyl

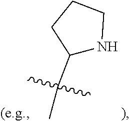 [SMILES: *C1CCCN1]

or N—(C₁-C₃ alkyl)-pyrrolidinyl

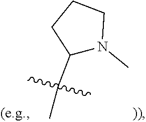 [SMILES: *C1CCCN1C]

(e.g. C₆-C₁₀) aryl, and C₃-C₅ heteroaryl (e.g., imidazolyl

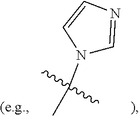 [SMILES: *n1ccnc1]

or pyridinyl

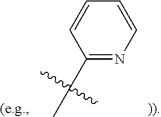 [SMILES: *c1ccccn1]

Embodiment 44. The method of Embodiment 43, wherein R^{1a}, R^{1b}, R^{1c}, R^{1d}, R^{1e}, R^{1f} and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

Embodiment 45. The method of any one of Embodiments 41-44, wherein R^{3a} and R^{3b} are each independently at each occurrence hydrogen.

Embodiment 46. The method of any one of Embodiments 41-45, wherein the plurality (N) of branches comprises at least 3 (e.g., at least 4, or at least 5) branches.

Embodiment 47. The method of any one of Embodiments 41-46, wherein g=1; G=0; and Z=1.

Embodiment 48. The method of Embodiment 47, wherein each branch of the plurality of branches comprises a structural formula *-(diacyl group)-(terminating group).

Embodiment 49. The method of any one of Embodiments 41-46, wherein g=2; G=1; and Z=2.

Embodiment 50. The method of Embodiment 49, wherein each branch of the plurality of branches comprises a structural formula

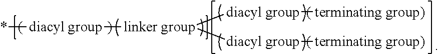

Embodiment 51. The method of any one of Embodiments 41-50, wherein the core comprises a structural formula:

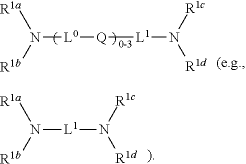

Embodiment 52. The method of any one of Embodiments 41-50, wherein the core comprises a structural formula:

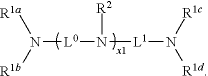

Embodiment 53. The method of Embodiment 52, wherein the core comprises a structural formula:

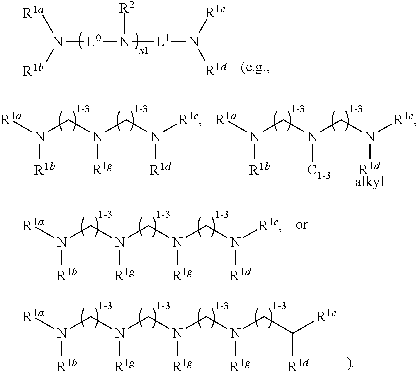

Embodiment 54. The method of Embodiment 52, wherein the core comprises a structural formula:

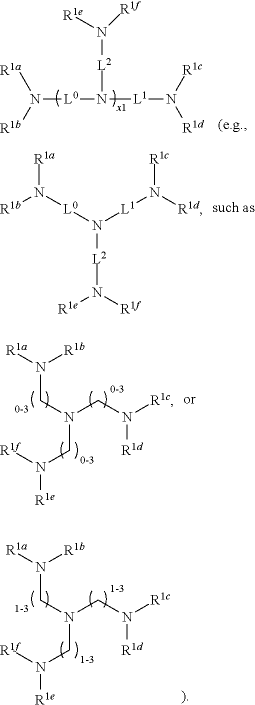

Embodiment 55. The method of any one of Embodiments 41-50, wherein the core comprises a structural formula:

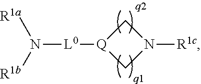

wherein Q′ is —NR²— or —CR^{3a}R^{3b}—; q¹ and q² are each independently 1 or 2.

Embodiment 56. The method of Embodiment 55, wherein the core comprises a structural formula:

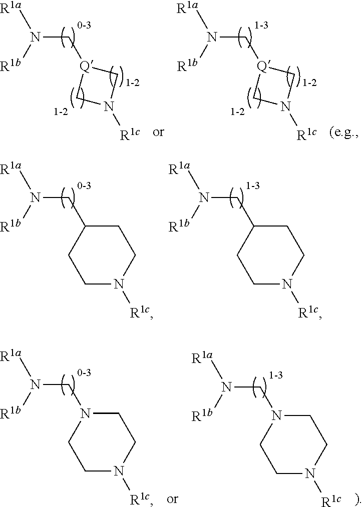

Embodiment 57. The method of any one of Embodiments 41-50, wherein the core comprises a structural formula

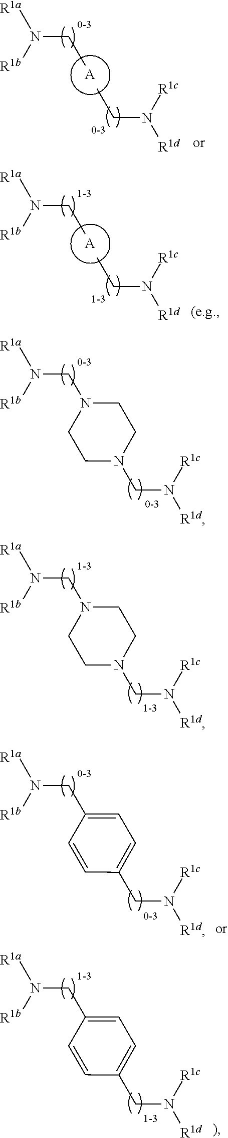

wherein ring A is an optionally substituted aryl or an optionally substituted (e.g., C₃-C₁₂, such as C₃-C₅) heteroaryl.

Embodiment 58. The method of any one of Embodiments 41-50, wherein the core comprises has a structural formula

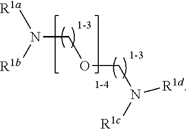

Embodiment 59. The method of any one of Embodiments 41-50, wherein the core is selected from those set forth in Table 1 or a subset thereof.

Embodiment 60. The method of any one of Embodiments 41-50, wherein the core comprises a structural formula selected from the group consisting of:

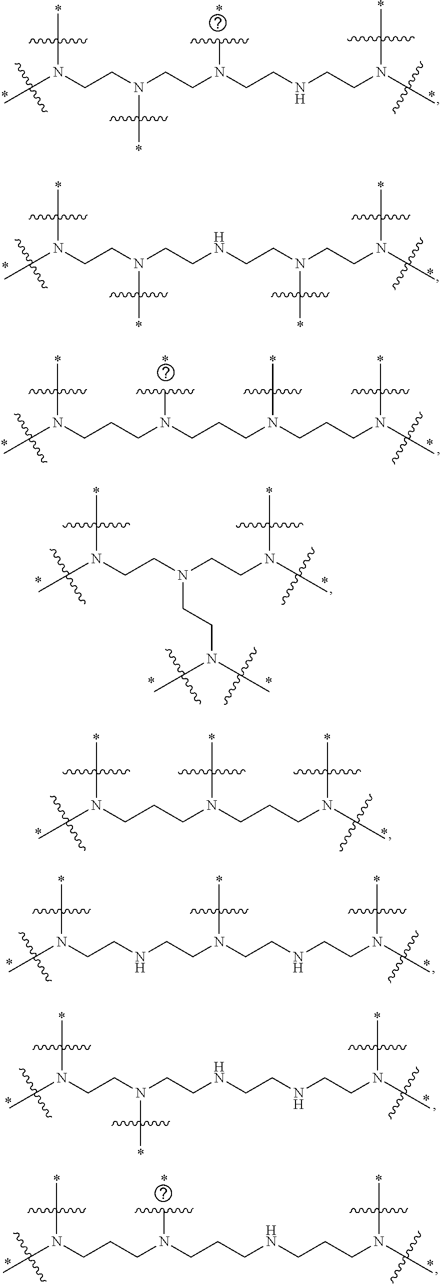

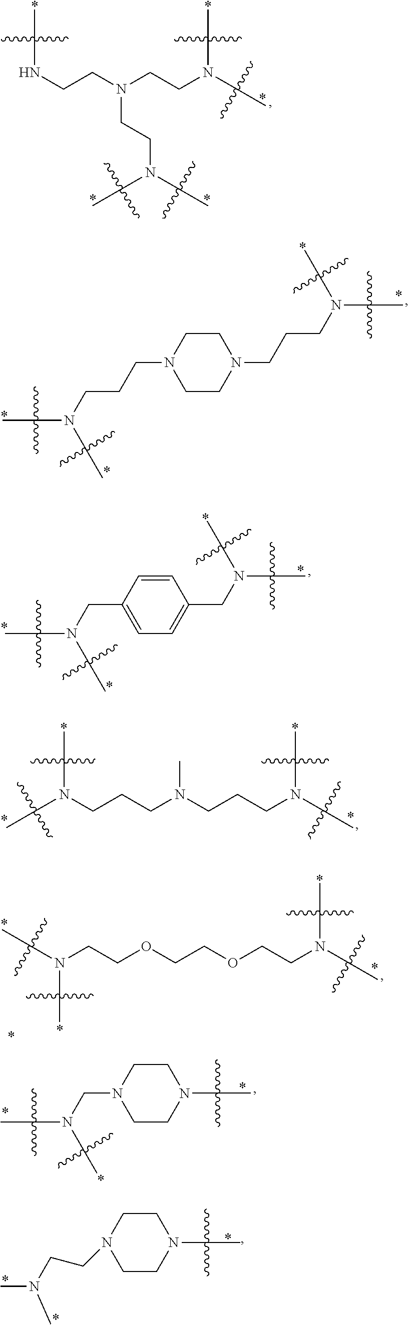

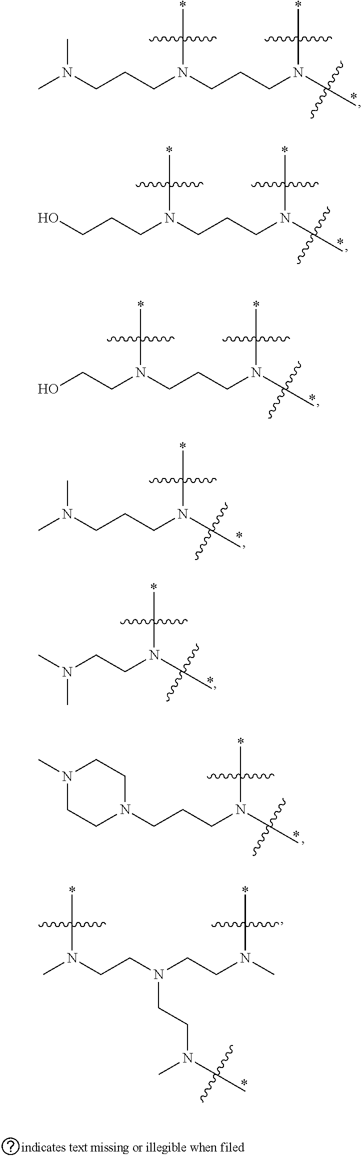

and pharmaceutically acceptable salts thereof, wherein * indicates a point of attachment of the core to a branch of the plurality of branches.

Embodiment 61. The method of any one of Embodiments 41-50, wherein the core comprises a structural formula selected from the group consisting of:

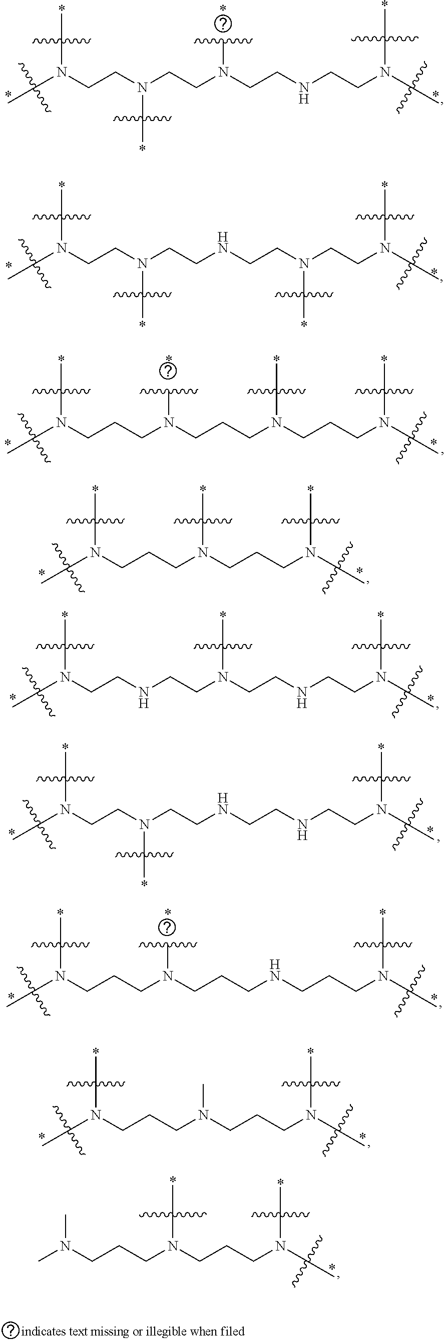

and pharmaceutically acceptable salts thereof, wherein * indicates a point of attachment of the core to a branch of the plurality of branches.

Embodiment 62. The method of any one of Embodiments 41-50, wherein the core has the structure

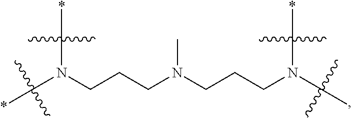

wherein * indicates a point of attachment of the core to a branch of the plurality of branches or H.

Embodiment 63. The method of Embodiment 62, wherein at least 2 branches are attached to the core.

Embodiment 64. The method of Embodiment 62, wherein at least 3 branches are attached to the core.

Embodiment 65. The method of Embodiment 62, wherein at least 4 branches are attached to the core.

Embodiment 66. The method of any one of Embodiments 41-50, wherein the core has the structure

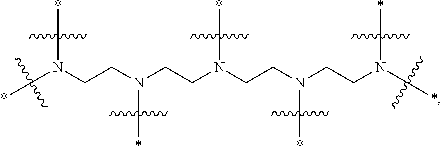

wherein * indicates a point of attachment of the core to a branch of the plurality of branches or H.

Embodiment 67. The method of Embodiment 65, wherein at least 4 branches are attached to the core.

Embodiment 68. The method of Embodiment 65, wherein at least 5 branches are attached to the core.

Embodiment 69. The method of Embodiment 65, wherein at least 6 branches are attached to the core.

Embodiment 70. The method of any one of Embodiments 41-69, wherein A¹ is —O— or —NH—.

Embodiment 71. The method of Embodiment 70, wherein A¹ is —O—.

Embodiment 72. The method of any one of Embodiments 41-70, wherein A² is —O— or —NH—.

Embodiment 73. The method of any Embodiment 72, wherein A² is —O—.

Embodiment 74. The method of any one of Embodiments 41-73, wherein Y³ is C₁-C₁₂ (e.g., C₁-C₆, such as C₁-C₃) alkylene.

Embodiment 75. The method of any one of Embodiments 41-74, wherein the diacyl group independently at each occurrence comprises a structural formula

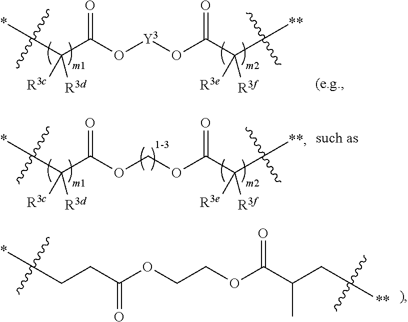

optionally wherein R^{3c}, R^{3d}, R^{3e} and R^{3f} are each independently at each occurrence hydrogen or C₁-C₃ alkyl.

Embodiment 76. The method of any one of Embodiments 41-70, wherein L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

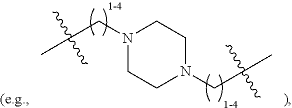

and [(C₁-C₄) alkylene]-phenylene-[(C₁-C₄) alkylene]

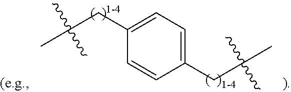

Embodiment 77. The method of Embodiment 76, wherein L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-.

Embodiment 78. The method of Embodiment 76, wherein L⁰, L¹, and L² are each independently at each occurrence C₁-C₆ alkylene (e.g., C₁-C₃ alkylene).

Embodiment 79. The method of Embodiment 76, wherein L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)).

Embodiment 80. The method of Embodiment 76, wherein L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

Embodiment 81. The method of any one of Embodiments 41-80, wherein each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino

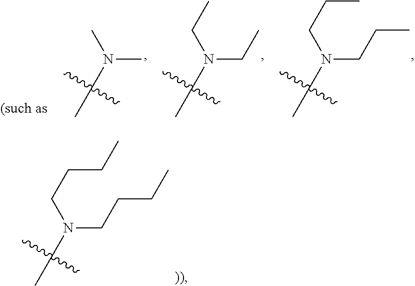

C₄-C₆ N-heterocycloalkyl (e.g., N-pyrrolidinyl

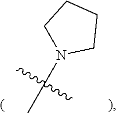 [SMILES: *N1CCCC1]

### N-piperidinyl

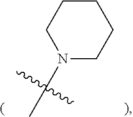 [SMILES: *N1CCCCC1]

### N-azepanyl

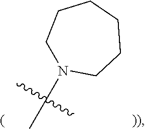 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

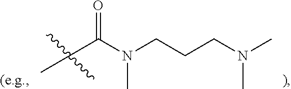 [SMILES: *C(=O)N(C)CCCN(C)C]

—C(O)N(C₁-C₃ alkyl)-(C₁-C₃ alkylene)-(C₄-C₆ N-heterocycloalkyl)

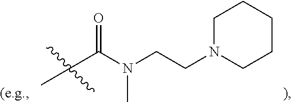 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

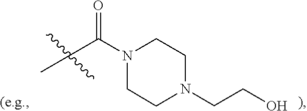 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 82. The method of Embodiment 81, wherein each terminating group is independently C₁-C₁₈ (e.g., C₄-C₁₈) alkylthiol, wherein the alkyl moiety is optionally substituted with one or more (e.g., one) substituents each independently selected from C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino

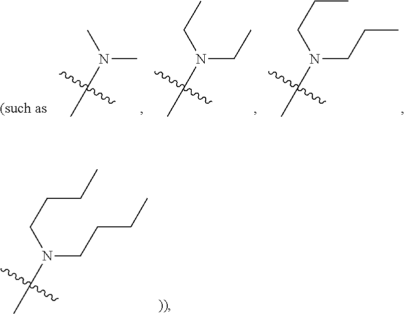

C₄-C₆ N-heterocycloalkyl (e.g., N-pyrrolidinyl

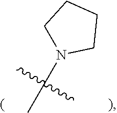 [SMILES: *N1CCCC1]

### N-piperidinyl

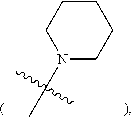 [SMILES: *N1CCCCC1]

### N-azepanyl

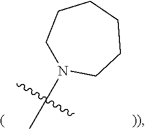 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

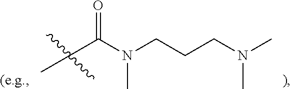 [SMILES: *C(=O)N(C)CCCN(C)C]

—C(O)N(C₁-C₃ alkyl)-(C₁-C₆ alkylene)-(C₄-C₆ N-heterocycloalkyl) and

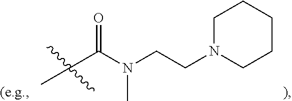 [SMILES: *C(=O)N(C)CCN1CCCCC1]

—C(O)—(C₄-C₆ N-heterocycloalkyl)

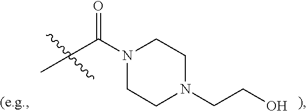 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 83. The method of Embodiment 82, wherein each terminating group is independently C₁-C₁₈ (e.g., C₄-C₁₈) alkylthiol, wherein the alkyl moiety is optionally substituted with one substituent —OH.

Embodiment 84. The method of Embodiment 82, wherein each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino

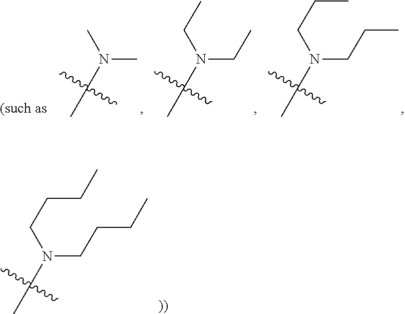

and C₄-C₆ N-heterocycloalkyl (e.g., N-pyrrolidinyl

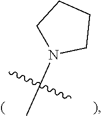 [SMILES: *N1CCCC1]

### N-piperidinyl

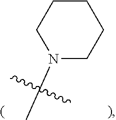 [SMILES: *N1CCCCC1]

### N-azepanyl

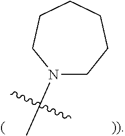 [SMILES: *N1CCCCCC1]

Embodiment 85. The method of Embodiment 81, wherein each terminating group is independently C₁-C₁₈ (e.g., C₄-C₁₈) alkenylthiol or C₁-C₁₈ (e.g., C₄-C₁₈) alkylthiol.

Embodiment 86. The method of Embodiment 85, wherein each terminating group is independently C₁-C₁₈ (e.g., C₄-C₁₈) alkylthiol.

Embodiment 87. The method of Embodiment 86, wherein each terminating group is independently selected from the group consisting of:

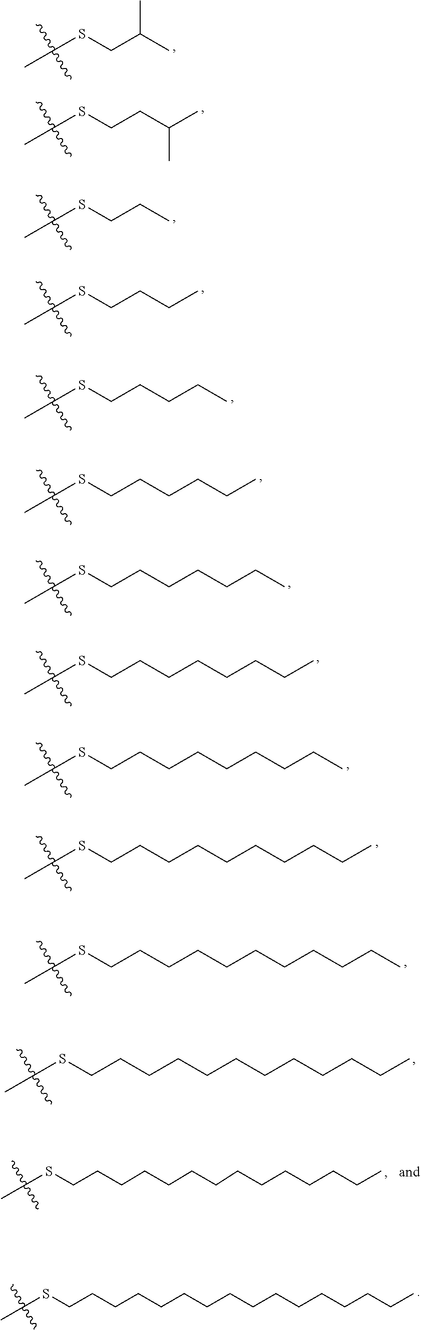

Embodiment 88. The method of any one of Embodiments 41-80, wherein each terminating group is independently selected from those set forth in Table 3 or a subset thereof.

Embodiment 89. The method of Embodiment 41, wherein the dendrimer or dendron is selected from the group consisting of

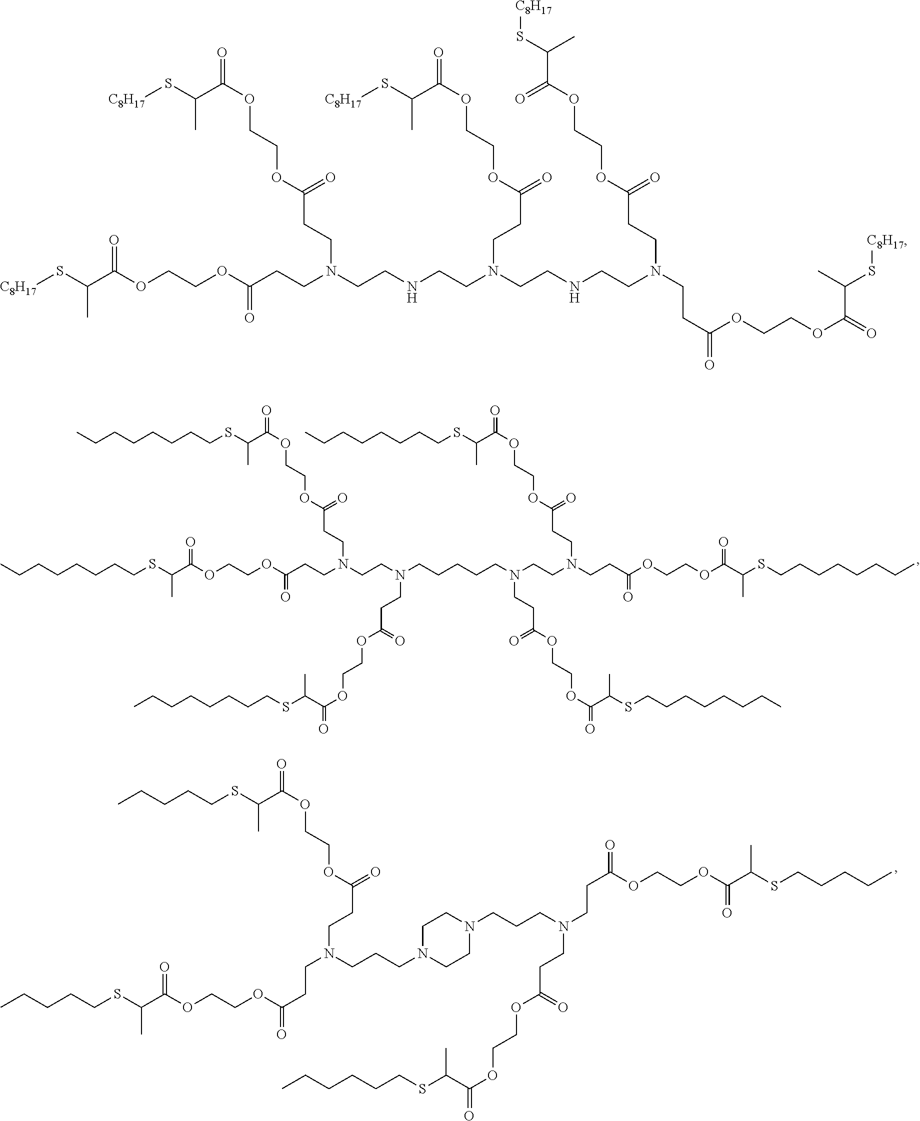

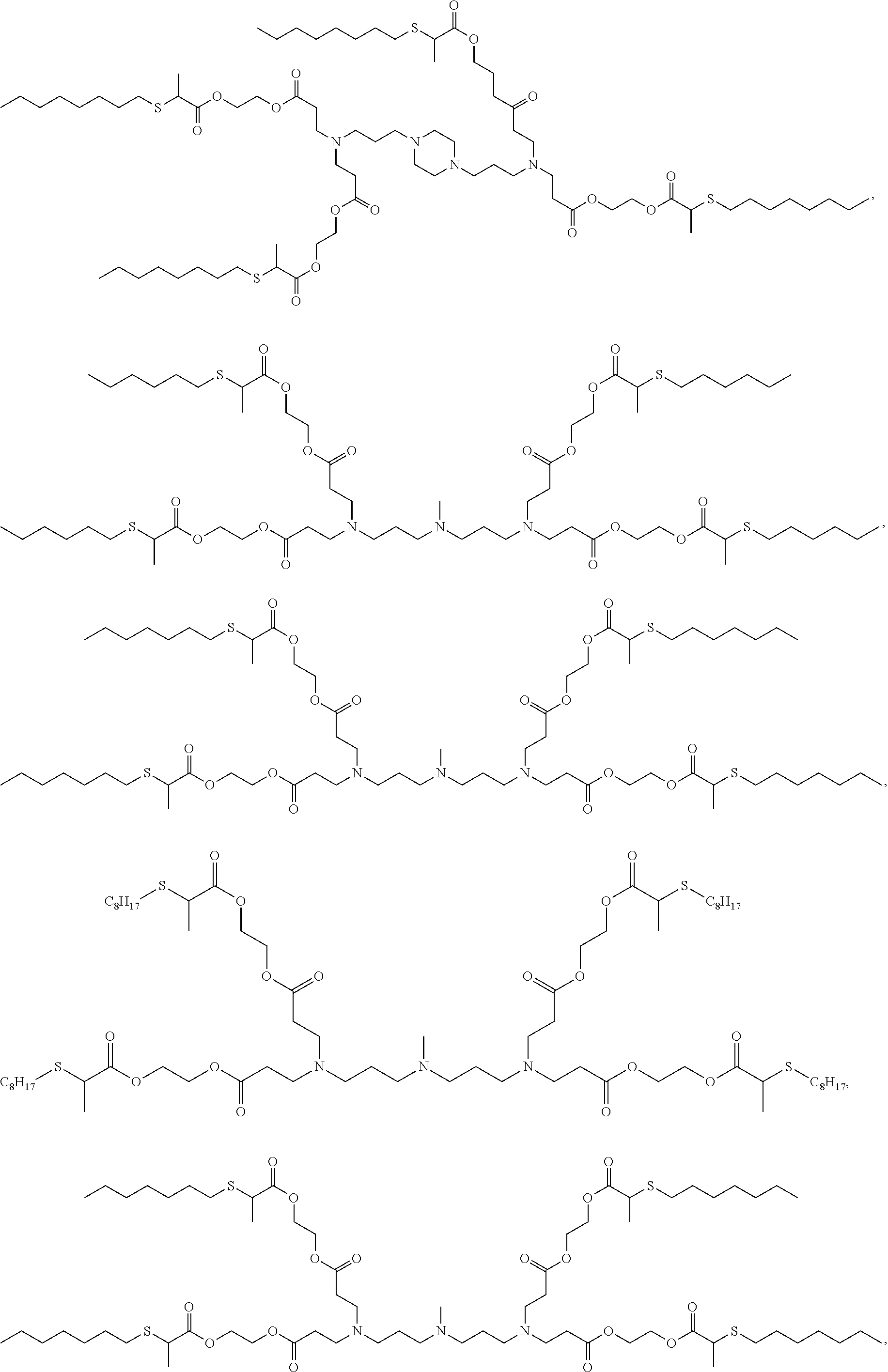

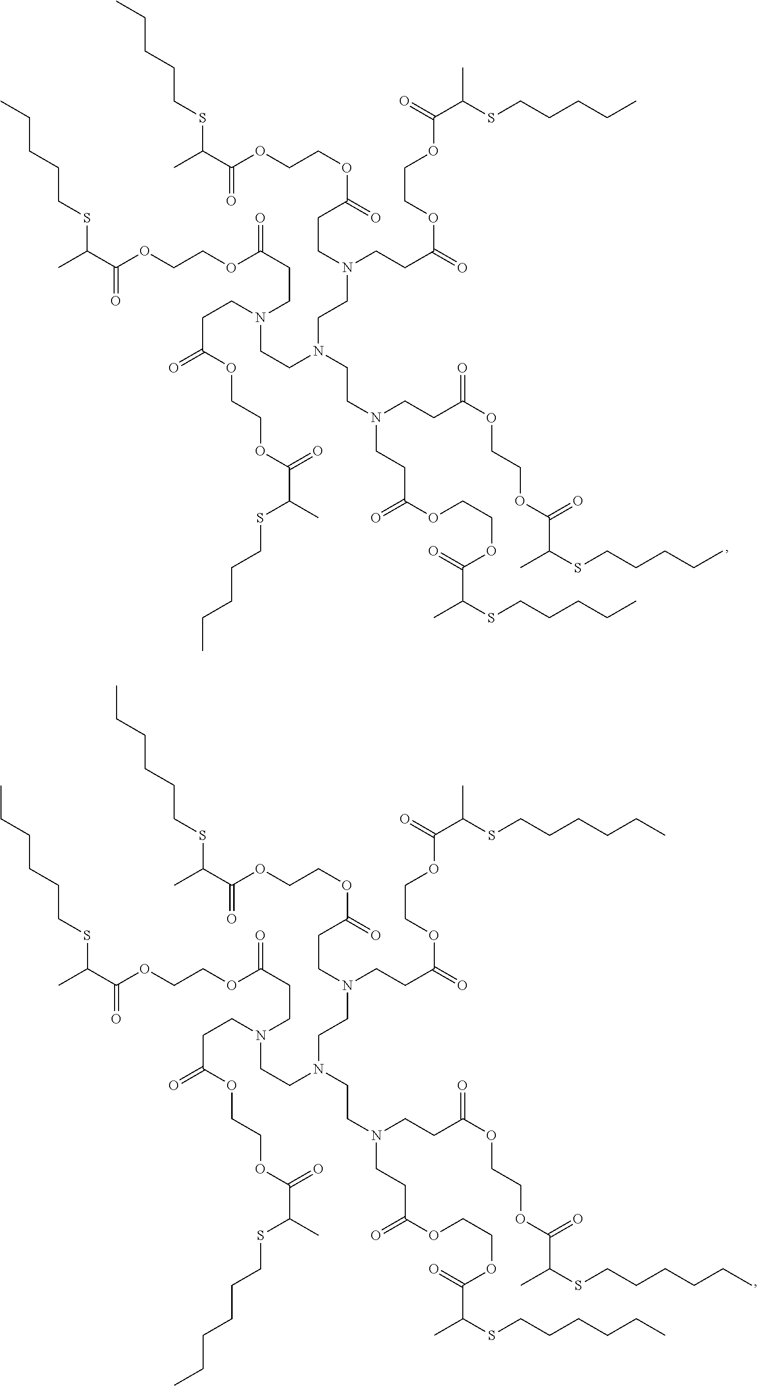 [SMILES: CCCCCSC(C)C(=O)OCCOC(=O)CCN(CCC(=O)OCCOC(=O)C(C)SCCCCC)CCN(CCN(CCC(=O)OCCOC(=O)C(C)SCCCCC)CCC(=O)OCCOC(=O)C(C)SCCCCC)CCN(CCC(=O)OCCOC(=O)C(C)SCCCCC)CCC(=O)OCCOC(=O)C(C)SCCCCC ; CCCCCCSC(C)C(=O)OCCOC(=O)CCN(CCC(=O)OCCOC(=O)C(C)SCCCCCC)CCN(CCN(CCC(=O)OCCOC(=O)C(C)SCCCCCC)CCC(=O)OCCOC(=O)C(C)SCCCCCC)CCN(CCC(=O)OCCOC(=O)C(C)SCCCCCC)CCC(=O)OCCOC(=O)C(C)SCCCCCC]

and pharmaceutically acceptable salts thereof.

Embodiment 90. The method of any one of Embodiments 1-40, wherein the ionizable cationic lipid is selected from those set forth in Table 4, or pharmaceutically acceptable salts thereof, or a subset of the lipids and the pharmaceutically acceptable salts thereof.

Embodiment 91. The method of any one of Embodiments 1-40, wherein the ionizable cationic lipid is selected from those set forth in Table 4 or Table 5, or pharmaceutically acceptable salts thereof, or a subset of the lipids and the pharmaceutically acceptable salts thereof.

Embodiment 92. The method of any one of Embodiments 1-91, wherein said subject has been determined to likely respond to said therapeutic agent.

Embodiment 93. The method of any one of Embodiments 1-92, wherein said subject has been determined to have a (e.g., missense or nonsense) mutation in a target gene.

Embodiment 94. The method of Embodiment 93, wherein said mutation in said target gene is associated with a genetic disease or disorder.

Embodiment 95. The method of any one of Embodiments 1-94, wherein said subject has been determined to exhibit an aberrant expression or activity of a protein or polynucleotide that corresponds to a target gene.

Embodiment 96. The method of Embodiment 95, wherein said aberrant expression or activity of said protein or polynucleotide is associated with a genetic disease or disorder.

Embodiment 97. The method of any one of Embodiments 1-96, wherein said subject is selected from the group consisting of mouse, rat, monkey, and human.

Embodiment 98. The method of Embodiment 97, wherein said subject is a human.

Embodiment 99. The method of any one of Embodiments 1-98, wherein said therapeutic agent comprises a compound, a polynucleotide, a polypeptide, or a combination thereof.

Embodiment 100. The method of Embodiment 99, wherein said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRSIPR-associated (Cas) protein, or a combination thereof.

Embodiment 101. The method of Embodiment 100, wherein said therapeutic agent comprises a heterologous messenger ribonucleotide (mRNA); and wherein said intravenous administration results in an expression, activity, or effect of a protein encoded by said heterologous mRNA detectable in said at least about at least about 1%, 2%, 3%, 4%, 5%, 6%. 7%, 8%, 9%, 10%. 11%, 12%, 13%, 14%, or 15% lung basal cells of said subject.

Embodiment 102. The method of Embodiment 101, wherein said protein is any one selected from the group consisting of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, and FBN1.

Embodiment 103. The method of Embodiment 101, wherein said protein corresponds to a target gene in a lung cell (e.g., a lung basal cell) of said subject.

Embodiment 104. The method of Embodiment 101, wherein an expression of said heterologous mRNA produces a functional variant of said protein.

Embodiment 105. The method of Embodiment 101, wherein an expression of said heterologous mRNA increases an amount of a functional variant of said protein as compared to an amount of said functional variant of said protein generated in absence of said intravenous administration.

Embodiment 106. The method of Embodiment 100, wherein said therapeutic agent comprises a heterologous transfer ribonucleotide (tRNA) that introduces an amino acid into a growing peptide chain of a protein of a target gene (e.g., at a position corresponding to a mutation in said target gene encoding said protein); and wherein said intravenous administration results in an expression or activity of said protein detectable in said at least about at least about 1%. 2%. 3%, 4%, 5%, 6%, 7%. 8%, 9%, 10%, 11%. 12%. 13%, 14%, or 15% lung basal cells of said subject.

Embodiment 107. The method of Embodiment 106, wherein said protein is any one selected from the group consisting of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, and FBN1.

Embodiment 108. The method of Embodiment 106, wherein said target gene is present in a lung cell (e.g., a lung basal cell) of said subject.

Embodiment 109. The method of Embodiment 106, wherein said tRNA reduces an amount of a non-functional variant of said protein in said cell as compared to an amount of said non-functional variant of said protein generated in absence of said contacting.

Embodiment 110. The method of Embodiment 99, wherein said therapeutic agent comprises a heterologous polypeptide comprising an actuator moiety, which actuator moiety is configured to complex with a target polynucleotide corresponding to a target gene; and wherein said intravenous administration results in a modified expression or activity of said target gene detectable in said at least about at least about 1%. 2%, 3%, 4%, 5%, 6%, 7%, 8%, 9%, 10%, 11%, 12%, 13%, 14%, or 15% lung basal cells of said subject.

Embodiment 111. The method of Embodiment 99, wherein said therapeutic agent comprises a heterologous polynucleotide encoding an actuator moiety, which actuator moiety is configured to complex with a target polynucleotide corresponding to a target gene; and wherein said intravenous administration results in a modified expression or activity of said target gene detectable in said at least about at least about 1%, 2%, 3%, 4%, 5%, 6%, 7%, 8%, 9%. 10%, 11%, 12%, 13%, 14%, or 15% lung basal cells of said subject.

Embodiment 112. The method of Embodiment 111, wherein said heterologous polynucleotide encodes a guide polynucleotide configured to direct said actuator moiety to said target polynucleotide.

Embodiment 113. The method of Embodiment 111, wherein said actuator moiety comprises a heterologous endonuclease or a fragment thereof (e.g., directed by a guide polynucleotide to specifically bind said target polynucleotide).

Embodiment 114. The method of Embodiment 113, wherein said heterologous endonuclease is (1) part of a ribonucleoprotein (RNP) and (2) complexed with said guide polynucleotide.

Embodiment 115. The method of Embodiment 113, wherein said heterologous endonuclease is part of a clustered regularly interspaced short palindromic repeats (CRISPR)/CRISPR-associated (Cas) protein complex.

Embodiment 116. The method of Embodiment 113, wherein said heterologous endonuclease is a clustered regularly interspaced short palindromic repeats (CRISPR)-associated (Cas) endonuclease.

Embodiment 117. The method of Embodiment 113, wherein said heterologous endonuclease is selected from C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas 11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14. Csx10, Csx16, CsaX, Csx3, Csx1, Csx15, Csf1, Csf2, Csf3, Csf4, or a fragment thereof.

Embodiment 118. The method of Embodiment 113, wherein said heterologous endonuclease comprises a deactivated endonuclease, optionally fused to a regulatory moiety (e.g., comprising a transcription activator, a transcription repressor, an epigenetic modifier, or a fragment thereof).

Embodiment 119. The method of Embodiment 111, wherein said target polynucleotide corresponds to a gene encoding any protein selected from the group consisting of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, and FBN1.

Embodiment 120. The method of Embodiment 111, wherein said target polynucleotide corresponds to a gene in a lung cell (e.g., a lung basal cell) of said subject.

Embodiment 121. The method of Embodiment 111, wherein said expression or activity or said modified expression or activity is detectable at least about 4 hours after said intravenous administering.

Embodiment 122. The method of any one of Embodiments 1-121, wherein said therapeutic effect is characterized by an (e.g., therapeutically effective) amount, activity, or effect of said therapeutic agent (e.g., in a lung, a lung cell, a plurality of lung cells, or a lung cell type of said subject).

Embodiment 123. The method of any one of Embodiments 1-122, wherein said greater therapeutic effect is characterized by a greater (e.g., therapeutic) amount, activity, or effect of said therapeutic agent.

Embodiment 124. The method of any one of Embodiments 1-123, wherein said reference lipid composition does not comprise said amount of said SORT lipid.

Embodiment 125. The method of Embodiment 124, wherein said reference lipid composition does not comprise said SORT lipid.

Embodiment 126. The method of any one of Embodiments 1-125, wherein said reference lipid composition comprises 13,16,20-tris(2-hydroxydodecyl)-13,16,20,23-tetraazapentatricontane-11,25-diol (“LF92”), a phospholipid, cholesterol, and a PEG-lipid.

Embodiment 127. The method of any one of Embodiments 1-126, wherein said cell comprises a lung airway cell (e.g., a basal cell).

Embodiment 128. The method of any one of Embodiments 1-127, wherein said first cell type is basal cell.

Embodiment 129. The method of any one of Embodiments 1-128, wherein said first cell type is lung (e.g., airway) cell.

Embodiment 130. The method of any one of Embodiments 1-128, wherein said second cell type is lung (e.g., airway) cell.

Embodiment 131. A high-potency intravenous dosage form of a therapeutic agent formulated with a selective organ targeting (SORT) lipid, the dosage form comprising:
said therapeutic agent assembled with a lipid composition that comprises:
   (i) an ionizable cationic lipid; and
   (ii) said SORT lipid separate from said ionizable cationic lipid, wherein said SORT lipid is present in said dosage form in an amount sufficient to achieve a therapeutic effect at a dose of said therapeutic agent (e.g., at least about 1.1- or 10-fold) lower than that required with a reference lipid composition.

Embodiment 132. A high-potency intravenous dosage form of a therapeutic agent formulated with a selective organ targeting (SORT) lipid, the dosage form comprising:
said therapeutic agent assembled with a lipid composition that comprises:
   (i) an ionizable cationic lipid; and
   (ii) said SORT lipid separate from said ionizable cationic lipid, wherein said therapeutic agent (e.g., heterologous polynucleotide) is present in said dosage form at a dose of no more than about 2 milligram per kilogram (mg/kg, or mpk) body weight.

Embodiment 133. The dosage form of Embodiment 131 or 132, wherein said lipid composition further comprises (iii) a phospholipid.

Embodiment 134. The dosage form of any one of Embodiments 131-133, wherein said therapeutic agent (e.g., heterologous polynucleotide) is present in said intravenous dosage form at a dose of no more than about 1.0, 0.5, 0.1, 0.05, or 0.01 mg/kg body weight.

Embodiment 135. The dosage form of any one of Embodiments 131-134, wherein said therapeutic agent (e.g., heterologous polynucleotide) is present in said intravenous dosage form at a concentration of no more than about 5 or 2 milligram per milliliter (mg/mL).

Embodiment 136. The dosage form of any one of Embodiments 131-135, wherein said lipid composition comprises said SORT lipid at a molar percentage from about 20% to about 65%.

Embodiment 137. The dosage form of any one of Embodiments 131-136, wherein said lipid composition comprises said ionizable cationic lipid at a molar percentage from about 5% to about 30%.

Embodiment 138. The dosage form of any one of Embodiments 131-137, wherein said lipid composition comprises said phospholipid at a molar percentage from about 8% to about 23%.

Embodiment 139. The dosage form of any one of Embodiments 131-138, wherein said phospholipid is not an ethylphosphocholine.

Embodiment 140. The dosage form of any one of Embodiments 131-139, wherein said lipid composition further comprises a steroid or steroid derivative.

Embodiment 141. The dosage form of Embodiment 140, wherein said lipid composition comprises said steroid or steroid derivative at a molar percentage from about 15% to about 46%.

Embodiment 142. The dosage form of any one of Embodiments 131-141, wherein said lipid composition further comprises a polymer-conjugated lipid (e.g., poly(ethylene glycol) (PEG)-conjugated lipid).

Embodiment 143. The dosage form of Embodiment 142, wherein said lipid composition comprises said polymer-conjugated lipid at a molar percentage from about 0.5% to about 10%.

Embodiment 144. The dosage form of Embodiment 142, wherein said lipid composition comprises said polymer-conjugated lipid at a molar percentage from about 1% to about 10%.

Embodiment 145. The dosage form of Embodiment 142, wherein said lipid composition comprises said polymer-conjugated lipid at a molar percentage from about 2% to about 10%.

Embodiment 146. The dosage form of any one of Embodiments 131-145, wherein said therapeutic agent is a polynucleotide; and wherein a molar ratio of nitrogen in said lipid composition to phosphate in said polynucleotide (N/P ratio) is no more than about 20:1.

Embodiment 147. The dosage form of Embodiment 146, wherein said N/P ratio is from about 5:1 to about 20:1.

Embodiment 148. The dosage form of any one of Embodiments 131-147, wherein a molar ratio of said therapeutic agent to total lipids of said lipid composition is no more than about 1:1, 1:10, 1:50, or 1:100.

Embodiment 149. The dosage form of any one of Embodiments 131-148, wherein at least about 85% of said therapeutic agent is encapsulated in particles of said lipid compositions.

Embodiment 150. The dosage form of any one of Embodiments 131-149, wherein said lipid composition comprises a plurality of particles characterized by one or more characteristics of the following:
(1) a (e.g., average) size of 100 nanometers (nm) or less;
(2) a polydispersity index (PDI) of no more than about 0.2; and
(3) a negative zeta potential of −10 millivolts (mV) to 10 mV.

Embodiment 151. The dosage form of any one of Embodiments 131-150, wherein said lipid composition has an apparent ionization constant (pKa) outside a range of 6 to 7.

Embodiment 152. The dosage form of Embodiment 151, wherein said apparent pKa of said lipid composition is of about 7 or higher.

Embodiment 153. The dosage form of Embodiment 151, wherein said apparent pKa of said lipid composition is of about 8 or higher.

Embodiment 154. The dosage form of Embodiment 151, wherein said apparent pKa of said lipid composition is from about 8 to about 13.

Embodiment 155. The dosage form of any one of Embodiments 131-154, wherein said SORT lipid comprises a permanently positively charged moiety (e.g., a quaternary ammonium ion).

Embodiment 156. The dosage form of Embodiment 155, wherein said SORT lipid comprises a counterion.

Embodiment 157. The dosage form of any one of Embodiments 131-156, wherein said SORT lipid is a phosphocholine lipid (e.g., saturated or unsaturated).

Embodiment 158. The dosage form of any one of Embodiments 157, wherein said SORT lipid is an ethylphosphocholine.

Embodiment 159. The dosage form of any one of Embodiments 131-158, wherein said SORT lipid comprises a headgroup having a structural formula:

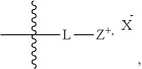

wherein L is a (e.g., biodegradable) linker; Z⁺ is positively charged moiety (e.g., a quaternary ammonium ion); and X⁻ is a counterion.

Embodiment 160. The dosage form of Embodiment 159, wherein said SORT lipid has a structural formula:

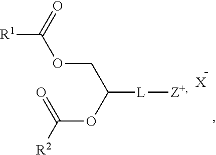

wherein R¹ and R² are each independently an optionally substituted C₆-C₂₄ alkyl, or an optionally substituted C₆-C₂₄ alkenyl.

Embodiment 161. The dosage form of Embodiment 159, wherein said SORT lipid has a structural formula:

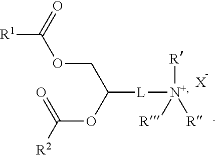

Embodiment 162. The dosage form of any one of Embodiments 159-162, wherein L is

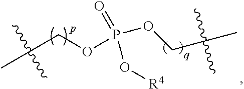

wherein:
p and q are each independently 1, 2, or 3; and
R⁴ is an optionally substituted C₁-C₆ alkyl.

Embodiment 163. The dosage form of Embodiment 159, wherein said SORT lipid has a structural formula:

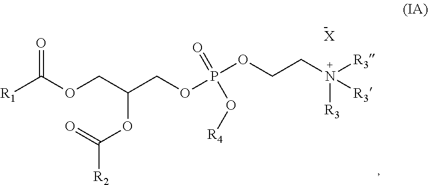

wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
R₄ is alkyl_{(C≤6)} or substituted alkyl_{(C≤6)}; and
X⁻ is a monovalent anion.

Embodiment 164. The dosage form of any one of Embodiments 131-154, wherein said SORT lipid has a structural formula:

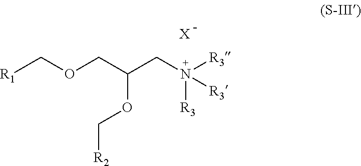

wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
X⁻ is a monovalent anion.

Embodiment 165. The dosage form of any one of Embodiments 131-154, wherein said SORT lipid has a structural formula:

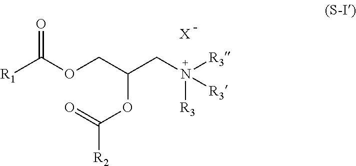

wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
X⁻ is a monovalent anion.

Embodiment 166. The dosage form of any one of Embodiments 131-154, wherein said SORT lipid has a structural formula:

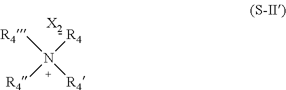

wherein:
R₄ and R_{4′} are each independently alkyl_{(C6-C24)}, alkenyl_{(C6-C24)}, or a substituted version of either group;
R_{4″} is alkyl_{(C≤24)}, alkenyl_{(C≤24)}, or a substituted version of either group;
R_{4″} is alkyl_{(C2-C8)}, alkenyl_{(C2-C8)}, or a substituted version of either group; and
X₂ is a monovalent anion.

Embodiment 167. The dosage form of any one of Embodiments 131-154, wherein said SORT lipid is selected from those set forth in Table 6, or pharmaceutically acceptable salts thereof, or a subset of the lipids and the pharmaceutically acceptable salts thereof.

Embodiment 168. The dosage form of any one of Embodiments 131-167, wherein the ionizable cationic lipid is a dendrimer or dendron of a generation (g) having a structural formula:

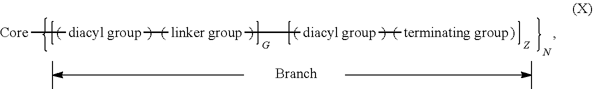

or a pharmaceutically acceptable salt thereof, wherein:
(a) the core comprises a structural formula (X_{Core}):

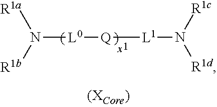

wherein:
Q is independently at each occurrence a covalent bond, —O—, —S—, —NR²—, or —CR^{3a}R^{3b}—;
R₂ is independently at each occurrence R^{1g} or -L²-NR^{1e}R^{1f};
R^{3a} and R^{3b} are each independently at each occurrence hydrogen or an optionally substituted (e.g., C₁-C₆, such as C₁-C₃) alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
x¹ is 0, 1, 2, 3, 4, 5, or 6; and
(b) each branch of the plurality (N) of branches independently comprises a structural formula (X_{Branch}):

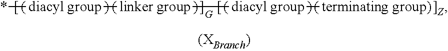

wherein:
* indicates a point of attachment of the branch to the core;
g is 1, 2, 3, or 4;
Z=2^{(g-1)}.
G=0, when g=1; or G=Σᵢ₌₀^{i=g-2}2ⁱ, when g≠1;
(c) each diacyl group independently comprises a structural formula

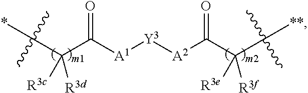

wherein:
* indicates a point of attachment of the diacyl group at the proximal end thereof;
** indicates a point of attachment of the diacyl group at the distal end thereof;
Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR₄—, wherein:
   R₄ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula

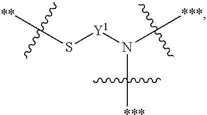

wherein:
** indicates a point of attachment of the linker to a proximal diacyl group;
*** indicates a point of attachment of the linker to a distal diacyl group; and
Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

Embodiment 169. The dosage form of Embodiment 168, wherein x¹ is 0, 1, 2, or 3.

Embodiment 170. The dosage form of Embodiment 168 or 169, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl

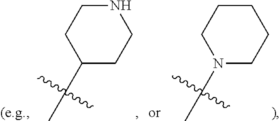 [SMILES: *C1CCNCC1 ; *N1CCCCC1]

N—(C₁-C₃ alkyl)-piperidinyl

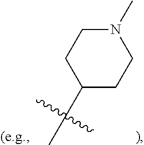 [SMILES: *C1CCN(C)CC1]

piperazinyl

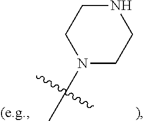 [SMILES: *N1CCNCC1]

N—(C₁-C₃ alkyl)-piperadizinyl

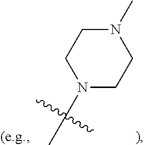 [SMILES: *N1CCN(C)CC1]

morpholinyl

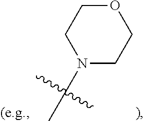 [SMILES: *N1CCOCC1]

### N-pyrrolidinyl

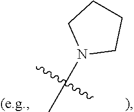 [SMILES: *N1CCCC1]

pyrrolidinyl

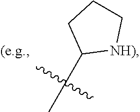

or N—(C₁-C₃ alkyl)-pyrrolidinyl

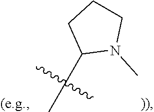 [SMILES: *C1CCCN1C]

(e.g., C₆-C₁₀) aryl, and C₃-C₅ heteroaryl (e.g., imidazolyl

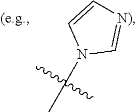

or pyridinyl

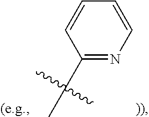 [SMILES: *c1ccccn1]

Embodiment 171. The dosage form of Embodiment 170, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

Embodiment 172. The dosage form of any one of Embodiments 168-171, wherein R^{3a} and R₃₆ are each independently at each occurrence hydrogen.

Embodiment 173. The dosage form of any one of Embodiments 168-172, wherein the plurality (N) of branches comprises at least 3 (e.g., at least 4, or at least 5) branches.

Embodiment 174. The dosage form of any one of Embodiments 168-173, wherein g=1; G=0; and Z=1.

Embodiment 175. The dosage form of Embodiment 174, wherein each branch of the plurality of branches comprises a structural formula *-(diacyl group)-(terminating group).

Embodiment 176. The dosage form of any one of Embodiments 168-173, wherein g=2; G=1; and Z=2.

Embodiment 177. The dosage form of Embodiment 176, wherein each branch of the plurality of branches comprises a structural formula

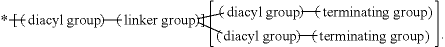

Embodiment 178. The dosage form of any one of Embodiments 168-177, wherein the core comprises a structural formula:

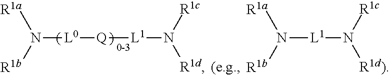

Embodiment 179. The dosage form of any one of Embodiments 168-177, wherein the core comprises a structural formula:

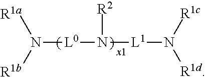

Embodiment 180. The dosage form of Embodiment 179, wherein the core comprises a structural formula:

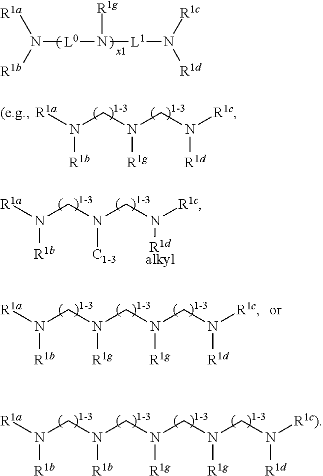

Embodiment 181. The dosage form of Embodiment 179, wherein the core comprises a structural formula:

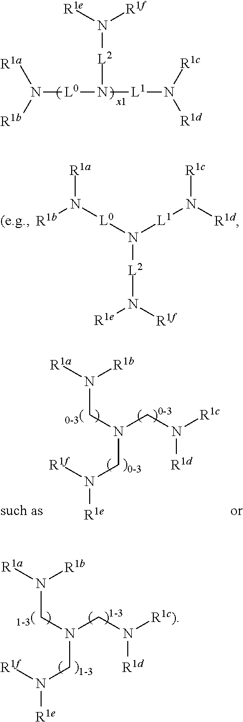

Embodiment 182. The dosage form of any one of Embodiments 168-177, wherein the core comprises a structural formula:

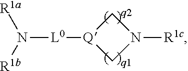

wherein Q′ is —NR²— or —CR^{3a}R^{3b}—; q¹ and q² are each independently 1 or 2.

Embodiment 183. The dosage form of Embodiment 182, wherein the core comprises a structural formula

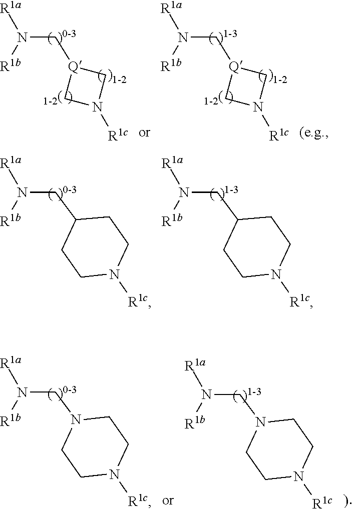

Embodiment 184. The dosage form of any one of Embodiments 168-177, wherein the core comprises structural formula

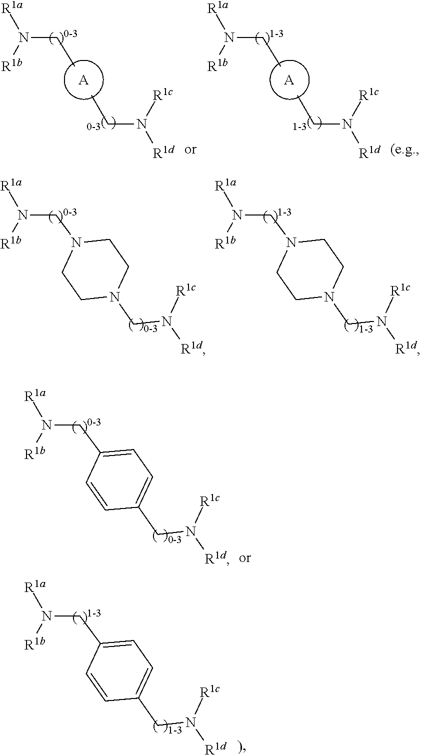

wherein ring A is an optionally substituted aryl or an optionally substituted (e.g., C₃-C₁₂, such as C₃-C₅) heteroaryl.

Embodiment 185. The dosage form of any one of Embodiments 168-177, wherein the core comprises has a structural formula

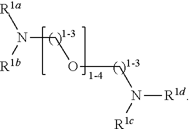

Embodiment 186. The dosage form of any one of Embodiments 168-177, wherein the core is selected from those set forth in Table 1 or a subset thereof.

Embodiment 187. The dosage form of any one of Embodiments 168-177, wherein the core comprises a structural formula selected from the group consisting of:

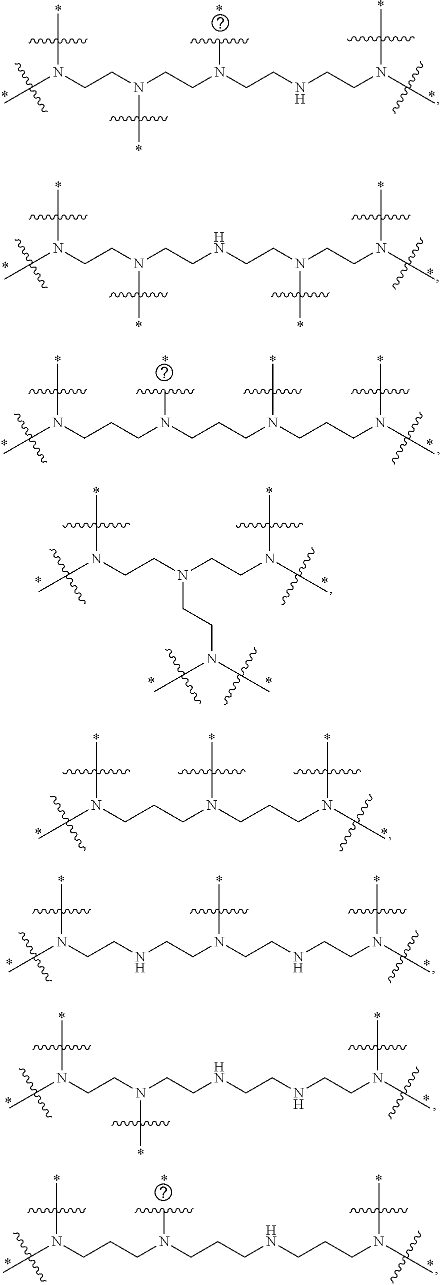

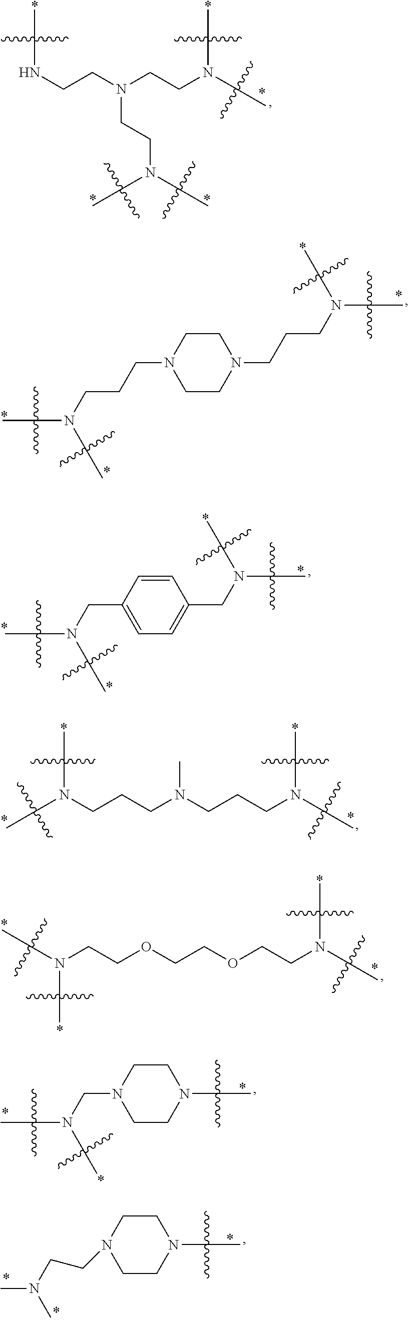

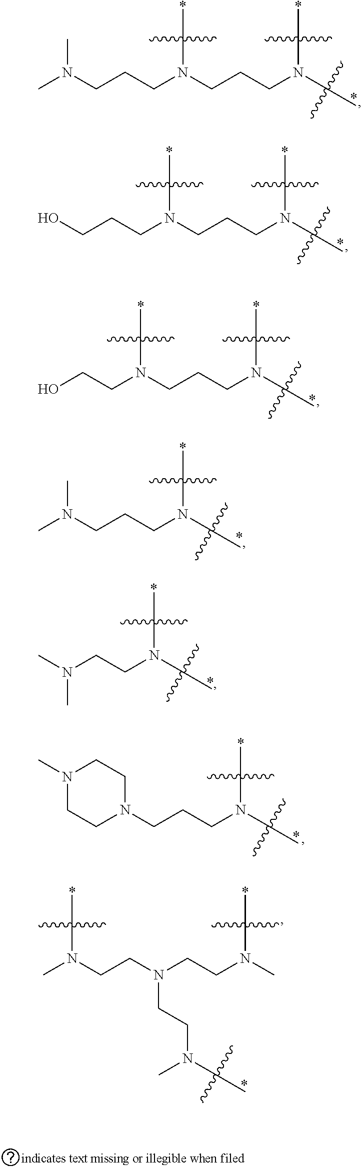

and pharmaceutically acceptable salts thereof, wherein * indicates a point of attachment of the core to a branch of the plurality of branches.

Embodiment 188. The dosage form of any one of Embodiments 168-177, wherein the core comprises a structural formula selected from the group consisting of:

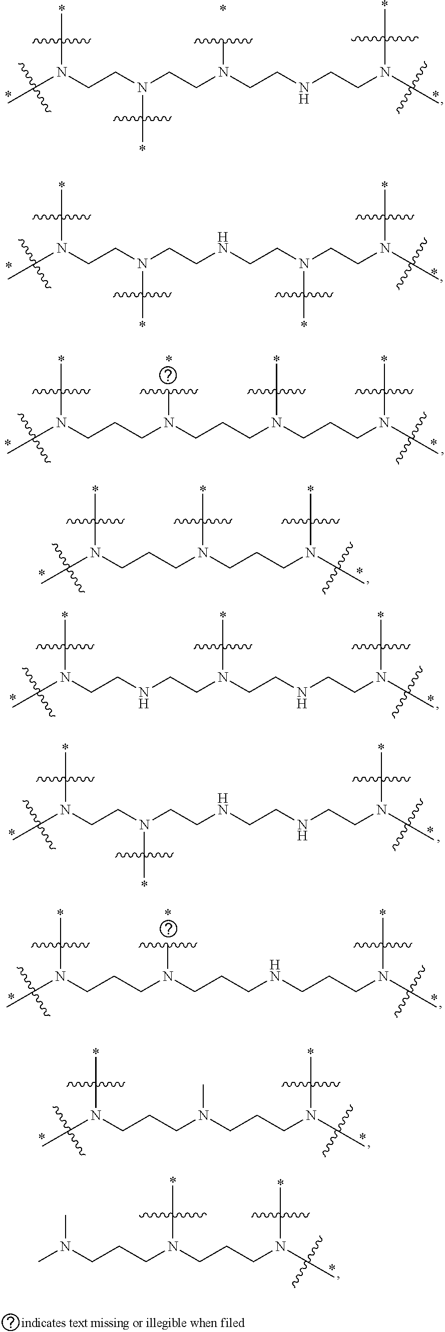

and pharmaceutically acceptable salts thereof, wherein * indicates a point of attachment of the core to a branch of the plurality of branches.

Embodiment 189. The dosage form of any one of Embodiments 168-177, wherein the core has the structure

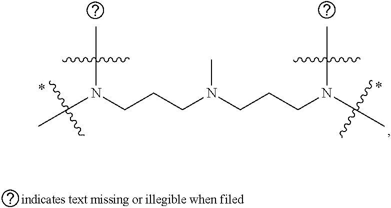 [SMILES: *N(*)CCCN(C)CCCN(*)*]

wherein * indicates a point of attachment of the core to a branch of the plurality of branches or H.

Embodiment 190. The dosage form of Embodiment 189, wherein at least 2 branches are attached to the core.

Embodiment 191. The dosage form of Embodiment 189, wherein at least 3 branches are attached to the core.

Embodiment 192. The dosage form of Embodiment 189, wherein at least 4 branches are attached to the core.

Embodiment 193. The dosage form of any one of Embodiments 168-177, wherein the core has the structure

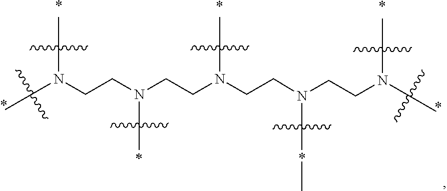

wherein * indicates a point of attachment of the core to a branch of the plurality of branches or H.

Embodiment 194. The dosage form of Embodiment 193, wherein at least 4 branches are attached to the core.

Embodiment 195. The dosage form of Embodiment 193, wherein at least 5 branches are attached to the core.

Embodiment 196. The dosage form of Embodiment 193, wherein at least 6 branches are attached to the core.

Embodiment 197. The dosage form of any one of Embodiments 168-196, wherein A¹ is —O— or —NH—.

Embodiment 198. The dosage form of Embodiment 197, wherein A¹ is —O—.

Embodiment 199. The dosage form of any one of Embodiments 168-198, wherein A² is —O— or —NH—.

Embodiment 200. The dosage form of any Embodiment 199, wherein A² is —O—.

Embodiment 201. The dosage form of any one of Embodiments 168-200, wherein Y³ is C₁-C₁₂ (e.g., C₁-C₆, such as C₁-C₃) alkylene.

Embodiment 202. The dosage form of any one of Embodiments 168-201, wherein the diacyl group independently at each occurrence comprises a structural formula

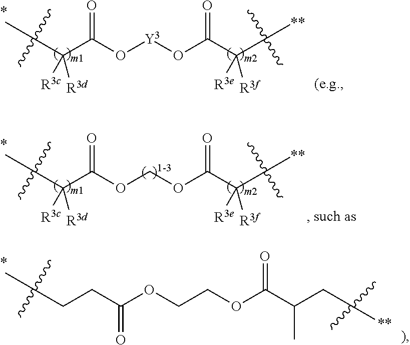

optionally wherein R^{3c}, R^{3d}, R^{3e}, and R^{3f} are each independently at each occurrence hydrogen or C₁-C₃ alkyl.

Embodiment 203. The dosage form of any one of Embodiments 168-202, wherein L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

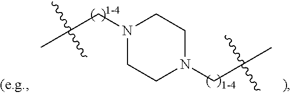

and [(C₁-C₄) alkylene]-phenylene-[(C₁-C₄) alkylene]

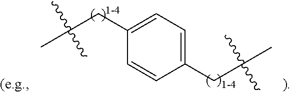

Embodiment 204. The dosage form of Embodiment 203, wherein L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-.

Embodiment 205. The dosage form of Embodiment 203, wherein L⁰, L¹, and L² are each independently at each occurrence C₁-C₆ alkylene (e.g., C₁-C₃ alkylene).

Embodiment 206. The dosage form of Embodiment 203, wherein L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)).

Embodiment 207. The dosage form of Embodiment 203, wherein L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

Embodiment 208. The dosage form of any one of Embodiments 168-207, wherein each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₈ (e.g., C₁-C₈) alkylamino (e.g., C₁-C₆ mono-alkylamino (such as —NHCH₂CH₂CH₂CH₃) or C₁-C₈ di-alkylamino

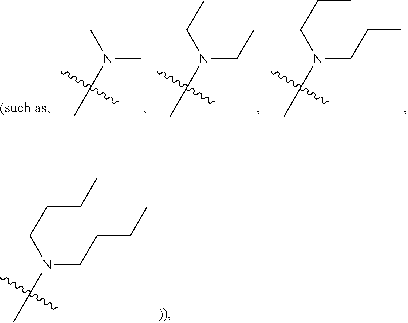

C₄-C₆ N-heterocycloalkyl (e.g., N-pyrrolidinyl

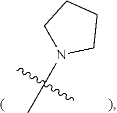 [SMILES: *N1CCCC1]

### N-piperidinyl

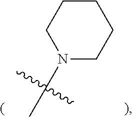 [SMILES: *N1CCCCC1]

### N-azepanyl

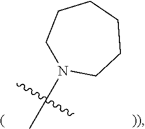 [SMILES: *N1CCCCCC1]

—OH, —C(O)OH, —C(O)N(C₁-C₃ alkyl)-(C₁-C₆ alkylene)-(C₁-C₁₂ alkyl-amino (e.g., mono- or di-alkylamino))

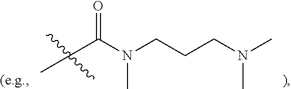 [SMILES: *C(=O)N(C)CCCN(C)C]

—C(O)N(C₁-C₃ alkyl) (C₁-C₆ alkylene)-(C₄-C₆ N-heterocycloalkyl)

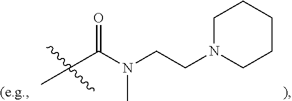 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

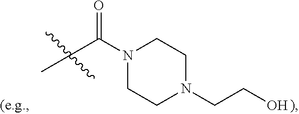 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 209. The dosage form of Embodiment 208, wherein each terminating group is independently C₁-C₁₈ (e.g., C₄-C₁₈) alkylthiol, wherein the alkyl moiety is optionally substituted with one or more (e.g., one) substituents each independently selected from C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino

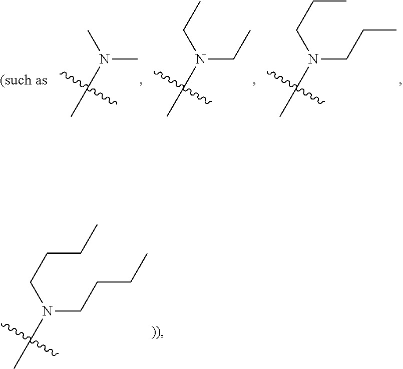

C₄-C₆ N-heterocycloalkyl (e.g., N-pyrrolidinyl

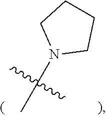 [SMILES: *N1CCCC1]

### N-piperidinyl

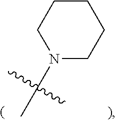 [SMILES: *N1CCCCC1]

### N-azepanyl

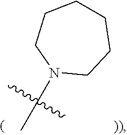 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

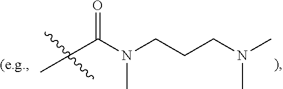 [SMILES: *C(=O)N(C)CCCN(C)C]

—C(O)N(C₁-C₃ alkyl)-(C₁-C₆ alkylene)-(C₄-C₆ N-heterocycloalkyl)

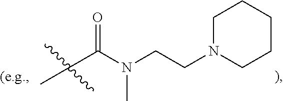 [SMILES: *C(=O)N(C)CCN1CCCCC1]

and —C(O)—(C₄-C₆ N-heterocycloalkyl)

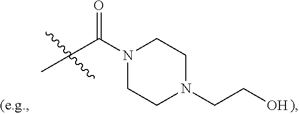 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 210. The dosage form of Embodiment 209, wherein each terminating group is independently C₁-C₁₈ (e.g., C₄-C₁₈) alkylthiol, wherein the alkyl moiety is optionally substituted with one substituent —OH.

Embodiment 211. The dosage form of Embodiment 209, wherein each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino

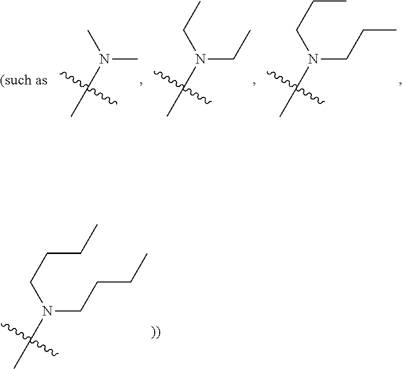

and C₄-C₆ N-heterocycloalkyl (e.g., N-pyrrolidinyl

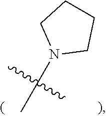 [SMILES: *N1CCCC1]

### N-piperidinyl

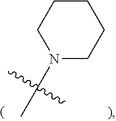 [SMILES: *N1CCCCC1]

### N-azepanyl

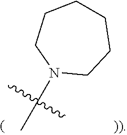 [SMILES: *N1CCCCCC1]

Embodiment 212. The dosage form of Embodiment 208, wherein each terminating group is independently C₁-C₁₈ (e.g., C₄-C₁₈) alkenylthiol or C₁-C₁₈ (e.g., C₄-C₁₈) alkylthiol.

Embodiment 213. The dosage form of Embodiment 212, wherein each terminating group is independently C₁-C₁₈ (e.g., C₄-C₁₈) alkylthiol.

Embodiment 214. The dosage form of Embodiment 213, wherein each terminating group is independently selected from the group consisting of:

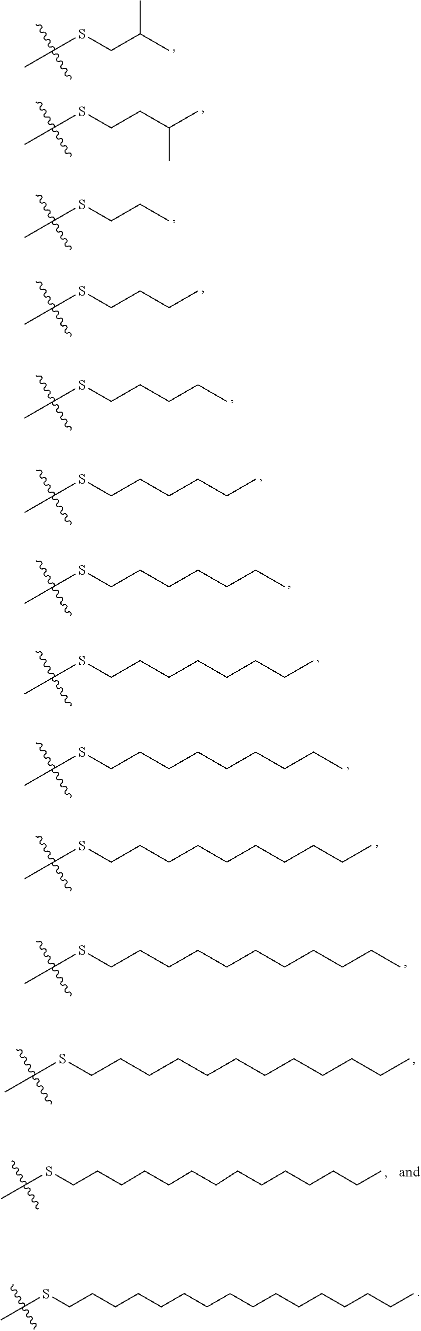

Embodiment 215. The dosage form of any one of Embodiments 168-214, wherein each terminating group is independently selected from those set forth in Table 3 or a subset thereof.

Embodiment 216. The dosage form of any one of Embodiments 131-167, wherein the ionizable cationic lipid is selected from those set forth in Table 4, or pharmaceutically acceptable salts thereof, or a subset of the lipids and the pharmaceutically acceptable salts thereof.

Embodiment 217. The dosage form of any one of Embodiments 131-167, wherein the ionizable cationic lipid is selected from those set forth in Table 4 or Table 5, or pharmaceutically acceptable salts thereof, or a subset of the lipids and the pharmaceutically acceptable salts thereof.

### EXAMPLES

### Example 1: Preparation of DOTAP or DODAP Modified Lipid Nanoparticles

Lipid nanoparticles (LNPs) are the most efficacious carrier class for in vivo nucleic acid delivery. Historically, effective LNPs are composed of 4 components: an ionizable cationic lipid, zwitterionic phospholipid, cholesterol, and lipid poly(ethylene glycol) (PEG). However, these LNPs result in only general delivery of nucleic acids, rather than organ or tissue targeted delivery. LNPs typically delivery RNAs only to the liver. Therefore, new formulations of LNPs were sought in an effort to provide targeted nucleic acid delivery.

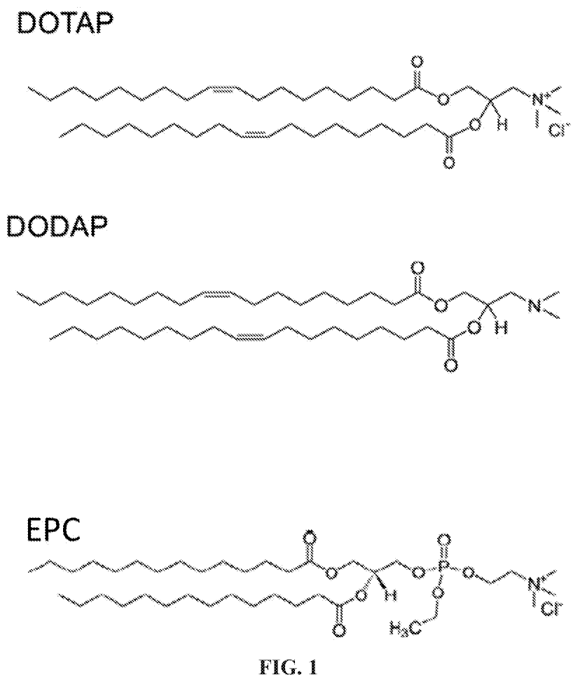

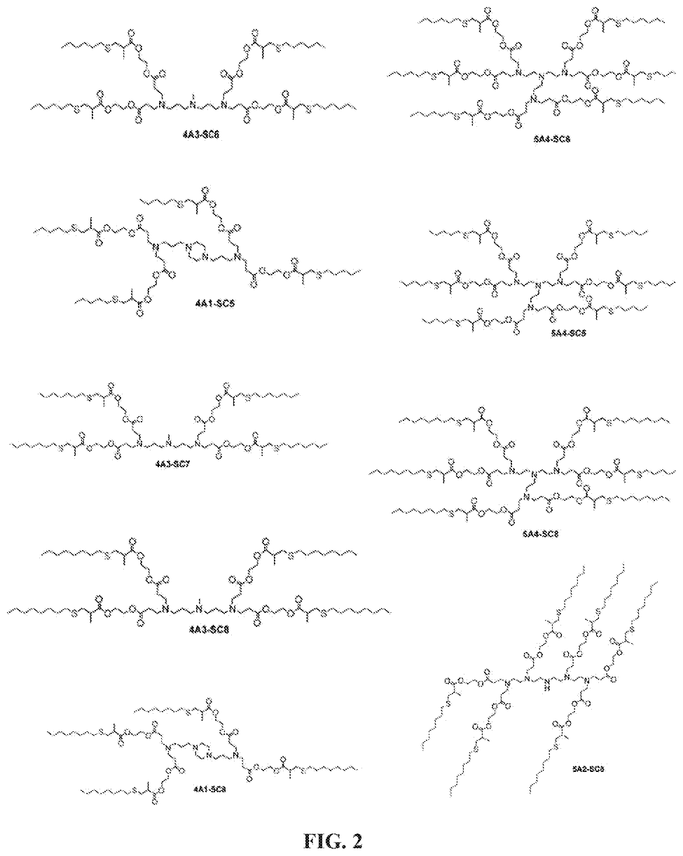

The four canonical types of lipids, in an example, were mixed in a 15:15:30:3 molar ratio, with or without the addition of a permanently cationic lipid. Briefly, example lipid composition as described herein were prepared by mixing a dendrimer or dendron lipid (ionizable cationic) as described herein, DOPE (zwitterionic), cholesterol, DMG-PEG, and DOTAP (permanently cationic). Alternatively, DOTAP can be substituted for DODAP to generate an LNP comprising DODAP. The structure of DODAP and DODAP are shown in FIG. 1. Various example dendrimer or dendron lipids that may be used are shown in FIG. 2.

For preparation of the LNP formulation, a dendrimer or dendron lipid, DOPE, Cholesterol and DMG-PEG were dissolved in ethanol at desired molar ratios. The mRNA was dissolved in citrate buffer (10 mM. pH 4.0). The mRNA was then diluted into the lipids solution to achieve a weight ratio of 40:1 (total lipids:mRNA) by rapidly mixing the mRNA into the lipid solution at a volume ratio of 3:1 (mRNA:lipids, v/v). This solution was then incubated for 10 min at room temperature. For formation of DOTAP modified LNP formulations, mRNA was dissolved in 1×PBS or citrate buffer (10 mM, pH 4.0), and mixed rapidly into ethanol containing 5A2-SC8, DOPE, Cholesterol. DMG-PEG and DOTAP, fixing the weight ratio of 40:1 (total lipids:mRNA) and volume ratio of 3:1 (mRNA:lipids). Formulations are named X % DOTAP Y (or X % DODAP Y) where X represents the DOTAP (or DODAP) molar percentage in total lipids, and Y represents the type of dendrimer or dendron lipid. Alternatively, formulation may be named Y X % DOTAP or Y X % DODAP where X represents the DOTAP (or DODAP) molar percentage in total lipids, and Y represents the type of dendrimer or dendron lipid.

### Example 2: SORT LNP Stability

Lipid (LNP) compositions are tested for stability. Lipid compositions as described herein, such as those comprising a dendrimer or dendron (e.g., 5A2-SC8), as an ionizable cationic lipid, and a selective organ-targeting (SORT) lipid (e.g., DODAP), e.g., at a molar percentage in total lipids from 20% to 50%, are generated using either a microfluidic mixing method or a cross/tee mixing method. Size, polydispersity index (PDI) and zeta-potential of different LNP formulations are characterized by dynamic light scattering (DLS) (3 separate times for each formulation).

Encapsulation efficiency of the LNPs is tested using a Ribogreen RNA assay (Zhao et al., 2016). Briefly, mRNA is encapsulated with >90% (e.g., >95%) efficiency in LNPs when the mRNA is dissolved in acidic buffer (e.g., 10 mM citrate, pH 4). The characteristics are observed over 28 days for the tested LNPs, e.g., over the course of 28 days.

In addition, stability of the lipid compositions as described herein (LNPs) in solution and resulting mRNA expression are observed in mice. Briefly, mice are injected intravenously with less than 1 mg/kg and observed in vivo. Luciferin is added 5 hours after injection and visualized. SORT (e.g., lung-SORT) LNP generated tissue specific radiance in the lungs remain highly detectable even after 14 day with a slight decay in signal by the 21^{st} and 28^{th} day. Images of organs of the tested mice at specific time periods after treated with example SORT LNP (as described herein) are taken.

### Example 3: Gene Editing Ability of Example SORT LNP after IV Administration

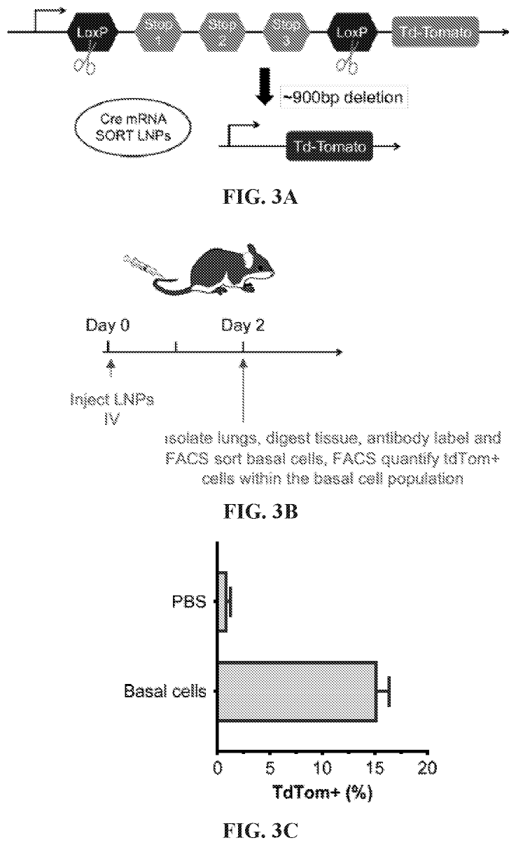

To examine and quantify the ability of example SORT LNP(s) (either D50 (5A2-SC8/DOTAP/DOPE/Cholesterol/PEG DMG in a molar ratio of 12/50/12/23/3) or D40 (5A2-SC8/DOTAP/DOPE/Cholesterol/PEG DMG in a molar ratio of 21.6/40/12/24/2.4)) to mediate organ-specific gene editing, genetically engineered tdTomato (tdTom) reporter mice containing a LoxP flanked stop cassette that prevents expression of the tdTom protein were utilized. Once the stop cassette is deleted, tdTom fluorescence is turned on, allowing detection of gene edited cells, as shown in FIG. 3A. The example Cre recombinase mRNA (Cre mRNA) SORT LNPs of the present application were intravenously delivered to mice on Day 0 to activate tdTom. On Day 2, lungs of the mice were isolated, and lung tissues were digested. Then lung basal cells in the digested tissues were labelled with antibody and sorted by FACS, tdTom positive cells within the basal cell population were further quantified by FACS, as shown in FIG. 3B.

FIG. 3C illustrates that when the stop cassette LoxP is edited by cre mRAN through the delivery of the SORT LNPs as described in the present application, expression of TdTom was successfully turned on in lung basal cell of the mice, indicating the tested SORT LNP(s) allow lung basal cell gene editing following IV administration.

### Example 4. SORT LNP Localization

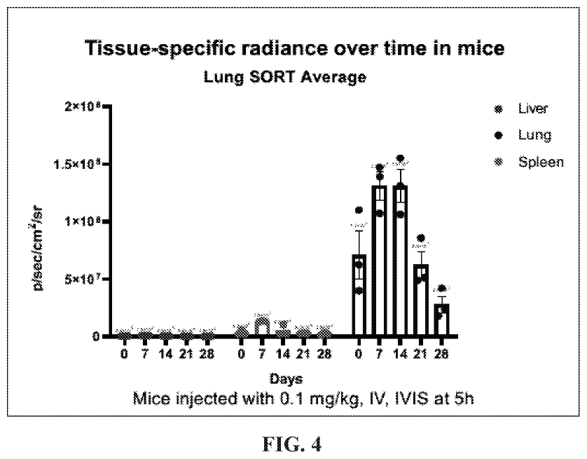

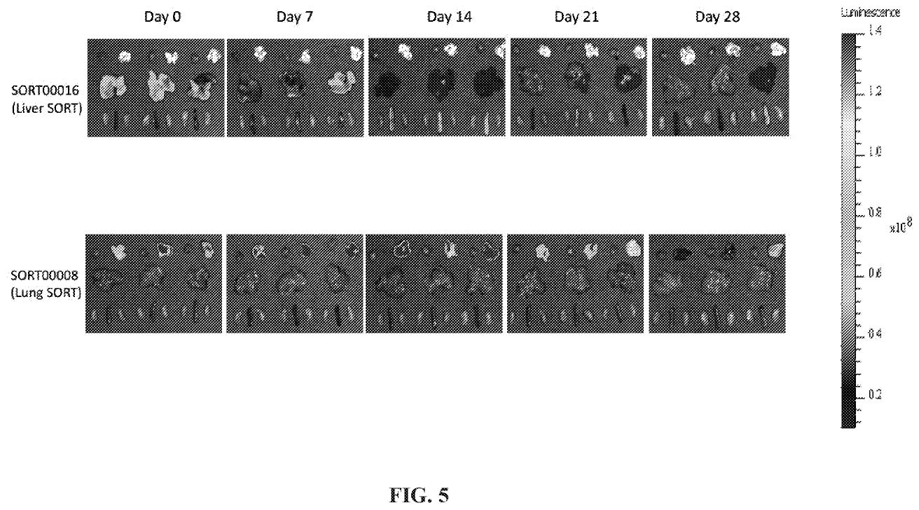

The localization of example SORT LNP(s) (as described herein) and the resulting mRNA expression was observed in mice. Briefly, mice were injected intravenously with 0.1 mg/kg and observed in vivo. Luciferin was added 5 hrs. after injection and visualized. As shown in FIG. 4, the example Lung-SORT LNP (as described herein) generated tissue specific radiance in the lungs which remained high even after 14 day with a slight decay in signal by the 21^{st} and 28^{th} day. FIG. 5 shows images of the organs of the mouse at specific times periods after treated with Lung-SORT or Liver-SORT.

### Example 5. SORT LNP Localization in Mice, Dog and Non-Human Primate (NHP)

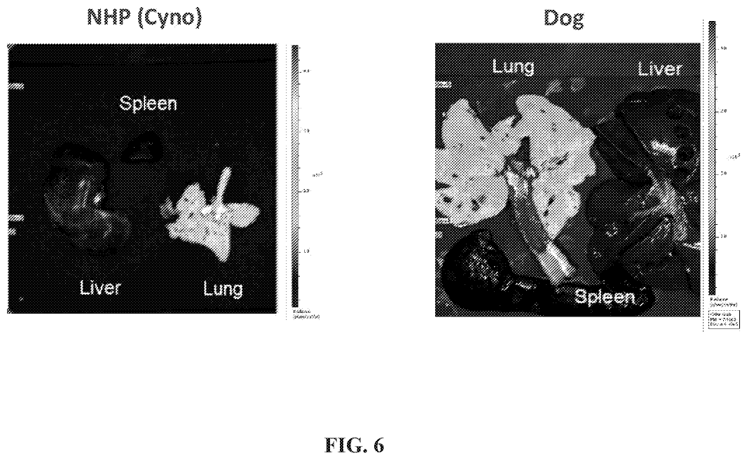

The localization of example SORT LNP and the resulting mRNA expression was observed in dogs and non-human primates. Delivering via IV bolus a lipid composition comprising DOTAP described herein showed a localization to the lungs of the animals. FIG. 6 shows the mRNA expression as localized to the lung.

Another LNP formulation with DOTAP was used to deliver mRNA to the lungs. When administered to mice and dogs, the delivery was preferential to lung(s).

While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for potent delivery to a non-liver basal cell of a subject, comprising:
intravenously administering to said subject a composition comprising a therapeutic agent assembled with a lipid composition which comprises:
(i) an ionizable cationic lipid; and
(ii) a selective organ targeting (SORT) lipid separate from said ionizable cationic lipid, wherein said SORT lipid effects delivery of said therapeutic agent to said non-liver basal cell of said subject characterized by a greater amount or activity of said therapeutic agent in said non-liver basal cell compared to that achieved with a reference lipid composition.

2. The method of claim 1, wherein said non-liver basal cell is a lung basal cell.

3. A method for potent delivery to non-liver basal cells of a subject, comprising:
intravenously administering to said subject a composition comprising a therapeutic agent assembled with a lipid composition which comprises:
(i) an ionizable cationic lipid; and
(ii) a selective organ targeting (SORT) lipid separate from said ionizable cationic lipid, wherein said SORT lipid effects delivery of said therapeutic agent to said non-liver basal cells of said subject characterized by an amount or activity of said therapeutic agent in a greater proportion of said non-liver basal cells compared to that achieved with a reference lipid composition.

4. The method of claim 3, wherein said non-liver basal cells are lung basal cells.

5. A method for targeted delivery to lung cells of a subject, comprising:
intravenously administering to said subject a composition comprising a therapeutic agent assembled with a lipid composition which comprises:
(i) an ionizable cationic lipid; and
(ii) a selective organ targeting (SORT) lipid separate from said ionizable cationic lipid, wherein said SORT lipid effects delivery of said therapeutic agent to a greater proportion of cell types of a lung of said subject as compared to that achieved with a reference lipid composition.

6. The method of claim 5, wherein said greater proportion of cell types of said lung comprises lung basal cells.

7. A method for targeted delivery to lung cells of a subject, comprising:
intravenously administering to said subject a composition comprising a therapeutic agent assembled with a lipid composition which comprises:
(i) an ionizable cationic lipid; and
(ii) a selective organ targeting (SORT) lipid separate from said ionizable cationic lipid, wherein said SORT lipid effects delivery of said therapeutic agent to said lung cells of said subject characterized by an amount or activity of said therapeutic agent in lung non-basal cells and in a greater plurality or proportion of lung basal cells as compared to that achieved with a reference lipid composition.

8. The method of claim 7, wherein said lung non-basal cells are lung epithelial cells, lung ciliated cells, lung club cells, or lung goblet cells.

9. A method for targeted delivery to lung cells of a subject, comprising:
intravenously administering to said subject a composition comprising a therapeutic agent assembled with a lipid composition which comprises:
(i) an ionizable cationic lipid; and
(ii) a selective organ targeting (SORT) lipid separate from said ionizable cationic lipid, wherein said SORT lipid effects a delivery of said therapeutic agent to cells of said subject characterized by a greater amount or activity of said therapeutic agent in a first lung cell of a first cell type of said subject compared to that in a second lung cell of a second cell type of said subject, wherein said first cell type is different from said second cell type.

10. The method of claim 9, wherein said first lung cell of said first cell type is a lung basal cell.

11. The method of claim 10, wherein said second lung cell of said second cell type is a lung epithelial cell, a lung ciliated cell, a lung club cell, or a lung goblet cell.

12. A method for delivery to lung basal cells of a subject, comprising:
intravenously administering to said subject a therapeutic agent assembled with a lipid composition that comprises:
(i) an ionizable cationic lipid; and
(ii) a selective organ targeting (SORT) lipid separate from said ionizable cationic lipid, thereby delivering said therapeutic agent to a lung of said subject to provide an amount or activity of said therapeutic agent detectable in at least about 5%, 10%, or 15% basal cells in said lung of said subject.

13. The method of claim 12, wherein said therapeutic effect is characterized by an amount or activity of said agent detectable in said at least about 5%, 10%, or 15% basal cells in said lung of said subject.

14. The method of any one of claims 1-13, wherein said lipid composition further comprises (iii) a phospholipid.

15. The method of any one of claims 1-14, wherein said lipid composition comprises said SORT lipid at a molar percentage from about 20% to about 65%.

16. The method of any one of claims 1-15, wherein said lipid composition comprises said ionizable cationic lipid at a molar percentage from about 5% to about 30%.

17. The method of any one of claims 1-16, wherein said lipid composition comprises said phospholipid at a molar percentage from about 8% to about 23%.

18. The method of any one of claims 1-17, wherein said phospholipid is not an ethylphosphocholine.

19. The method of any one of claims 1-18, wherein said lipid composition further comprises a steroid or steroid derivative.

20. The method of claim 19, wherein said lipid composition comprises said steroid or steroid derivative at a molar percentage from about 15% to about 46%.

21. The method of any one of claims 1-20, wherein said lipid composition further comprises a polymer-conjugated lipid.

22. The method of claim 21, wherein said lipid composition comprises said polymer-conjugated lipid at a molar percentage from about 0.5% to about 10%, about 1% to about 10%, or about 2% to about 10%.

23. The method of any one of claims 1-22, wherein said therapeutic agent is a polynucleotide; and wherein a molar ratio of nitrogen in said lipid composition to phosphate in said polynucleotide (N/P ratio) is no more than about 20:1.

24. The method of claim 23, wherein said N/P ratio is from about 5:1 to about 20:1.

25. The method of any one of claims 1-24, wherein a molar ratio of said therapeutic agent to total lipids of said lipid composition is no more than about 1:1, 1:10, 1:50, or 1:100.

26. The method of any one of claims 1-25, wherein at least about 85% of said therapeutic agent is encapsulated in particles of said lipid compositions.

27. The method of any one of claims 1-26, wherein said lipid composition comprises a plurality of particles characterized by one or more characteristics of the following:
(1) an average size of 100 nanometers (nm) or less;
(2) a polydispersity index (PDI) of no more than about 0.2; and
(3) a negative zeta potential of −10 millivolts (mV) to 10 mV.

28. The method of any one of claims 1-27, wherein said lipid composition has an apparent ionization constant (pKa) outside a range of 6 to 7.

29. The method of claim 28, wherein said apparent pKa of said lipid composition is of about 7 or higher, or about 8 or higher.

30. The method of claim 29, wherein said apparent pKa of said lipid composition is from about 8 to about 13.

31. The method of any one of claims 1-30, wherein said SORT lipid comprises a permanently positively charged moiety.

32. The method of claim 31, wherein said SORT lipid comprises a counterion.

33. The method of any one of claims 1-32, wherein said SORT lipid is a phosphocholine lipid.

34. The method of any one of claim 33, wherein said SORT lipid is an ethylphosphocholine.

35. The method of any one of claims 1-32, wherein said SORT lipid comprises a headgroup having a structural formula: 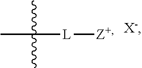
wherein L is a linker; Z⁺ is positively charged moiety; and X⁻ is a counterion.

36. The method of claim 35, wherein said SORT lipid has a structural formula: 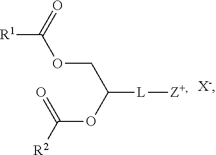
wherein R¹ and R² are each independently an optionally substituted C₆-C₂₄ alkyl, or an optionally substituted C₆-C₂₄ alkenyl.

37. The method of claim 35, wherein said SORT lipid has a structural formula:

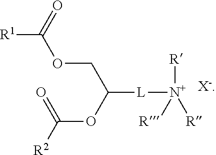

38. The method of claim 37, wherein L is 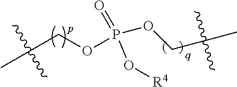
wherein:
p and q are each independently 1, 2, or 3; and
R⁴ is an optionally substituted C₁-C₆ alkyl.

39. The method of claim 35, wherein said SORT lipid has a structural formula: 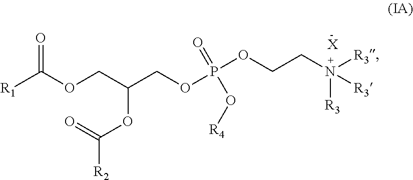
wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
R₄ is alkyl_{(C≤6)} or substituted alkyl_{(C≤6)}; and
X⁻ is a monovalent anion.

40. The method of any one of claims 1-32, wherein said SORT lipid has a structural formula: 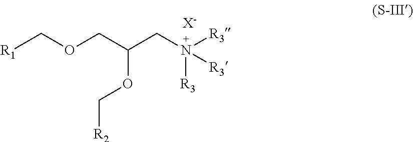
wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
X⁻ is a monovalent anion.

41. The method of any one of claims 1-32, wherein said SORT lipid has a structural formula: 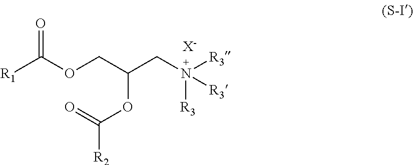
wherein:
R₁ and R₂ are each independently alkyl_{(C8-C24)}, alkenyl_{(C8-C24)}, or a substituted version of either group;
R₃, R_{3′}, and R_{3″} are each independently alkyl_{(C≤6)} or substituted alkyl_{(C≤6)};
X⁻ is a monovalent anion.

42. The method of any one of claims 1-32, wherein said SORT lipid has a structural formula: 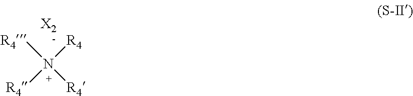
wherein:
R₄ and R_{4′} are each independently alkyl_{(C6-C24)}, alkenyl_{(C6-C24)}, or a substituted version of either group;
R_{4″} is alkyl_{(C≤24)}, alkenyl_{(C≤24)}, or a substituted version of either group;
R_{4″} is alkyl_{(C2-C8)}, alkenyl_{(C2-C8)}, or a substituted version of either group; and
X₂ is a monovalent anion.

43. The method of any one of claims 1-42, wherein the ionizable cationic lipid is a dendrimer or dendron of a generation (g) having a structural formula: 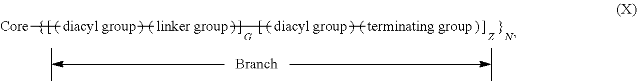
or a pharmaceutically acceptable salt thereof, wherein:
(a) the core comprises a structural formula (X_{Core}): 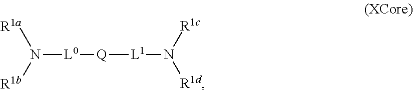
wherein:
Q is independently at each occurrence a covalent bond, —O—, —S—, —NR²—, or —CR^{3a}R^{3b}—;
R² is independently at each occurrence R^{1g} or -L²-NR^{1e}R^{1f},
R^{3a} and R^{3b} are each independently at each occurrence hydrogen or an optionally substituted C₁-C₆ alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted C₁-C₁₂ alkyl;
L⁰, L¹, and L² are each independently at each occurrence selected from a covalent bond, C₁-C₁₂ alkylene, C₁-C₁₂ heteroalkylene, [(C₁-C₆) alkylene]-[(C₄-C₆) heterocycloalkyl]-[(C₁-C₆) alkylene], [(C₁-C₆) alkylene]-(arylene)-[(C₁-C₆) alkylene], (C₄-C₆) heterocycloalkyl, and arylene; or,
alternatively, part of L¹ form a (C₄-C₆) heterocycloalkyl with one of R^{1c} and R^{1d}; and
x¹ is 0, 1, 2, 3, 4, 5, or 6; and
(b) each branch of the plurality (N) of branches independently comprises a structural formula (X_{Branch}): 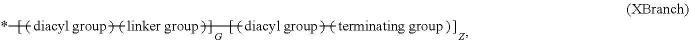
wherein:
* indicates a point of attachment of the branch to the core;
g is 1, 2, 3, or 4;
Z=2^{(g-1)};
G=0, when g=1; or G=Σᵢ₌₀^{i=g-2}2ⁱ, when g≠1;
(c) each diacyl group independently comprises a structural formula 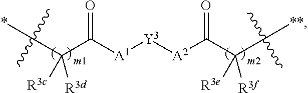
wherein:
* indicates a point of attachment of the diacyl group at the proximal end thereof;
** indicates a point of attachment of the diacyl group at the distal end thereof;
Y³ is independently at each occurrence an optionally substituted (C₁-C₁₂);
alkylene, an optionally substituted (C₁-C₁₂) alkenylene, or an optionally substituted (C₁-C₁₂) arenylene;
A¹ and A² are each independently at each occurrence —O—, —S—, or —NR₄—, wherein:
R₄ is hydrogen or optionally substituted (C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (C₁-C₈) alkyl; and
(d) each linker group independently comprises a structural formula 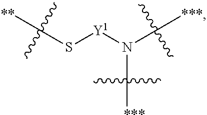
wherein:
** indicates a point of attachment of the linker to a proximal diacyl group;
*** indicates a point of attachment of the linker to a distal diacyl group; and
Y₁ is independently at each occurrence an optionally substituted (C₁-C₁₂) alkylene, an optionally substituted (C₁-C₁₂) alkenylene, or an optionally substituted (C₁-C₁₂) arenylene; and
(e) each terminating group is independently selected from optionally substituted (C₁-C₁₈, such as C₄-C₁₈) alkylthiol, and optionally substituted (C₁-C₁₈) alkenylthiol.

44. The method of claim 43, wherein R^{1a}, R^{1b}, R^{1c}, R^{1d}, R^{1e}, R^{1f}, and R^{1g} (if present) are each independently at each occurrence a point of connection to a branch (as indicated by *), hydrogen, or C₁-C₁₂ alkyl, wherein the alkyl moiety is optionally substituted with one or more substituents each independently selected from —OH, C₄-C₈ heterocycloalkyl, N—(C₁-C₃ alkyl)-piperidinyl, piperazinyl, N—(C₁-C₁₂ alkyl)-piperadizinyl, morpholinyl, N-pyrrolidinyl, pyrrolidinyl, or N—(C₁-C₃ alkyl)-pyrrolidinyl, (C₆-C₁₀) aryl, and C₃-C₅ heteroaryl, or pyridinyl.

45. The method of claim 44, wherein R^{1a}, R^{1b}, R^{1c}, R^{1d}, R^{1e}, R^{1f}, and R^{1g} (if present) are each independently at each occurrence a point of connection to a branch (as indicated by *), hydrogen, or C₁-C₁₂ alkyl, wherein the alkyl moiety is optionally substituted with one substituent —OH.

46. The method of any one of claims 43-45, wherein R^{3a} and R^{3b} are each independently at each occurrence hydrogen.

47. The method of any one of claims 43-46, wherein the plurality (N) of branches comprises at least 3 branches.

48. The method of any one of claims 43-47, wherein g=1; G=0; and Z=1.

49. The method of claim 48, wherein each branch of the plurality of branches comprises a structural formula *-(diacyl group)-(terminating group).

50. The method of any one of claims 43-47, wherein g=2; G=1; and Z=2.

51. The method of claim 50, wherein each branch of the plurality of branches comprises a structural formula

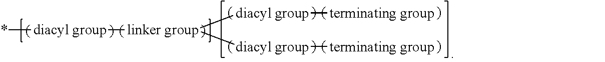

52. The method of any one of claims 43-51, wherein the core comprises a structural formula selected from the group consisting of: 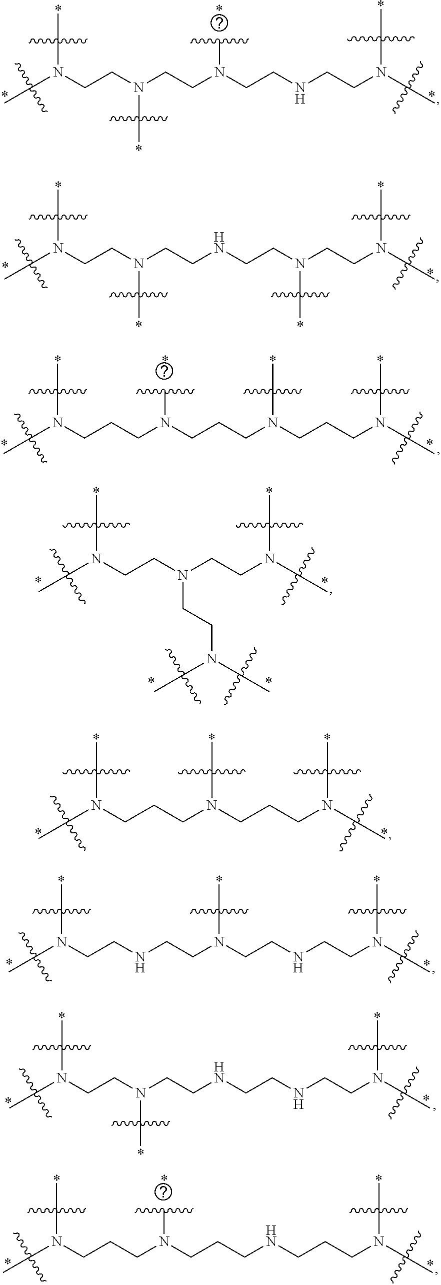 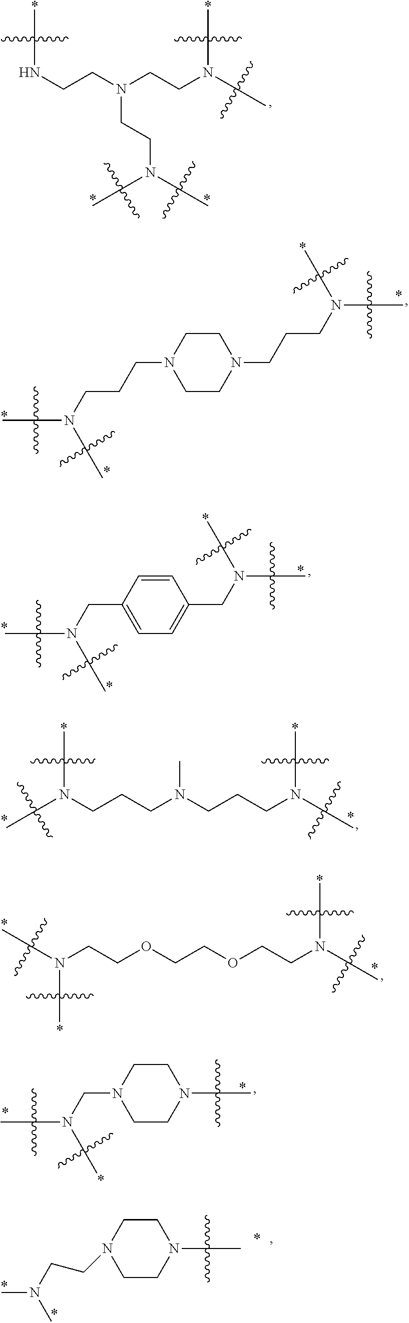 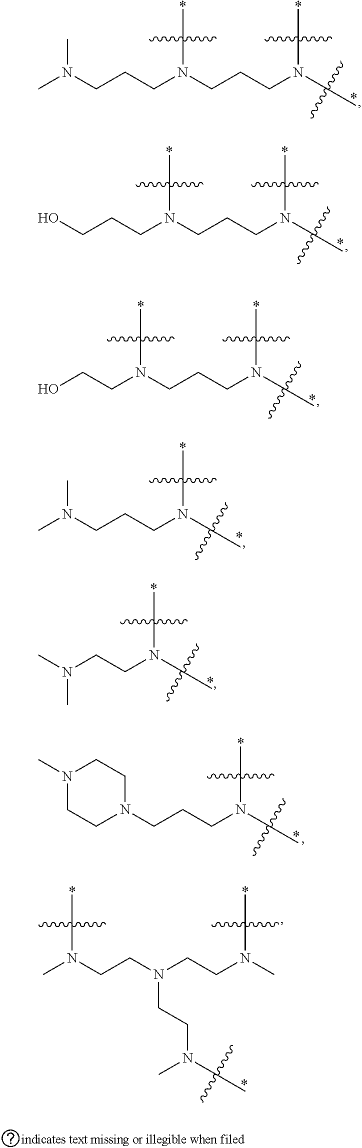
and pharmaceutically acceptable salts thereof, wherein * indicates a point of attachment of the core to a branch of the plurality of branches.

53. The method of any one of claims 43-51, wherein the core comprises a structural formula selected from the group consisting of: 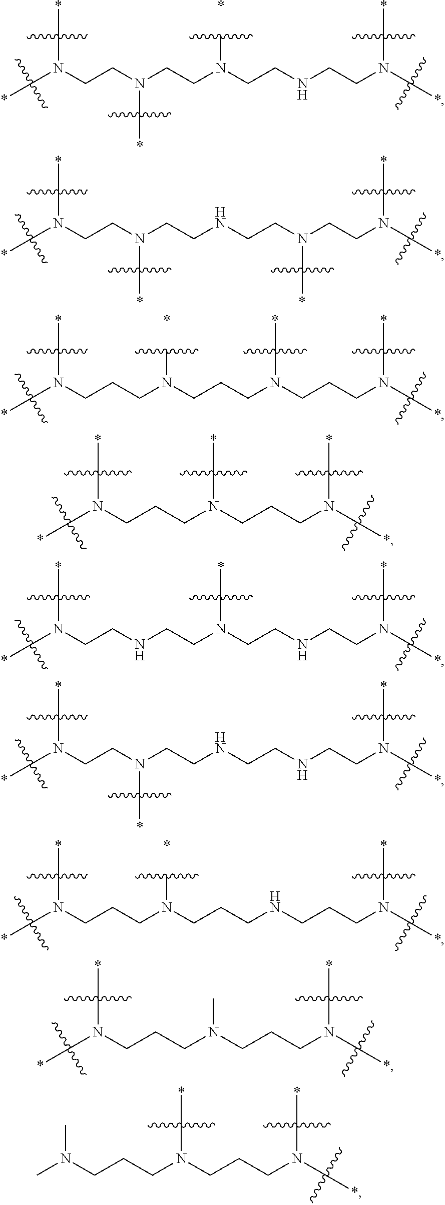
and pharmaceutically acceptable salts thereof, wherein * indicates a point of attachment of the core to a branch of the plurality of branches.

54. The method of any one of claims 43-51, wherein the core has the structure 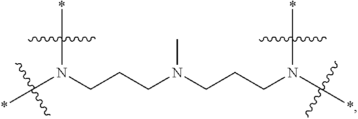
wherein * indicates a point of attachment of the core to a branch of the plurality of branches or H, wherein at least 2, at least 3, or at least 4 branches are attached to the core.

55. The method of any one of claims 43-51, wherein the core has the structure 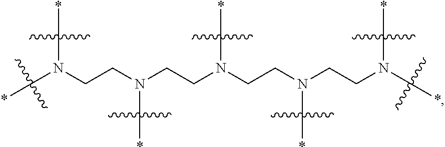
wherein * indicates a point of attachment of the core to a branch of the plurality of branches or H, wherein at least 4, at least 5, or at least 6 branches are attached to the core.

56. The method of any one of claims 43-55, wherein A¹ is —O— or —NH—.

57. The method of claim 56, wherein A¹ is —O—.

58. The method of any one of claims 43-57, wherein A² is —O— or —NH—.

59. The method of any claim 58, wherein A² is —O—.

60. The method of any one of claims 43-59, wherein Y³ is C₁-C₁₂ alkylene.

61. The method of any one of claims 43-60, wherein the diacyl group independently at each occurrence comprises a structural formula

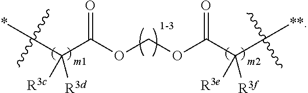

62. The method of any one of claims 43-61, wherein each terminating group is independently C₁-C₁₈ alkenylthiol or C₁-C₁₈ alkylthiol.

63. The method of claim 62, wherein each terminating group is independently selected from the group consisting of:

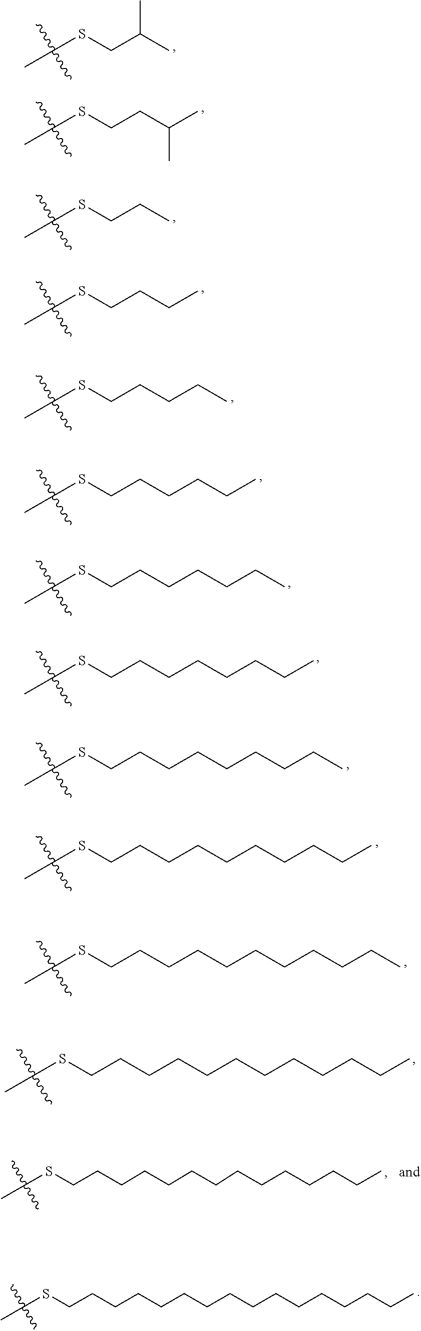

64. The method of any one of claims 1-63, wherein said subject has been determined to have a mutation in a target gene.

65. The method of claim 64, wherein said mutation in said target gene is associated with a genetic disease or disorder.

66. The method of any one of claims 1-65, wherein said subject has been determined to exhibit an aberrant expression or activity of a protein or polynucleotide that corresponds to a target gene.

67. The method of claim 66, wherein said aberrant expression or activity of said protein or polynucleotide is associated with a genetic disease or disorder.

68. The method of any one of claims 1-67, wherein said subject is selected from the group consisting of mouse, rat, monkey, and human.

69. The method of claim 68, wherein said subject is a human.

70. The method of any one of claims 1-69, wherein said therapeutic agent comprises a compound, a polynucleotide, a polypeptide, or a combination thereof.

71. The method of claim 70, wherein said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRSIPR-associated (Cas) protein, or a combination thereof.

72. The method of claim 71, wherein said therapeutic agent comprises a heterologous messenger ribonucleotide (mRNA); and wherein said intravenous administration results in an expression, activity, or effect of a protein encoded by said heterologous mRNA detectable in said at least about 1% lung basal cells of said subject.

73. The method of claim 72, wherein said protein is any one selected from the group consisting of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, and FBN1.

74. The method of claim 72, wherein said protein corresponds to a target gene in a lung cell of said subject.

75. The method of claim 72, wherein an expression of said heterologous mRNA produces a functional variant of said protein.

76. The method of claim 72, wherein an expression of said heterologous mRNA increases an amount of a functional variant of said protein as compared to an amount of said functional variant of said protein generated in absence of said intravenous administration.

77. The method of claim 71, wherein said therapeutic agent comprises a heterologous transfer ribonucleotide (tRNA) that introduces an amino acid into a growing peptide chain of a protein of a target gene; and wherein said intravenous administration results in an expression or activity of said protein detectable in said at least about 1% lung basal cells of said subject.

78. The method of claim 77, wherein said protein is any one selected from the group consisting of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, and FBN1.

79. The method of claim 77, wherein said target gene is present in a lung cell of said subject.

80. The method of claim 77, wherein said tRNA reduces an amount of a non-functional variant of said protein in said cell as compared to an amount of said non-functional variant of said protein generated in absence of said contacting.

81. The method of claim 70, wherein said therapeutic agent comprises a heterologous polypeptide comprising an actuator moiety, which actuator moiety is configured to complex with a target polynucleotide corresponding to a target gene; and wherein said intravenous administration results in a modified expression or activity of said target gene detectable in said at least about 1% lung basal cells of said subject.

82. The method of claim 70, wherein said therapeutic agent comprises a heterologous polynucleotide encoding an actuator moiety, which actuator moiety is configured to complex with a target polynucleotide corresponding to a target gene; and wherein said intravenous administration results in a modified expression or activity of said target gene detectable in said at least about 1% lung basal cells of said subject.

83. The method of claim 82, wherein said heterologous polynucleotide encodes a guide polynucleotide configured to direct said actuator moiety to said target polynucleotide.

84. The method of claim 82, wherein said actuator moiety comprises a heterologous endonuclease or a fragment thereof.

85. The method of claim 84, wherein said heterologous endonuclease is (1) part of a ribonucleoprotein (RNP) and (2) complexed with said guide polynucleotide.

86. The method of claim 84, wherein said heterologous endonuclease is part of a clustered regularly interspaced short palindromic repeats (CRISPR)/CRISPR-associated (Cas) protein complex.

87. The method of claim 84, wherein said heterologous endonuclease is a clustered regularly interspaced short palindromic repeats (CRISPR)-associated (Cas) endonuclease.

88. The method of claim 84, wherein said heterologous endonuclease is selected from C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas 11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or a fragment thereof.

89. The method of claim 84, wherein said heterologous endonuclease comprises a deactivated endonuclease.

90. The method of claim 82, wherein said target polynucleotide corresponds to a gene encoding any protein selected from the group consisting of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, and FBN1.

91. The method of claim 82, wherein said target polynucleotide corresponds to a gene in a lung cell of said subject.

92. The method of claim 82, wherein said expression or activity or said modified expression or activity is detectable at least about 4 hours after said intravenous administering.

93. The method of any one of claims 1-92, wherein said reference lipid composition does not comprise said amount of said SORT lipid.

94. The method of any one of claims 1-92, wherein said reference lipid composition does not comprise said SORT lipid.

95. The method of any one of claims 1-92, wherein said reference lipid composition comprises 13,16,20-tris(2-hydroxydodecyl)-13,16,20,23-tetraazapentatricontane-11,25-diol (“LF92”), a phospholipid, cholesterol, and a PEG-lipid.

96. A high-potency intravenous dosage form of a therapeutic agent formulated with a selective organ targeting (SORT) lipid, the dosage form comprising:
said therapeutic agent assembled with a lipid composition that comprises:
(i) an ionizable cationic lipid; and
(ii) said SORT lipid separate from said ionizable cationic lipid, wherein said SORT lipid is present in said dosage form in an amount sufficient to achieve a therapeutic effect at a dose of said therapeutic agent lower than that required with a reference lipid composition.

97. high-potency intravenous dosage form of a therapeutic agent formulated with a selective organ targeting (SORT) lipid, the dosage form comprising:
said therapeutic agent assembled with a lipid composition that comprises:
(i) an ionizable cationic lipid; and
(ii) said SORT lipid separate from said ionizable cationic lipid, wherein said therapeutic agent is present in said dosage form at a dose of no more than about 2 milligram per kilogram (mg/kg, or mpk) body weight.

98. The dosage form of claim 96 or 97, wherein said lipid composition further comprises (iii) a phospholipid.

99. The dosage form of any one of claims 96-98, wherein said therapeutic agent is present in said intravenous dosage form at a dose of no more than about 1.0, 0.5, 0.1, 0.05, or 0.01 mg/kg body weight.

100. The dosage form of any one of claims 96-99, wherein said therapeutic agent is present in said intravenous dosage form at a concentration of no more than about 5 milligram per milliliter (mg/mL).
